(19) United States
(12) Patent Application Publication
(10) Pub. No.: US 2009/0042964 A1
(43) Pub. Date: Feb. 12, 2009

(54) AMINO-5-[SUBSTITUTED-4-(DIFLUOROMETHOXY)PHENYL]-5-PHENYLIMIDAZOLONE COMPOUNDS AS BETA-SECRETASE INHIBITORS

(75) Inventors: Michael Sotirios Malamas, Jamison, PA (US); Albert Jean Robichaud, Ringoes, NJ (US); Alexander Michael Porte, Pennington, NJ (US); William R. Solvibile, East Windsor, NJ (US); Koi Michele Morris, Plainsboro, NJ (US); Schuyler Adam Antane, Princeton Junction, NJ (US); Ji-In Kim, Princeton, NJ (US); Robert Emmett McDevitt, Freehold, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/052,098

(22) Filed: Mar. 20, 2008

Related U.S. Application Data
(60) Provisional application No. 60/919,087, filed on Mar. 20, 2007.

(51) Int. Cl. A61K 31/4168; C07D 233/32; A61P 25/00

(52) U.S. Cl. 514/386; 548/321.5

(57) ABSTRACT

The present invention provides a 2-amino-5-[substituted-4-(difluoromethoxy)phenyl]-5-phenylimidazolone compound of formula I

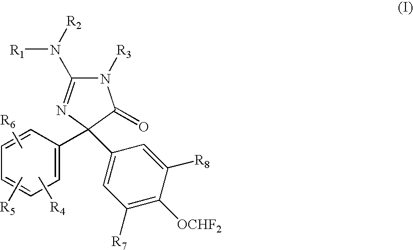

The present invention also provides methods for the use thereof to inhibit β-secretase (BACE) and treat β-amyloid deposits and neurofibrillary tangles.

### CROSS-REFERENCE TO RELATED APPLICATIONS

This application claims the benefit under 35 U.S.C. §119(e) to co-pending U.S. Provisional Application Ser. No. 60/919,087, filed Mar. 20, 2007, which is hereby incorporated by reference in its entirety.

### FIELD OF THE INVENTION

The present invention relates to amino-5-[substituted-4-(difluoromethoxy)phenyl]-5-phenylimidazolone compounds, which are inhibitors of β-secretase, compositions and kits containing these derivatives, and methods of their preparation and use for the prevention and treatment of diseases or disorders associated with β-Amyloid deposits and neurofibrillary tangles, including Alzheimer's disease, Trisomy 21 (Down's Syndrome), Hereditary Cerebral Hemorrhage with Amyloidosis of the Dutch-type (HCHWA-D), and other neurodegenerative disorders.

### BACKGROUND

β-Amyloid deposits and neurofibrillary tangles are two major pathologic characterizations associated with Alzheimer's disease (AD). Clinically, AD is characterized by the of loss of memory, cognition, reasoning, judgment, and orientation. Also affected, as the disease progresses, are motor, sensory, and linguistic abilities until global impairment of multiple cognitive functions occurs. These cognitive losses take place gradually, but typically lead to severe impairment and eventual death in 4-12 years.

Amyloidogenic plaques and vascular amyloid angiopathy also characterize the brains of patients with Trisomy 21 (Down's Syndrome), Hereditary Cerebral Hemorrhage with Amyloidosis of the Dutch-type (HCHWA-D), and other neurodegenerative disorders. Neurofibrillary tangles also occur in other neurodegenerative disorders including dementia-inducing disorders (Varghese, J., et al, Journal of Medicinal Chemistry, 2003, 46, 4625-4630).

β-Amyloid deposits are predominately an aggregate of Aβ peptide, which in turn is a product of the proteolysis of amyloid precursor protein (APP). More specifically, Aβ peptide results from the cleavage of APP at the C-terminus by one or more γ-secretases, and at the N-terminus by β-secretase enzyme (BACE), also known as aspartyl protease, as part of the β-amyloidogenic pathway.

BACE activity is correlated directly to the generation of Aβ peptide from AβP (Sinha, et al, Nature, 1999, 402, 537-540), and studies increasingly indicate that the inhibition of BACE inhibits the production of Aβ peptide (Roberds, S. L., et al, Human Molecular Genetics, 2001, 10, 1317-1324).

Therefore, it is an object of this invention to provide compounds which are inhibitors of β-secretase and are useful as therapeutic agents in the treatment, prevention or amelioration of a disease or disorder characterized by elevated β-amyloid deposits or β-amyloid levels in a patient.

In addition to potent inhibitory BACE activity, a successful drug candidate must pass a myriad of tests associated with toxicity and safety. One such test is the so-called “hERG-test.” The hERG (human Ether-a-go-go Related Gene) channel is an important potassium (K) channel responsible for cardiac action potential. Drug interaction with the hERG channel can decrease channel function causing an acquired long QT syndrome and potentially death as a result of heart malfunction. Consequently, hERG-blocking properties will end the prospects of a potential drug. Frustratingly, there is now no way to a priori predict whether or not a particular class of compounds will block hERG channels.

Accordingly, it is another object of the invention to provide compounds that do not substantially block hERG channels. It is another object of this invention to provide therapeutic methods and pharmaceutical compositions useful for the treatment, prevention or amelioration of a disease or disorder characterized by elevated β-amyloid deposits or β-amyloid levels in a patient.

It is a feature of this invention that the compounds provided may also be useful to further study and elucidate the β-secretase enzyme.

These and other objects and features of the invention will become more apparent by the detailed description set forth hereinbelow.

### SUMMARY OF THE INVENTION

The present invention provides a compound of formula I

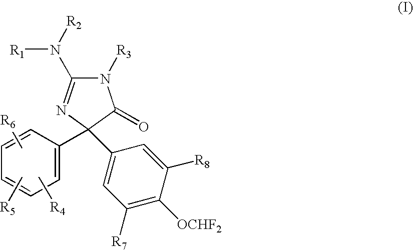

wherein
R₁ and R₂ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.

The present invention also relates to the use of such compounds for the treatment of β-amyloid deposits and neurofibrillary tangles. The formula I compounds are particularly useful in treating Alzheimer's disease, cognitive impairment, Down's Syndrome, HCHWA-D, cognitive decline, senile dementia, cerebral amyloid angiopathy, degenerative dementia, or other neurodegenerative disorders.

### DETAILED DESCRIPTION OF THE INVENTION

Alzheimer's disease (AD) is a major degenerative disease of the brain which presents clinically by progressive loss of memory, cognition, reasoning, judgement and emotional stability and gradually leads to profound mental deterioration and death. The exact cause of AD is unknown, but increasing evidence indicates that amyloid beta peptide (A-beta) plays a central role in the pathogenesis of the disease. (D. B. Schenk; R. E. Rydel et al, Journal of Medicinal Chemistry, 1995, 21, 4141 and D. J. Selkoe, Physiology Review, 2001, 81, 741). Patients with AD exhibit characteristic neuropathological markers such as neuritic plaques (and in β-amyloid angiopathy, deposits in cerebral blood vessels) as well as neurofibrillary tangles detected in the brain at autopsy. A-beta is a major component of neuritic plaques in AD brains. In addition, β-amyloid deposits and vascular β-amyloid angiopathy also characterize individuals with Downs Syndrome, Hereditary Cerebral Hemmorrhage with Amyloidosis of the Dutch type and other neurodegenerative and dementia-inducing disorders. Overexpression of the amyloid precursor protein (APP), altered cleavage of APP to A-beta or a decrease in the clearance of A-beta from a patient's brain may increase the levels of soluble or fibrullar forms of A-beta in the brain. The β-site APP cleaving enzyme, BACE1, also called memapsin-2 or Asp-2, was identified in 1999 (R. Vassar, B. D. Bennett, et al, Nature, 1999, 402, 537). BACE1 is a membrane-bound aspartic protease with all the known functional properties and characteristics of β-secretase. Low molecular weight, non-peptide, non-substrate-related inhibitors of BACE1 or β-secretase are earnestly sought both as an aid in the study of the β-secretase enzyme and as potential therapeutic agents.

Co-pending patent application Ser. No. 11/526,511 discloses amino-5-[4-(difluoromethoxy)phenyl]-5-phenylimidazolone compounds which demonstrate BACE activity and which contain a 5-[4-(difluoromethoxy)phenyl] group having no further substitution on the phenyl ring. Surprisingly, it has now been found that the amino-5-[substituted-4-(difluoromethoxy)phenyl]-5-phenylimidazolone compounds of the invention demonstrate increased inhibition of β-secretase over those compounds wherein the 4-(difluoromethoxy)phenyl ring is unsubstituted. Additionally, the 5-[substituted-4-(difluoromethoxy)phenyl]-5-phenylimidazolone compounds, particularly those compounds of the present invention substituted at R₇ with an alkyl group, are surprisingly shown to have favorable hERG properties, whereby potential complications associated with blocking hERG channels, and/or a decrease of channel function causing an acquired long QT syndrome are reduced or eliminated. Advantageously, said 5-[substituted-4-(difluoromethoxy)phenyl]-5-phenylimidazolone compounds of the invention may be used as safe and effective therapeutic agents for the treatment, prevention or amelioration of a disease or disorder characterized by elevated β-amyloid deposits or β-amyloid levels in a patient. Accordingly, the present invention provides an amino-5-[substituted-4-(difluoromethoxy)phenyl]-5-phenylimidazolone compound of formula I

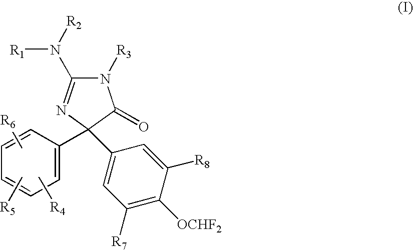

wherein
R₁ and R₂ are each independently H or an alkyl, cycloalkyl, cycloheteroalkyl, aryl or heteroaryl group each optionally substituted or R₁ and R₂ may be taken together with the atom to which they are attached form an optionally substituted 5- to 7-membered ring optionally interrupted by an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
   a tautomer thereof, a stereoisomer thereof or a pharmaceutically acceptable salt thereof.

In another embodiment, the compound has the formula IA:

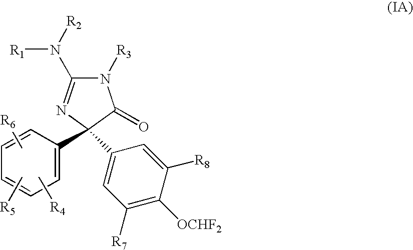

wherein R₁, R₂, R₃, R₄, R₅, R₆, R₇ and R₈ are the same as defined for the compound of formula I.

In another embodiment, the compound has the formula IB:

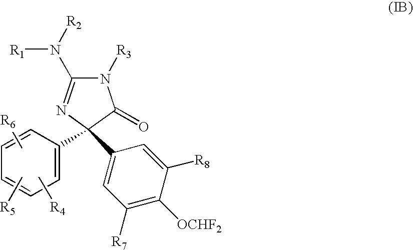

wherein R₁, R₂, R₃, R₄, R₅, R₆, R₇ and R₈ are the same as defined for the compound of formula I.

In another embodiment, if two of R₄, R₅ and R₆ are H, then the other group is not a para —OCHF₂ group. In another embodiment, neither R₄, R₅ nor R₆ is a para —OCHF₂ group.

It is understood that the claims encompass all possible stereoisomers and prodrugs. Moreover, unless stated otherwise, each alkyl, alkoxy, alkenyl, alkynyl, cycloalkyl, cycloheteroalkyl, aryl or heteroaryl group is contemplated as being optionally substituted.

An optionally substituted moiety may be substituted with one or more substituents. The substituent groups which are optionally present may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, aryloxy, amino, alkylamino, dialkylamino, formyl, carbonyl, alkoxycarbonyl, carboxyl, alkanoyl, alkylthio, alkylsulfinyl, alkylsulfonyl, carbamoyl, alkylamido, phenyl, phenoxy, benzyl, benzyloxy, cycloalkyl or cycloheteroalkyl groups, preferably halogen atoms, lower alkyl or lower alkoxy groups, wherein ‘lower’ is from 1 to 4 carbon atoms.

In one embodiment the substituent groups may be selected from halo, cyano, hydroxy, alkyl, haloalkyl, alkenyl, alkynyl, alkoxy, cycloalkyl, or halo-substituted cycloalkyl. Unless otherwise specified, typically, 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms. Substituent groups that have one or more available hydrogen atoms can in turn optionally bear further independently selected substituents, to a maximum of three levels of substitutions. For example, the term “optionally substituted aryl” is intended to mean an aryl group that can optionaly have up to four of its hydrogen atoms replaced with substituent groups as defined above (i.e., a first level of substitution), wherein each of the substituent groups attached to the aryl group can optionally have up to four of its hydrogen atoms replaced by substituent groups as defined above (i.e., a second level of substitution), and each of the substituent groups of the second level of substitution can optionally have up to four of its hydrogen atoms replaced by substituent groups as defined above (i.e., a third level of substitution).

As used herein, the term “alkyl” includes both straight chain and branched-chain (unless defined otherwise) monovalent saturated hydrocarbon moieties of 1-12 carbon atoms, preferably 1-6 carbon atoms (C₁-C₆ alkyl), more preferably ‘lower’ alkyl of 1-4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, and the like. Alkyl groups can be optionally substituted. Suitable alkyl substitutions include, but are not limited to, CN, OH, halogen, alkenyl, alkynyl, cycloalkyl, phenyl, carbamoyl, carbonyl, alkoxy or aryloxy.

As used herein the term “haloalkyl” designates a CₙH₂ₙ₊₁ group having from one to 2n+1 halogen atoms which may be the same or different. Examples of haloalkyl groups include CF₃, CH₂C₁, C₂H₃BrCl, C₃H₅F₂, or the like. Similarly, the term haloalkoxy designates an OCₙH₂ₙ₊₁ group having from one to 2n+1 halogen atoms which may be the same or different. Preferably the haloalkyl groups are C₁-C₆ haloalkyl groups.

The term “alkoxyalkyl” as used herein, refers to an alkyl group as hereinbefore defined substituted with at least one C₁-C₄ alkoxy group or C₁-C₆ alkoxy group.

The term “alkenyl”, as used herein, refers to either a straight chain or branched-chain hydrocarbon moiety containing at least one double bond and having from 2-12 carbon atoms, preferably 2-6 carbon atoms (C₂-C₆ alkenyl), more preferably 2-4 carbon atoms. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like. Preferred alkenyl groups are C₂-C₆ alkenyl.

The term “haloalkenyl” as used herein, designates an alkenyl group as defined hereinabove substituted with one or more halogen atoms which may be the same or different.

The term “alkynyl”, as used herein, refers to an alkyl group having one or more triple carbon-carbon bonds. Alkynyl groups preferably contain 2 to 6 carbon atoms (C₂-C₆ alkynyl). Examples of alkynyl groups include, but are not limited to, ethynyl, propynyl, butynyl, pentynyl, and the like. In some embodiments, alkynyl groups can be substituted with up to four substituent groups, as described hereinabove. Preferred alkynyl groups are C₂-C₆ alkynyl.

The terms “alkoxy”, “alkenyloxy” and “alkynyloxy” as used herein, refers to —O-alkyl, —O-alkenyl and —O-alkynyl, respectively, wherein the alkyl, alkenyl and alkynyl groups therein are as defined herein.

The term “cycloalkyl”, as used herein, refers to a monocyclic, bicyclic, tricyclic, fused, bridged, or spiro saturated carbocyclic moiety of 3-10 carbon atoms (C₃-C₁₀ cycloalkyl). Any suitable ring position of the cycloalkyl moiety may be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and homologs, isomers, or the like.

The term “cycloheteroalkyl” as used herein designates a 5- to 7-membered cycloalkyl ring system containing 1, 2 or 3 heteroatoms, which may be the same or different, selected from N, O or S, and optionally containing one double bond. Exemplary of the cycloheteroalkyl ring systems included in the term as designated herein are the following rings wherein X₁ is NR′, O or S, and R′ is H or an optional substituent as defined hereinabove.

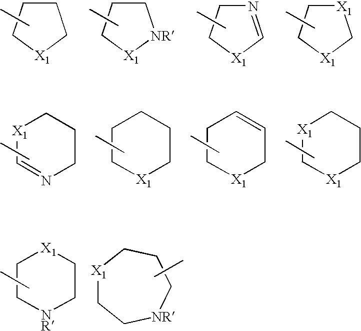

The term “aryl”, as used herein, designates an aromatic carbocyclic moiety of up to 20 carbon atoms, e.g. 6-20 carbon atoms, which may be a single ring (monocyclic) or multiple rings (bicyclic, up to three rings) fused together or linked covalently. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like. In some embodiments “aryl” groups can be substituted with from 1-5 substituents. Preferred aryl groups are C₆-C₁₀ aryl.

The term “heteroaryl” as used herein designates an aromatic heterocyclic ring system, e.g. having from 5-20 ring atoms, which may be a single ring (monocyclic) or multiple rings (bicyclic, up to three rings) fused together or linked covalently. Preferably, heteroaryl is a 5- to 6-membered ring. The rings may contain from one to four hetero atoms selected from nitrogen, oxygen, or sulfur, wherein the nitrogen or sulfur atom(s) are optionally oxidized, or the nitrogen atom(s) are optionally quaternized. Examples of heteroaryl moieties include, but are not limited to, heterocycles such as furan, thiophene, pyrrole, pyrazole, imidazole, oxazole, isoxazole, thiazole, isothiazole, 1H-tetrazole, 1,3,4-oxadiazole, 1H-1,2,4-triazole, 1,3,4-triazole, pyridine, pyrimidine, pyrazine, pyridazine, benzoxazole, benzisoxazole, benzothiazole, benzofuran, benzothiophene, thianthrene, benzimidazole, indole, indazole, quinoline, isoquinoline, quinazoline, quinoxaline, purine, pteridine, 9H-carbazole, α-carboline, or the like.

The term “halogen”, as used herein, designates fluorine, chlorine, bromine, or iodine.

The compounds of the present invention may be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived form organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Compounds of the invention may exist as one or more tautomers. One skilled in the art will recognize that compounds of formula I may also exist as the tautomer It as shown below.

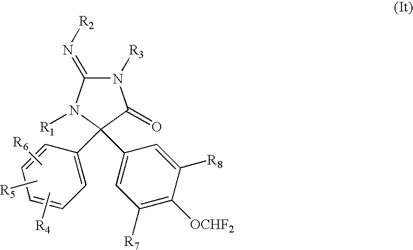

Tautomers often exist in equilibrium with each other. As these tautomers interconvert under environmental and physiological conditions, they provide the same useful biological effects. The present invention includes mixtures of such tautomers as well as the individual tautomers.

The compounds of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof. Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one steriosomer, preferably less than about 50%, more preferably less than about 75%, and even more preferably less than about 90%.

Preferred compounds of formula I are those compounds wherein R₁ and R₂ are H. Another group of preferred compounds are those compounds of formula I wherein R₃ is C₁-C₄alkyl. More preferably R₃ is methyl. Also preferred are those compounds of formula I wherein R₄, R₅ and R₆ are each independently H, halogen, or an alkenyl, alkynyl, alkoxy, alkenyloxy, or alkynyloxy group each optionally substituted.

More preferred compounds of the invention are those compounds of formula I wherein R₇ is halogen, C₁-C₄alkyl or C₃-C₆cycloalkyl. More preferably R₇ is halogen, methyl, ethyl, propyl or cyclopropyl. Also more preferred are those compounds wherein R₄ is an alkenyl, alkynyl, alkoxy, alkenyloxy, or alkynyloxy group each optionally substituted. Preferably R₅ and R₆ are each independently H or halogen. Preferably R₈ is H or C₁-C₄alkyl. Also more preferred are those compounds wherein R₇ is halogen, C₁-C₄alkyl or C₃-C₆cycloalkyl; R₁ and R₂ are H and R₃ is methyl. Another group of more preferred compounds of the invention are those compounds of formula I wherein R₇ is halogen, methyl, ethyl, propyl or cyclopropyl; R₄ is an alkenyl, alkynyl, alkoxy, alkenyloxy, or alkynyloxy group each optionally substituted; and R₅ and R₆ are each independently H or halogen. In one embodiment R₄ is alkynyl optionally substituted with cycloalkyl. In another embodiment R₄ is at the 3-position of the phenyl ring.

A further group of more preferred compounds of the invention are those compounds of formula I wherein R₁ and R₂ are H; R₃ is methyl; R₄ is an alkenyl, alkynyl, alkoxy, alkenyloxy, or alkynyloxy group each optionally substituted; R₅ and R₆ are each independently H or halogen; R₇ is halogen, methyl, ethyl, propyl or cyclopropyl; and R₄ is at the 3-position of the phenyl ring.

An addition group of preferred compounds of the invention are those of formula I, wherein R₄ is:

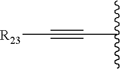

wherein,

R₂₃ is selected from the group consisting of H, alkyl, haloalkyl, cycloalkyl, halogen or alkoxyalkyl.

More particularly, R₂₃ is methyl, ethyl, cyclopropyl, methoxymethyl, methoxyethyl, propyl, fluoroethyl, fluoromethyl, isopropyl, isobutyl or 1,1-difluoroethyl.

In another preferred embodiment, R₆ is H and R₅ is fluoro substituted at the 4-position of the phenyl ring.

Preferred compounds of the invention include:
(5R)-2-Amino-5-[3-(cyclopropylethynyl)-4-fluorophenyl]-5-[4-(difluoromethoxy)-3-methylphenyl]-3-methyl-3,5-dihydro-4H-imidazol-4-one
2-Amino-5-[4-(difluoromethoxy)-3-methylphenyl]-3-methyl-5-phenyl-3,5-dihydro-4H-imidazol-4-one;
(5S)-2-Amino-5-[4-(difluoromethoxy)-3-methylphenyl]-3-methyl-5-phenyl-3,5-dihydro-4H-imidazol-4-one;
(5R)-2-Amino-5-[4-(difluoromethoxy)-3-methylphenyl]-3-methyl-5-phenyl-3,5-dihydro-4H-imidazol-4-one;
2-Amino-4-(4-(difluoromethoxy)-3-methylphenyl)-4-(4-fluorophenyl)-1-methyl-1H-imidazol-5(4H)-one;
(5S)-2-Amino-4-(4-(difluoromethoxy)-3-methylphenyl)-4-(4-fluorophenyl)-1-methyl-1H-imidazol-5(4H)-one;
(5R)-2-Amino-4-(4-(difluoromethoxy)-3-methylphenyl)-4-(4-fluorophenyl)-1-methyl-1H-imidazol-5(4H)-one;
2-Amino-5-(3-butoxyphenyl)-5-[3-chloro-4-(difluoromethoxy)-phenyl]-3-methyl-3,5-dihydro-4H-imidazol-4-one;
2-Amino-5-[3-(cyclopropylethynyl)phenyl]-5-[4-(difluoro-methoxy)-3-methylphenyl]-3-methyl-3,5-dihydro-4H-imidazol-4-one;
2-Amino-5-[4-(difluoromethoxy)-3-methylphenyl]-5-[4-fluoro-3-(3-methylbut-1-yn-1-yl)phenyl]-3-methyl-3,5-dihydro-4H-imidazol-4-one;
2-Amino-5-[4-(difluoromethoxy)-3-methylphenyl]-5-[4-fluoro-3-((E)-3-methoxypropenyl)phenyl]-3-methyl-3,5-dihydro-imidazol-4-one;
2-Amino-5-[4-(difluoromethoxy)-3-methylphenyl]-5-[4-fluoro-3-((E)-4-fluorobut-1-enyl)phenyl]-3-methyl-3,5-dihydro-4H-imidazol-4-one;
2-Amino-5-[4-(difluoromethoxy)-3-methylphenyl]-5-[4-fluoro-3-((E)-prop-1-enyl)phenyl]-3-methyl-3,5-dihydro-4H-imidazol-4-one;
2-Amino-5-[4-(difluoromethoxy)-3-methylphenyl]-5-[4-fluoro-3-((E)-4-methoxy-but-1-enyl)phenyl]-3-methyl-3,5-dihydro-4H-imidazol-4-one;
2-Amino-5-[4-(difluoromethoxy)-3-methylphenyl]-3-methyl-5-[((E)-3-prop-1-enyl)phenyl]-3,5-dihydro-4H-imidazol-4-one;
2-Amino-5-[4-(difluoromethoxy)-3-methylphenyl]-5-[3-((E)-3-methoxyprop-1-enyl)phenyl]-3-methyl-3,5-dihydro-4H-imidazol-4-one;
2-Amino-5-[4-(difluoromethoxy)-3-methylphenyl]-5-[3-((E)-4-fluorobut-1-enyl)phenyl]-3-methyl-3,5-dihydro-4H-imidazol-4-one;
2-Amino-5-[4-(difluoromethoxy)-3-methylphenyl]-5-[3-((E)-4-methoxybut-1-enyl)phenyl]-3-methyl-3,5-dihydro-4H-imidazol-4-one;
2-Amino-5-[3-chloro-4-(difluoromethoxy)phenyl]-3-methyl-5-[((E)-3-prop-1-enyl)phenyl]-3,5-dihydro-4H-imidazol-4-one;
2-Amino-5-[3-chloro-4-(difluoromethoxy)phenyl]-5-[3-((E)-3-methoxyprop-1-enyl)-phenyl]-3-methyl-3,5-dihydro-4H-imidazol-4-one;
2-Amino-5-[3-chloro-4-(difluoromethoxy)phenyl)-5-[3-((E)-4-methoxybut-1-enyl)phenyl]-3-methyl-3,5-dihydro-4H-imidazol-4-one;
2-Amino-5-[3-chloro-4-(difluoromethoxy)phenyl)-5-[3-((E)-4-fluoro-but-1-enyl)phenyl]-3-methyl-3,5-dihydro-4H-imidazol-4-one;
2-Amino-5,5-bis-[4-(difluoromethoxy)-3-methylphenyl]-3-methyl-3,5-dihydro-4H-imidazol-4-one;
2-Amino-5-[4-(difluoromethoxy)-3,5-dimethylphenyl]-3-methyl-5-phenyl-3,5-dihydro-4H-imidazol-4-one;
2-Amino-5-[4-(difluoromethoxy)-3-ethylphenyl]-3-methyl-5-phenyl-3,5-dihydro-4H-imidazol-4-one;
2-Amino-4-[4-(difluoromethoxy)-3-ethylphenyl]-4-(4-fluorophenyl)-1-methyl-1H-imidazol-5(4H)-one;
2-Amino-5-[4-(difluoromethoxy)-3-propylphenyl]-3-methyl-5-phenyl-3,5-dihydro-4H-imidazol-4-one;
2-Amino-5-[4-(difluoromethoxy)-3-isopropylphenyl]-3-methyl-5-phenyl-3,5-dihydro-4H-imidazol-4-one;
2-Amino-5-[3-cyclopropyl-4-(difluoromethoxy)phenyl]-3-methyl-5-phenyl-3,5-dihydro-4H-imidazol-4-one;
2-Amino-5-[4-(difluoromethoxy)-3-vinylphenyl)-3-methyl-5-phenyl-3,5-dihydro-4H-imidazol-4-one;
2-Amino-5-[4-(difluoromethoxy)-3-(trifluoromethyl)phenyl]-3-methyl-5-phenyl-3,5-dihydro-4H-imidazol-4-one;
2-Amino-5-[4-(difluoromethoxy)-3-(fluoromethyl)phenyl]-3-methyl-5-phenyl-3,5-dihydro-4H-imidazol-4-one;
2-Amino-5-[4-(difluoromethoxy)-3-(fluoromethyl)phenyl]-3-methyl-5-phenyl-3,5-dihydro-4H-imidazol-4-one;
2-Amino-5-[4-(difluoromethoxy)-3-methoxyphenyl]-3-methyl-5-phenyl-3,5-dihydro-4H-imidazol-4-one;
2-Amino-5-[3-chloro-4-(difluoromethoxy)phenyl]-3-methyl-5-phenyl-3,5-dihydro-4H-imidazol-4-one;
2-Amino-5-[4-(difluoromethoxy)-3-fluorophenyl]-3-methyl-5-phenyl-3,5-dihydro-4H-imidazol-4-one;
2-Amino-5-(3-bromophenyl)-5-[4-(difluoromethoxy)-3-methylphenyl]-3-methyl-3,5-dihydro-4H-imidazol-4-one;
2-Amino-5-(3-bromo-4-fluorophenyl)-5-[4-(difluoromethoxy)-3-methylphenyl]-3-methyl-3,5-dihydro-4H-imidazol-4-one;
2-Amino-5-[4-(difluoromethoxy)-3-methylphenyl]-5-(4-fluoro-3-methylphenyl)-3-methyl-3,5-dihydro-4H-imidazol-4-one;
5-{2-Amino-4-[4-(difluoromethoxy)-3-methylphenyl]-1-methyl-5-oxo-4,5-dihydro-1H-imidazol-4-yl}-2-methoxybenzonitrile;
2-Amino-5-[4-(difluoromethoxy)-3-methylphenyl]-5-[4-fluoro-3-(fluoromethyl)phenyl]-3-methyl-3,5-dihydro-4H-imidazol-4-one;
(5S)-2-Amino-4-[4-(difluoromethoxy)-3-ethylphenyl]-1-methyl-4-phenyl-1H-imidazol-5(4H)-one;
(5R)-2-Amino-4-[4-(difluoromethoxy)-3-ethylphenyl]-1-methyl-4-phenyl-1H-imidazol-5(4H)-one;
(5R)-2-Amino-5-[3-cyclopropyl-4-(difluoromethoxy)phenyl]-3-methyl-5-phenyl-3,5-dihydro-4H-imidazol-4-one;
(5S)-2-Amino-5-[3-cyclopropyl-4-(difluoromethoxy)phenyl]-3-methyl-5-phenyl-3,5-dihydro-4H-imidazol-4-one;
(5S)-2-Amino-5-[3-chloro-4-(difluoromethoxy)phenyl]-1-methyl-5-phenyl-3,5-dihydro-4H-imidazol-4-one;
(5R)-2-Amino-5-[3-chloro-4-(difluoromethoxy)phenyl]-1-methyl-5-phenyl-3,5-dihydro-4H-imidazol-4-one;
(5S)-2-Amino-5-(3-bromophenyl)-5-[4-(difluoromethoxy)-3-methylphenyl]-3-methyl-3,5-dihydro-4H-imidazol-4-one;
(5R)-2-Amino-5-(3-bromophenyl)-5-[4-(difluoromethoxy)-3-methylphenyl]-3-methyl-3,5-dihydro-4H-imidazol-4-one;
2-Amino-5-[3-chloro-4-(difluoromethoxy)phenyl]-5-[3-(2-fluoroethoxy)phenyl]-3-methyl-3,5-dihydro-4H-imidazol-4-one;
2-Amino-5-[3-chloro-4-(difluoromethoxy)phenyl]-5-(3-ethoxyphenyl)-3-methyl-3,5-dihydro-4H-imidazol-4-one;
2-Amino-5-[3-chloro-4-(difluoromethoxy)phenyl]-5-[3-(2,2-difluoroethoxy)phenyl]-3-methyl-3,5-dihydro-4H-imidazol-4-one;
2-Amino-5-[3-chloro-4-(difluoromethoxy)phenyl]-3-methyl-5-(3-propoxyphenyl)-3,5-dihydro-4H-imidazol-4-one;
2-Amino-5-[3-chloro-4-(difluoromethoxy)phenyl]-5-[3-(3-fluoropropoxy)phenyl]-3-methyl-3,5-dihydro-4H-imidazol-4-one;
2-Amino-5-[4-(difluoromethoxy)-3-methylphenyl]-5-(3-ethoxyphenyl)-3-methyl-3,5-dihydro-4H-imidazol-4-one;
2-Amino-5-[4-(difluoromethoxy)-3-methylphenyl]-3-methyl-5-(3-propoxyphenyl)-3,5-dihydro-4H-imidazol-4-one;
2-Amino-5-[3-(2,2-difluoroethoxy)phenyl]-5-[4-(difluoromethoxy)-3-methylphenyl]-3-methyl-3,5-dihydro-4H-imidazol-4-one;
2-Amino-5-[4-(difluoromethoxy)-3-methylphenyl]-3-methyl-5-(3-propoxyphenyl)-3,5-dihydro-4H-imidazol-4-one;
2-Amino-5-[4-(difluoromethoxy)-3-methylphenyl]-5-[3-(3-fluoropropoxy)phenyl]-3-methyl-3,5-dihydro-4H-imidazol-4-one;
2-Amino-5-[3-(cyclopropylethynyl)-4-fluorophenyl]-5-[4-(difluoromethoxy)-3-methylphenyl]-3-methyl-3,5-dihydro-4H-imidazol-4-one;
2-Amino-5-[4-(difluoromethoxy)-3-methylphenyl]-5-[4-fluoro-3-(3-methylbut-1-yn-1-yl)phenyl]-3-methyl-3,5-dihydro-4H-imidazol-4-one;
(5R)-2-Amino-5-[3-(cyclopropylethynyl)phenyl]-5-[4-(difluoromethoxy)-3-methylphenyl]-3-methyl-3,5-dihydro-4H-imidazol-4-one;
(5S)-2-Amino-5-[3-(cyclopropylethynyl)phenyl]-5-[4-(difluoromethoxy)-3-methylphenyl]-3-methyl-3,5-dihydro-4H-imidazol-4-one;
(5S)-2-Amino-5-[3-(cyclopropylethynyl)-4-fluorophenyl]-5-[4-(difluoromethoxy)-3-methylphenyl]-3-methyl-3,5-dihydro-4H-imidazol-4-one;
(5S)-2-Amino-5-[4-(difluoromethoxy)-3-vinylphenyl]-3-methyl-5-phenyl-3,5-dihydro-4H-imidazol-4-one;
(5R)-2-Amino-5-[4-(difluoromethoxy)-3-vinylphenyl]-3-methyl-5-phenyl-3,5-dihydro-4H-imidazol-4-one;
(5S)-2-Amino-5-[4-(difluoromethoxy)-3-methylphenyl]-5-[3-(3-methoxyprop-1-yn-1-yl)phenyl]-3-methyl-3,5-dihydro-4H-imidazol-4-one;
(5R)-2-Amino-5-[4-(difluoromethoxy)-3-methylphenyl]-5-[3-(3-methoxyprop-1-yn-1-yl)phenyl]-3-methyl-3,5-dihydro-4H-imidazol-4-one;
(5S)-2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one;
(5R)-2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one;
2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one;
2-Amino-5-[4-(difluoromethoxy)-3-methylphenyl]-5-[3-(3-methoxyprop-1-yn-1-yl)phenyl]-3-methyl-3,5-dihydro-4H-imidazol-4-one; or a tautomer thereof, a stereoisomer thereof or a pharmaceutically acceptable salt thereof.

Additional preferred compounds of the present invention include:
(5R)-2-amino-5-[4-(difluoromethoxy)-3-methylphenyl]-5-(4-fluoro-3-pent-1-yn-1-ylphenyl)-3-methyl-3,5-dihydro-4H-imidazol-4-one;
(5S)-2-amino-5-[4-(difluoromethoxy)-3-methylphenyl]-5-(4-fluoro-3-pent-1-yn-1-ylphenyl)-3-methyl-3,5-dihydro-4H-imidazol-4-one;
2-amino-5-[4-(difluoromethoxy)-3-(2-fluoroethyl)phenyl]-3-methyl-5-phenyl-3,5-dihydro-4H-imidazol-4-one;
(5S)-2-amino-5-[4-(difluoromethoxy)-3-(2-fluoroethyl)phenyl]-3-methyl-5-phenyl-3,5-dihydro-4H-imidazol-4-one;
(5R)-2-amino-5-[4-(difluoromethoxy)-3-(2-fluoroethyl)phenyl]-3-methyl-5-phenyl-3,5-dihydro-4H-imidazol-4-one;
2-amino-5-[4-(difluoromethoxy)-3-methylphenyl]-3-methyl-5-(3-methylphenyl)-3,5-dihydro-4H-imidazol-4-one;
(5S)-2-amino-5-[4-(difluoromethoxy)-3-methylphenyl]-3-methyl-5-(3-pent-1-yn-1-ylphenyl)-3,5-dihydro-4H-imidazol-4-one;
(5S)-2-amino-5-[4-(difluoromethoxy)-3-propylphenyl]-3-methyl-5-phenyl-3,5-dihydro-4H-imidazol-4-one;
(5R)-2-amino-5-[4-(difluoromethoxy)-3-propylphenyl]-3-methyl-5-phenyl-3,5-dihydro-4H-imidazol-4-one;
2-amino-5-(3-but-1-yn-1-ylphenyl)-5-[4-(difluoromethoxy)-3-methylphenyl]-3-methyl-3,5-dihydro-4H-imidazol-4-one;
2-amino-5-(3-but-1-yn-1-yl-4-fluorophenyl)-5-[4-(difluoromethoxy)-3-methylphenyl]-3-methyl-3,5-dihydro-4H-imidazol-4-one;
(5R)-2-amino-5-(3-but-1-yn-1-yl-4-fluorophenyl)-5-[4-(difluoromethoxy)-3-methylphenyl]-3-methyl-3,5-dihydro-4H-imidazol-4-one;
(5S)-2-amino-5-(3-but-1-yn-1-yl-4-fluorophenyl)-5-[4-(difluoromethoxy)-3-methylphenyl]-3-methyl-3,5-dihydro-4H-imidazol-4-one;
2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
(5R)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
(5S)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
2-amino-5-[4-(difluoromethoxy)-3-(2-fluoroethyl)phenyl]-5-(4-fluoro-3-pent-1-yn-1-ylphenyl)-3-methyl-3,5-dihydro-4H-imidazol-4-one;
2-amino-5-(3-but-1-yn-1-yl-4-fluorophenyl)-5-[4-(difluoromethoxy)-3-(2-fluoroethyl)phenyl]-3-methyl-3,5-dihydro-4H-imidazol-4-one;
2-amino-5-[4-(difluoromethoxy)-3-(2-fluoroethyl)phenyl]-5-[4-fluoro-3-(4-methylpent-1-yn-1-yl)phenyl]-3-methyl-3,5-dihydro-4H-imidazol-4-one;
2-amino-5-[4-(difluoromethoxy)-3-(2-fluoroethyl)phenyl]-5-[4-fluoro-3-(4-fluorobut-1-yn-1-yl)phenyl]-3-methyl-3,5-dihydro-4H-imidazol-4-one;
2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
(5R)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
(5S)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
2-amino-5-[3-chloro-4-(difluoromethoxy)phenyl]-5-(4-fluoro-3-hydroxyphenyl)-3-methyl-3,5-dihydro-4H-imidazol-4-one;
2-amino-5-[3-chloro-4-(difluoromethoxy)phenyl]-5-(3-ethoxy-4-fluorophenyl)-3-methyl-3,5-dihydro-4H-imidazol-4-one;
2-amino-5-[3-chloro-4-(difluoromethoxy)phenyl]-5-(4-fluoro-3-propoxyphenyl)-3-methyl-3,5-dihydro-4H-imidazol-4-one;
2-amino-5-[3-chloro-4-(difluoromethoxy)phenyl]-5-[4-fluoro-3-(3-fluoropropoxy)phenyl]-3-methyl-3,5-dihydro-4H-imidazol-4-one;
2-amino-5-[3-chloro-4-(difluoromethoxy)phenyl]-5-[4-fluoro-3-(2-fluoroethoxy)phenyl]-3-methyl-3,5-dihydro-4H-imidazol-4-one;
2-amino-5-[3-chloro-4-(difluoromethoxy)phenyl]-5-[3-(2,2-difluoroethoxy)-4-fluorophenyl]-3-methyl-3,5-dihydro-4H-imidazol-4-one;
2-amino-5-[4-(difluoromethoxy)-3-methylphenyl]-5-(3-ethoxy-4-fluorophenyl)-3-methyl-3,5-dihydro-4H-imidazol-4-one;
2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
(5S)-2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
(5R)-2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
2-amino-5-[3-(cyclopropylmethoxy)-4-fluorophenyl]-5-[4-(difluoromethoxy)-3-methylphenyl]-3-methyl-3,5-dihydro-4H-imidazol-4-one;
(5R)-2-amino-5-[3-(cyclopropylmethoxy)-4-fluorophenyl]-5-[4-(difluoromethoxy)-3-methylphenyl]-3-methyl-3,5-dihydro-4H-imidazol-4-one;
(5S)-2-amino-5-[3-(cyclopropylmethoxy)-4-fluorophenyl]-5-[4-(difluoromethoxy)-3-methylphenyl]-3-methyl-3,5-dihydro-4H-imidazol-4-one;
2-amino-5-[4-(difluoromethoxy)-3-methylphenyl]-5-[4-fluoro-3-(3-fluoropropoxy)phenyl]-3-methyl-3,5-dihydro-4H-imidazol-4-one;
2-amino-5-[4-(difluoromethoxy)-3-methylphenyl]-5-[4-fluoro-3-(2-fluoroethoxy)phenyl]-3-methyl-3,5-dihydro-4H-imidazol-4-one;
(5S)-2-amino-5-[4-(difluoromethoxy)-3-methylphenyl]-5-[4-fluoro-3-(3-fluoropropoxy)phenyl]-3-methyl-3,5-dihydro-4H-imidazol-4-one;
(5R)-2-amino-5-[4-(difluoromethoxy)-3-methylphenyl]-5-[4-fluoro-3-(3-fluoropropoxy)phenyl]-3-methyl-3,5-dihydro-4H-imidazol-4-one;
(5S)-2-amino-5-[4-(difluoromethoxy)-3-methylphenyl]-5-[4-fluoro-3-(2-fluoroethoxy)phenyl]-3-methyl-3,5-dihydro-4H-imidazol-4-one;
(5R)-2-amino-5-[4-(difluoromethoxy)-3-methylphenyl]-5-[4-fluoro-3-(2-fluoroethoxy)phenyl]-3-methyl-3,5-dihydro-4H-imidazol-4-one;
2-amino-5-[4-(difluoromethoxy)-3-methylphenyl]-5-(4-fluoro-3-propoxyphenyl)-3-methyl-3,5-dihydro-4H-imidazol-4-one;
(5S)-2-amino-5-[4-(difluoromethoxy)-3-methylphenyl]-5-(4-fluoro-3-propoxyphenyl)-3-methyl-3,5-dihydro-4H-imidazol-4-one;
3-(2-{[tert-butyl(dimethyl)silyl]oxy}ethyl)-6-fluoro-1-(2-fluorophenyl)-3,4-dihydro-1H-2,1-benzothiazine 2,2-dioxide;
2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
(5S)-2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
(5R)-2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
(5S)-2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
(5R)-2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one;
(5S)-2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one;
(5R)-2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one
2-amino-5-[4-(difluoromethoxy)-3-methylphenyl]-5-[3-(3,3-difluoropropoxy)phenyl]-3-methyl-3,5-dihydro-4H-imidazol-4-one;
(5R)-2-amino-5-[4-(difluoromethoxy)-3-methylphenyl]-5-[3-(3,3-difluoropropoxy)phenyl]-3-methyl-3,5-dihydro-4H-imidazol-4-one;
(5S)-2-amino-5-[4-(difluoromethoxy)-3-methylphenyl]-5-[3-(3,3-difluoropropoxy)phenyl]-3-methyl-3,5-dihydro-4H-imidazol-4-one;
2-amino-5-[3-(2,2-difluoroethoxy)-4-fluorophenyl]-5-[4-(difluoromethoxy)-3-(2-fluoroethyl)phenyl]-3-methyl-3,5-dihydro-4H-imidazol-4-one;
2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
(5S)-2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
(5R)-2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
2-amino-5-[3-cyclopropyl-4-(difluoromethoxy)phenyl]-5-[3-(3,3-difluoropropoxy)-4-fluorophenyl]-3-methyl-3,5-dihydro-4H-imidazol-4-one;
2-amino-5-[4-(difluoromethoxy)-3-ethylphenyl]-5-[3-(3,3-difluoropropoxy)-4-fluorophenyl]-3-methyl-3,5-dihydro-4H-imidazol-4-one;
(5S)-2-amino-5-[4-(difluoromethoxy)-3-ethylphenyl]-5-[3-(3,3-difluoropropoxy)-4-fluorophenyl]-3-methyl-3,5-dihydro-4H-imidazol-4-one
(5R)-2-amino-5-[4-(difluoromethoxy)-3-ethylphenyl]-5-[3-(3,3-difluoropropoxy)-4-fluorophenyl]-3-methyl-3,5-dihydro-4H-imidazol-4-one;
2-amino-5-[4-(difluoromethoxy)-3-isopropylphenyl]-3-methyl-5-phenyl-3,5-dihydro-4H-imidazol-4-one;
(5S)-2-amino-5-[4-(difluoromethoxy)-3-isopropylphenyl]-3-methyl-5-phenyl-3,5-dihydro-4H-imidazol-4-one;
(5R)-2-amino-5-[4-(difluoromethoxy)-3-isopropylphenyl]-3-methyl-5-phenyl-3,5-dihydro-4H-imidazol-4-one;
2-amino-5-[4-(difluoromethoxy)-3-(2-hydroxyethyl)phenyl]-3-methyl-5-phenyl-3,5-dihydro-4H-imidazol-4-one;
2-amino-5-[3-(2-chloroethyl)-4-(difluoromethoxy)phenyl]-3-methyl-5-phenyl-3,5-dihydro-4H-imidazol-4-one;
2-amino-5-[4-(difluoromethoxy)-3-(2-methoxyethyl)phenyl]-3-methyl-5-phenyl-3,5-dihydro-4H-imidazol-4-one;
(5S)-2-amino-5-[3-(2-chloroethyl)-4-(difluoromethoxy)phenyl]-3-methyl-5-phenyl-3,5-dihydro-4H-imidazol-4-one;
(5R)-2-amino-5-[3-(2-chloroethyl)-4-(difluoromethoxy)phenyl]-3-methyl-5-phenyl-3,5-dihydro-4H-imidazol-4-one;
2-amino-5-[3-(cyclopropylethynyl)-4-fluorophenyl]-5-[4-(difluoromethoxy)-3-isopropylphenyl]-3-methyl-3,5-dihydro-4H-imidazol-4-one;
2-amino-5-[3-(cyclopropylethynyl)-4-fluorophenyl]-5-[4-(difluoromethoxy)-3-ethylphenyl]-3-methyl-3,5-dihydro-4H-imidazol-4-one;
(5R)-2-amino-5-[3-(cyclopropylethynyl)-4-fluorophenyl]-5-[4-(difluoromethoxy)-3-ethylphenyl]-3-methyl-3,5-dihydro-4H-imidazol-4-one;
(5S)-2-amino-5-[3-(cyclopropylethynyl)-4-fluorophenyl]-5-[4-(difluoromethoxy)-3-ethylphenyl]-3-methyl-3,5-dihydro-4H-imidazol-4-one;
2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
(5S)-2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
2-amino-5-(3-bromophenyl)-5-[3-cyclopropyl-4-(difluoromethoxy)phenyl]-3-methyl-3,5-dihydro-4H-imidazol-4-one;
(5R)-2-amino-5-(3-bromophenyl)-5-[3-cyclopropyl-4-(difluoromethoxy)phenyl]-3-methyl-3,5-dihydro-4H-imidazol-4-one;
(5S)-2-amino-5-(3-bromophenyl)-5-[3-cyclopropyl-4-(difluoromethoxy)phenyl]-3-methyl-3,5-dihydro-4H-imidazol-4-one;
2-amino-5-[3-cyclopropyl-4-(difluoromethoxy)phenyl]-5-(3-ethynylphenyl)-3-methyl-3,5-dihydro-4H-imidazol-4-one;
2-amino-5-[3-cyclopropyl-4-(difluoromethoxy)phenyl]-3-methyl-5-(3-prop-1-yn-1-ylphenyl)-3,5-dihydro-4H-imidazol-4-one;
(5R)-2-amino-5-[3-cyclopropyl-4-(difluoromethoxy)phenyl]-3-methyl-5-(3-prop-1-yn-1-ylphenyl)-3,5-dihydro-4H-imidazol-4-one;
(5S)-2-amino-5-[3-cyclopropyl-4-(difluoromethoxy)phenyl]-3-methyl-5-(3-prop-1-yn-1-ylphenyl)-3,5-dihydro-4H-imidazol-4-one;
2-amino-5-(3-but-1-yn-1-ylphenyl)-5-[3-cyclopropyl-4-(difluoromethoxy)phenyl]-3-methyl-3,5-dihydro-4H-imidazol-4-one;
2-amino-5-[3-cyclopropyl-4-(difluoromethoxy)phenyl]-3-methyl-5-(3-pent-1-yn-1-ylphenyl)-3,5-dihydro-4H-imidazol-4-one;
2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
(5S)-2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
(5R)-2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
2-amino-5-[3-cyclopropyl-4-(difluoromethoxy)phenyl]-5-[3-(cyclopropylethynyl)phenyl]-3-methyl-3,5-dihydro-4H-imidazol-4-one;
(5S)-2-amino-5-[3-cyclopropyl-4-(difluoromethoxy)phenyl]-5-[3-(cyclopropylethynyl)phenyl]-3-methyl-3,5-dihydro-4H-imidazol-4-one;
(5R)-2-amino-5-[3-cyclopropyl-4-(difluoromethoxy)phenyl]-5-[3-(cyclopropylethynyl)phenyl]-3-methyl-3,5-dihydro-4H-imidazol-4-one;
2-amino-5-[3-cyclopropyl-4-(difluoromethoxy)phenyl]-5-[3-(3-hydroxy-3-methylbut-1-yn-1-yl)phenyl]-3-methyl-3,5-dihydro-4H-imidazol-4-one;
2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
2-amino-5-[3-cyclopropyl-4-(difluoromethoxy)phenyl]-5-[3-(5-methoxypent-1-yn-1-yl)phenyl]-3-methyl-3,5-dihydro-4H-imidazol-4-one;
2-amino-5-[3-cyclopropyl-4-(difluoromethoxy)phenyl]-5-(3-cyclopropylphenyl)-3-methyl-3,5-dihydro-4H-imidazol-4-one;
2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
2-amino-5,5-bis[3-cyclopropyl-4-(difluoromethoxy)phenyl]-3-methyl-3,5-dihydro-4H-imidazol-4-one;
2-amino-5-[4-(difluoromethoxy)-3-methylphenyl]-5-(4-fluoro-3-isopropoxyphenyl)-3-methyl-3,5-dihydro-4H-imidazol-4-one;
2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5S)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
(5S)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
2-amino-5-[4-(difluoromethoxy)-3-methylphenyl]-5-[3-(4-fluorobut-1-yn-1-yl)phenyl]-3-methyl-3,5-dihydro-4H-imidazol-4-one;
2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
(5R)-2-amino-5-[4-(difluoromethoxy)-3-methylphenyl]-5-[3-(4-fluorobut-1-yn-1-yl)phenyl]-3-methyl-3,5-dihydro-4H-imidazol-4-one;
(5R)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
(5S)-2-amino-5-[4-(difluoromethoxy)-3-methylphenyl]-5-[3-(4-fluorobut-1-yn-1-yl)phenyl]-3-methyl-3,5-dihydro-4H-imidazol-4-one;
(5S)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one;
2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
2-amino-5-[3-cyclopropyl-4-(difluoromethoxy)phenyl]-5-[4-fluoro-3-(3-fluoroprop-1-yn-1-yl)phenyl]-3-methyl-3,5-dihydro-4H-imidazol-4-one;
2-amino-5-[3-cyclopropyl-4-(difluoromethoxy)phenyl]-5-[3-(4-fluorobut-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one; and
(5R)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one;
   a tautomer thereof;
   a stereoisomer thereof; or
   a pharmaceutically acceptable salt thereof.

Additional preferred compounds of the present invention include:

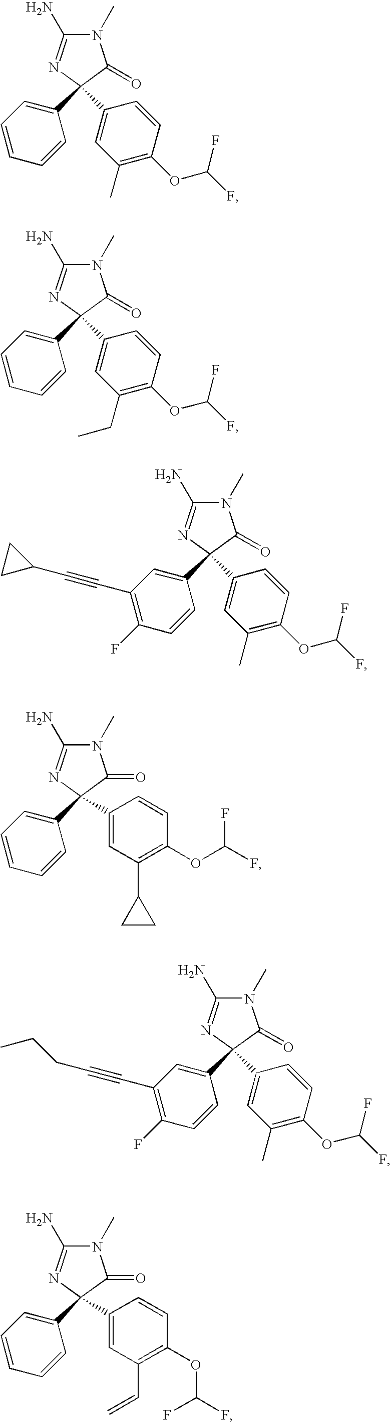

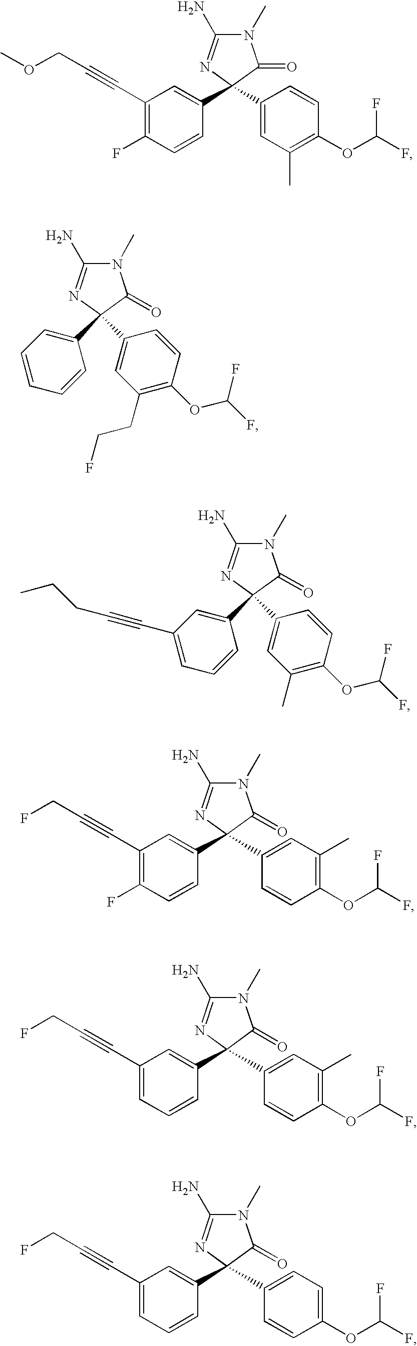

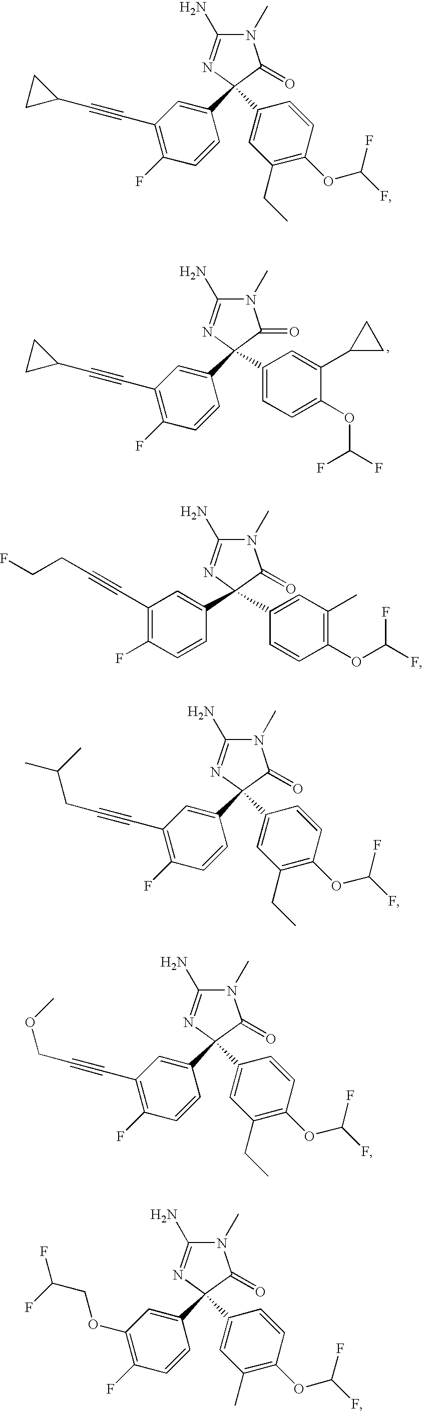

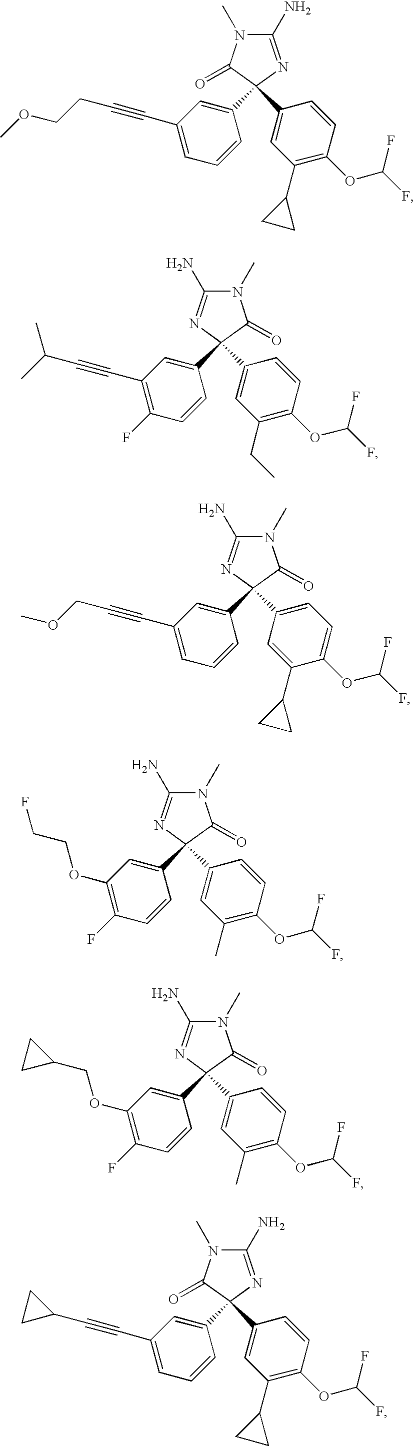

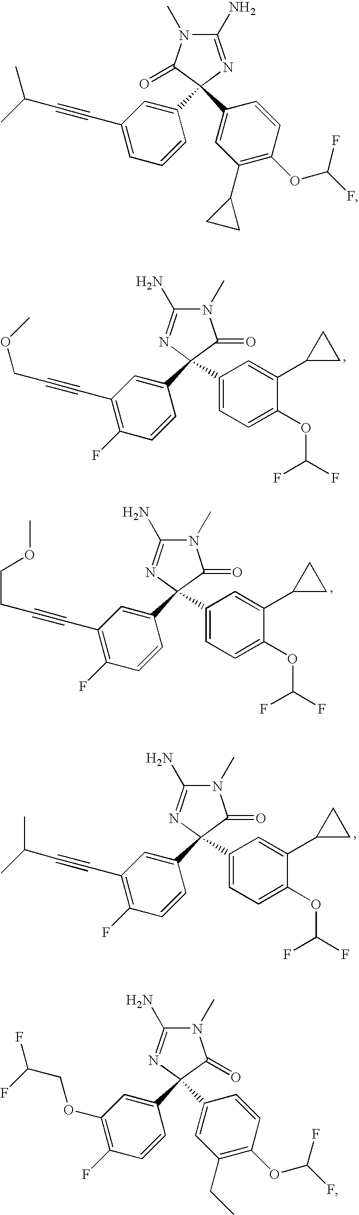

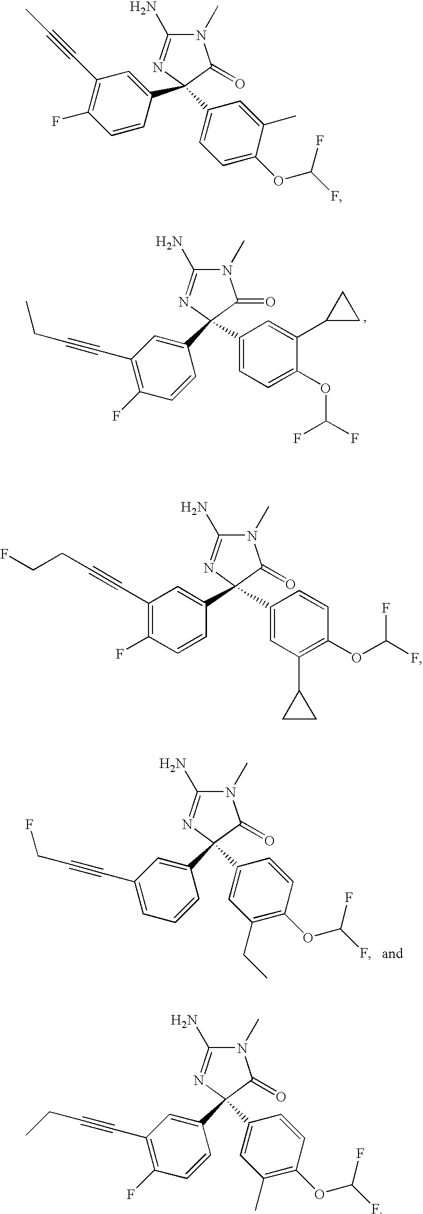

Compounds of formula I may be prepared using conventional synthetic methods and, if required, standard separation or isolation techniques. For example, compounds of formula I may be prepared by reacting a diketone of formula II with an aminoguanidine derivative of formula III in the presence of a base such as a metal carbonate to give the desired formula I compound. The reaction is shown below in flow diagram I.

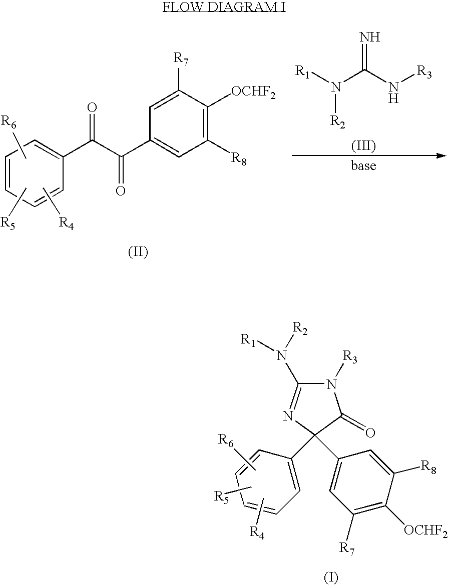

Diketone compounds of formula II may be prepared by reacting an alkyne of formula IV with an oxidizing agent such as Pd(II)Cl₂/DMSO, N-bromosuccinimide/DMSO, ozone, sodium periodate with ruthenium (IV) oxide hydrate, sulfur trioxide, KMnO₄, I₂/DMSO, or combinations thereof, preferable KMnO₄ and I₂/DMSO. The reaction is shown in flow diagram II.

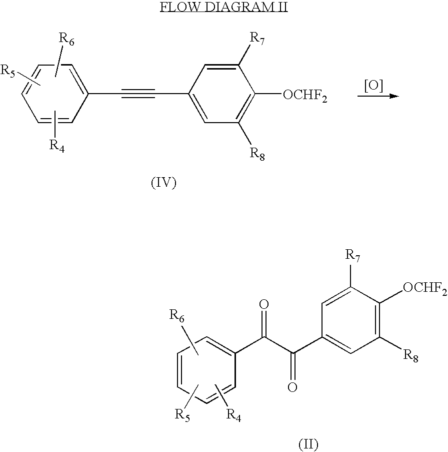

Alkyne compounds of formula IV may be prepared by reacting an ethynylbenzene compound of formula V with a substituted-4-(difluoromethoxy)-1-halobenzene compound of formula VI in the presence of a Pd catalyst, such as dichlorobis(triphenylphosphine)palladium (II), and CuI to give the desired phenylethynylbenzene compound of formula IV. The reaction is shown in flow diagram III wherein Hal represents Br or I.

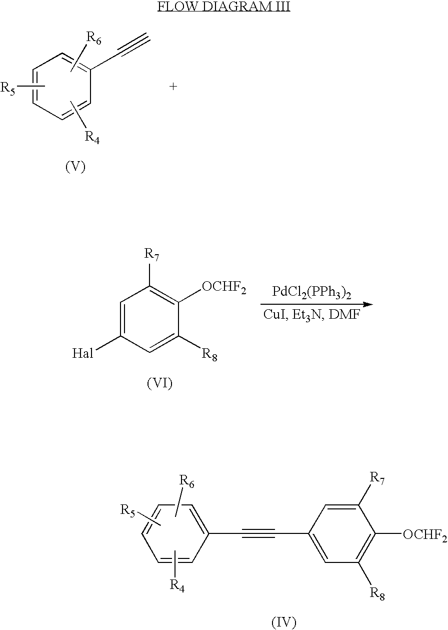

Advantageously, the compounds of formula I act as BACE inhibitors for the treatment of β-amyloid deposits and neurofibrillary tangles associated with such diseases as Alzheimer's disease, Trisomy 21 (Down's Syndrome), Hereditary Cerebral Hemorrhage with Amyloidosis of the Dutch-type (HCHWA-D), and other neurodegenerative disorders. Accordingly, the present invention provides methods for modulating BACE and treating, preventing, or ameliorating β-amyloid deposits and neurofibrillary tangles associated with diseases and disorders such as Alzheimer's disease, Trisomy 21 (Down's Syndrome), Hereditary Cerebral Hemorrhage with Amyloidosis of the Dutch-type (HCHWA-D), or other neurodegenerative disorders. Such methods include providing a patient suffering from or being susceptible to a disease or injury associated with excessive BACE activity an effective amount of a compound of formula I. Also according to the present invention there is provided a method of treating Alzheimer's disease and related senile dementia's in humans or other mammals which comprises administering to a human or other mammal an effective amount of a compound of the present invention.

The present invention also provides a method for the treatment of a disorder related to or associated with excessive BACE activity in a patient in need thereof which comprises providing said patient a therapeutically effective amount of at least one compound of formula I. Representative disorders include Alzheimer's disease, cognitive impairment, Down's Syndrome, HCHWA-D, cognitive decline, senile dementia, cerebral amyloid angiopathy, degenerative dementia, or other neurodegenerative disorders. Certain of these diseases are characterized by production of β-amyloid deposits or neurofibrillary tangles.

The present invention also provides a method for inhibiting the activity of BACE, comprising administering to a patient or contacting a receptor thereof with an effective amount of at least one compound of formula I. Certain methods further comprise determining BACE activity, either before or after said contacting step.

The present invention also provides a method of ameliorating β-amyloid deposits or neurofibrillary tangles in a mammal which comprises providing said mammal an effective amount of at least one compound of formula I.

Also provided are methods of ameliorating symptoms of Alzheimer's disease, cognitive impairment, Down's Syndrome, HCHWA-D, cognitive decline, senile dementia, cerebral amyloid angiopathy, degenerative dementia, or other neurodegenerative disorders in a mammal which comprises providing said mammal an effective amount of at least one compound of formula I.

Further methods prevent Alzheimer's disease, cognitive impairment, Down's Syndrome, HCHWA-D, cognitive decline, senile dementia, cerebral amyloid angiopathy, degenerative dementia, or other neurodegenerative disorders in a mammal that is known to suffer from or suspected to be at risk of suffering from such diseases. These methods comprise providing said mammal an effective amount of at least one compound of formula I.

As used in accordance with this invention, the term “providing,” with respect to providing a compound or substance covered by this invention, means either directly administering such a compound or substance, or administering a prodrug, derivative, or analog which will form the effective amount of the compound or substance within the body. This invention also covers providing the compounds of this invention to treat the disease states disclosed herein that the compounds are useful for treating.

The term “patient”, as used herein, refers to a mammal, preferably a human.

The terms “administerdministering”, or “administration”, as used herein, refer to either directly administering a compound or composition to a patient, or administering a prodrug derivative or analog of the compound to the patient, which will form an equivalent amount of the active compound or substance within the patient's body.

The terms “effective amount”, “therapeutically effective amount” and “effective dosage” as used herein, refer to the amount of a compound that, when administered to a patient, is effective to at least partially ameliorate (and, in preferred embodiments, cure) a condition from which the patient is suspected to suffer.

It is understood that the effective dosage of the active compounds of this invention may vary depending upon the particular compound utilized, the mode of administration, the condition, and severity thereof, of the condition being treated, as well as the various physical factors related to the individual being treated. For treating Alzheimer's disease and other related senile dementia's, generally, satisfactory results may be obtained when the compounds of this invention are administered to the individual in need at a daily dosage of from about 0.1 mg to about 1 mg per kilogram of body weight, preferably administered in divided doses two to six times per day, or in a sustained release form. For most large mammals, the total daily dosage is from about 3.5 mg to about 140 mg preferably from about 3.5 to about 5 mg. In the case of a 70 kg human adult, the total daily dose will generally be from about 7 mg to about 70 mg and may be adjusted to provide the optimal therapeutic result. This regimen may be adjusted to provide the optimal therapeutic response.

In one aspect, the present invention is directed to compositions comprising one or more compounds of formula I and one or more pharmaceutically acceptable carriers.

The present invention also comprises pharmaceutical compositions comprising compounds of the above-described formula I and a pharmaceutically acceptable carrier.

The term “carrier”, as used herein, shall encompass carriers, excipients, and diluents. Examples of carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time-release formulations to alter the absorption of the active compound(s). The oral formulation may also consist of administering the active ingredient in water or fruit juice, containing appropriate solubilizers or emulsifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. Such administrations may be carried out using the present compounds, or pharmaceutically acceptable salts thereof, in lotions, creams, foams, patches, suspensions, solutions, and suppositories (rectal and vaginal).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, thransdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

In certain embodiments, the present invention is directed to prodrugs. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975).

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved.

For a more clear understanding, and in order to illustrate the invention more clearly, specific examples thereof are set forth hereinbelow. The following examples are merely illustrative and are not to be understood as limiting the scope and underlying principles of the invention in any way.

Unless otherwise stated, all parts are parts by weight. The terms DMSO and DMF designate dimethyl sulfoxide and N,N-dimethylformamide, respectively. The terms EtOAc and THF designate ethyl acetate and tetrahydrofuran, respectively. The term NMR designates proton nuclear magnetic resonance and the term MS designates mass spectroscopy with (+) referring to the positive mode which generally gives a M+1 (or M+H) absorption where M=the molecular mass. All compounds are analyzed at least by MS and NMR.

### EXAMPLE 1

### Preparation of 2-Amino-5-[4-(difluoromethoxy)-3-methylphenyl]-3-methyl-5-phenyl-3,5-dihydro-4H-imidazol-4-one

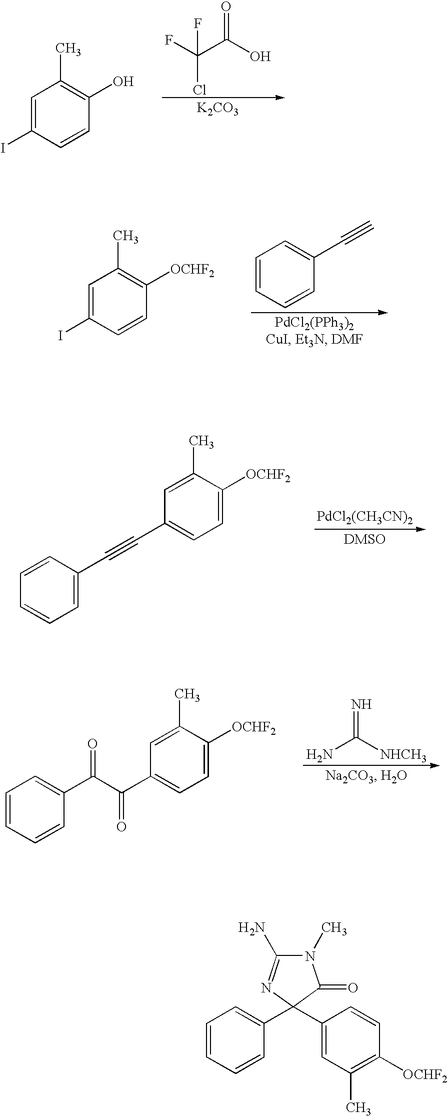

### Step 1: 1-(difluoromethoxy)-4-iodo-2-methylbenzene

A mixture of 4-iodo-2-methylphenol (10 g, 42.7 mmol) in DMF and water was treated with 2-chloro-2,2-difluoroacetic acid (3.61 mL, 42.7 mmol) and potassium carbonate (23.62 g, 171 mmol), heated to 120° C. for 12 h, cooled to room temperature and diluted with EtOAc and water. The organic phase was separated, washed sequentially with water and brine, dried over Na₂SO₄ and concentrated in vacuo. The resultant residue was purified by flash chromatography (0-10% EtOAc/hexanes) to give a 1-(difluoromethoxy)-4-iodo-2-methylbenzene (3 g, 10.56 mmol, 24.72% yield) as a clear oil. ¹H NMR (400 MHz, DMSO-d₆) [symbol] 7.66 (d, J=1.5 Hz, 1H), 7.56 (dd, J=8.47 and 2.09 Hz, 1H), 7.15 (t, J_{H-F}=74 Hz, 1H), 6.92 (d, J=8.47 Hz, 1H), 2.15 (s, 3H).

### Step 2: 1-(difluoromethoxy)-2-methyl-4-(phenylethynyl)benzene

A mixture of 1-(difluoromethoxy)-4-iodo-2-methylbenzene (3 g, 10.56 mmol), ethynylbenzene (1.160 mL, 10.56 mmol), and triethylamine (7.36 mL, 52.8 mmol) in DMF (21.12 mL) was treated with bis(triphenylphosphine)dichloropalladium (0.371 g, 0.528 mmol) and copper(I) iodide (0.201 g, 1.056 mmol), stirred at 25° C. for 2 h and partitioned between ether and 1M HCl. The organic phase was separated, washed sequentially with 1M HCl and brine, dried over Na₂SO₄ and concentrated in vacuo. The resultant residue was purified by flash chromatography (100% hexanes) to provide 1-(difluoromethoxy)-2-methyl-4-(phenylethynyl)benzene (2.13 g, 8.25 mmol, 78% yield) as a dark brown oil. This oil was used as is in the next step.

### Step 3: 1-(4-(difluoromethoxy)-3-methylphenyl)-2-phenylethane-1,2-dione

A solution of 1-(difluoromethoxy)-2-methyl-4-(phenylethynyl)benzene (2.13 g, 8.25 mmol) in DMSO was treated with palladium dichlorobisacetonitrile (0.214 g, 0.825 mmol), heated at 145° C. for 1 h, allowed to cool to room temperature and partitioned between water and ether. The organic phase was seqarated, washed sequentially with water and brine, dried over Na₂SO₄ and concentrated in vacuo. The resultant residue was purified by flash chromatography (0-20% EtOAc/hexanes) to give 1-(4-(difluoromethoxy)-3-methylphenyl)-2-phenylethane-1,2-dione (2.04 g, 7.03 mmol, 85% yield) as an orange oil that solidified upon standing. MS m/e (M−H)⁻ 289.05

### Step 4: 2-Amino-4-(4-(difluoromethoxy)-3-methylphenyl)-1-methyl-4-phenyl-1H-imidazol-5(4H)-one

A solution of 1-(4-(difluoromethoxy)-3-methylphenyl)-2-phenylethane-1,2-dione (2. g, 6.89 mmol) in ethanol was treated with sodium carbonate (1.095 g, 10.34 mmol) and 1-methylguanidine hydrochloride (1.132 g, 10.34 mmol), heated to 80° C., cooled to room temperature and filtered. The filter cake was washed with EtOH. The filtdrates were combined and concentrated in vacuo. The resultant residue was dissolved in CH₂Cl₂ (10 mL) and purified by flash chromatography (0-10% MeOH in CH₂Cl₂) to provide the title product as an off white solid, 2.04 g, 5.91 mmol, 86% yield, identified by NMR and mass spectral analyses. MS m/e (M+H)⁺ 346.00.

### EXAMPLE 2

### Preparation of (5S)-2-Amino-5-[4-(difluoromethoxy)-3-methylphenyl]-3-methyl-5-phenyl-3,5-dihydro-4H-imidazol-4-one [A] and (5R)-2-Amino-5-[4-(difluoromethoxy)-3-methylphenyl]-3-methyl-5-phenyl-3,5-dihydro-4H-imidazol-4-one [B]

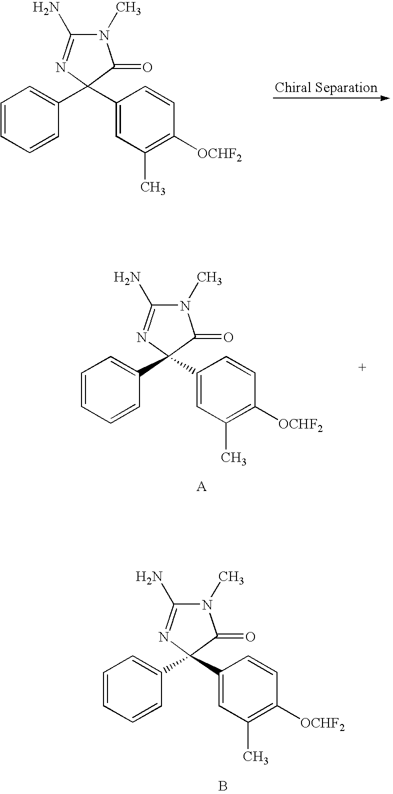

A racemic mixture of 2-amino-5-[4-(difluoromethoxy)-3-methylphenyl]-3-methyl-5-phenyl-3,5-dihydro-4H-imidazol-4-one (1.8 g, 5.21 mmol) was separated by chiral chromatography (Chiral Cel OJ 5×50 cm Mobile phase 15% 2-butanol in hexane (0.1% DEA)) to provide the title product A (S-enantiomer) peak 1, RT=8.5 min, (0.9 g, 2.61 mmol, 50.0% yield) as a white solid, MS m/e (M+H)⁺ 346.10, [α]_{D}²⁵=+11.2 (c=1% in MeOH); and the title product B (R-enantiomer) peak 2, RT=11.8 min, (0.84 g, 2.432 mmol, 46.7% yield) as a white solid, MS m/e (M+H)⁺ 346.10, [α]_{D}²⁵=−9.2 (c=1% in MeOH).

### EXAMPLE 3

### Preparation of 2-Amino-4-(4-(difluoromethoxy)-3-methylphenyl)-4-(4-fluorophenyl)-1-methyl-1H-imidazol-5(4H)-one

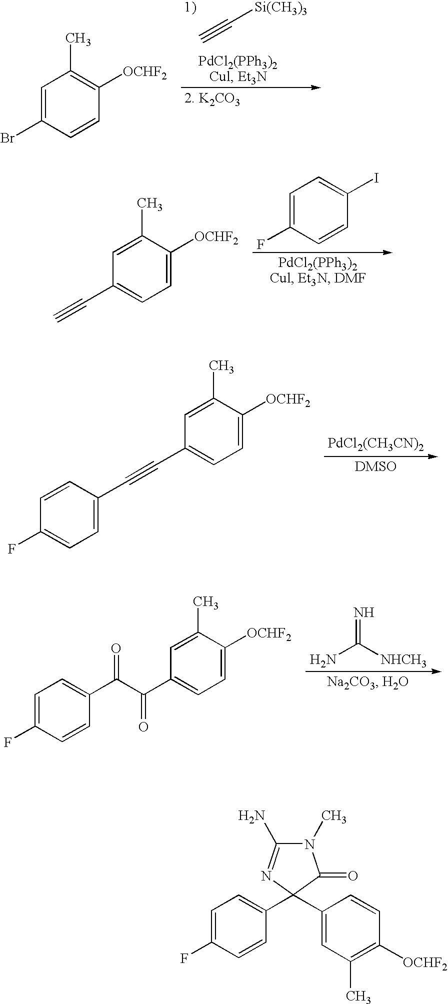

### Step 1: {[4-(difluoromethoxy)-3-methylphenyl]ethynyl}trimethylsilane

A solution of 4-bromo-1-(difluoromethoxy)-2-methylbenzene (5.3 g, 22.36 mmol), ethynyltrimethylsilane (4.74 mL, 33.5 mmol), and triethylamine (15.58 mL, 112 mmol) in DMF was degassed by bubbling with N₂ for 30 min, treated with bis(triphen-ylphosphine)dichloropalladium (0.785 g, 1.118 mmol) with continued N₂ bubbling, treated with copper(I) iodide (0.426 g, 2.236 mmol), warmed to 65° C. for 12 h, cooled toroom temperature, partitioned between ether and 2M HCl and filtered through Celite. The filtrate was separated and the organic phase was washed sequentially with 2M HCl and brine, dried over Na₂SO₄ and concentrated in vacuo. The resultant residue was purified by flash chromatography (100% hexanes) to provide ((4-(difluoromethoxy)-3-methylphenyl)ethynyl)trimethylsilane (5.49 g, 21.58 mmol, 97% yield). ¹H NMR (400 MHz, DMSO-d₆) [symbol] 7.39 (d, J=1.5 Hz, 1H), 7.32 (dd, J=8.47 and 1.5 Hz, 1H), 7.20 (t, J_{H-F}=74 Hz, 1H), 7.08 (d, J=8.47 Hz, 1H), 2.15 (s, 3H), 0.18 (s, 9H).

### Step 2: 1-(difluoromethoxy)-4-ethynyl-2-methylbenzene

A solution of ((4-(difluoromethoxy)-3-methylphenyl)ethynyl)trimethylsilane (5.49 g, 21.58 mmol) in CH₃OH was treated with potassium carbonate (29.8 g, 216 mmol), stirred for 3 h and partitioned between hexanes and water. The organic phase was separated, dried over Na₂SO₄ and concentrated in vacuo to provide 1-(difluoro-methoxy)-4-ethynyl-2-methylbenzene (2.55 g, 14.00 mmol, 64.9% yield) as a brown oil. ¹H NMR (400 MHz, DMSO-d₆) [symbol] 7.39 (d, J=1.4 Hz, 1H), 7.32 (dd, J=8.46 and 1.1 Hz, 1H), 7.18 (t, J_{H-F}=74 Hz, 1H), 7.09 (d, J=8.35 Hz, 1H), 2.16 (s, 3H).

### Step 3: 1-(difluoromethoxy)-4-((4-fluorophenyl)ethynyl)-2-methylbenzene

A mixture of 4-fluoro-1-iodobenzene (0.6 g, 2.7 mmol) and 1-(difluoro-methoxy)-4-ethynyl-2-methylbenzene (0.74 g, 4.05 mmol) is treated with 1 mL of DMF and triethylamine (2.6 mL, 19 mmol), followed by copper iodide (26 mg, 0.14 mmol), bis(triphenylphosphine)dichloropalladium (0.29 g, 0.41 mmol) and 2 mL of DMF. The reaction is stirred under nitrogen at room temperature overnight and concentrated under vacuum. The resultant concentrate was purified by flash chromatography (EtOAC: Hexane) to give 1-(difluoromethoxy)-4-((4-fluorophenyl)ethynyl)-2-methylbenzene as an oil (>700 mg, 93% yield).

### Steps 4 and 5: 2-amino-4-(4-(difluoromethoxy)-3-methylphenyl)-4-(4-fluorophenyl)-1-methyl-1H-imidazol-5(4H)-one

Using essentially the same procedure described in Example 1, steps 3 and 4 and employing 1-(difluoromethoxy)-4-((4-fluorophenyl)ethynyl)-2-methylbenzene and 1-(difluoromethoxy)-4-iodo-2-methylbenzene, the title product was obtained as a white solid, identified by NMR and mass spectral analyses. MS m/e (M+H)+ 364.3.

### EXAMPLE 4

### Preparation of (5S)-2-Amino-4-(4-(difluoromethoxy)-3-methylphenyl)-4-(4-fluorophenyl)-1-methyl-1H-imidazol-5(4H)-one [A] and (5R)-2-Amino-4-(4-(difluoromethoxy)-3-methylphenyl)-4-(4-fluorophenyl)-1-methyl-1H-imidazol-5(4H)-one [B]

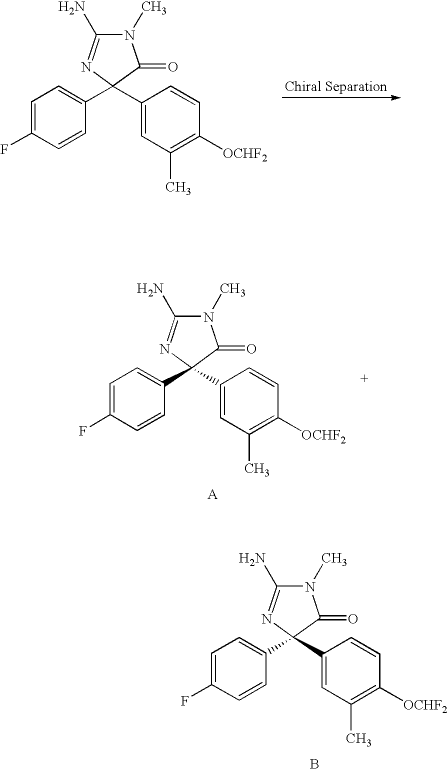

Using essentially the same procedure described in Example 2 and employing a racemic mixture of 2-amino-4-(4-(difluoromethoxy)-3-methylphenyl)-4-(4-fluorophenyl)-1-methyl-1H-imidazol-5(4H)-one, the title products were obtained and identified by NMR and mass spectral analyses. Title product A (S-enantiomer), MS m/e (M+H)+ 364.3; [α]_{D}²⁵=+12.9 (c=1% in MeOH); and title product B (R-enantiomer), MS m/e (M+H)+ 364.3, [U]_{D}²⁵=−12.0 (c=1% in MeOH).

### EXAMPLES 5-22

### Preparation of 2-Amino-5-[substituted-4-(difluoromethoxy)phenyl]-3-methyl-3,5-dihydro-4H-imidazol-4-one Compounds

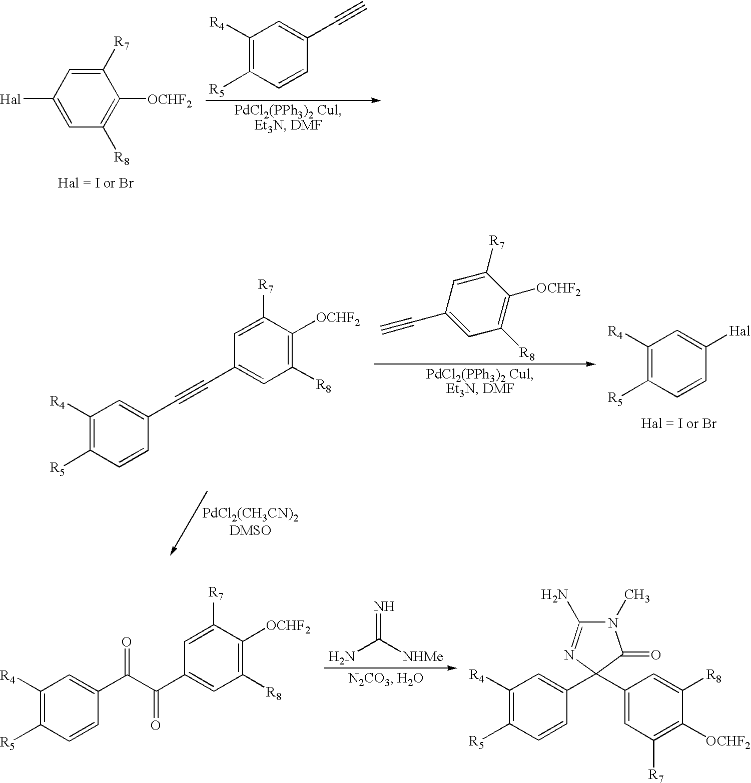

Using essentially the same procedures described in Examples 1 and 3 employing the appropriate halobenzene and phenylethyne, the compounds shown on Table I were obtained and identified by NMR and mass spectral analyses.

TABLE I

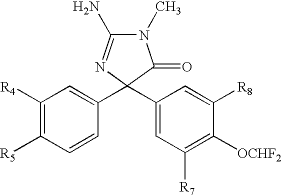

| Ex. No. | R4 | R5 | R7 | R8 | [M + H]⁺ |
|---|---|---|---|---|---|
| 5 | H | H | CH₃ | CH₃ | 360.1 |
| 6 | H | H | C₂H₅ | H | 360.1 |
| 7 | H | F | C₂H₅ | H | 378.4 |
| 8 | H | H | n-propyl | H | 374.2 |
| 9 | H | H | i-propyl | H | 374 |
| 10 | H | H | cyclopropyl | H | 370* |
| 11 | H | H | CH═CH₂ | H | 358 |
| 12 | H | H | CF₃ | H | 400.1 |
| 13 | H | H | CH₂F | H | 364.1 |
| 14 | H | H | CH₂F | H | 382.1 |
| 15 | H | H | OCH₃ | H | 360* |
| 16 | H | H | Cl | H | 311 |
| 17 | H | H | F | H | 350.05 |
| 18 | Br | H | CH₃ | H | 429.3 |
| 19 | Br | F | CH₃ | H | 442.1 |
| 20 | CH₃ | F | CH₃ | H | 378.14 |
| 21 | CN | OCH₃ | CH₃ | H | 401.14 |
| 22 | CH₂F | F | CH₃ | H | 396.13 |

*[M + H]⁺

### EXAMPLES 23-34

### Preparation of 5(S)-2-Amino-5-[substituted-4-(difluoromethoxy)phenyl]-3-methyl-3,5-dihydro-4H-imidazol-4-one and 5(R)-2-Amino-5-[substituted-4-(difluoromethoxy)phenyl]-3-methyl-3,5-dihydro-4H-imidazol-4-one Compounds

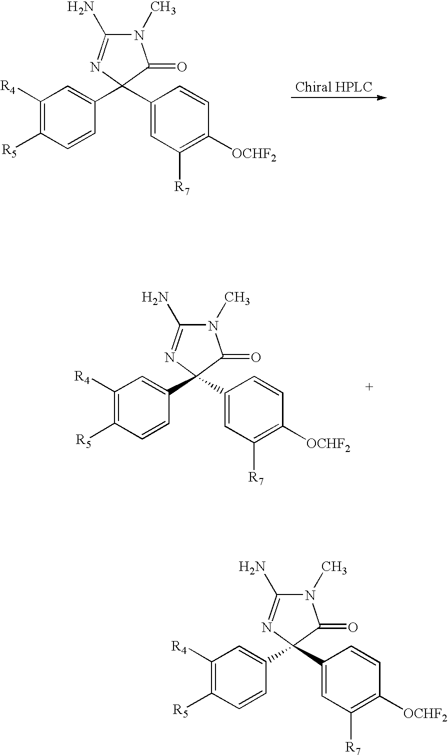

Using essentially the same procedure described in Example 2 and employing the appropriate racemic 2-amino-5-[substituted-4-(difluoromethoxy)phenyl]-3-methyl-3,5-dihydro-4H-imidazol-4-one, the enantiomeric compounds shown in Table II were obtained and identified by NMR and mass spectral analyses.

TABLE II

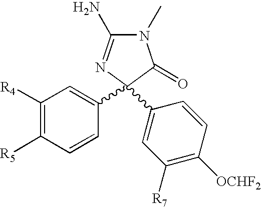

| Ex. No. | Chiral | R4 | R5 | R7 | [M + H]⁺ | [α]_{D}²⁵* |
|---|---|---|---|---|---|---|
| 23 | 5-S | H | H | C₂H₅ | 360.20 | +11.8 |
| 24 | 5-R | H | H | C₂H₅ | 360.20 | −13.6 |
| 25 | 5-R | H | H | cyclopropyl | 370** | +18.0 |
| 26 | 5-S | H | H | cyclopropyl | 372.15 | −13.0 |
| 27 | 5-S | H | H | Cl | 366.10 | +32.6 |
| 28 | 5-R | H | H | Cl | 366.10 | −29.8 |
| 29 | 5-S | H | H | Br | 424 | −11 |
| 30 | 5-R | H | H | Br | 424.1 | +18 |
| 31 | 5-S | Br | F | CH₃ | — | +31 |
| 32 | 5-R | Br | F | CH₃ | — | −23 |
| 33 | 5-R | H | H | CH═CH₂ | 356** | +17 |
| 34 | 5-S | H | H | CH═CH₂ | 358 | −19 |

*1% Methanol Solution
**[M − H]⁻

### EXAMPLE 35

### Preparation of 2-Amino-5-(3-butoxyphenyl)-5-[3-chloro-4-(difluoromethoxy)-phenyl]-3-methyl-3,5-dihydro-4H-imidazol-4-one

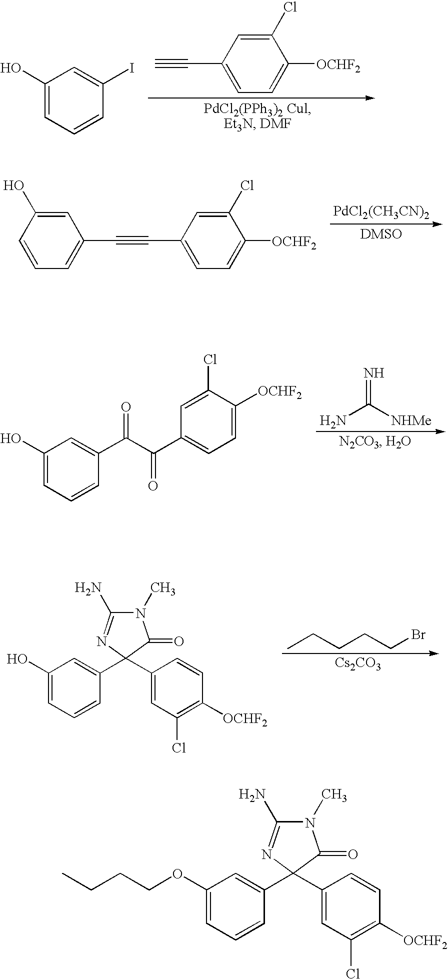

### Step 1: 3-((3-Chloro-4-(difluoromethoxy)phenyl)ethynyl)phenol

A mixture of bis(triphenylphosphine)dichloropalladium(II) (242 mg, 0.345 mmol), CuI (39 mg, 0.207 mmol), triethylamine (3.49 g, 4.8 mL, 34.5 mmol), and 2-chloro-1-(difluoromethoxy)-4-ethynylbenzene (1.54 g, 7.6 mmol) in DMF was treated with 3-iodophenol (1.52 g, 6.9 mmol), stirred for 3 h, diluted with water and extracted with EtOAc. The extracts were combined, washed with water, dried over Na₂SO₄, and concentrated in vacuo to give a residue. The residue was purified by flash chromatography (SiO₂, 1:9 to 3:7 EtOAc:hexanes) to yield 3-((3-chloro-4-(difluoromethoxy)phenyl)ethynyl)phenol as a red oil, 1.55 g, 73% yield, MS (−ESI): m/z 293 ([M−H]⁻)

### Step 2: 1-(3-Chloro-4-(difluoromethoxy)phenyl)-2-(3-hydroxyphenyl)ethane-1,2-dione

A solution of 3-((3-chloro-4-(difluoromethoxy)phenyl)ethynyl)phenol (1.55 g, 5.25 mmol) in dry DMSO was treated with bis(acetonitrile)dichloropalladium(II) (135 mg, 0.525 mmol), heated overnight at 145° C., cooled to room temperature, diluted with water and extracted with EtOAc. The combined extracts were washed with water, dried over Na₂SO₄, and concentrated in vacuo over 5 g Celite. the resultant residue was purified by flash chromatography (SiO₂, 1:9 to 1:4 EtOAc:Hexanes) to give 1-(3-chloro-4-(difluoromethoxy)phenyl)-2-(3-hydroxyphenyl)ethane-1,2-dione as a yellow solid, 1.45 g, 84% yield, MS (+ESI): m/z 327 ([M+H]⁺).

### Step 3: 2-Amino-4-(3-chloro-4-(difluoromethoxy)phenyl)-4-(3-hydroxyphenyl)-1-methyl-1H-imidazol-5(4H)-one

A mixture of 1-(3-chloro-4-(difluoromethoxy)phenyl)-2-(3-hydroxyphenyl)ethane-1,2-dione (1.45 g, 4.43 mmol) and 1-methylguanidine (727 mg, 6.65 mmol) in EtOH was treated with Na₂CO₃ (705 mg, 6.65 mmol), heated at reflux temperature for 1 h, cooled to room temperature and filtered. The filtercake was washed with EtOH, and the combined filtrates were concentrated onto Celite. The resultant residue was purified by flash chromatography (SiO₂, DCM to 1:9 MeOH:DCM) to afford 2-amino-4-(3-chloro-4-(difluoromethoxy)phenyl)-4-(3-hydroxyphenyl)-1-methyl-1H-imidazol-5(4H)-one as a grey foam, 965 mg, 56% yield.

### Step 4: 2-Amino-4-(3-butoxyphenyl)-4-(3-chloro-4-(difluoromethoxy)phenyl)-1-methyl-1H-imidazol-5(4H)-one

A mixture of 2-amino-4-(3-chloro-4-(difluoromethoxy)phenyl)-4-(3-hydroxyphenyl)-1-methyl-1H-imidazol-5(4H)-one (50 mg, 0.131 mmol) in DMF was treated with Cs₂CO₃ (43 mg, 0.131 mmol), followed by n-butylbromide (20 mg, 15.4 μL, 0.144 mmol). The mixture was stirred at room temperature overnight, diluted with water and extracted with EtOAc. The combined extracts were dried over Na₂SO₄, and concentrated in vacuo to give an oil. The oil was absorbed onto 250 mg Celite and purified by flash chromatography (SiO₂, DCM to 1:9 MeOH:DCM) to give the title product as a white sticky oil, 25 mg, 43% yield, identified by NMR and mass spectral analyses. MS (+ESI): m/z 438 ([M+H]⁺)

### EXAMPLES 36-45

### Preparation of 2-Amino-5-(3-alkoxyphenyl)-5-[substituted-4-(difluoromethoxy)-phenyl]-3-methyl-3,5-dihydro-4H-imidazol-4-one Compounds

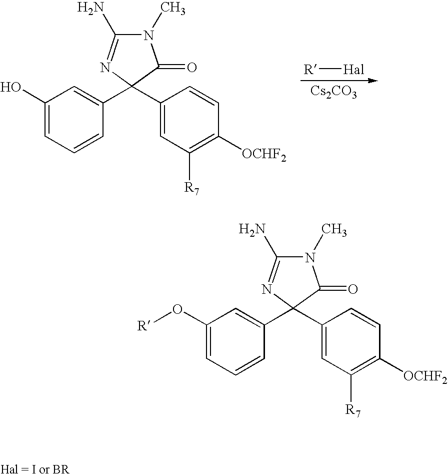

Using essentially the same procedure described in Example 35 and employing the appropriate 2-amino-5-(3-hydroxyphenyl)-5-[substituted-4-(difluoromethoxy)phenyl]-3-methyl-3,5-dihydro-4H-imidazol-4-one compound and desired alkyliodide or alkylbromide, the compounds shown in Table III were obtained and identified by NMR and mass spectral analyses.

TABLE III

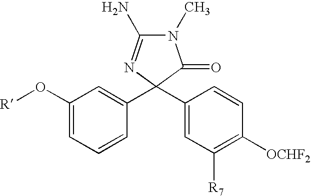

| Ex. No. | R′ | R7 | [M + H]⁺ |
|---|---|---|---|
| 36 | C₂H₅ | Cl | 410.1 |
| 37 | CH₂CH₂F | Cl | 428.1 |
| 38 | CH₂CHF₂ | Cl | 446.1 |
| 39 | n-propyl | Cl | 424.1 |
| 40 | 3-fluoropropyl | Cl | 442.1 |
| 41 | C₂H₅ | CH₃ | 390.1 |
| 42 | CH₂CH₂F | CH₃ | 408.1 |
| 43 | CH₂CHF₂ | CH₃ | 426.1 |
| 44 | n-propyl | CH₃ | 404 |
| 45 | 3-fluoropropyl | CH₃ | 422.1 |

### EXAMPLE 46

### Preparation of 2-Amino-5-[3-(cyclopropylethynyl)phenyl]-5-[4-(difluoro-methoxy)-3-methylphenyl]-3-methyl-3,5-dihydro-4H-imidazol-4-one

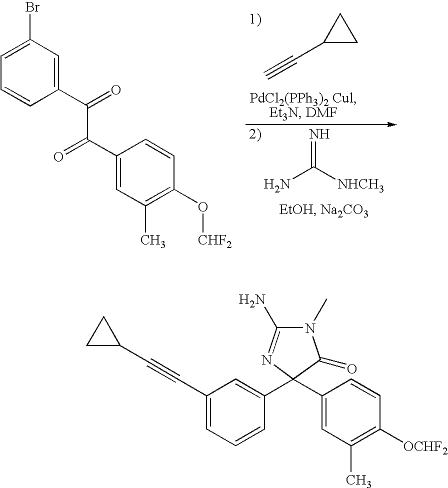

### Step 1: 1-(3-(cyclopropylethynyl)phenyl)-2-(4-(difluoromethoxy)-3-methyl-phenyl)ethane-1,2-dione

A mixture of 1-(3-bromophenyl)-2-(4-(difluoromethoxy)-3-methylphenyl)-ethane-1,2-dione (1 g, 2.71 mmol), ethynylcyclopropane (0.385 mL, 3.25 mmol), and triethylamine (1.888 mL, 13.54 mmol) in DMF was degassed by bubbling with N₂ for 30 min, treated with bis(triphenylphosphine)dichloropalladium (0.095 g, 0.135 mmol), N₂ bubbling was continued, then treated with copper(I) iodide (0.052 g, 0.271 mmol), warmed to 65° C. for 2 h (reaction was complete by LC/MS), cooled to room temperature and partitioned between ether and 1M HCl. The organic phase was washed sequentially with 1M HCl and brine, dried over Na₂SO₄ and concentrated in vacuo. The resultant residue was purified by flash chromatography (gradient 0-30% EtOAc:hexanes) to give 1-(3-(cyclopropylethynyl)phenyl)-2-(4-(difluoromethoxy)-3-methylphenyl)ethane-1,2-dione (0.92 g, 2.60 mmol, 96% yield) as an orange solid.

MS m/e (M−H)⁻ 353.0

### Step 2: 2-amino-4-(3-(cyclopropylethynyl)phenyl)-4-(4-(difluoromethoxy)-3-methyl-1-1H-imidazol-5(4H)-one

A solution of 1-(3-(cyclopropylethynyl)phenyl)-2-(4-(difluoromethoxy)-3-methylphenyl)ethane-1,2-dione (0.92 g, 2.60 mmol) in ethanol was treated with 1-methylguanidine hydrochloride (0.284 g, 2.60 mmol), followed by sodium carbonate (0.275 g, 2.60 mmol), heated to 80° C. for 2 h, cooled to room temperature and concentrated in vacuo. The resultant oil residue was purified by flash chromatography (gradient 0-10% MeOH:CH₂Cl₂) to provide the title product as an tan solid, 0.78 g, 1.905 mmol, 73.4% yield, identified by NMR and mass spectral analyses. MS m/e (M−H)⁻ 408.2

### EXAMPLES 47 AND 48

### Preparation of 2-Amino-5-[3-(alkynyl)-4-fluorophenyl]-5-[4-(difluoromethoxy)-3-methylphenyl]-3-methyl-3,5-dihydro-4H-imidazol-4-one Compounds

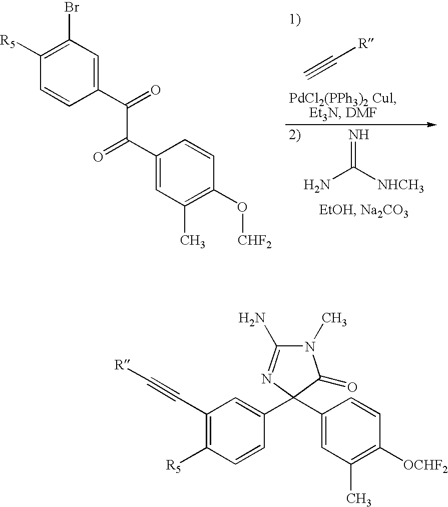

Using essentially the same the procedure described in Example 46 and employing the appropriate dione and the desired alkyne in step 1, the compounds shown on Table IV were obtained and identified by NMR and mass spectral analyses.

TABLE IV

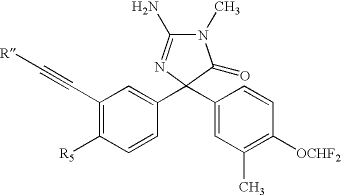

| Ex. No. | R″ | R5 | [M + H]⁺ |
|---|---|---|---|
| 47 | cyclopropyl | F | 426.1* |
| 48 | isopropyl | F | 430.1 |

*[M − H]⁻

### EXAMPLE 49

### Preparation of 2-Amino-5-[4-(difluoromethoxy)-3-methylphenyl]-5-[4-fluoro-3-(3-methylbut-1-yn-1-yl)phenyl]-3-methyl-3,5-dihydro-4H-imidazol-4-one

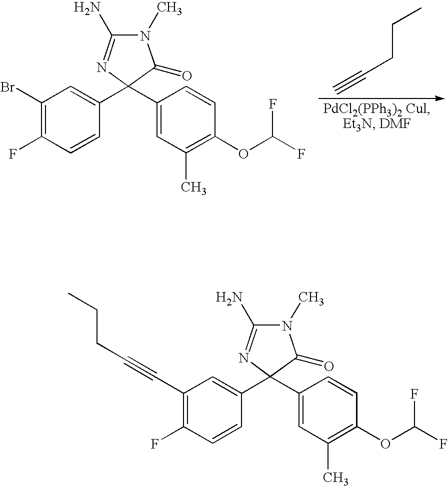

A mixture of 2-amino-4-(3-bromo-4-fluorophenyl)-4-(4-(difluoromethoxy)-3-methylphenyl)-1-methyl-1H-imidazol-5(4H)-one (0.75 g, 1.696 mmol), acetonitrile and pyrrolidine was degassed by bubbling through with N₂, treated with pent-1-yne (0.251 mL, 2.54 mmol) with continued N₂ bubbling, then treated with bis(triphenylphosphine)-dichloropalladium (0.119 g, 0.170 mmol) and copper(I) iodide (0.016 g, 0.085 mmol), heated to 60° C. for 30 min. The reaction mixture was treated with an additional amount of pent-1-yne (0.251 mL, 2.54 mmol), cooled to room temperature and partitioned between EtOAc and saturated NaHCO₃. The organic phase was separated, washed with brine, dried over Na₂SO₄ and concentrated in vacuo. The resultant residue was purified by flash chromatography (gradient 0-10% MeOH, CHCl2) to provide the title product as a light colored solid, 0.635 g, 1.479 mmol, 87% yield, identified by NMR and mass spectral analyses. MS m/e (M+H)⁺ 430.2

### EXAMPLE 50

### Preparation of 2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one

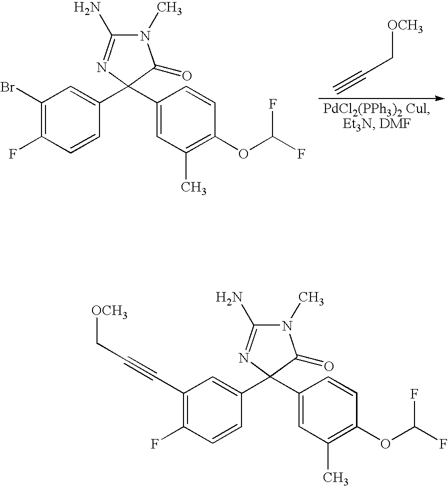

Using essentially the same procedure described in Example 49 and employing 2-amino-4-(3-bromo-4-fluorophenyl)-4-(4-(difluoromethoxy)-3-methylphenyl)-1-methyl-1H-imidazol-5(4H)-one and methyl propargyl ether, the title product was obtained and identified by NMR and mass spectral analyses.

### EXAMPLES 51-56

### Preparation of (5R)-2-Amino-5-[3-(alkynyl)phenyl]-5-[4-(difluoromethoxy)-3-methylphenyl]-3-methyl-3,5-dihydro-4H-imidazol-4-one and (5S)-2-Amino-5-[3-(alkynyl)phenyl]-5-[4-(difluoromethoxy)-3-methylphenyl]-3-methyl-3,5-dihydro-4H-imidazol-4-one Compounds

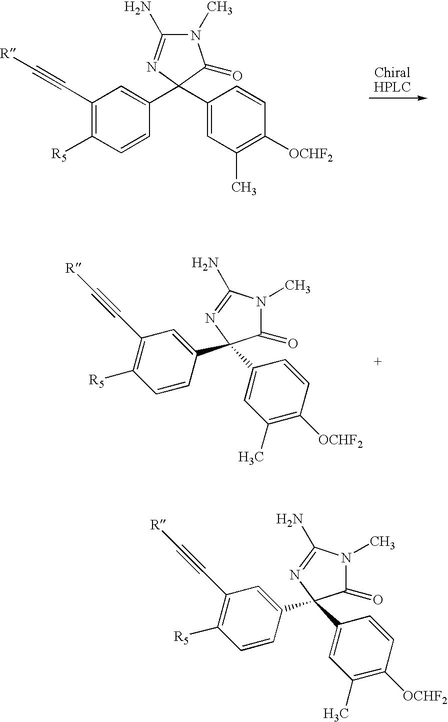

Using essentially the same procedure described in Example 2 and employing the appropriate racemic 2-amino-5-[3-(alkynyl)phenyl]-5-[4-(difluoromethoxy)-3-methylphenyl]-3-methyl-3,5-dihydro-4H-imidazol-4-one, the enantiomeric compounds shown in Table V were obtained and identified by NMR and mass spectral analyses.

TABLE V

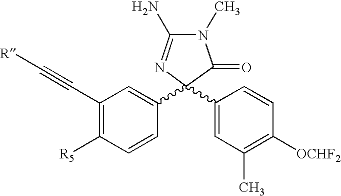

| Ex. No. | Chiral | R″ | R5 | [M + H] | [α]_{D}²⁵* |
|---|---|---|---|---|---|
| 51 | 5-R | cyclopropyl | H | 410.2 | −7.2 |
| 52 | 5-S | cyclopropyl | H | 410.2 | +7.2 |
| 53 | 5-R | cyclopropyl | F | 428.1 | −14 |
| 54 | 5-S | cyclopropyl | F | 428.1 | +15 |
| 55 | 5-S | CH₃OCH₂ | F | 432.4 | −24 |
| 56 | 5-R | CH₃OCH₂ | F | 432.4 | +22 |

*1% Methanol

### EXAMPLE 57

### Preparation of 2-Amino-5-(4-difluoromethoxy-3-methylphenyl)-5-[4-fluoro-3-((E)-3-methoxypropenyl)phenyl]-3-methyl-3,5-dihydro-imidazol-4-one

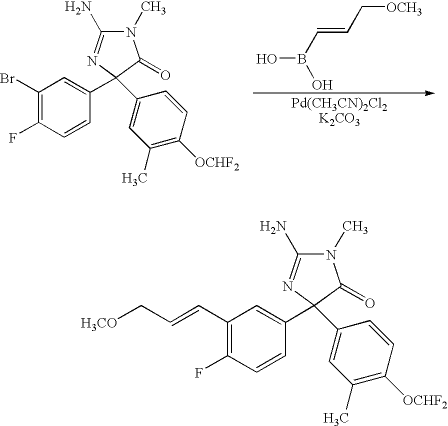

A degassed solution of 2-amino-5-(3-bromo-4-fluorophenyl)-5-[4-(difluoromethoxy)-3-methylphenyl]-3-methyl-1H-imidazol-4-one (1 equiv) and 3-methoxypropen-1-ylboronic acid (1.5 equiv) in a 1:1 mixture of 2 M K₂CO₃ and DME is treated with Pd(CH₃CN)₂Cl₂ (0.05 equiv), heated at 95° C. for 16 h under a nitrogen atmosphere, cooled to room temperature, diluted with water and extracted with CH₂Cl₂. The extracts are combined, washed with brine, dried over Na₂SO₄ and concentrated in vacuo to afford the title product, identified by NMR and mass spectral analyses. ¹H NMR (300 MHz, DMSO-d6) δ ppm 7.63 (dd, 1H), 7.35-7.41 (m, 1H), 7.25-7.35 (m, 2H), 7.13 (dd, 1H), 7.03-7.10 (m, 1H), 7.10 (t, 1H), 6.65-6.81 (m, 3H), 6.25 (dt, 1H), 4.06 (dd, 2H), 3.29 (s, 3H), 2.98 (s, 3H), 2.18 (s, 3H)

### EXAMPLES 58-68

### Preparation of 2-Amino-5-[4-(difluoromethoxy)phenyl]-3-methyl-5-(3-substituted-phenyl)-3,5-dihydro-4H-imidazol-4-one Compounds

Using essentially the same procedure described in Example 57 and employing the appropriate 2-amino-5-(3-bromophenyl)-5-[4-(difluoromethoxy)-3-methylphenyl]-3-methyl-1H-imidazol-4-one and desired alkenylboronic acid, the compounds shown below were obtained and identified by NMR and mass spectral analyses.

### EXAMPLE 58

### 2-Amino-5-[4-(difluoromethoxy)-3-methylphenyl]-5-[4-fluoro-3-((E)-4-fluorobut-1-enyl)phenyl]-3-methyl-3,5-dihydro-4H-imidazol-4-one

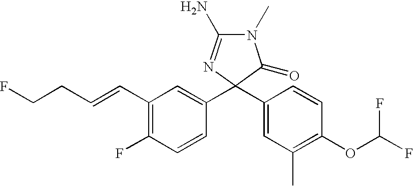

¹H NMR (300 MHz, DMSO-d6) δ ppm 7.62 (dd, 1H), 7.21-7.46 (m, 3H), 7.12 (dd, 1H), 7.07 (d, 1H), 7.10 (t, 1H), 6.68 (br. s., 2H), 6.58 (d, 1H), 6.22 (dt, 1H), 4.56 (dt, 2H), 2.98 (s, 3H), 2.53-2.70 (m, 2H), 2.17 (s, 3H)

### EXAMPLE 59

### 2-Amino-5-[4-(difluoromethoxy)-3-methylphenyl]-5-[4-fluoro-3-((E)-prop-1-enyl)phenyl]-3-methyl-3,5-dihydro-4H-imidazol-4-one

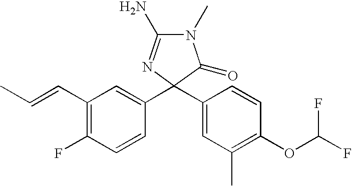

¹H NMR (300 MHz, DMSO-d6) δ ppm 7.58 (dd, 1H), 7.22-7.38 (m, 3H), 6.99-7.15 (m, 2H), 7.10 (t, 1H), 6.66 (br. s., 2H), 6.46 (dd, 1H), 6.22 (dq, 1H), 2.98 (s, 3H), 2.18 (s, 3H), 1.87 (dd, 3H)

### EXAMPLE 60

### 2-Amino-5-[4-(difluoromethoxy)-3-methylphenyl]-5-[4-fluoro-3-((E)-4-methoxy-but-1-enyl)phenyl]-3-methyl-3,5-dihydro-4H-imidazol-4-one

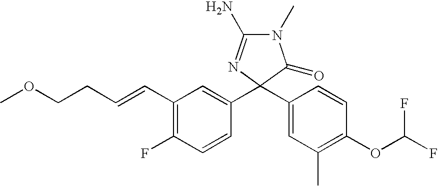

¹H NMR (300 MHz, DMSO-d6) δ ppm 7.60 (dd, 1H), 6.82-7.41 (m, 6H), 6.68 (br. s., 2H), 6.51 (d, 1H), 6.21 (dt, 1H), 3.44 (t, 2H), 3.25 (s, 3H), 2.98 (s, 3H), 2.39-2.48 (m, 2H), 2.18 (s, 3H)

### EXAMPLE 61

### 2-Amino-5-[4-(difluoromethoxy)-3-methylphenyl]-3-methyl-5-[((E)-3-prop-1-enyl)phenyl]-3,5-dihydro-4H-imidazol-4-one

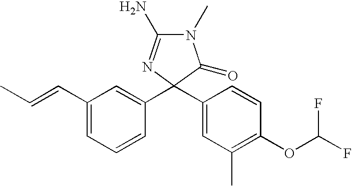

¹H NMR (300 MHz, DMSO-d6) δ ppm 7.17-7.47 (m, 6H), 7.06 (d, 1H), 7.10 (t, 1H), 6.63 (br. s., 2H), 6.37 (dd, 1H), 6.18 (dq, 1H), 2.98 (s, 3H), 2.17 (s, 3H), 1.82 (dd, 3H)

### EXAMPLE 62

### 2-Amino-5-[4-(difluoromethoxy)-3-methylphenyl]-5-[3-((E)-3-methoxyprop-1-enyl)phenyl]-3-methyl-3,5-dihydro-4H-imidazol-4-one

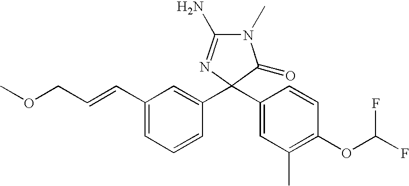

¹H NMR (300 MHz, DMSO-d6) d ppm 7.44-7.56 (m, 1H), 7.17-7.44 (m, 5H), 7.07 (d, 1H), 7.10 (t, 1H), 6.64 (br. s., 2H), 6.56 (d, 1H), 6.22 (dt, 1H), 4.02 (dd, 2H), 3.27 (s, 3H), 2.98 (s, 3H), 2.18 (s, 3H)

### EXAMPLE 63

### 2-Amino-5-[4-(difluoromethoxy)-3-methylphenyl]-5-[3-((E)-4-fluorobut-1-enyl)phenyl]-3-methyl-3,5-dihydro-4H-imidazol-4-one

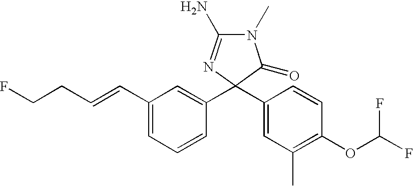

¹H NMR (300 MHz, DMSO-d6) δ ppm 7.42-7.51 (m, 1H), 7.17-7.37 (m, 5H), 7.07 (d, 1H), 7.10 (t, 1H), 6.64 (br. s., 2H), 6.47 (d, 1H), 6.17 (dt, 1H), 4.54 (dt, 2H), 2.98 (s, 3H), 2.52-2.67 (m, 2H), 2.17 (s, 3H)

### EXAMPLE 64

### 2-Amino-5-[4-(difluoromethoxy)-3-methylphenyl]-5-[3-((E)-4-methoxybut-1-enyl)phenyl]-3-methyl-3,5-dihydro-4H-imidazol-4-one

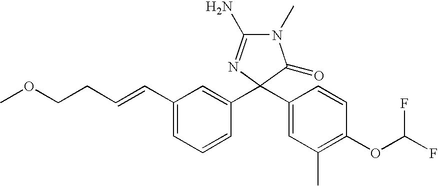

¹H NMR (300 MHz, DMSO-d6) δ ppm 7.39-7.46 (m, 1H), 7.15-7.38 (m, 5H), 7.06 (d, 1H), 7.10 (t, 1H), 6.66 (s, 2H), 6.40 (d, 1H), 6.13 (dt, 1H), 3.43 (t, 2H), 3.24 (s, 3H), 2.98 (s, 3H), 2.39 (dt, 2H), 2.17 (s, 3H)

### EXAMPLE 65

### 2-Amino-5-[3-chloro-4-(difluoromethoxy)phenyl]-3-methyl-5-[((E)-3-prop-1-enyl)phenyl]-3,5-dihydro-4H-imidazol-4-one

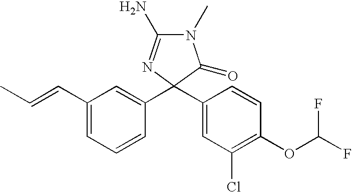

¹H NMR (300 MHz, DMSO-d6) δ ppm 7.59 (d, 1H), 7.48 (dd, 1H), 6.91-7.46 (m, 6H), 6.77 (br. s., 2H), 6.38 (dq, 1H), 6.20 (dq, 1H), 2.99 (s, 3H), 1.83 (dd, 3H)

### EXAMPLE 66

### 2-Amino-5-[3-chloro-4-(difluoromethoxy)phenyl]-5-[3-((E)-3-methoxyprop-1-enyl)phenyl]-3-methyl-3,5-dihydro-4H-imidazol-4-one

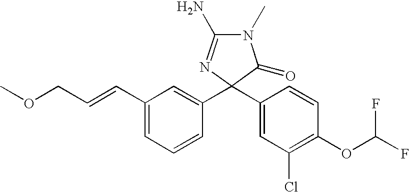

¹H NMR (300 MHz, DMSO-d6) δ ppm 7.60 (d, 1H), 7.49 (dd, 1H), 6.94-7.47 (m, 6H), 6.78 (br. s., 2H), 6.57 (d, 1H), 6.24 (dt, 1H), 4.02 (dd, 2H), 3.23-3.28 (m, 3H), 3.00 (s, 3H)

### EXAMPLE 67

### 2-Amino-5-[3-chloro-4-(difluoromethoxy)phenyl)-5-[3-((E)-4-methoxybut-1-enyl)phenyl]-3-methyl-3,5-dihydro-4H-imidazol-4-one

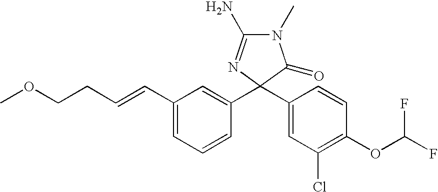

¹H NMR (300 MHz, DMSO-d6) δ ppm 7.59 (d, 1H), 7.48 (dd, 1H), 6.92-7.47 (m, 6H), 6.77 (br. s., 2H), 6.42 (d, 1H), 6.18 (dt, 1H), 3.43 (t, 2H), 3.25 (s, 3H), 2.99 (s, 3H), 2.40 (qd, 2H)

### EXAMPLE 68

### 2-Amino-5-[3-chloro-4-(difluoromethoxy)phenyl)-5-[3-((E)-4-fluoro-but-1-enyl)phenyl]-3-methyl-3,5-dihydro-4H-imidazol-4-one

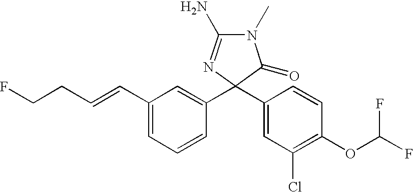

¹H NMR (300 MHz, DMSO-d6) δ ppm 7.59 (d, 1H), 7.48 (dd, 1H), 7.41-7.46 (m, 1H), 7.23-7.38 (m, 4H), 7.22 (d, 1H), 6.77 (br. s., 2H), 6.49 (d, 1H), 6.19 (dt, 1H), 4.54 (dt, 2H), 2.99 (s, 3H), 2.53-268 (m, 2H)

### EXAMPLE 69

### Preparation of (5S)-2-Amino-4-(4-(difluoromethoxy)-3-methylphenyl)-4-(3-(3-methoxyprop-1-ynyl)phenyl)-1-methyl-1H-imidazol-5(4H)-one

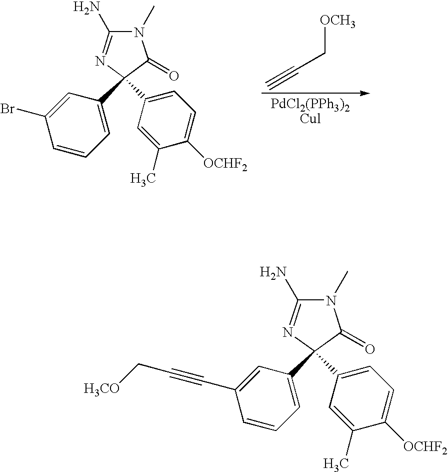

A solution of (5R)-2-amino-4-(3-bromophenyl)-4-(4-(difluoromethoxy)-3-methylphenyl)-1-methyl-1H-imidazol-5(4H)-one (0.25 g, 0.59 mmol) and 1 mL of pyrrolidine in 2 mL of acetonitrile is degassed by bubbling nitrogen through the solution for 15 minutes. While the purging is continuing, methyl propargyl ether (0.16 g, 2.36 mmol, 4 eq) is added followed by PdCl₂(PPh₃)₂(0.041 g, 0.06 mmol, 10 mol %) and CuI (0.006 g, 0.03 mmol, 5 mol %) in that order. The mixture is stirred and heated to 60° C. for 18 h, cooled to room temperature and partitioned between EtOAc and aqueous NaHCO₃ solution. The organic phase was separated, washed sequentially with NaHCO₃ solution and brine, dried over Mg₂SO₄ and concentrated in vacuo. The resultant residue was purified by flash chromatography on the Isco Companion (0-5% DCM:MeOH gradient) to afford the title product as an off white solid, 60% yield, identified by NMR and mass spectral analyses. (MS m/e (M+H)⁺ 414.4), [α]_{D}²⁵=+1 (c=1% in MeOH).

### EXAMPLE 70

### Preparation of (5R)-2-Amino-4-(4-(difluoromethoxy)-3-methylphenyl)-4-(3-(3-methoxyprop-1-ynyl)phenyl)-1-methyl-1H-imidazol-5(4H)-one

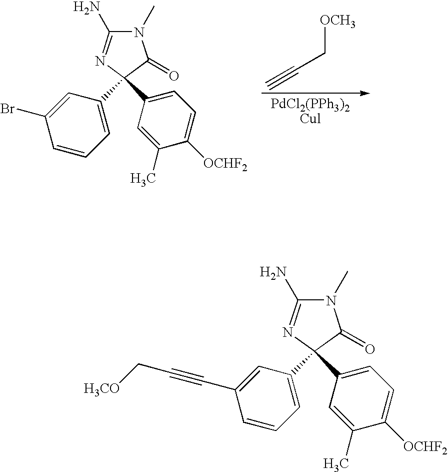

Using essentially the same procedure described in Example 69 and employing (5S)-2-amino-4-(3-bromophenyl)-4-(4-(difluoromethoxy)-3-methylphenyl)-1-methyl-1H-imidazol-5(4H)-one, the title product was obtained as an off-white solid, 65% yield, identified by NMR and mass spectral analyses. (MS m/e (M+H)⁺ 414.4), [α]_{D}²⁵=−1 (c=1% in MeOH).

### EXAMPLE 71

### Preparation of 2-Amino-5,5-bis-[4-(difluoromethoxy)-3-methylphenyl]-3-methyl-3,5-dihydro-4H-imidazol-4-one

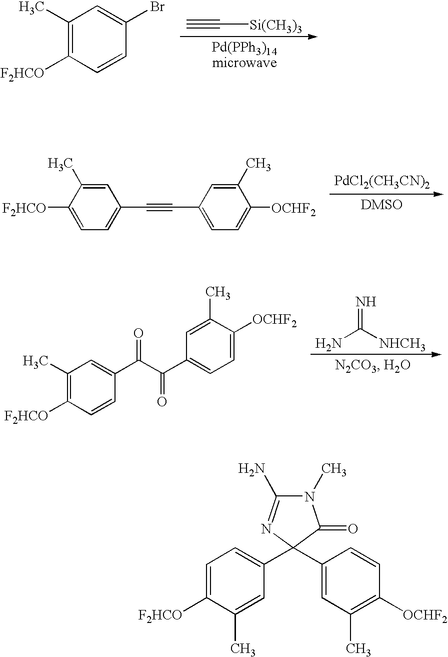

### Step 1: bis(4-difluoromethoxy-3-methyl-phenyl)acetylene

In a CEM snap top microwave vial were combined trimethylsilylacetylene (0.207 g, 2.11 mmol), 4-Bromo-1-difluoromethoxy-2-methyl-benzene (1.00 g, 4.22 mmol), tetrakis(triphenylphosphine)palladium (56 mg, 0.0485 mmol) and pyrrolidine (1 mL, 12 mmol). The reaction vial was placed in a CEM Explorer™ microwave and irradiated for 30 minutes at 80° C. The crude reaction mixture was poured directly onto silica gel and purified by column chromatography (hexanes) to yield 0.519 g of 1,1′-(1,2-ethynediyl)bis[4-difluoromethoxy-3-methylbenzene] as a clear oil (73%).

¹H NMR (400 MHz, DMSO-d₆) δ ppm 2.24 (s, 6H) 7.19 (d, 2H) 7.26 (t, J=73.7 Hz, 2H) 7.44 (q, J=8.6, 2.1 Hz, 2H) 7.51 (d, J=1.4 Hz, 2H); MS (EI) m/z 338 [M⁺.]

### Step 2: 1,2-bis-(4-difluoromethoxy-3-methyl-phenyl)-ethane-1,2-dione

A solution of bis(4-difluoromethoxy-3-methyl-phenyl)acetylene (0.494 g, 1.46 mmol) in DMSO was treated with bis(acetonitrile)dichloropalladium (43 mg, 0.166 mmol), heated for 7 h at 145° C., cooled to room temperature, diluted with water and extracted with dichloromethane. The extracts were combined, dried over magnesium sulfate, and concentrated onto silica gel. This residue was purified by column chromatography (10% EtOAc in hexanes) to afford 1,2-bis-(4-difluoromethoxy-3-methyl-phenyl)-ethane-1,2-dione as an oil, 0.447, ¹H NMR (400 MHz, DMSO-d₆) δ ppm 2.29 (s, 6H) 7.35 (d, J=8.6 Hz, 2H) 7.42 (t, J=74.2 Hz, 2H) 7.82 (dd, J=8.6, 2.3 Hz, 2H) 7.90 (d, 2H); MS (APPI) m/z 371 [M−H]⁻

### Step 3: 2-Amino-5,5-bis-(4-difluoromethoxy-3-methyl-phenyl)-3-methyl-3,5-dihydro-imidazol-4-one

A solution of 1,2-bis-[4-(difluoromethoxy)-3-methylphenyl]ethane-1,2-dione (0.367 g, 0.991 mmol) in isopropanol was treated with methylguanidine hydrochloride (0.163 g, 1.48 mmol), followed by sodium carbonate (0.157 g, 105.99 g/mol, 1.48 mmol), heated at 86° C. for 14 h and concentrated in vacuo. The resultant residue was partitioned between water and chloroform. The organic phase was separated, dried over sodium sulfate and concentrated onto silica gel. This residue was purified by column chromatography [step gradient; 1:1 (EtOAc/hexanes) then 100% EtOAc] to afford a clear oil, 0.300 gm. This oil was redissolved in diethylether and concentrated, twice then placed under vacuum to give the title product as a white foam (71%), identified by NMR and mass spectral analyses. ¹H NMR (400 MHz, DMSO-d₆) δ ppm 2.17 (s, 6H) 2.96 (s, 3H) 6.68 (s, 2H) 7.06 (d, J=8.4 Hz, 2H) 7.11 (t, J=74.2 Hz, 2H) 7.30 (dd, J=8.6, 2.3 Hz, 2H) 7.34 (d, 2H); MS (ES) m/z 424 [M−H]⁻

### EXAMPLE 72

### Preparation of: (5R)-2-amino-5-[4-(difluoromethoxy)-3-methylphenyl]-5-(4-fluoro-3-pent-1-ylphenyl)-3-methyl-3,5-dihydro-4H-imidazol-4-one

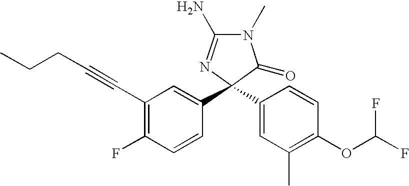

A racemic mixture of 2-amino-5-[4-(difluoromethoxy)-3-methylphenyl]-5-(4-fluoro-3-pent-1-yn-1-ylphenyl)-3-methyl-3,5-dihydro-4H-imidazol-4-one (0.625 g, 1.46 mmol) was separated by chiral chromatography (Chiralpak OD 2×25 cm Mobile phase 30% MeOH/DEA in CO2) to provide peak 2, RT=3.41 min, (5R)-2-amino-5-[4-(difluoromethoxy)-3-methylphenyl]-5-(4-fluoro-3-pent-1-yn-1-ylphenyl)-3-methyl-3,5-dihydro-4H-imidazol-4-one (0.267 g, 0.622 mmol, 43% yield) as a white solid.

MS m/e (M+H)⁺ 430.2, [α]_{D}²⁵=−12.8 (c=1% in MeOH)

### EXAMPLE 73

### Preparation of: (5S)-2-amino-5-[4-(difluoromethoxy)-3-methylphenyl]-5-(4-fluoro-3-pent-1-yn-1-ylphenyl)-3-methyl-3,5-dihydro-4H-imidazol-4-one

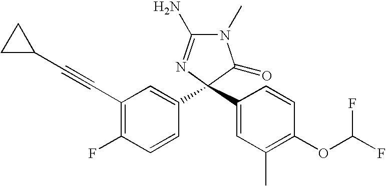

A racemic mixture of 2-amino-5-[4-(difluoromethoxy)-3-methylphenyl]-5-(4-fluoro-3-pent-1-yn-1-ylphenyl)-3-methyl-3,5-dihydro-4H-imidazol-4-one (0.625 g, 1.46 mmol) was separated by chiral chromatography (Chiralpak OD 2×25 cm Mobile phase 30% MeOH/DEA in CO2) to provide peak 1, RT=3.055 min, (5S)-2-amino-5-[4-(difluoromethoxy)-3-methylphenyl]-5-(4-fluoro-3-pent-1-yn-1-ylphenyl)-3-methyl-3,5-dihydro-4H-imidazol-4-one (0.289 g, 0.673 mmol, 46% yield) as a white solid.

MS m/e (M+H)⁺ 430.2, [α]_{D}²⁵=+14 (c=1% in MeOH)

### EXAMPLE 74

### Preparation of: 2-amino-5-[4-(difluoromethoxy)-3-(2-fluoroethyl)phenyl]-3-methyl-5-phenyl-3,5-dihydro-4H-imidazol-4-one

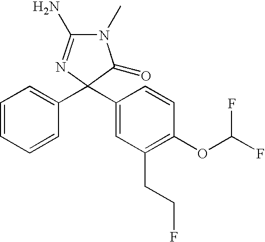

### Step 1:4-bromo-2-(2-hydroxyethyl)phenol

In a 1 L round-bottomed flask was placed 2-(2-hydroxyethyl)phenol (50 g, 362 mmol) and THF (724 mL) was added to give a brown solution. The reaction was cooled to-25° C. Sulfuric acid (0.964 mL, 18.09 mmol) was added. N-Bromosuccinimide (70.9 g, 398 mmol) was added and reaction stirred for 1 h at −25° C. and then allowed to warm to RT overnight. A 10% sodium thiosulfite solution was added (100 mL). EtOAc (500 mL) was added and the organic was washed with water (2×200 mL) and brine (200 mL). The organic layer was dried over Na2SO4. The crude material was purified by flash chromatography (0-100% EtOAc/hex) to provide the desired product in quantitative yield and used directly in subsequent reaction.

¹H NMR (400 MHz, DMSO-d₆) [symbol] 9.60 (s, 1H), 7.20 (s, 1H), 7.15 (d, J=8.0 Hz, 1H), 6.71 (d, J=8.0 Hz, 1H), 4.65 (b, 1H), 3.55 (t, J=6.8 Hz, 2H), 2.64 (t, J=7.0 Hz, 2H)

### Step 2: 2-(5-bromo-2-(difluoromethoxy)phenyl)ethanol

In a 1 L round-bottomed flask was placed 4-bromo-2-(2-hydroxyethyl)phenol (79 g, 362 mmol) and DMF (326 mL) and water (36.2 mL) were added to give a colorless solution. Potassium carbonate (200 g, 1448 mmol) was added. Sodium 2-chloro-2,2-difluoroacetate (60.7 g, 398 mmol) was added. The reaction was warmed to 120° C. overnight. The reaction was allowed to cool. The solution was diluted with EtOAc (1 L) and washed with water (500 mL). The organic was washed with brine (3×300 mL). The organic layer was dried over Na2SO4. The crude material was purified by flash chromatography (0-100% EtOAc/hex) to provide 2-(5-bromo-2-(difluoromethoxy)phenyl)ethanol (30 g, 112 mmol, 31.0% yield) as a clear oil.

¹H NMR (400 MHz, DMSO-d₆) [symbol] 7.49 (d, J=8.7 Hz, 1H), 7.41 (t, J_{H-F}=74 Hz, 1H), 7.07 (d, J=8.7 Hz, 1H), 4.67 (b, 1H), 3.55 (t, J=6.8 Hz, 2H), 2.70 (t, J=7.0 Hz, 2H)

### Step 3: 4-bromo-1-(difluoromethoxy)-2-(2-fluoroethyl)benzene

In a 250 mL round-bottomed flask was placed 2-(5-bromo-2-(difluoromethoxy)phenyl)ethanol (20 g, 74.9 mmol) and CH₂Cl₂ (150 mL) was added to give a light orange solution. The solution was cooled to −40° C. DAST (11.87 mL, 90 mmol) was added. The reaction was allowed to gradually warm to RT. Solvent was removed and crude material purified by flash chromatography (0-60% EtOAc/hex) to provide 4-bromo-1-(difluoromethoxy)-2-(2-fluoroethyl)benzene (7.69 g, 28.6 mmol, 38.2% yield) as a light yellow oil.

¹H NMR (400 MHz, DMSO-d₆) [symbol] 7.59 (s, 1H), 7.51 (d, J=8.7 Hz, 1H), 7.20 (t, J_{H-F}=74 Hz, 1H), 7.14 (d, J=8.7 Hz, 1H), 4.62 (dt, JH-F=57.1, J=6.3 Hz, 2H), 3.00 (dt, JH-F=24.3, J=6.3 Hz, 2H)

### Step 4: 1-(difluoromethoxy)-2-(2-fluoroethyl)-4-(phenylethynyl)benzene

In a 50 mL round-bottomed flask was placed 4-bromo-1-(difluoromethoxy)-2-(2-fluoroethyl)benzene (1 g, 3.72 mmol) and acetonitrile (4.5 mL) and pyrrolidine (3.0 mL) were added to give a colorless solution. Ethynylbenzene (0.490 mL, 4.46 mmol) was added was added and N2 was bubbled through reaction for 20 min. Bis(triphenylphosphine)palladium(II) chloride (0.261 g, 0.372 mmol) was added and N2 bubbling continued. Copper(I) iodide (0.035 g, 0.186 mmol) was added and reaction heated to 60° C. for 3 h. The reaction was cooled to RT. The reaction was partitioned between ether (50 mL) and 1M HCl (25 mL). The organic was washed with 1M HCl (25 mL) and brine (25 mL). The organic layer was dried over Na₂SO₄. The crude material was dried onto silica. The crude was purified by flash chromatography (gradient 0-10% EtOAc/hex) to provide 1-(difluoromethoxy)-2-(2-fluoroethyl)-4-(phenylethynyl)benzene (0.39 g, 1.344 mmol, 36.1% yield) as an orange oil.

¹H NMR (400 MHz, DMSO-d₆) [symbol] 7.35-7.55 (m, 7H), 7.27 (t, JH-F=74 Hz, 1H), 7.21 (d, J=8.5 Hz, 1H), 4.62 (dt, JH-F=57.1, J=6.3 Hz, 2H), 3.01 (dt, JH-F=24.3, J=6.3 Hz, 2H)

### Step 5: 1-(4-(difluoromethoxy)-3-(2-fluoroethyl)phenyl)-2-phenylethane-1,2-dione

In a 50 mL round-bottomed flask was placed 1-(difluoromethoxy)-2-(2-fluoroethyl)-4-(phenylethynyl)benzene (0.39 g, 1.344 mmol) and DMSO (2.69 mL) was added to give a orange solution. Palladiumdichlorobisacetonitrile (0.035 g, 0.134 mmol) was added and reaction was heated to 120° C. for 18 h until complete by LC/MS. The reaction was cooled to RT. The reaction was partitioned between EtOAc (70 mL) and water (50 mL). The organic was washed with water (2×50 mL) and brine (2×50 mL). The organic layer was dried over Na₂SO₄. The crude material was purified by flash chromatography (gradient 0-20% EtOAc/hex) to providel-(4-(difluoromethoxy)-3-(2-fluoroethyl)phenyl)-2-phenylethane-1,2-dione (0.266 g, 0.825 mmol, 61.4% yield) as a orange solid.

¹H NMR (400 MHz, DMSO-d₆) [symbol] 7.35-7.60 (m, 7H), 7.27 (t, JH-F=74 Hz, 1H), 7.21 (d, J=8.5 Hz, 1H), 4.62 (dt, JH-F=57.1, J=6.3 Hz, 2H), 3.01 (dt, JH-F=24.3, J=6.3 Hz, 2H)

### Step 6: 2-amino-4-(4-(difluoromethoxy)-3-(2-fluoroethyl)phenyl)-1-methyl-4-phenyl-1H-imidazol-5(4H)-one

In a 50 mL round-bottomed flask was placed 1-(4-(difluoromethoxy)-3-(2-fluoroethyl)phenyl)-2-phenylethane-1,2-dione (0.26 g, 0.807 mmol) and ethanol (3.23 mL) was added to give a yellow solution. Sodium carbonate (0.086 g, 0.807 mmol) and 1-methylguanidine hydrochloride (0.088 g, 0.807 mmol) were added and reaction heated to 80° C. The reaction was heated for 4 h and then cooled. The solvent was removed. The crude material was purified by flash chromatography (gradient 0-10% MeOH/CH2Cl2) to provide 2-amino-4-(4-(difluoromethoxy)-3-(2-fluoroethyl)phenyl)-1-methyl-4-phenyl-1H-imidazol-5(4H)-one (0.19 g, 0.503 mmol, 62.4% yield) as an off white solid.

MS m/e (M+H)⁺ 378.2

### EXAMPLE 75

### Preparation of: (5R)-2-amino-4-(4-(difluoromethoxy)-3-(2-fluoroethyl)phenyl)-1-methyl-4-phenyl-1H-imidazol-5(4H)-one

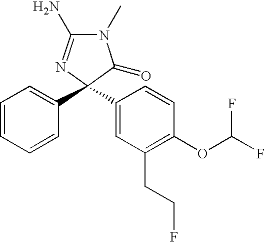

A racemic mixture of 2-amino-4-(4-(difluoromethoxy)-3-(2-fluoroethyl)phenyl)-1-methyl-4-phenyl-1H-imidazol-5(4H)-one (0.160 g, 0.424 mmol) was separated by chiral chromatography (Chiralpak OJ 2×25 cm Mobile phase 12% EtOH/DEA in hexane/DEA) to provide peak 1, RT=5.68 min, (5R)-2-amino-4-(4-(difluoromethoxy)-3-(2-fluoroethyl)phenyl)-1-methyl-4-phenyl-1H-imidazol-5(4H)-one (0.053 g, 0.14 mmol, 33% yield) as a white solid.

MS m/e (M+H)⁺ 378.2, [α]_{D}²⁵=+16.6 (c=1% in MeOH)

### EXAMPLE 76

### Preparation of: (5S)-2-amino-4-(4-(difluoromethoxy)-3-(2-fluoroethyl)phenyl)-1-methyl-4-phenyl-1H-imidazol-5(4H)-one

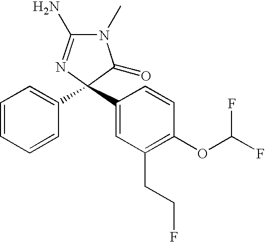

A racemic mixture of 2-amino-4-(4-(difluoromethoxy)-3-(2-fluoroethyl)phenyl)-1-methyl-4-phenyl-1H-imidazol-5(4H)-one (0.160 g, 0.424 mmol) was separated by chiral chromatography (Chiralcel OJ 2×25 cm Mobile phase 12% EtOH/DEA in hexane/DEA) to provide peak 2, RT=6.49 min, (5S)-2-amino-4-(4-(difluoromethoxy)-3-(2-fluoroethyl)phenyl)-1-methyl-4-phenyl-1H-imidazol-5(4H)-one (0.048 g, 0.13 mmol, 31% yield) as a white solid.

MS m/e (M+H)⁺ 378.2, [α]_{D}²⁵=−17.4 (c=1% in MeOH)

### EXAMPLE 77

### Preparation of: 2-amino-5-[4-(difluoromethoxy)-3-methylphenyl]-3-methyl-5-(3-methylphenyl)-3,5-dihydro-4H-imidazol-4-one

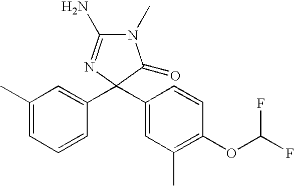

### Step 1: 1-(difluoromethoxy)-2-(2-fluoroethyl)-4-(phenylethynyl)benzene

In a 50 mL round-bottomed flask was placed 4-bromo-1-(difluoromethoxy)-2-methylbenzene (0.8 g, 3.37 mmol) and DMF (6.75 mL) was added to give a colorless solution. 1-Ethynyl-3-methylbenzene (0.436 mL, 3.37 mmol) and triethylamine (2.352 mL, 16.87 mmol) were added. The reaction was degassed by bubbling with N2. Bis(triphenylphosphine)palladium(II) chloride (0.118 g, 0.169 mmol) was added and N2 bubbling was continued. Copper iodide (0.032 g, 0.169 mmol) was added. The reaction was heated to 60° C. for 12 h. The reaction was cooled and partitioned between ether (70 mL) and 1 MHCl (50 mL). The organic was washed with 1M HCl (50 mL) and brine (2×50 mL). The organic layer was dried over Na₂SO₄. The crude was purified by flash chromatography (100% hexanes) to provide 1-(difluoromethoxy)-2-methyl-4-(m-tolylethynyl)benzene (0.88 g, 3.23 mmol, 96% yield) as a yellow oil with minor impurities. ¹H NMR (400 MHz, DMSO-d₆) [symbol] 7.05-7.70 (m, 7H), 7.18 (t, J_{H-F}=74 Hz, 1H), 2.30 (s, 3H), 2.20 (s, 3H)

### Step 2: 1-(4-(difluoromethoxy)-3-methylphenyl)-2-m-tolylethane-1,2-dione

In a 50 mL round-bottomed flask was placed 1-(difluoromethoxy)-2-methyl-4-(m-tolylethynyl)benzene (0.88 g, 3.23 mmol) and DMSO (6.46 mL) was added to give a yellow solution. Palladiumdichlorobisacetonitrile (0.084 g, 0.323 mmol) was added. The reaction was heated to 120° C. for 4 h. The reaction was partitioned between EtOAc (50 mL) and water (20 mL). The organic was washed with water (20 mL) and brine (2×20 mL). The organic layer was dried over Na2SO4. The crude material was purified by flash chromatography (0-40% EtOAc/hex) to provide 1-(4-(difluoromethoxy)-3-methylphenyl)-2-m-tolylethane-1,2-dione (0.4 g, 1.315 mmol, 40.7% yield) as an yellow solid.

¹H NMR (400 MHz, DMSO-d₆) [symbol] 7.87 (s, 1H), 7.79 (d, J=8.5 Hz, 1H), 7.70 (s, 1H), 7.66 (d, J=7.7 Hz, 1H), 7.58 (d, J=7.6 Hz, 1 h), 7.48 (T, j=7.7 Hz, 1H), 7.39 (t, J_{H-F}=74 Hz, 1H), 7.32 (d, J=8.6 Hz, 1H), 2.35 (s, 3H), 2.26 (s, 3H)

### Step 3: 2-amino-5-[4-(difluoromethoxy)-3-methylphenyl]-3-methyl-5-(3-methylphenyl)-3,5-dihydro-4H-imidazol-4-one

In a 50 mL round-bottomed flask was placed 1-(4-(difluoromethoxy)-3-(2-fluoroethyl)phenyl)-2-phenylethane-1,2-dione (0.26 g, 0.807 mmol) and ethanol (3.23 mL) was added to give a yellow solution. Sodium carbonate (0.086 g, 0.807 mmol) and 1-methylguanidine hydrochloride (0.088 g, 0.807 mmol) were added and reaction heated to 80° C. The reaction was heated for 4 h and then cooled. The solvent was removed. The crude material was purified by flash chromatography (gradient 0-10% MeOH/CH2Cl2) to provide 2-amino-4-(4-(difluoromethoxy)-3-(2-fluoroethyl)phenyl)-1-methyl-4-phenyl-1H-imidazol-5(4H)-one (0.19 g, 0.503 mmol, 62.4% yield) as an off white solid.

MS m/e (M+H)⁺ 360.2

### EXAMPLE 78

### Preparation of: (5S)-2-amino-5-[4-(difluoromethoxy)-3-methylphenyl]-3-methyl-5-(3-pent-1-yn-1-ylphenyl)-3,5-dihydro-4H-imidazol-4-one

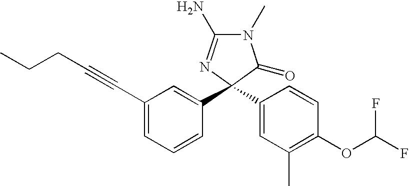

In a 5 mL vial was placed (R)-2-amino-4-(3-bromophenyl)-4-(4-(difluoromethoxy)-3-methylphenyl)-1-methyl-1H-imidazol-5(4H)-one (0.2 g, 0.471 mmol) and acetonitrile (0.566 mL) and pyrrolidine (0.377 mL) was added to give a colorless solution. The solution was degassed with N2 bubbling for 20 min. Pent-1-yne (0.070 mL, 0.707 mmol) was added. Bistriphenylphosphinedichloropalladium (0.017 g, 0.024 mmol) was added. Copper(I) iodide (4.49 mg, 0.024 mmol) was added. The reaction was warmed to 60° C. for 2 h. An additional amount of pent-1-yne (0.070 mL, 0.707 mmol) was added and reaction continued for another 2 h. Solvent was removed. The crude material was partitioned between EtOAc (10 mL) and sat NaHCO₃ (5 mL). The organic was washed with water. The organic layer was dried over Na₂SO₄. Solvent was removed and crude material purified by flash chromatography (gradient 10% MeOH/CH₂Cl₂) to provide (S)-2-amino-4-(4-(difluoromethoxy)-3-methylphenyl)-1-methyl-4-(3-(pent-1-ynyl)phenyl)-1H-imidazol-5(4H)-one (0.115 g, 0.280 mmol, 59.3% yield) as a white solid.

MS m/e (M+H)⁺ 412.1, [α]_{D}²⁵=+3.6 (c=1% in MeOH)

### EXAMPLE 79

### Preparation of: (5S)-2-amino-5-[4-(difluoromethoxy)-3-propylphenyl]-3-methyl-5-phenyl-3,5-dihydro-4H-imidazol-4-one

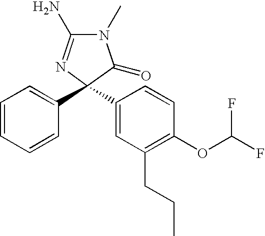

A racemic mixture of 2-amino-5-[4-(difluoromethoxy)-3-propylphenyl]-3-methyl-5-phenyl-3,5-dihydro-4H-imidazol-4-one (0.211 g, 0.424 mmol) was separated by chiral chromatography (Chiralpak OJ 2×25 cm Mobile phase 8% iPrOH/DEA in hexane/DEA) to provide peak 1, RT=7.05 min, (5R)-2-amino-4-(4-(difluoromethoxy)-3-(2-fluoroethyl)phenyl)-1-methyl-4-phenyl-1H-imidazol-5(4H)-one (0.08 g, 0.21 mmol, 38% yield) as a white solid.

MS m/e (M+H)⁺ 374.2, [α]_{D}²⁵=+15.2 (c=1% in MeOH)

### EXAMPLE 80

### Preparation of: (5R)-2-amino-5-[4-(difluoromethoxy)-3-propylphenyl]-3-methyl-5-phenyl-3,5-dihydro-4H-imidazol-4-one

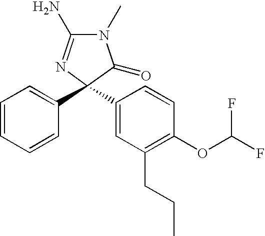

A racemic mixture of 2-amino-5-[4-(difluoromethoxy)-3-propylphenyl]-3-methyl-5-phenyl-3,5-dihydro-4H-imidazol-4-one (0.21 μg, 0.424 mmol) was separated by chiral chromatography (Chiralcel OJ 2×25 cm Mobile phase 8% iPrOH/DEA in hexane/DEA) to provide peak 2, RT=9.02 min, (5S)-2-amino-4-(4-(difluoromethoxy)-3-(2-fluoroethyl)phenyl)-1-methyl-4-phenyl-1H-imidazol-5(4H)-one (0.078 g, 0.21 mmol, 37% yield) as a white solid.

MS m/e (M+H)⁺ 374.2, [α]_{D}²⁵=−14.0 (c=1% in MeOH)

Using the procedures described above, the enantiomeric compounds shown in Table VI were obtained and identified by mass spectral analyses.

TABLE VI

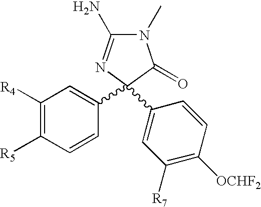

| Ex. No. | Chiral | R7 | [M + H]⁺ | [α]_{D}²⁵* |
|---|---|---|---|---|
| 75 | 5-S | CH₂CH₂F | 378.1 | +16.6 |
| 76 | 5-R | CH₂CH₂F | 378.1 | −17.4 |
| 79 | 5-S | propyl | 374.2 | +15.2 |
| 80 | 5-R | propyl | 374.2 | −14.0 |

### EXAMPLE 81

### Preparation of: 2-amino-5-[4-(difluoromethoxy)-3-(2-fluoroethyl)phenyl]-3-methyl-5-phenyl-3,5-dihydro-4H-imidazol-4-one

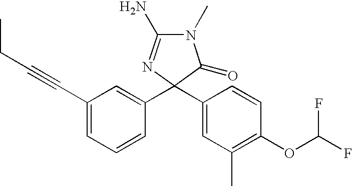

### Step 1: 1-(3-(but-1-ynyl)phenyl)-2-(4-(difluoromethoxy)-3-methylphenyl)ethane-1,2-dione

In a 50 mL round-bottomed flask was placed 1-(3-bromophenyl)-2-(4-(difluoromethoxy)-3-methylphenyl)ethane-1,2-dione (0.64 g, 1.734 mmol) and DMF (4.16 mL) and Et₃N (2.77 mL) were added to give a yellow solution. The reaction was degassed by bubbling with N2. but-1-yne (0.094 g, 1.734 mmol) was added by bubbling. Bis(triphenylphosphine)palladium(II) chloride (0.061 g, 0.087 mmol) was added. Copper(I) iodide (0.017 g, 0.087 mmol) was added. The reaction was sealed and warmed to 60° C. The reaction was diluted with EtOAc (30 mL) and washed with 1M HCl (20 mL). The organic was washed with brine (3×10 mL). The organic layer was dried over Na2SO4. The crude material was purified by flash chromatography (0-40% EtOAc/hex) to provide 1-(3-(but-1-ynyl)phenyl)-2-(4-(difluoromethoxy)-3-methylphenyl)ethane-1,2-dione (0.25 g, 0.730 mmol, 42.1% yield) as a yellow solid.

### Step 2: 2-amino-4-(3-(but-1-ynyl)phenyl)-4-(4-(difluoromethoxy)-3-methylphenyl)-1-methyl-1H-imidazol-5(4H)-one

In a 25 mL round-bottomed flask was placed 1-(3-(but-1-ynyl)phenyl)-2-(4-(difluoromethoxy)-3-methylphenyl)ethane-1,2-dione (0.2 g, 0.584 mmol) and Ethanol (4.67 mL) was added to give a orange solution. Sodium carbonate (0.062 g, 0.584 mmol) and 1-methylguanidine hydrochloride (0.064 g, 0.584 mmol) were added. The reaction was heated to 80° C. for 4 h. The solvent was removed and crude material purified by flash chromatography (0-10% MeOH/CH₂Cl₂) to provide 2-amino-4-(3-(but-1-ynyl)phenyl)-4-(4-(difluoromethoxy)-3-methylphenyl)-1-methyl-1H-imidazol-5(4H)-one (0.13 g, 0.327 mmol, 56.0% yield) as a white solid.

MS m/e (M+H)⁺ 398.2

### EXAMPLE 82

### Preparation of: 2-amino-5-(3-but-1-yn-1-yl-4-fluorophenyl)-5-[4-(difluoromethoxy)-3-methylphenyl]-3-methyl-3,5-dihydro-4H-imidazol-4-one

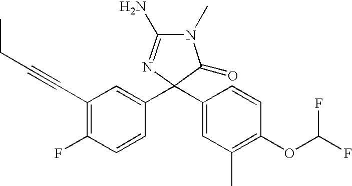

In a 50 mL round-bottomed flask was placed 2-amino-4-(3-bromo-4-fluorophenyl)-4-(4-(difluoromethoxy)-3-methylphenyl)-1-methyl-1H-imidazol-5(4H)-one (1.2 g, 2.71 mmol) and acetonitrile (6.51 mL) and pyrrolidine (4.34 mL) were added to give a colorless solution. The reaction was flushed with N2. The reaction was cooled to 0° C. Bis(triphenylphosphine)palladium(II) chloride (0.095 g, 0.136 mmol) was added. Copper(I) iodide (0.026 g, 0.136 mmol) was added. But-1-yne was bubbled through reaction. The reaction was allowed to warm to RT. After 24 the reaction was not complete and but-1-yne was bubbled again through reaction. After 12 the reaction was complete. Solvent was removed in vacuo. The crude material was purified by flash chromatography (0-10% MeOH/CH2Cl2) providing a mixture of two products. The mixture was purified by reverse phase chromatography utilizing the Gilson HPLC and the Gemini 30×50 column 10-100% acetonitrile/water (0.5% NH4OH) to isolate two products. The first peak corresponded to desired product 2-amino-4-(3-(but-1-ynyl)-4-fluorophenyl)-4-(4-(difluoromethoxy)-3-methylphenyl)-1-methyl-1H-imidazol-5(4H)-one (0.225 g, 0.542 mmol, 19.96% yield).

MS m/e (M+H)⁺ 416.1

### EXAMPLE 83

### Preparation of: (5R)-2-amino-5-(3-but-1-yn-1-yl-4-fluorophenyl)-5-[4-(difluoromethoxy)-3-methylphenyl]-3-methyl-3,5-dihydro-4H-imidazol-4-one

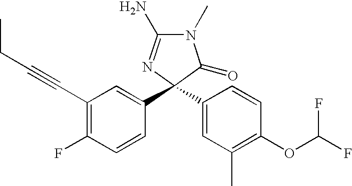

A racemic mixture of 2-amino-5-(3-but-1-yn-1-yl-4-fluorophenyl)-5-[4-(difluoromethoxy)-3-methylphenyl]-3-methyl-3,5-dihydro-4H-imidazol-4-one (0.200 g, 0.424 mmol) was separated by chiral chromatography (Chiralpak AD-H 2×25 cm Mobile phase 5% MeOH/EtOH/DEA in hexane/DEA) to provide peak 1, RT=8.027 min, (5R)-2-amino-5-(3-but-1-yn-1-yl-4-fluorophenyl)-5-[4-(difluoromethoxy)-3-methylphenyl]-3-methyl-3,5-dihydro-4H-imidazol-4-one (0.08 g, 0.19 mmol, 42% yield) as a white solid.

MS m/e (M+H)⁺ 416.3, [α]_{D}²⁵=−15.8 (c=1% in MeOH)

### EXAMPLE 84

### Preparation of: (5S)-2-amino-5-(3-but-1-yn-1-yl-4-fluorophenyl)-5-[4-(difluoromethoxy)-3-methylphenyl]-3-methyl-3,5-dihydro-4H-imidazol-4-one

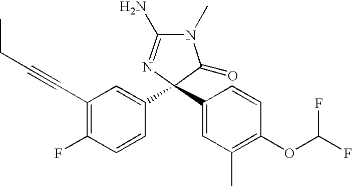

A racemic mixture of 2-amino-5-(3-but-1-yn-1-yl-4-fluorophenyl)-5-[4-(difluoromethoxy)-3-methylphenyl]-3-methyl-3,5-dihydro-4H-imidazol-4-one (0.211 g, 0.424 mmol) was separated by chiral chromatography (Chiralpak AD-H 2×25 cm Mobile phase 5% MeOH/EtOH/DEA in hexane/DEA) to provide peak 2, RT=10.07 min, (5S)-2-amino-5-(3-but-1-yn-1-yl-4-fluorophenyl)-5-[4-(difluoromethoxy)-3-methylphenyl]-3-methyl-3,5-dihydro-4H-imidazol-4-one (0.075 g, 0.18 mmol, 40% yield) as a white solid.

MS m/e (M+H)⁺ 416.3, [α]_{D}²⁵=+16.6 (c=1% in MeOH)

### EXAMPLE 85

### Preparation of: 2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one

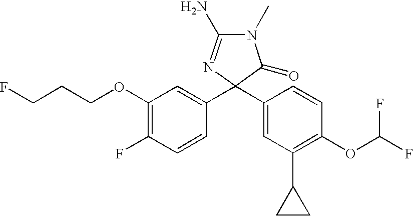

### Step 1: 2-cyclopropyl-1-(difluoromethoxy)-4-((4-fluoro-3-(3-fluoropropoxy)phenyl)ethynyl)benzene

In a 50 mL round-bottomed flask was placed 2-cyclopropyl-1-(difluoromethoxy)-4-ethynylbenzene (0.5 g, 2.401 mmol) and DMF (5.76 mL) and ET₃N (3.84 mL) were added to give a light yellow solution. 4-bromo-1-fluoro-2-(3-fluoropropoxy)benzene (0.603 g, 2.401 mmol) was added. The reaction was degassed by bubbling N2 for 20 min. Bis(triphenylphosphine)palladium(II) chloride (0.084 g, 0.120 mmol) was added. Copper(I) iodide (0.023 g, 0.120 mmol) was added. The N2 bubbling was stopped and r×n heated to 50° C. The reaction became dark brown. The reaction was heated overnight. The reaction was cooled to RT. The reaction was partitioned between ether (50 mL) and 1M HCl (25 mL) and layers separated. The organic was washed with 1M HCl (25 mL) and brine (3×20 mL). The organic layer was dried over Na2SO4. The crude material was purified by flash chromatography (0-20% ethyl acetate/hexanes) to provide 2-cyclopropyl-1-(difluoromethoxy)-4-((4-fluoro-3-(3-fluoropropoxy)phenyl)ethynyl)benzene (0.6 g, 1.586 mmol, 66.0% yield) as a yellow oil.

### Step 2: 1-(3-cyclopropyl-4-(difluoromethoxy)phenyl)-2-(4-fluoro-3-(3-fluoropropoxy)phenyl)ethane-1,2-dione

In a 50 mL round-bottomed flask was placed 2-cyclopropyl-1-(difluoromethoxy)-4-((4-fluoro-3-(3-fluoropropoxy)phenyl)ethynyl)benzene (0.6 g, 1.586 mmol) and DMSO (6.34 mL) was added to give a yellow solution. Bisacetonitriledichloropalladium (0.041 g, 0.159 mmol) was added. The reaction was heated to 120° C. for 4 h. The reaction was cooled to RT. The reaction was partitioned between EtOAc (50 mL) and water (50 ml). The organic was washed with water (25 mL) and brine (25 mL). The organic layer was dried over Na2SO4. The crude material was purified by flash chromatography (0-20% EtOAc/hexanes) to provide 1-(3-cyclopropyl-4-(difluoromethoxy)phenyl)-2-(4-fluoro-3-(3-fluoropropoxy)phenyl)ethane-1,2-dione (0.276 g, 0.673 mmol, 42.4% yield) as a yellow oil that solidified upon standing.

### Step 3: 2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one

In a 25 mL round-bottomed flask was placed 1-(3-cyclopropyl-4-(difluoromethoxy)phenyl)-2-(4-fluoro-3-(3-fluoropropoxy)phenyl)ethane-1,2-dione (0.276 g, 0.673 mmol) and EtOH (2.69 mL) was added to give a yellow solution. Sodium carbonate (0.071 g, 0.673 mmol) was added. 1-methylguanidine hydrochloride (0.074 g, 0.673 mmol) was added. The reaction was heated to 90° C. for 3 h. The solvent was removed. The crude material was loaded onto silica dissolving in small amount of CH₂Cl₂ and purfied by flash chromatography (0-10% MeOH/CH2Cl2) to provide 2-amino-4-(3-cyclopropyl-4-(difluoromethoxy)phenyl)-4-(4-fluoro-3-(3-fluoropropoxy)phenyl)-1-methyl-1H-imidazol-5(4H)-one (0.251 g, 0.539 mmol, 80% yield) as a light yellow solid.

### EXAMPLE 86

### Preparation of: (5R)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one

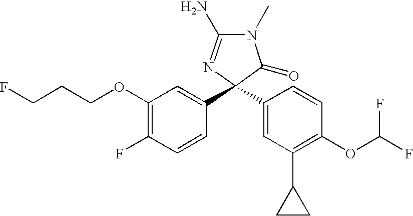

A racemic mixture of 2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one (0.210 g, 0.424 mmol) was separated by chiral chromatography (Chiralpak AD-H 0.46×25 cm Mobile phase 8% EtOH/DEA in hexane/DEA) to provide peak 2, RT=9.83 min, (5R)-2-amino-5-(3-but-1-yn-1-yl-4-fluorophenyl)-5-[4-(difluoromethoxy)-3-methylphenyl]-3-methyl-3,5-dihydro-4H-imidazol-4-one (0.082 g, 0.18 mmol, 39% yield) as a white solid.

MS m/e (M+H)⁺ 466.1, [α]_{D}²⁵=+11 (C=1% in MeOH)

### EXAMPLE 87

### Preparation of: (5S)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one

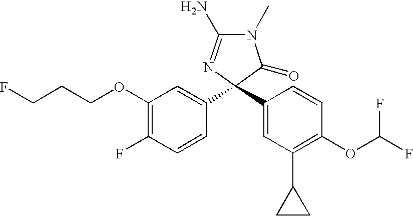

A racemic mixture of 2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one (0.211 g, 0.424 mmol) was separated by chiral chromatography (Chiralpak AD-H 0.46×25 cm Mobile phase 8% EtOH/DEA in hexane/DEA) to provide peak 1, RT=8.46 min, (5S)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one (0.083 g, 0.18 mmol, 40% yield) as a white solid.

MS m/e (M+H)⁺ 466.1, [α]_{D}²⁵=−10.8 (c=1% in MeOH)

### EXAMPLE 88

### Preparation of: 2-amino-5-[4-(difluoromethoxy)-3-(2-fluoroethyl)phenyl]-5-(4-fluoro-3-pent-1-yn-1-ylphenyl)-3-methyl-3,5-dihydro-4H-imidazol-4-one

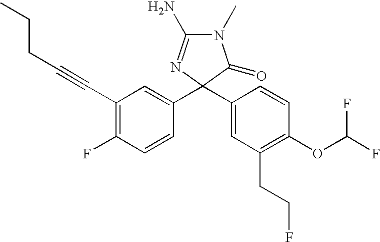

### Step 1: ((4-(difluoromethoxy)-3-(2-fluoroethyl)phenyl)ethynyl)trimethylsilane

In a 250 mL round-bottomed flask was placed 4-bromo-1-(difluoromethoxy)-2-(2-fluoroethyl)benzene (10 g, 37.2 mmol) and DMF (44.6 mL) and ET₃N (29.7 mL) were added to give a colorless solution. The reaction was degassed by bubbling with N2. ethynyltrimethylsilane (6.17 mL, 44.6 mmol) was added. Bis(triphenylphosphine)palladium(II) chloride (1.304 g, 1.858 mmol) was added. copper(I) iodide (0.354 g, 1.858 mmol) was added. The reaction was heated to 60° C. for 4 h. The reaction was partitioned between EtOAc (300 mL) and 1M HCl (100 mL). The organic was washed with brine (3×100 mL). The organic layer was dried over Na2SO4. The crude material was purified by flash chromatography (100% hexane) to provide ((4-(difluoromethoxy)-3-(2-fluoroethyl)phenyl)ethynyl)trimethylsilane (7.63 g, 26.6 mmol, 71.7% yield) as an oil that was used as is in subsequent reaction.

### Step 2: 1-(difluoromethoxy)-4-ethynyl-2-(2-fluoroethyl)benzene

In a 250 mL round-bottomed flask was placed ((4-(difluoromethoxy)-3-(2-fluoroethyl)phenyl)ethynyl)trimethylsilane (7.6 g, 26.5 mmol) and MeOH (53.1 mL) was added to give a light brown solution. POTASSIUM CARBONATE (14.67 g, 106 mmol) was added. The reaction was stirred for 3 h at 25° C. The reaction was diluted with hexanes (300 mL). The organic was washed with water. The organic layer was dried over Na2SO4. The solvent was removed in vacuo providing 1-(difluoromethoxy)-4-ethynyl-2-(2-fluoroethyl)benzene (5 g, 23.34 mmol, 88% yield) as a dark brown oil.

¹H NMR (400 MHz, DMSO-d₆) [symbol] 7.47 (s, 1H), 7.41 (d, J=8.7 Hz, 1H), 7.21 (t, J_{H-F}=74 Hz, 1H), 7.14 (d, J=8.7 Hz, 1H), 4.62 (dt, JH-F=57.1, J=6.3 Hz, 2H), 4.14 (s, 1H), 2.95 (dt, JH-F=24.3, J=6.3 Hz, 2H)

### Step 3: 2-bromo-4-((4-(difluoromethoxy)-3-(2-fluoroethyl)phenyl)ethynyl)-1-fluorobenzene

In a 250 mL round-bottomed flask was placed 1-(difluoromethoxy)-4-ethynyl-2-(2-fluoroethyl)benzene (5 g, 23.34 mmol) and DMF (28.0 mL) and Et₃N was added to give a brown solution. 2-bromo-1-fluoro-4-iodobenzene (7.02 g, 23.34 mmol) was added. The reaction was degassed by bubbling with N2. Bis(triphenylphosphine)palladium(II) chloride (0.819 g, 1.167 mmol) was added. copper(I) iodide (0.222 g, 1.167 mmol) was added. The reaction was stirred for 1 h. The reaction was partitioned between EtOAc (300 mL) and 1N HCl (100 mL). The organic was washed with brine (3×100 mL). The organic layer was dried over Na2SO4. The crude material was purified by flash chromatography 0 to provide 2-bromo-4-((4-(difluoromethoxy)-3-(2-fluoroethyl)phenyl)ethynyl)-1-fluorobenzene (7.11 g, 18.36 mmol, 79% yield) as light brown oil.

### Step 4: 1-(3-bromo-4-fluorophenyl)-2-(4-(difluoromethoxy)-3-(2-fluoroethyl)phenyl)ethane-1,2-dione

In a 100 mL round-bottomed flask was placed 2-bromo-4-((4-(difluoromethoxy)-3-(2-fluoroethyl)phenyl)ethynyl)-1-fluorobenzene (7.11 g, 18.36 mmol) and DMSO (36.7 mL) was added to give a yellow solution. Bis(acetonitrile)palladium(II) chloride (0.476 g, 1.836 mmol) was added. The reaction was heated to 140° C. for 4 h. The reaction was cooled. The reaction was partitioned between EtOAc (300 mL) and water (100 mL). The organic was washed with water (100 mL) and brine (3×100 mL). The organic layer was dried over Na₂SO₄. The crude material was purified by flash chromatography (0-40% EtOAc/hexanes) to provide 1-(3-bromo-4-fluorophenyl)-2-(4-(difluoromethoxy)-3-(2-fluoroethyl)phenyl)ethane-1,2-dione (3.18 g, 7.59 mmol, 41.3% yield) as a yellow solid.

### Step 5: 1-(3-bromo-4-(pent-1-ynyl)phenyl)-2-(4-(difluoromethoxy)-3-(2-fluoroethyl)phenyl)ethane-1,2-dione

In a 5 mL round-bottomed flask was placed 1-(3-bromo-4-fluorophenyl)-2-(4-(difluoromethoxy)-3-(2-fluoroethyl)phenyl)ethane-1,2-dione (0.5 g, 1.193 mmol) and DMF (1.431 mL) and Et₃N (0.954 mL) was added to give a yellow solution. The reaction was degassed by bubbling with N2. Acetylene was added. Bis(triphenylphosphine)palladium(II) chloride (0.042 g, 0.060 mmol) was added. Copper(I) iodide (0.011 g, 0.060 mmol) was added. The reaction was stirred at 60° C. for 4 h. The reaction was partitioned between EtOAc (10 mL) and 1N HCl (5 mL). The organic layer was washed with water (5 mL) and brine (3×5 mL). The organic layer was dried over Na2SO4. The crude material was purified by flash chromatography (0-40% EtOAc/hexanes) to provide 1-(3-bromo-4-(pent-1-ynyl)phenyl)-2-(4-(difluoromethoxy)-3-(2-fluoroethyl)phenyl)ethane-1,2-dione (0.390 g, 0.835 mmol, 70.0% yield) as a yellow solid.

### Step 6: 2-amino-5-[4-(difluoromethoxy)-3-(2-fluoroethyl)phenyl]-5-(4-fluoro-3-pent-1-yn-1-ylphenyl-3-methyl-3,5-dihydro-4H-imidazol-4-one

In a 10 mL vial was placed 1-(3-bromo-4-(pent-1-ynyl)phenyl)-2-(4-(difluoromethoxy)-3-(2-fluoroethyl)phenyl)ethane-1,2-dione (0.384 g, 0.822 mmol) and EtOH (1.644 mL) was added to give a brown solution. Sodium carbonate (0.087 g, 0.822 mmol) was added. 1-Methylguanidine hydrochloride (0.090 g, 0.822 mmol) was added. The reaction was heated to 90° C. After 4 h the reaction was cooled and solvent removed. The crude material was purified by flash chromatography (2-10% MeOH/CH₂Cl₂) to provide 2-amino-4-(4-(difluoromethoxy)-3-(2-fluoroethyl)phenyl)-4-(4-fluoro-3-(pent-1-ynyl)phenyl)-1-methyl-1H-imidazol-5(4H)-one (0.164 g, 0.355 mmol, 43.2% yield) as an off white solid.

MS m/e (M+H)⁺ 462.2

### EXAMPLE 89-92

Using essentially the same procedure described in Example 88 steps 5 and 6 and employing 1-(3-bromo-4-fluorophenyl)-2-(4-(difluoromethoxy)-3-(2-fluoroethyl)phenyl)ethane-1,2-dione and the appropriate alkyne the compounds in the following table were obtained.

TABLE VII

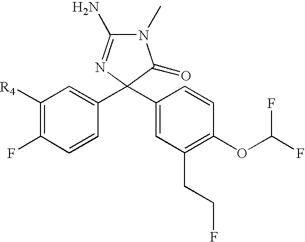

| Ex. No. | R4 | [M + H]⁺ |
|---|---|---|
| 89 | C≡CCH₂CH₃ | 448.2 |
| 90 | C≡CCH₂CH(CH₃)₂ | 476.2 |
| 91 | C≡CCH₂CH₂F | 466.1 |
| 92 | C≡Ccyclopropyl | 460.1 |

### EXAMPLE 93

### Preparation of: (5R)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one

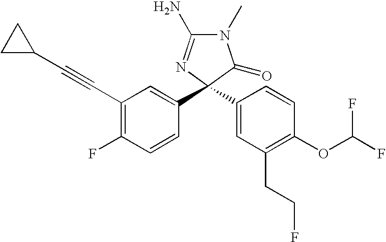

A racemic mixture of 2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one (0.430 g, 0.94 mmol) was separated by chiral chromatography (Chiralpak AD 5×50 cm Mobile phase 10% MeOH/EtOH/NPA in hexane/NPA) to provide peak 1, RT=5.57 min, (5R)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one (0.185 g, 0.40 mmol, 43% yield) as a white solid.

MS m/e (M+H)⁺ 460.1, [α]_{D}²⁵=−8.2 (c=1% in MeOH)

### EXAMPLE 94

### Preparation of: (5S)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one

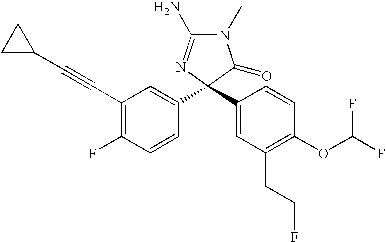

A racemic mixture of 2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one (0.430 g, 0.94 mmol) was separated by chiral chromatography (Chiralpak AD 5×50 cm Mobile phase 10% MeOH/EtOH/NPA in hexane/NPA) to provide peak 2, RT=6.68 min, (5S)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one (0.194 g, 0.42 mmol, 45% yield) as a white solid.

MS m/e (M+H)⁺ 460.1, [α]_{D}²⁵=+5.4 (c=1% in MeOH)

### EXAMPLE 95

### Preparation of: 2-Amino-5-[3-chloro-4-(difluoromethoxy)phenyl]-5-(4-fluoro-3-hydroxyphenyl)-3-methyl-3,5-dihydro-4H-imidazol-4-one

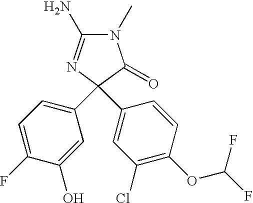

### Step 1: 4-Bromo-2-chloro-1-(difluoromethoxy)benzene

Into a 500 mL round-bottomed flask was charged 4-bromo-2-chlorophenol (10 g, 48.2 mmol), DMF (115 mL), and water (29 mL) were added to give a colorless solution. K₂CO₃ (40 g, 289.2 mmol) was added followed by 2-chloro-2,2-difluoroacetic acid (9.43 g, 72.3 mmol, 6.1 mL) and the reaction was heated at 120° C. overnight. The reaction was cooled and diluted with water and extracted with EtOAc. The organic was washed with 1N NaOH (3×100 mL) to remove unreacted phenol. The organic was washed with brine (100 mL), dried over Na₂SO₄, filtered, and concentrated to 16 g of an oil. This oil was absorbed onto 50 g Celite. Flash chromatography (SiO2, Hexanes to 5:95 EtOAc:Hexanes) provided 12.61 g, 67%, of the title compound as a colorless oil.

¹H NMR 500 MHz (CDCl₃) δ 6.49 (t, 1H J=73.02 Hz); 7.1 (dd, 1H, J=8.69 Hz, 0.81 Hz); 7.37 (dd, 1H, J=8.69 Hz, 2.32 Hz); 7.58 (d, 1H, J=2.32 Hz)

### Step 2: ((3-Chloro-4-(difluoromethoxy)phenyl)ethynyl)trimethylsilane

Into a 500 mL round bottom flask was charged 4-bromo-2-chloro-1-(difluoromethoxy)benzene (12.61 g, 48.98 mmol) from the previous step, trimethylsilylacetylene (7.22 g, 73.47 mmol, 10.4 mL), TEA (24.8 g, 245 mmol, 34.1 mL), and DMF (12.5 mL). The mixture was degassed for 30 minutes after which PdCl₂(PPh₃)₂ (1.72 g, 2.45 mmol) and CuI (933 mg, 4.90 mmol) were added. The mixture was heated under nitrogen at 65° C. until no more starting bromide was seen by tic (about 6 h). The cooled reaction mixture was diluted with EtOAc and water. The aqueous layer was separated and extracted twice with EtOAc. The combined organic layers were washed with water, dried over Na₂SO₄, filtered, and concentrated onto 72 g Celite. Flash chromatography (SiO₂, Hexanes to 5:95 EtOAc:Hexanes) provided 12 g, 89%, of the title compound as an orange oil.

¹H NMR 500 MHz (CDCl₃) δ 0.22 (s, 1H); 6.5 (t, J=73.14 Hz, 3H); 7.12 (d, J=8.46 Hz, 1H); 7.32 (dd, J=8.46 Hz, 1.97 Hz, 1H); 7.52 (D, J=1.97 Hz, 1H)

### Step 3: 2-Chloro-1-(difluoromethoxy)-4-ethynylbenzene

To a solution of ((3-chloro-4-(difluoromethoxy)phenyl)ethynyl)trimethylsilane (12.0 g, 43.67 mmol) from the previous step in MeOH (110 mL) was added K₂CO₃ (60.3 g, 436.7 mmol) at room temperature. The reaction mixture was stirred for 1.5 h after which the mixture was filtered. The filter cake was washed with MeOH and the combined filtrate was concentrated over 40 g Celite. Flash chromatography (SiO₂, Hexanes) provided 6.62 g, 75%, of the title compound as a yellow oil. ¹H NMR 500 MHz (CDCl₃) δ 3.08 (s, 1H); 6.51 (t, 1H, J=73.02 Hz); 7.16 (d, 1H, J=8.46 Hz); 7.35 (dd, 1H, J=8.46 Hz, 1.97 Hz); 7.55 (d, 1H, J=1.97 Hz)

### Step 4: 5-((3-Chloro-4-(difluoromethoxy)phenyl)ethynyl)-2-fluorophenol

To a degassed mixture of 2-chloro-1-(difluoromethoxy)-4-ethynylbenzene (500 mg, 2.54 mmol) from the previous step, 2-fluoro-5-bromophenol (5.67 g, 29.7 mmol), and TEA (16.5 g, 163.5 mmol, 22.8 mL) in DMF (75 mL) was added PdCl₂(PPh₃)₂ (1.15 g, 1.64 mmol) and CuI (629 mg, 3.28 mmol), in that order. The mixture was heated at 70° C. overnight then cooled to room temperature. The mixture was diluted with water, and extracted with EtOAc. The aqueous layer was separated and extracted a second time with EtOAc. The combined organic layers were dried over Na₂SO₄, filtered, and concentrated over 40 g Celite. Flash chromatography (SiO₂, 5:95 EtOAc:Hexanes to 1:4 EtOAc:Hexanes), provided 1.1 g of an inseparable mixture containing the title compound as the major component. This material is used as is in the next reaction.

### Step 5: 1-(3-Chloro-4-(difluoromethoxy)phenyl)-2-(4-fluoro-3-hydroxyphenyl)ethane-1,2-dione

To a solution of 5-((3-chloro-4-(difluoromethoxy)phenyl)ethynyl)-2-fluorophenol (1.0 g, 3.2 mmol) from the previous step in dry DMSO (13 mL) was added PdCl₂(ACN)₂ (83 mg, 0.32 mmol) and the mixture was heated at 120° C. overnight. The cooled reaction mixture was poured into water and extracted with EtOAc. The aqueous layer was separated and extracted with EtOAc twice. The combined organic layers were washed with water, dried over Na₂SO₄, filtered, and concentrated onto 5 g Celite. Flash chromatography (SiO₂, 5:95 EtOAc:Hexanes to 20:80 EtOAc:Hexanes) yielded 560 mg, 50%, of the title compound of a red-orange solid.

MS (−ESI): m/z 343 ([M−H]⁻);

### Step 6: 2-Amino-5-[3-chloro-4-(difluoromethoxy)phenyl]-5-(4-fluoro-3-hydroxyphenyl)-3-methyl-3,5-dihydro-4H-imidazol-4-one

To a solution of 1-(3-chloro-4-(difluoromethoxy)phenyl)-2-(4-fluoro-3-hydroxyphenyl)ethane-1,2-dione (555 mg, 1.61 mmol) from the previous step in 200P EtOH (4.4 mL) was added 1-methylguanidine hydrochloride (265 mg, 2.41 mmol) and Na₂CO₃ (256 mg, 2.41 mmol). The reaction mixture was heated at 90° C. for 1 h, cooled, and concentrated in vacuo onto 700 mg Celite. Flash chromatography (SiO₂, DCM to 1:9 MeOH:DCM) to yield 417 mg, 65%, of the title compound as a light yellow foam.

MS (+ESI): m/z 400.1 ([M+H]⁺)

### EXAMPLE 96

### Preparation of: 2-Amino-5-[3-chloro-4-(difluoromethoxy)phenyl]-5-(3-ethoxy-4-fluorophenyl)-3-methyl-3,5-dihydro-4H-imidazol-4-one

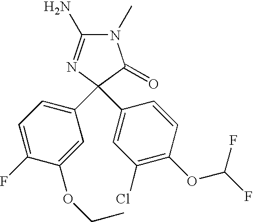

To a 1-dram vial was charged a small stirbar, 2-amino-5-[3-chloro-4-(difluoromethoxy)phenyl]-5-(4-fluoro-3-hydroxyphenyl)-3-methyl-3,5-dihydro-4H-imidazol-4-one (79 mg, 0.131 mmol) from Example 95 Step 6 and DMF (575 μL). Then Cs₂CO₃ (64 mg, 0.198 mmol) was added followed by ethyl iodide (37 mg, 19 μL, 0.218 mmol) was added and the mixture was stirred 1 to 2 d at room temperature. The reaction mixture was diluted with water and extracted with EtOAc. The aqueous layer was separated and extracted with EtOAc once. The combined organic layers were washed with water once, dried over Na₂SO₄, filtered, and concentrated over Celite. Flash chromatography (SiO₂, 100% A to 90% B, where A is DCM and B is 10% MeOH in DCM) provided 54.3 mg, 38%, of the title compound as a light yellow waxy solid.

MS (+ESI): m/z 428.1 ([M+H]⁺)

### EXAMPLE 97

### Preparation of: 2-Amino-5-[3-chloro-4-(difluoromethoxy)phenyl]-5-(4-fluoro-3-propoxyphenyl)-3-methyl-3,5-dihydro-4H-imidazol-4-one

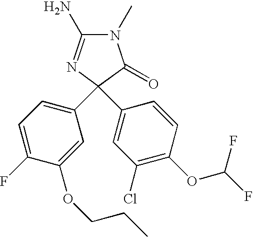

The title compound was made in a similar fashion to Example 96 using 2-amino-5-[3-chloro-4-(difluoromethoxy)phenyl]-5-(4-fluoro-3-hydroxyphenyl)-3-methyl-3,5-dihydro-4H-imidazol-4-one (79 mg, 0.131 mmol) from Example 95 Step 6, propyl iodide (41 mg, 23.5 μL, 0.218 mmol), Cs₂CO₃ (64 mg, 0.198 mmol), and 575 μL DMF to provide 16.7 mg, 11%, of the title compound as a beige wax.

MS (+ESI): m/z 422.1 ([M+H]⁺)

### EXAMPLE 98

### Preparation of: 2-Amino-5-[3-chloro-4-(difluoromethoxy)phenyl]-5-[4-fluoro-3-(3-fluoropropoxy)phenyl]-3-methyl-3,5-dihydro-4H-imidazol-4-one

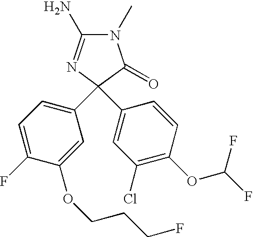

The title compound was made in a similar fashion to Example 96 using the phenol from Example 95 Step 6 (79 mg, 0.198 mmol), 1-bromo-3-fluoropropane (34 mg, 22.1 μL, 0.218 mmol), Cs₂CO₃ (64 mg, 0.198 mmol), and 575 μL DMF. Yield is 60 mg, 40%, of a golden wax.

MS (+ESI): m/z 460.1 ([M+H]⁺)

### EXAMPLE 99

### Preparation of: 2-Amino-5-[3-chloro-4-(difluoromethoxy)phenyl]-5-[4-fluoro-3-(2-fluoroethoxy)phenyl]-3-methyl-3,5-dihydro-4H-imidazol-4-one

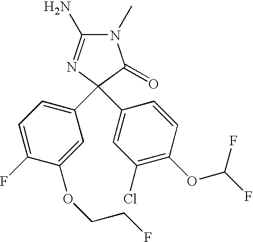

The title compound was made in a similar fashion to Example 96 using the phenol from Example 95 Step 6 (79 mg, 0.198 mmol), 1-fluoro-2-iodoethane (42 mg, 19 μL, 0.218 mmol), Cs₂CO₃ (64 mg, 0.198 mmol), and 575 μL DMF. Yield is 62 mg, 42%, of a beige foam.

MS (+ESI): m/z 446.1 ([M+H]⁺)

### EXAMPLE 100

### Preparation of: 2-Amino-5-[3-chloro-4-(difluoromethoxy)phenyl]-5-[3-(2,2-difluoroethoxy)-4-fluorophenyl]-3-methyl-3,5-dihydro-4H-imidazol-4-one

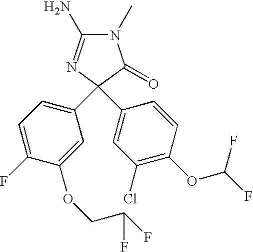

The title compound was made in a similar fashion to Example 96 using the phenol from Example 95 Step 6 (79 mg, 0.198 mmol), 3-bromo-2,2-difluoroethane (35 mg, 19.2 μL, 0.218 mmol), Cs₂CO₃ (64 mg, 0.198 mmol), and 575 μL DMF. Yield is 64 mg, 42%, of a golden wax.

MS (+ESI): m/z 464.1 ([M+H]⁺)

### EXAMPLE 101

### Preparation of: 2-Amino-5-[4-(difluoromethoxy)-3-methylphenyl]-5-(3-ethoxy-4-fluorophenyl)-3-methyl-3,5-dihydro-4H-imidazol-4-one

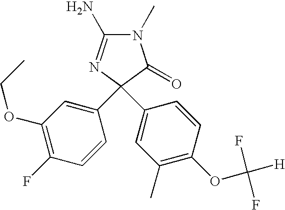

### Step 1: 4-Bromo-1-(difluoromethoxy)-2-methylbenzene

In a 250 mL round-bottomed flask was placed 4-bromo-2-methylphenol (50 g, 267 mmol) and DMF (241 mL). Water (26.7 mL) was added to give a colorless solution. K₂CO₃ (148 g, 1069 mmol) was then added. Sodium 2-chloro-2,2-difluoroacetate (61.1 g, 401 mmol) was added and reaction heated to 120° C. for 12 h. The reaction was cooled to room temperature and partitioned between EtOAc (1000 mL) and water (1000 mL). The layers were separated and organic washed with water (2×500 mL) and brine (2×500 mL). The organic layer was dried over Na₂SO₄ and filtered. The solvent was removed and crude material was passed through a plug of silica eluting with hexanes to provide 12.29 g, 19%, of the title compound as a clear oil.

### Step 2: ((4-(Difluoromethoxy)-3-methylphenyl)ethynyl)trimethylsilane

In a 250 mL round-bottomed flask was placed 4-bromo-1-(difluoromethoxy)-2-methylbenzene (14 g, 59.1 mmol) from the previous step. Pyrrolidine (23.6 mL) and acetonitrile (35.4 mL) were added to give a colorless solution. The reaction was degassed by bubbling with N₂. Ethynyltrimethylsilane (7.0 g, 10 mL, 70.9 mmol) was added followed by bis(triphenylphosphine)dichloropalladium (2.07 g, 2.95 mmol) and copper(I) iodide (0.56 g, 2.95 mmol) was added. The reaction was warmed to 65° C. for 4 h. The reaction was cooled. The solution was partitioned between EtOAc (200 mL) and 1M HCl (200 mL). The organic was washed with 1M HCl (200 mL) and brine (200 mL). The organic layer was dried over Na₂SO₄ and filtered. The solvent was removed and the resulting crude material was purified by flash chromatography (SiO₂, 100% hexanes) to provide 12.4 g, 83%, of the title compound as a light yellow oil.

### Step 3: 1-(Difluoromethoxy)-4-ethynyl-2-methylbenzene

In a 500 mL round-bottomed flask was placed ((4-(difluoromethoxy)-3-methylphenyl)ethynyl)trimethylsilane (12.4 g, 48.8 mmol) from the previous step and MeOH (98 mL) was added to give a colorless solution. Then K₂CO₃ (20.21 g, 146 mmol) was added. The reaction was stirred at room temperature for 3 h. The solution was partitioned between hexanes (300 mL) and water (500 mL). The organic layer was washed with brine, dried over Na₂SO₄, and filtered. The solvent was removed providing 8.2 g, 92%, of the title compound as a light yellow oil. This material was used as is in the next reaction.

### Step 4: 1-(Difluoromethoxy)-4-((3-ethoxy-4-fluorophenyl)ethynyl)-2-methylbenzene

In a 10 mL round-bottomed flask was placed 1-(difluoromethoxy)-4-ethynyl-2-methylbenzene (0.45 g, 2.47 mmol) from the previous step. DMF (3.0 mL) and Et₃N (2.0 mL) were added to give a colorless solution. Then 4-bromo-2-ethoxy-1-fluorobenzene (0.54 g, 2.47 mmol) was added to the reaction mixture. The reaction was degassed by bubbling with N₂. Bis(triphenylphosphine)palladium(II) chloride (0.087 g, 0.123 mmol) was added followed by copper(I) iodide (0.023 g, 0.123 mmol). The reaction was heated at 50° C. for 4 h. then the solution was cooled. The reaction was partitioned between ether (50 mL) and 1M HCl (50 mL). The organic was washed with 1M HCl (25 mL) and brine (25 mL). The crude material was purified by flash chromatography (gradient 0-15% EtOAc/hex) to provide a yellow oil. The yellow oil was subjected to second column (gradient 0-7.5% EtOAc/hex) to provide 250 mg, 32%, of the title compound as a light yellow oil.

### Step 5: 1-(4-(Difluoromethoxy)-3-methylphenyl)-2-(3-ethoxy-4-fluorophenyl)ethane-1,2-dione

This compound was made in a similar fashion to Example 95 Step 5 using 1-(difluoromethoxy)-4-((3-ethoxy-4-fluorophenyl)ethynyl)-2-methylbenzene (250 mg, 0.781 mmol) from the previous step, DMSO (1.56 mL), and bis(acetonitrile)palladium dichloride (20 mg, 0.078 mmol) to provide 175 mg, 64%, of the title compound as a yellow solid.

### Step 6: 2-Amino-5-[4-(difluoromethoxy)-3-methylphenyl]-5-(3-ethoxy-4-fluorophenyl)-3-methyl-3,5-dihydro-4H-imidazol-4-one

This compound was made in a similar fashion to Example 95 Step 6 using 1-(4-(Difluoromethoxy)-3-methylphenyl)-2-(3-ethoxy-4-fluorophenyl)ethane-1,2-dione (175 mg, 0.497 mmol) from the previous step, 1-methylguanidine.HCl (82 mg, 0.745 mmol), Na₂CO₃ (79 mg, 0.745 mmol), and 200P EtOH (1.5 mL) to provide 126 mg, 62%, of a beige foam.

MS (+ESI): m/z 408.1 ([M+H]⁺)

### EXAMPLE 102

### Preparation of: 2-Amino-5-[3-(2,2-difluoroethoxy)-4-fluorophenyl]-5-[4-(difluoromethoxy)-3-methylphenyl]-3-methyl-3,5-dihydro-4H-imidazol-4-one

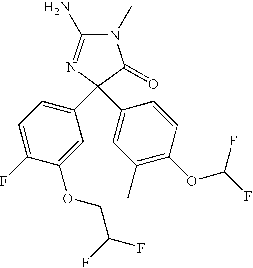

### Step 1: 4-Bromo-2-(2,2-difluoroethoxy)-1-fluorobenzene

To a solution of 2-fluoro-5-bromophenol (2.0 g, 10.5 mmol) in DMF (42 mL) was added Cs₂CO₃ (3.75 g, 11.5 mmol) at room temperature. Then 2-bromo-1,1-difluoroethane (1.67 g, 0.915 mL, 11.5 mmol) was added all at once. The reaction mixture was heated at 50° C. overnight after which the reaction mixture was cooled to room temperature. The mixture was diluted with water and extracted with EtOAc. The aqueous layer was separated and extracted once more with EtOAc. The combined organic layers were washed with 1N NaOH, water, and brine, in that order. The organic layer was dried over Na₂SO₄, filtered, and concentrated in vacuo to give 2.31, 85%, of the title compound as a golden brown oil.

MS (EI): m/z 254 (M^{+.});

### Step 2: 2-(2,2-Difluoroethoxy)-4-((4-(difluoromethoxy)-3-methylphenyl)ethynyl)-1-fluorobenzene

A 100 mL round bottom flask was charged with 4-bromo-2-(2,2-difluoroethoxy)-1-fluorobenzene (1.24 g, 4.86 mmol) from the previous step, 1-(difluoromethoxy)-4-ethynyl-2-methylbenzene (1.15 g, 6.33 mmol) from Example 101 Step 3, TEA (2.46 g, 3.4 mL, 24.3 mmol), and DMF (10.8 mL). The mixture was degassed with N₂ for 30 min after which PdCl₂(PPh₃)₂ (170 mg, 0.243 mmol), and CuI (93 mg, 0.486 mmol) were added. The reaction mixture was heated at 70° C. for 6 h then cooled to room temperature. The mixture was diluted with water then extracted with EtOAc. The aqueous layer was separated and extracted with EtOAc once. The combined organic layers were dried over Na₂SO₄, filtered, and concentrated onto 10 g Celite. Flash chromatography (SiO₂, Hexanes to 7.5% EtOAc 92.5% Hexanes) gave 1.6 g of an orange oil. This oil, by ¹H NMR, contains 2 components of which the major one is the title compound and the minor one is the starting bromide. This material is used as is in the next step.

MS (EI): m/z 356 (M^{+.});

### Step 3: 1-(4-(2,2-Difluoroethoxy)-3-fluorophenyl)-2-(4-(difluoromethoxy)-3-methylphenyl)ethane-1,2-dione

This compound was made in a similar manner to Example 95 Step 5 using 2-(difluoromethoxy)-4-((4-(difluoromethoxy)-3-methylphenyl)ethynyl)-1-fluorobenzene (808 mg, 2.26 mmol) from the previous step, PdCl₂(ACN)₂ (59 mg, 0.226 mmol), and DMSO (9 mL) to provide 261 mg, 30%, of the title compound as an orange solid.

MS (−ESI): m/z 387.1 ([M−H]⁻);

### Step 4: 2-Amino-5-[3-(2,2-difluoroethoxy)-4-fluorophenyl]-5-[4-(difluoromethoxy)-3-methylphenyl]-3-methyl-3,5-dihydro-4H-imidazol-4-one

This compound was made in a similar manner to Example 95 Step 6 using 1-(4-(2,2-difluoroethoxy)-3-fluorophenyl)-2-(4-(difluoromethoxy)-3-methylphenyl)ethane-1,2-dione (260 mg, 0.67 mmol) from the previous step, 1-methylguanidine.HCl (109 mg, 1.0 mmol), Na₂CO₃ (106 mg, 1.0 mmol), and 200P EtOH (1.9 mL) to provide 180 mg, 60%, of the title compound as an orange foam.

MS (+ESI): m/z 444.1 ([M+H]⁺)

### EXAMPLE 103

### Preparation of: (5S)-2-Amino-5-[3-(2,2-difluoroethoxy)-4-fluorophenyl]-5-[4-(difluoromethoxy)-3-methylphenyl]-3-methyl-3,5-dihydro-4H-imidazol-4-one

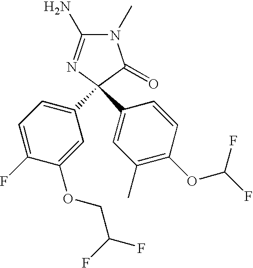

The compound from Example 102 Step 4 was separated by chiral HPLC (Chiralcel AD 5×50 cm; 10% EtOH in Hexane/DEA additive) to provide the title compound as a beige foam.

MS (+ESI): m/z 444.1 ([M+H]⁺)

### EXAMPLE 104

### Preparation of: (5R)-2-Amino-5-[3-(2,2-difluoroethoxy)-4-fluorophenyl]-5-[4-(difluoromethoxy)-3-methylphenyl]-3-methyl-3,5-dihydro-4H-imidazol-4-one

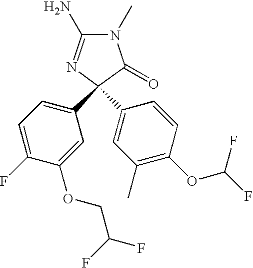

The compound from Example 102 Step 4 was separated by chiral HPLC (Chiralcel AD 5×50 cm; 10% EtOH in Hexane with DEA additive) to provide the title compound as a beige foam.

MS (+ESI): m/z 444.1 ([M+H]⁺)

### EXAMPLE 105

### Preparation of: 2-Amino-5-[3-(cyclopropylmethoxy)-4-fluorophenyl]-5-[4-(difluoromethoxy)-3-methylphenyl]-3-methyl-3,5-dihydro-4H-imidazol-4-one

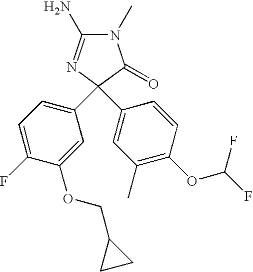

### Step 1:4-Bromo-2-(cyclopropylmethoxy)-1-fluorobenzene

In a 50 mL round-bottomed flask was placed 4-bromo-2-fluorophenol (1 g, 5.24 mmol) and DMF (5.24 mL) was added to give a colorless solution.cesium carbonate (5.12 g, 15.71 mmol) was added. Cyclopropylmethyl bromide (2.120 g, 15.71 mmol) was added. The reaction was stirred overnight. Reaction was diluted with EtOAc (50 mL). The organic was washed with water (20 mL) and brine (3×20 mL). The organic layer was dried over Na2SO4. The crude material was purified by flash chromatography (100% hexanes) to provide the desired product 4-bromo-2-(cyclopropylmethoxy)-1-fluorobenzene (1.152 g, 4.7 mmol, 90% yield) as an oil.

### Step 2: 2-(Cyclopropylmethoxy)-4-((4-(difluoromethoxy)-3-methylphenyl)ethynyl)-1-fluorobenzene

This compound was made in a similar manner to Example 102 Step 2 using 4-bromo-2-(cyclopropylmethoxy)-1-fluorobenzene (1.35 g, 5.51 mmol), 1-(difluoromethoxy)-4-ethynyl-2-methylbenzene (1.15 g, 6.33 mmol) from Example 101 Step 3, TEA (2.70 g, 3.84 mL, 27.55 mmol), PdCl₂(PPh₃)₂ (193 mg, 0.275 mmol), CuI (105 mg, 0.551 mmol), and DMF (12.2 mL) to provide 1.13 g of a yellow oil that contains the title compound and the starting bromide in a 2:1 ratio by ¹H NMR. This material is used as is in the next reaction.

### Step 3: 1-(3-(Cyclopropylmethoxy)-4-fluorophenyl)-2-(4-(difluoromethoxy)-3-methylphenyl)ethane-1,2-dione

This compound was made in a similar manner to Example 95 Step 5 using 2-(cyclopropylmethoxy)-4-((4-(difluoromethoxy)-3-methylphenyl)ethynyl)-1-fluorobenzene (835 mg, 2.41 mmol) from the previous step, PdCl₂(ACN)₂ (63 mg, 0.241 mmol), and DMSO (10 mL) to provide 526 mg, 57%, of the title compound as a yellow solid.

MS (EI): m/z 378 (M^{+.});

### Step 4: 2-Amino-5-[3-(cyclopropylmethoxy)-4-fluorophenyl]-5-[4-(difluoromethoxy)-3-methylphenyl]-3-methyl-3,5-dihydro-4H-imidazol-4-one

This compound was made in a similar fashion to Example 95 Step 6 using 1-(3-(cyclopropylmethoxy)-4-fluorophenyl)-2-(4-(difluoromethoxy)-3-methylphenyl)ethane-1,2-dione (525 mg, 1.38 mmol) from the previous step, 1-methylguanidine.HCl (228 mg, 2.08 mmol), Na₂CO₃ (220 mg, 2.08 mmol), and 200P EtOH (4 mL) to provide 464 mg, 77%, of a light yellow foam.

MS (+ESI): m/z 434.1 ([M+H]⁺)

### EXAMPLE 106

### Preparation of: (5R)-2-Amino-5-[3-(cyclopropylmethoxy)-4-fluorophenyl]-5-[4-(difluoromethoxy)-3-methylphenyl]-3-methyl-3,5-dihydro-4H-imidazol-4-one

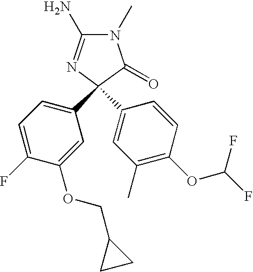

The compound from Example 105 Step 4 was separated by chiral HPLC (Chiralcel OD SFC 2×25 cm; 30% MeOH/DEA additive in CO₂) to provide the title compound as a beige to white foam.

MS (+ESI): m/z 434.1 ([M+H]⁺)

### EXAMPLE 107

### Preparation of: (5S)-2-Amino-5-[3-(cyclopropylmethoxy)-4-fluorophenyl]-5-[4-(difluoromethoxy)-3-methylphenyl]-3-methyl-3,5-dihydro-4H-imidazol-4-one

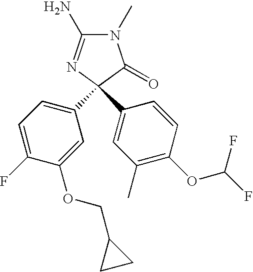

The compound from Example 105 Step 4 was separated by chiral HPLC (Chiralcel OD SFC 2×25 cm; 30% MeOH/DEA additive in CO₂) to provide the title compound as a beige to white foam.

MS (+ESI): m/z 434.1 ([M+H]⁺)

### EXAMPLE 108

### Preparation of: 2-Amino-5-[4-(difluoromethoxy)-3-methylphenyl]-5-[4-fluoro-3-(3-fluoropropoxy)phenyl]-3-methyl-3,5-dihydro-4H-imidazol-4-one

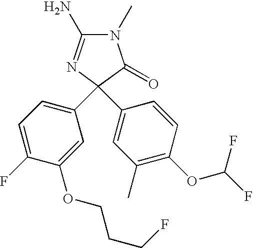

### Step 1 4-Bromo-1-fluoro-2-(3-fluoropropoxy)benzene

This compound was prepared in the same fashion as Example 105 Step 1 using 4-iodo fluorobutane and 4-bromo-2-fluorophenol to provide the desired compound.

### Step 2: 1-(Difluoromethoxy)-4-((4-fluoro-3-(3-fluoropropoxy)phenyl)ethynyl)-2-methylbenzene

This compound was made in a similar fashion to Example 102 Step 2 using 4-bromo-1-fluoro-2-(3-fluoropropoxy)benzene (1.35 g, 5.38 mmol), 1-(difluoromethoxy)-4-ethynyl-2-methylbenzene (1.12 g, 6.18 mmol) from Example 101 Step 3, TEA (2.72 g, 3.75 mL, 26.9 mmol), PdCl₂(PPh₃)₂ (189 mg, 0.275 mmol), CuI (102 mg, 0.538 mmol), and DMF (12 mL) to provide 855 mg, 45%, of the title compound as an orange-yellow oil.

MS (EI): m/z 352 (M^{+.});

### Step 3: 1-(4-(Difluoromethoxy)-3-methylphenyl)-2-(4-fluoro-3-(3-fluoropropoxy)phenyl)ethane-1,2-dione

This compound was made in a similar manner to Example 95 Step 5 using 1-(difluoromethoxy)-4-((4-fluoro-3-(3-fluoropropoxy)phenyl)ethynyl)-2-methylbenzene (850 mg, 2.41 mmol) from the previous step, PdCl₂(ACN)₂ (63 mg, 0.241 mmol), and DMSO (9.6 mL) to provide 575 mg, 62%, of the title compound as a yellow solid.

MS (EI): m/z 384 (M^{+.});

### Step 4: 2-Amino-5-[4-(difluoromethoxy)-3-methylphenyl]-5-[4-fluoro-3-(3-fluoropropoxy)phenyl]-3-methyl-3,5-dihydro-4H-imidazol-4-one

This compound was made in a similar manner to Example 95 Step 6 using 1-(4-(difluoromethoxy)-3-methylphenyl)-2-(4-fluoro-3-(3-fluoropropoxy)phenyl)ethane-1,2-dione (575 mg, 1.5 mmol) from the previous step, 1-methylguanidine.HCl (246 mg, 2.25 mmol), Na₂CO₃ (238 mg, 2.25 mmol), and 200P EtOH (4.3 mL) to provide 450 mg, 68%, of the title compound as a beige foam.

MS (+ESI): m/z 440.1 ([M+H]⁺)

### EXAMPLE 109

### Preparation of: 2-Amino-5-[4-(difluoromethoxy)-3-methylphenyl]-5-[4-fluoro-3-(2-fluoroethoxy)phenyl]-3-methyl-3,5-dihydro-4H-imidazol-4-one

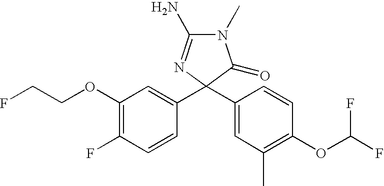

### Step 1: 4-Bromo-1-fluoro-2-(2-fluoroethoxy)benzene

This compound was prepared in the same fashion as Example 105 Step 1 using 2-bromofluoroethane and 4-bromo-2-fluorophenol to provide the desired compound.

### Step 2: 1-(Difluoromethoxy)-4-((4-fluoro-3-(2-fluoroethoxy)phenyl)ethynyl)-2-methylbenzene

This compound was made in a similar manner to Example 102 Step 2 using 4-bromo-1-fluoro-2-(2-fluoroethoxy)benzene (1.13 g, 4.76 mmol) from the previous step, 1-(difluoromethoxy)-4-ethynyl-2-methylbenzene (1.0 g, 5.48 mmol) from Example 101 Step 3, TEA (2.41 g, 3.3 mL, 23.8 mmol), PdCl₂(PPh₃)₂ (167 mg, 0.238 mmol), CuI (90 mg, 0.476 mmol), and DMF (10.6 mL) to provide 797 mg, 49%, of the title compound as a dark orange oil.

MS (EI): m/z 338 (M^{+.});

### Step 3: 1-(4-(Difluoromethoxy)-3-methylphenyl)-2-(4-fluoro-3-(2-fluoroethoxy)phenyl)ethane-1,2-dione

This compound was made in a similar manner to Example 95 Step 5 using 1-(difluoromethoxy)-4-((4-fluoro-3-(2-fluoroethoxy)phenyl)ethynyl)-2-methylbenzene (790 mg, 2.34 mmol) from the previous step, PdCl₂(ACN)₂ (61 mg, 0.234 mmol), and DMSO (9.4 mL) to provide 693 mg, 79%, of the title compound as a yellow-orange solid.

MS (EI): m/z 370 (M^{+.});

### Step 4: 2-Amino-5-[4-(difluoromethoxy)-3-methylphenyl]-5-[4-fluoro-3-(2-fluoroethoxy)phenyl]-3-methyl-3,5-dihydro-4H-imidazol-4-one

This compound was made in a similar manner to Example 95 Step 6 using 1-(4-(difluoromethoxy)-3-methylphenyl)-2-(4-fluoro-3-(2-fluoroethoxy)phenyl)ethane-1,2-dione (690 mg, 1.86 mmol) from the previous step, 1-methylguanidine.HCl (306 mg, 2.8 mmol), Na₂CO₃ (296 mg, 2.8 mmol), and 200P EtOH to provide 567 mg, 71%, of the title compound as a beige foam.

MS (+ESI): m/z 426.1 ([M+H]⁺)

### EXAMPLE 110

### Preparation of: (5S)-2-Amino-5-[4-(difluoromethoxy)-3-methylphenyl]-5-[4-fluoro-3-(3-fluoropropoxy)phenyl]-3-methyl-3,5-dihydro-4H-imidazol-4-one

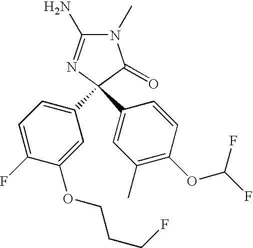

The compound from Example 108 Step 4 was separated by chiral HPLC (Chiralpak AD 5×50 mm; 15% EtOH in Hexane with DEA additive) to provide the title compound as a white foam.

MS (+ESI): m/z 440.1 ([M+H]⁺)

### EXAMPLE 111

### Preparation of: (5R)-2-Amino-5-[4-(difluoromethoxy)-3-methylphenyl]-5-[4-fluoro-3-(3-fluoropropoxy)phenyl]-3-methyl-3,5-dihydro-4H-imidazol-4-one

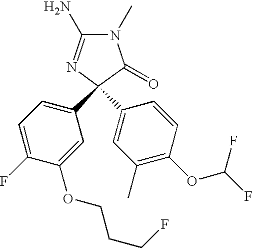

The compound from Example 108 Step 4 was separated by chiral HPLC (Chiralpak AD 5×50 cm; 15% EtOH in Hexane with DEA additive) to provide the title compound as a white foam.

MS (+ESI): m/z 440.1 ([M+H]⁺)

### EXAMPLE 112

### Preparation of: (5S)-2-Amino-5-[4-(difluoromethoxy)-3-methylphenyl]-5-[4-fluoro-3-(2-fluoroethoxy)phenyl]-3-methyl-3,5-dihydro-4H-imidazol-4-one

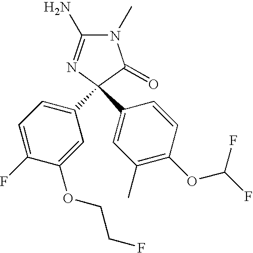

The compound from Example 109 Step 4 was separated by chiral HPLC (Chiralpak AD 5×50 cm; 15% EtOH in Hexane with DEA additive) to provide the title compound as a beige foam.

MS (+ESI): m/z 426.1 ([M+H]⁺)

### EXAMPLE 113

### Preparation of: (5R)-2-Amino-5-[4-(difluoromethoxy)-3-methylphenyl]-5-[4-fluoro-3-(2-fluoroethoxy)phenyl]-3-methyl-3,5-dihydro-4H-imidazol-4-one

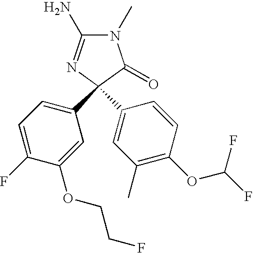

The compound from Example 109 Step 4 was separated by chiral HPLC (Chiralpak AD 5×50 cm; 15% EtOH in Hexane with DEA additive) to provide the title compound as a white foam.

MS (+ESI): m/z 426.1 ([M+H]⁺)

### EXAMPLE 114

### Preparation of: 2-Amino-5-[4-(difluoromethoxy)-3-methylphenyl]-5-(4-fluoro-3-propoxyphenyl)-3-methyl-3,5-dihydro-4H-imidazol-4-one

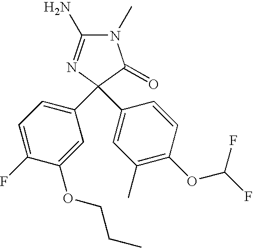

### Step 1: 4-Bromo-1-fluoro-2-propoxybenzene

This compound was prepared in the same fashion as Example 105 Step 1 using 3-iodo propane and 4-bromo-2-fluorophenol to provide the desired compound.

### Step 2: 1-(Difluoromethoxy)-4-((4-fluoro-3-propoxyphenyl)ethynyl)-2-methylbenzene

This compound was made in a similar fashion to Example 102 Step 2 using 4-bromo-1-fluoro-2-propoxybenzene (1.11 g, 4.76 mmol) from the previous step, 1-(difluoromethoxy)-4-ethynyl-2-methylbenzene (998 mg, 5.48 mmol) from Example 101 Step 3, TEA (2.40 g, 3.31 mL, 23.8 mmol), PdCl₂(PPh₃)₂ (91 mg, 0.238 mmol), CuI (91 mg, 0.476 mmol) and DMF (10.5 mL) to provide 690 mg, 43%, of the title compound as a yellow oil.

¹H NMR 500 MHz (CDCl₃) δ 1.04 (t, J=7.42 Hz, 3H); 1.84 (sextet, J=7.10 Hz, 2H); 2.27 (s, 3H); 3.99 (t, J=6.61 Hz, 2H); 6.51 (t, J=73.77 Hz, 1H); 7.00-7.05 (m, 3H); 7.08 (d, J=8.58 Hz, 1H); 7.32 (dd, J=1.85 Hz, 8.35 Hz, 1H); 7.38 (d, J=1.27 Hz, 1H)

### Step 3: 1-(Difluoromethoxy)-4-((4-fluoro-3-propoxphenyl)ethynyl)-2-methylbenzene

This compound was made in a similar fashion to Example 95 Step 5 using 1-(difluoromethoxy)-4-((4-fluoro-3-propoxyphenyl)ethynyl)-2-methylbenzene (690 mg, 2.06 mmol) from the previous step, PdCl₂(ACN)₂ (54 mg, 0.206 mmol), and DMSO (8.2 mL) to provide 629 mg, 83%, of the title compound as a yellow solid.

MS (EI): m/z 366 (M^{+.});

### Step 4: 2-Amino-5-[4-(difluoromethoxy)-3-methylphenyl]-5-(4-fluoro-3-propoxyphenyl)-3-methyl-3,5-dihydro-4H-imidazol-4-one

This compound was made in a similar fashion to Example 95 Step 6 using 1-(difluoromethoxy)-4-((4-fluoro-3-propoxyphenyl)ethynyl)-2-methylbenzene (625 mg, 1.7 mmol) from the previous step, 1-methylguanidine.HCl (305 mg, 2.79 mmol), Na₂CO₃ (296 mg, 2.79 mmol), and 200P EtOH (5.3 mL) to provide 494 mg, 69%, of the title compound as a white foam.

MS (+ESI): m/z 422.1 ([M+H]⁺)

### EXAMPLE 115

### Preparation of: (5S)-2-Amino-5-[4-(difluoromethoxy)-3-methylphenyl]-5-(4-fluoro-3-propoxyphenyl)-3-methyl-3,5-dihydro-4H-imidazol-4-one

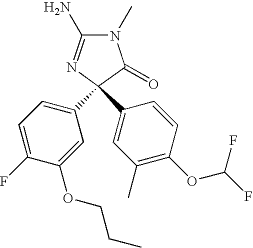

The compound from Example 114 Step 4 was separated by chiral HPLC (Chiralcel AD 5×50 cm; 5% EtOH in Hexane with DEA additive) to provide the title compound as a white foam.

MS (+ESI): m/z 422.1 ([M+H]⁺)

### EXAMPLE 116

### Preparation of: (5S)-2-Amino-5-[4-(difluoromethoxy)-3-methylphenyl]-5-(4-fluoro-3-propoxyphenyl)-3-methyl-3,5-dihydro-4H-imidazol-4-one

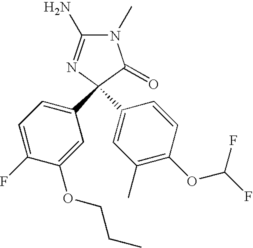

The compound from Example 114 Step 4 was separated by chiral HPLC (Chiralcel AD 5×50 cm; 5% EtOH in Hexane with DEA additive) to provide the title compound as a white foam.

MS (+ESI): m/z 422.1 ([M+H]⁺)

### EXAMPLE 117

### Preparation of: 2-Amino-5-[3-(2,2-difluoroethoxy)-4-fluorophenyl]-5-[4-(difluoromethoxy)-3-ethylphenyl]-3-methyl-3,5-dihydro-4H-imidazol-4-one

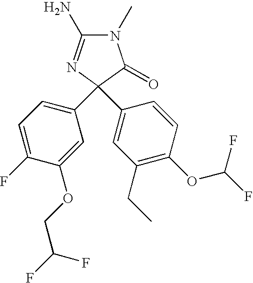

### Step 1: 4-Bromo-2-ethylphenol

To a solution of 2-ethylphenol (12.2 g, 11.76 mL, 100 mmol) in CHCl₃ (200 mL) was added a solution of tetrabutylammonium bromide (48.2 g, 100 mmol) in CHCl₃ (200 mL). The reaction was stirred overnight at room temperature. Then 5% Na₂S₂O₃ solution (500 mL) was added and the biphasic mixture was stirred for 30 minutes. The organic layer was separated and washed with 1N HCl (400 mL). The organic layer was dried over Na₂SO₄, filtered, and concentrated in vacuo to give 51 g of a thick orange oil. Flash chromatography (SiO₂, 1:9 EtOAc:Hexanes to 1:4 EtOAc:Hexanes) provided 10 g, 50%, of the title compound as an orange-brown oil.

¹H NMR 500 MHz (CDCl₃) δ 1.19 (t, J=7.54 Hz, 3H); 2.57 (quartet, 7.53 Hz, 2H); 4.67 (s, 1H); 6.61 (d, J=8.46 Hz, 1H); 7.14 (dd, J=8.46 Hz, 2.44 Hz, 1H); 7.21 (d, J=2.32 Hz, 1H)

### Step 2: 4-Bromo-1-(difluoromethoxy)-2-ethylbenzene

To 4-bromo-2-ethylylphenol (10.0 g, 49.7 mmol) was added 60 mL DMF followed by 6.5 mL water. To this solution was added sodium chlorodifluoroacetate (28.2 g, 171.4 mmol) followed by Cs₂CO₃ (45.3 g, 139.2 mmol). The reaction mixture was heated at 110° C. overnight. Then the reaction mixture was cooled to 0° C. and 60 mL conc. HCl was added followed by water at room temperature. The aqueous mixture was extracted with diethyl ether twice. The combined ether layers were washed with water once, dried over Na₂SO₄, filtered, and concentrated in vacuo over 20 g Celite. Flash chromatography (SiO₂, Hexanes to 20% EtOAc 80% Hexanes) provided 3.6 g, 29%, of the title compound as a light yellow oily semisolid.

MS (EI): m/z 250 (M^{+.})

### Step 3: ((4-(Difluoromethoxy)-3-ethylphenyl)ethynyl)trimethylsilane

This compound was made in a similar manner to Example 95 Step 2 using 4-bromo-1-(difluoromethoxy)-2-ethylbenzene (3.6 g, 14.34 mmol) from the previous step, trimethylsilyl acetylene (2.11 g, 3.0 mL, 21.51 mmol), TEA (7.26 g, 10 mL, 71.7 mmol), PdCl₂(PPh₃)₂ (503 mg, 0.717 mmol), and CuI (273 mg, 1.43 mmol), and DMF (30 mL) to provide 3.12 g, 81%, of the title compound as an orange oil.

MS (EI): m/z 268 (M^{+.})

### Step 4: 1-(Difluoromethoxy)-2-ethyl-4-ethynylbenzene

This compound was made in a similar manner to Example 95 Step 3 using ((4-(difluoromethoxy)-3-ethylphenyl)ethynyl)trimethylsilane (3.1 g, 11.55 mmol), K₂CO₃ (16 g, 115.5 mmol), and MeOH (30 mL) to provide 1.84 g, 84%, of the title compound as a light orange oil.

¹H NMR 500 MHz (CDCl₃) δ 1.19 (t, J=7.54 Hz, 3H); 2.64 (quartet, J=7.54 Hz, 2H); 6.49 (t, J=73.78 Hz, 1H); 7.31 (dd, J=8.46 Hz, 2.09 Hz, 1H); 7.37 (d, J=1.98 Hz, 1H)

### Step 5: 2-(2,2-Difluoroethoxy)-4-((4-(difluoromethoxy)-3-ethylphenyl)ethynyl)-1-fluorobenzene

This compound was made in a similar fashion to Example 102 Step 2 using 4-bromo-2-(2,2-difluoroethoxy)-1-fluorobenzene (1.9 g, 7.44 mmol) from Example 102 Step 1,1-(difluoromethoxy)-2-ethyl-4-ethynylbenzene (1.68 g, 8.56 mmol) from the previous step, TEA (3.76 g, 5.18 mL, 37.2 mmol), PdCl₂(PPh₃)₂ (261 mg, 0.372 mmol), CuI (142 mg, 0.744 mmol), and DMF (10 mL) to provide 1.68 g, 61%, of the title compound as an orange oil.

MS (EI): m/z 370 (M^{+.})

### Step 6: 1-(3-(2,2-difluoroethoxy)-4-fluorophenyl)-2-(4-(difluoromethoxy)-3-ethylphenyl)ethane-1,2-dione

This compound was made in a similar fashion to Example 95 Step 5 using 2-(2,2-difluoroethoxy)-4-((4-(difluoromethoxy)-3-ethylphenyl)ethynyl)-1-fluorobenzene (1.68 g, 4.54 mmol) from the previous step, PdCl₂(ACN)₂ (117 mg, 0.454 mmol), and DMSO (18.2 mL) to provide 829 mg, 45%, of the title compound as a burnt orange solid.

MS (EI): m/z 402 (M^{+.})

### Step 7: 2-Amino-5-[3-(2,2-difluoroethoxy)-4-fluorophenyl]-5-[4-(difluoromethoxy)-3-ethylphenyl]-3-methyl-3,5-dihydro-4H-imidazol-4-one

This compound was made in a similar fashion to Example 95 Step 6 using 1-(3-(2,2-difluoroethoxy)-4-fluorophenyl)-2-(4-(difluoromethoxy)-3-ethylphenyl)ethane-1,2-dione (825 mg, 2.05 mmol) from the previous step, 1-methylguanidine.HCl (337 mg, 3.07 mmol), Na₂CO₃ (326 mg, 3.07 mmol), and 200P EtOH (8.2 mL) to provide 634 mg, 67%, of the title compound as a cream colored foam.

MS (+ESI): m/z 458 ([M+H]⁺)

### EXAMPLE 118

### Preparation of: (5S)-2-Amino-5-[3-(2,2-difluoroethoxy)-4-fluorophenyl]-5-[4-(difluoromethoxy)-3-ethylphenyl]-3-methyl-3,5-dihydro-4H-imidazol-4-one

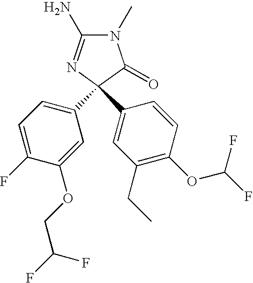

The compound from Example 117 Step 7 was separated by chiral HPLC (Chiralcel AD, 5×50 cm; 9% (80/20 MeOH/EtOH) in Hexanes with DEA additive) to provide the title compound as an off-white foam.

MS (+ESI): m/z 458 ([M+H]⁺)

### EXAMPLE 119

### Preparation of: (5R)-2-Amino-5-[3-(2,2-difluoroethoxy)-4-fluorophenyl]-5-[4-(difluoromethoxy)-3-ethylphenyl]-3-methyl-3,5-dihydro-4H-imidazol-4-one

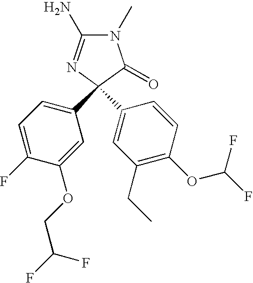

The compound from Example 117 Step 7 was separated by chiral HPLC (Chiralcel AD, 5×50 cm; 9% (80/20 MeOH/EtOH) in Hexanes with DEA additive) to provide the title compound as an off-white foam.

MS (+ESI): m/z 458 ([M+H]⁺)

### EXAMPLE 120

### Preparation of: 2-Amino-5-[4-(difluoromethoxy)-3-ethylphenyl]-5-[4-fluoro-3-(3-fluoropropoxy)phenyl]-3-methyl-3,5-dihydro-4H-imidazol-4-one

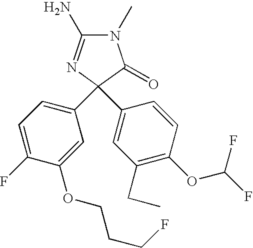

### Step 1: 4-Bromo-1-fluoro-2-(3-fluoropropoxy)benzene

This compound was made in a similar fashion to Example 102 Step 1 using 5-bromo-2-fluorophenol (3.82 g, 20 mmol), 1-iodo-3-fluoropropane (4.13 g, 22 mmol), Cs₂CO₃ (7.17 g, 22 mmol), and DMF (80 mL) to provide 4.5 g, 89%, of the title compound as a brown-orange oil.

MS (EI): m/z 250 (M^{+.})

### Step 2: 4-Ethynyl-1-fluoro-2-(3-fluoropropoxy)benzene

This compound was made in a similar manner to Example 95 Step 2 using 4-bromo-1-fluoro-2-(3-fluoropropoxy)benzene (2.0 g, 8.0 mmol), trimethylsilylacetylene (1.18 g, 1.7 mL, 12 mmol), TEA (4.05 g, 5.58 mmol, 40 mmol), PdCl₂(PPh₃)₂ (281 mg, 0.40 mmol), CuI (152 mg, 0.8 mmol), and DMF (16 mL) to provide 2.0 g, 93%, of the title compound as an orange oil.

MS (EI): m/z 268 (M^{+.})

### Step 3: ((4-Fluoro-3-(3-fluoropropoxy)phenyl)ethynyl)trimethylsilane

This compound was made in a similar manner to Example 95 Step 3 using 4-ethynyl-1-fluoro-2-(3-fluoropropoxy)benzene (1.95 g, 7.27 mmol) from the previous step, K₂CO₃ (10 g, 72.7 mmol), and MeOH (18 mL) to provide 1.17 g, 82%, of the title compound as an orange oil.

MS (EI): m/z 196 (M⁺)

### Step 4: 1-(Difluoromethoxy)-2-ethyl-4-((4-fluoro-3-(3-fluoropropoxy)phenyl)ethynyl)benzene

This compound was made in a similar manner to Example 95 Step 2 using ((4-fluoro-3-(3-fluoropropoxy)phenyl)ethynyl)trimethylsilane (1.1 g, 5.6 mmol) from the previous step, 4-bromo-1-(difluoromethoxy)-2-ethylbenzene (937 mg, 3.73 mmol) from Example 117 Step 2, TEA (1.89 g, 2.6 mL, 18.67 mmol), PdCl₂(PPh₃)₂ (131 mg, 0.187 mmol), CuI (71 mg, 0.373 mmol), and DMF (7.5 mL) to provide 716 mg, 34%, of the title compound as a light yellow oil.

MS (EI): m/z 366 (M^{+.})

### Step 5: 1-(4-(Difluoromethoxy)-3-ethylphenyl)-2-(4-fluoro-3-(3-fluoropropoxy)phenyl)ethane-1,2-dione

This compound was made in a similar manner to Example 95 Step 5 using 1-(difluoromethoxy)-2-ethyl-4-((4-fluoro-3-(3-fluoropropoxy)phenyl)ethynyl)benzene (700 mg, 1.91 mmol) from the previous step, PdCl₂(ACN)₂ (50 mg, 0.191 mmol), and DMSO (7.6 mL) to provide 629 mg, 82%, of the title compound as a burnt orange solid.

MS (−ESI): m/z 397.2 ([M−H]⁻)

### Step 6: 2-Amino-5-[4-(difluoromethoxy)-3-ethylphenyl]-5-[4-fluoro-3-(3-fluoropropoxy)phenyl]-3-methyl-3,5-dihydro-4H-imidazol-4-one

This compound was made in a similar manner to Example 95 Step 6 using 1-(4-(difluoromethoxy)-3-ethylphenyl)-2-(4-fluoro-3-(3-fluoropropoxy)phenyl)ethane-1,2-dione (620 mg, 1.55 mmol) from the previous step, 1-methylguanidine.HCl (256 mg, 2.33 mmol), Na₂CO₃ (247 mg, 2.33 mmol), and iPrOH (6.2 mL) to provide 634 mg, 67%, of the title compound as a cream colored foam.

MS (+ESI): m/z 454.2 ([M+H]⁺)

### EXAMPLE 121

### Preparation of: (5S)-2-Amino-5-[4-(difluoromethoxy)-3-ethylphenyl]-5-[4-fluoro-3-(3-fluoropropoxy)phenyl]-3-methyl-3,5-dihydro-4H-imidazol-4-one

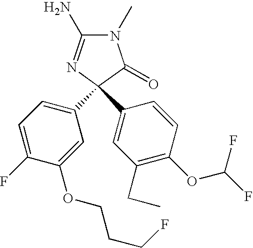

The title compound from Example 120 Step 6 was separated by chiral HPLC (Chiralcel AD 5×50 cm; 9% EtOH in Hexanes with DEA additive) to provide the title compound as a white foam.

MS (+ESI): m/z 454.2 ([M+H]⁺)

### EXAMPLE 122

### Preparation of: (5S)-2-Amino-5-[4-(difluoromethoxy)-3-ethylphenyl]-5-[4-fluoro-3-(3-fluoropropoxy)phenyl]-3-methyl-3,5-dihydro-4H-imidazol-4-one

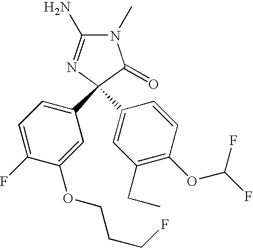

The title compound from Example 120 Step 6 was separated by chiral HPLC (Chiralcel AD 5×50 cm; 9% EtOH in Hexanes with DEA additive) to provide the title compound as a white foam.

MS (+ESI): m/z 454.2 ([M+H]⁺)

### EXAMPLE 123

### Preparation of: 2-Amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one

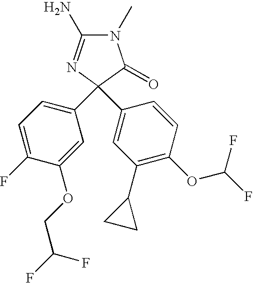

### Step 1: 2-Bromo-4-iodo-phenol

[Reference: Jon Clardy in Org. Lett. 2006, 8(19) 4251].

In a 250 ml round bottom flask 4-iodo-phenol (10 g, 45.4 mmol) was dissolved in methanol (60 mL). Bromine (2.55 mL) was added dropwise at 0° C. After 30 minutes sodium thiosulfate solution was added, the reaction mixture was extracted with diethyl ether, washed with water, dried with Na₂SO₄, and concentrated onto silica gel. Purification by column chromatography (SiO₂, 2:1 hexanes/dichloromethane) yielded 3.24 g of the title compound and mixed fractions were re-subjected to column chromatography 20-35% dichloromethane in hexanes to give another 6.50 g of the title compound for a total of 9.75 g (72%).

¹H NMR (400 MHz, DMSO-d) δ 6.73 (d, J=8.3 Hz, 1H); 7.43 (dd, J=8.6 Hz, 2.1 Hz, 1H); 7.72 (d, 6 J=2.1 Hz, 1H); 10.47 (s, 1H).

### Step 2: 2-Bromo-1-difluoromethoxy-4-iodo-benzene

In a 100 mL round bottom flask equipped with a large magnetic egg-shaped stir bar were combined 2-bromo-4-iodo-phenol (3.45 g, 11.5 mmol), sodium chlorodifluoroacetate (1.76 g, 11.5 mmol), and potassium carbonate (6.38 g, 46.2 mmol) in 10% aqueous dimethylformamide (25 mL). The reaction mixture was immersed in a pre-heated oil bath at 110° C. After 8 hours the crude reaction mixture was partitioned between ethyl acetate and water, washed with 1N NaOH, brine and dried with MgSO₄. Purification by column chromatography (SiO₂, hexanes) yielded 2.68 g, 67%, of the title compound as a low melting white solid.

¹H NMR (400 MHz, DMSO-d₆) δ 7.09 (d, 1H); 7.23 (t, J=73.02 Hz, 1H); 7.75 (dd, J=8.6 Hz, 2.1 Hz, 1H); 8.05 (d, J=2.1 Hz, 1H).

### Step 3: (3-Bromo-4-difluoromethoxy-phenylethynyl)-triisopropyl-silane

In a 100 mL round bottom flask were combined 2-bromo-1-difluoromethoxy-4-iodo-benzene (4.28 g, 12.2 mmol) from the previous step and triisopropyl-silyl-acetylene (5.45 mL, 14.4 mmol), in triethylamine (12 mL) and dimethylformamide (24 mL). The contents were chilled in an ice-water bath to 0° C. Cuprous iodide (117 mg, 0.614 mmol) and palladium dichlorobis(triphenyl-phosphine) (432 mg, 0.615 mmol) were added and the mixture stirred at 0 C. After 1 hour the crude reaction mixture was partitioned between diethyl ether and a saturated solution of ammonium chloride, then washed with a saturated solution of ammonium chloride and dried with Na₂SO₄. Purification by column chromatography (SiO₂, hexanes) and isolation by concentration of desired fractions by rotary evaporation (bath temperature <5° C.) yielded 4.67 g, 94%, of the title compound as an oil.

¹H NMR (400 MHz, DMSO-d₆) δ 1.05 (s, 21H); 7.25-7.30 (m, 1H); 7.29 (t, J=72.90 Hz, 1H); 7.50 (dd, J=8.6 Hz, 2.1 Hz, 1H); 7.77 (d, J=2.1 Hz, 1H);

MS (EI) m/z 402 (M^{+.}).

### Step 4: (3-Cyclopropyl-4-difluoromethoxy-phenylethynyl)-triisopropyl-silane

[Reference: Debra Wallace in Tetra. Lett. 2002, 43(39) 6987].

In a 100 mL round bottom flask equipped with a magnetic stir egg were combined (3-bromo-4-difluoromethoxy-phenylethynyl)-triisopropyl-silane (1.17 g, 2.90 mmol), cyclopropylboronic acid (0.500 g, 5.80 mmol), potassium phosphate (2.16 g, 10.2 mmol), palladium acetate (32 mg, 0.143 mmol) and tricylclohexyl-phosphine (81 mg, 0.290 mmol) in toluene (13 mL) and water (0.65 mL). The reaction mixture was immersed in a pre-heated oil bath at 100° C. After 3 hours add more cyclopropyl boronic acid, Pd(OAc)₂, P(cHex)₃ and phosphate base and continue heating another three hours. The crude reaction mixture was partitioned between ethyl acetate and water, extracted with ethyl acetate and dried with MgSO₄. Purification by column chromatography (SiO₂, 0-25% ethyl acetate in hexanes) yielded 1.01 g, 96%, of the title compound as an oil.

¹H NMR (400 MHz, DMSO-d₆) δ 0.69 (quartet, J=5.2 Hz, 2H); 0.92 (dquartet, J=8.5 Hz, 2.8 Hz, 2H); 1.05 (s, 21H); 2.01 (quintet, J=6.8 Hz, 1H); 6.97 (d, J=2.1 Hz, 1H); 7.10 (d, J=8.6 Hz, 1H); 7.22 (t, J=73.89 Hz, 1H); 7.28 (dd, J=8.4 Hz, 2.1 Hz, 1H).

### Step 5: 2-Cyclopropyl-1-difluoromethoxy-4-ethynyl-benzene

To a cooled (0° C.) solution of (3-cyclopropyl-4-difluoromethoxy-phenylethynyl)-triisopropyl-silane (2.0 g, 5.48 mmol) from the previous step in THF (3.8 mL) was added TBAF in THF (1.0M, 5.7 mL, 5.7 mmol). The reaction mixture was stirred for 1 h at 0° C. then allowed to warm to room temperature. The reaction was diluted with hexanes (100 mL) and washed with water. The hexanes layer was separated and the aqueous layer was extracted with hexanes once. The combined organic extracts were dried over Na₂SO₄, filtered, and concentrated onto 8.6 g Celite. Flash chromatography (SiO₂, hexanes to 2% EtOAc 98% Hexanes) provided 1.0 g, 85%, of the title compound as a colorless oil.

¹H NMR (400 MHz, DMSO-d₆) δ 0.68 (quintet, J=3.9 Hz, 2H); 0.91 (dquartet, J=8.8 Hz, 3.5 Hz, 2H); 2.01 (dquartet, J=12.4 Hz, 5.1 Hz, 1H); 4.10 (s, 1H); 7.01 (d, J=2.1 Hz, 1H); 7.09 (d, J=8.6 Hz, 1H); 7.20 (t, J=73.95 Hz, 1H); 7.28 (dd, J=8.3 Hz, 2.1 Hz, 1H)

### Step 6: 2-Cyclopropyl-4-((3-(2,2-difluoroethoxy)-4-fluorophenyl)ethynyl)-1-(difluoromethoxy)benzene

This compound was made in a similar fashion to Example 102 Step 2 using 2-cyclopropyl-1-difluoromethoxy-4-ethynyl-benzene (500 mg, 2.4 mmol) from the previous step), 4-bromo-2-(2,2-difluoroethoxy)-1-fluorobenzene (532 mg, 2.09 mmol) from Example 102 Step 1, TEA (1.21 g, 1.67 mL, 12 mmol), PdCl₂(PPh₃)₂ (84 mg, 0.12 mmol), CuI (46 mg, 0.24 mmol), and DMF (4.6 mL) to provide 330 mg, 41%, of the title compound as a light yellow oil.

MS (EI): m/z 382 (M^{+.}).

### Step 7: 1-(3-Cyclopropyl-4-(difluoromethoxy)phenyl)-2-(3-(2,2-difluoroethoxy)-4-fluorophenyl)ethane-1,2-dione

This compound was made in a similar manner to Example 95 Step 5 using 2-cyclopropyl-4-((3-(2,2-difluoroethoxy)-4-fluorophenyl)ethynyl)-1-(difluoromethoxy)benzene (330 mg, 0.863 mmol) from the previous step, PdCl₂(ACN)₂ (22 mg, 0.0863 mmol), and DMSO (3.5 mL) to provide 229 mg, 64%, of the title compound as a yellow solid.

MS (EI): m/z 414 (M^{+.}).

### Step 8: 2-Amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one

This compound was made in a similar manner to Example 95 Step 6 using 1-(3-cyclopropyl-4-(difluoromethoxy)phenyl)-2-(3-(2,2-difluoroethoxy)-4-fluorophenyl)ethane-1,2-dione (227 mg, 0.548 mmol) from the previous step, 1-methylguanidine.HCl (90 mg, 0.822 mmol), Na₂CO₃ (87 mg, 0.822 mmol), and 200P EtOH (1.6 mL) to provide 170 mg, 66%, of the title compound as a beige foam.

MS (+ESI): m/z 470.2 ([M+H]⁺)

### EXAMPLE 124

### Preparation of: (5S)-2-Amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one

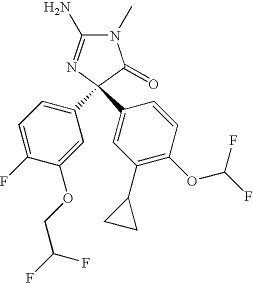

The title compound from Example 123 Step 8 was separated by chiral HPLC (Chiralcel AD 5×25 cm; 9% EtOH in hexanes with DEA additive) to provide the title compound as a white solid.

MS (+ESI): m/z 470.2 ([M+H]⁺)

### EXAMPLE 125

### Preparation of: (5R)-2-Amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one

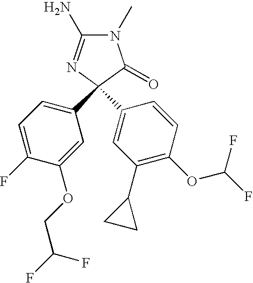

The title compound from Example 123 Step 8 was separated by chiral HPLC (Chiralcel AD 5×25 cm; 9% EtOH in hexanes with DEA additive) to provide the title compound as a white solid.

MS (+ESI): m/z 470.2 ([M+H]⁺)

### EXAMPLE 126

### Preparation of: 2-Amino-5-[4-(difluoromethoxy)-3-methylphenyl]-5-[3-(3,3-difluoropropoxy)phenyl]-3-methyl-3,5-dihydro-4H-imidazol-4-one

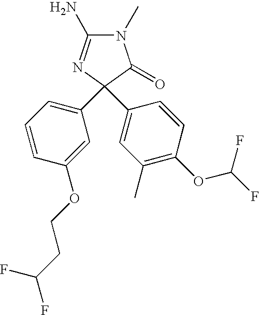

### Step 1: 1-(Difluoromethoxy)-4-((3-(3,3-difluoropropoxy)phenyl)ethynyl)-2-methylbenzene

This compound was made in a similar manner to Example 95 Step 2 using 1-(3,3-difluoropropoxy)-3-ethynylbenzene (650 mg, 3.13 mmol) from Example 127 Step 5, 1-(difluoromethoxy)-4-iodo-2-methylbenzene (818 mg, 2.88 mmol), TEA (1.45 g, 2.0 mL, 14.5 mmol), PdCl₂(PPh₃)₂ (101 mg, 0.144 mmol), and CuI (16.4 mg, 0.0864 mmol), and DMF (4.4 mL) to provide the title compound as a yellow oil.

MS (EI): m/z 352 (M^{+.}).

### Step 2: 1-(4-(Difluoromethoxy)-3-methylphenyl)-2-(3-(3,3-difluoropropoxy)phenyl)ethane-1,2-dione

This compound was made in a similar manner to Example 95 Step 5 using 1-(difluoromethoxy)-4-((3-(3,3-difluoropropoxy)phenyl)ethynyl)-2-methylbenzene (727 mg, 2.06 mmol) from the previous step, PdCl₂(ACN)₂ (54 mg, 0.206 mmol), and DMSO (8.2 mL) to provide 506 mg, 64%, of the title compound as an orange oil.

MS (−ESI): m/z 383 ([M−H]⁻).

### Step 3: 2-Amino-5-[4-(difluoromethoxy)-3-methylphenyl]-5-[3-(3,3-difluoropropoxy)phenyl]-3-methyl-3,5-dihydro-4H-imidazol-4-one

This compound was made in a similar manner to Example 95 Step 6 using 1-(4-(difluoromethoxy)-3-methylphenyl)-2-(3-(3,3-difluoropropoxy)phenyl)ethane-1,2-dione (720 mg, 1.87 mmol), 1-methylguanidine hydrochloride (308 mg, 2.81 mmol), Na₂CO₃ (298 mg, 2.81 mmol), and 200P EtOH (5.3 mL) to provide 635 mg of a tan solid/foam. By ¹H NMR this is the acetate salt of the title compound. The foam was dissolved in DCM and washed with saturated NaHCO₃ to release the free base. There resulted in 335 mg, 43%, of the title compound as a light yellow foam. There resulted in 492 mg, 60%, of the title compound as a tan foam.

MS (EI): m/z 440.1 ([M+H]⁺)

### EXAMPLE 127

### Preparation of: (5R)-2-Amino-5-[4-(difluoromethoxy)-3-methylphenyl]-5-[3-(3,3-difluoropropoxy)phenyl]-3-methyl-3,5-dihydro-4H-imidazol-4-one

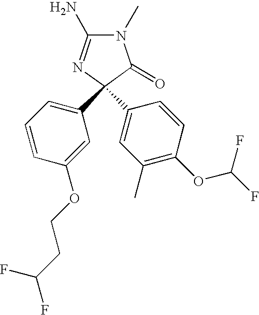

The compound from Example 126 Step 3 was separated by chiral HPLC (Chiralcel OD-H, 2×25 cm; 15% IPA in Hexanes with DEA additive) to provide the title compound as a white foam.

MS (+ESI): m/z 440.1 ([M+H]⁺)

### EXAMPLE 128

### Preparation of: (5S)-2-Amino-5-[4-(difluoromethoxy)-3-methylphenyl]-5-[3-(3,3-difluoropropoxy)phenyl]-3-methyl-3,5-dihydro-4H-imidazol-4-one

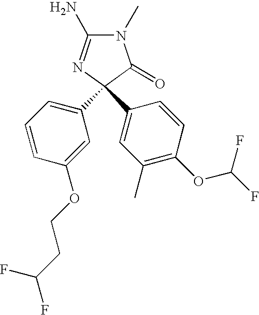

The compound from Example 126 Step 3 was separated by chiral HPLC (Chiralcel OD-H, 2×25 cm; 15% IPA in Hexanes with DEA additive) to provide the title compound as a white foam.

MS (+ESI): m/z 440.1 ([M+H]⁺).

### EXAMPLE 129

### Preparation of: 2-Amino-5-[3-(2,2-difluoroethoxy)-4-fluorophenyl]-5-[4-(difluoromethoxy)-3-(2-fluoroethyl)phenyl]-3-methyl-3,5-dihydro-4H-imidazol-4-one

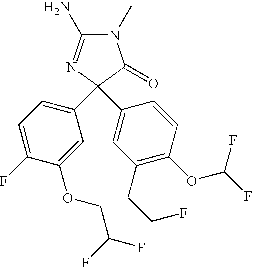

### Step 1: 2-(2,2-Difluoroethoxy)-4-((4-(difluoromethoxy)-3-(2-fluoroethyl)phenyl)ethynyl)-1-fluorobenzene

This compound was made in a similar manner to Example 102 Step 2 using 1-(difluoromethoxy)-4-ethynyl-2-(2-fluoroethyl)benzene (660 mg, 3.08 mmol), 4-bromo-2-(2,2-difluoroethoxy)-1-fluorobenzene (532 mg, 2.09 mmol) from Example 102 Step 1, TEA (1.35 g, 1.86 mmol, 13.4 mmol), PdCl₂(PPh₃)₂ (94 mg, 0.134 mmol), CuI (57 mg, 0.268 mmol), and DMF (6 mL) to provide 338 mg, 32%, of the title compound as a turbid light yellow oil.

MS (EI): m/z 388 (M^{+.}).

### Step 2: 1-(3-(2,2-Difluoroethoxy)-4-fluorophenyl)-2-(4-(difluoromethoxy)-3-(2-fluoroethyl)phenyl)ethane-1,2-dione

This compound was made in a similar manner to Example 95 Step 5 using 2-(2,2-difluoroethoxy)-4-((4-(difluoromethoxy)-3-(2-fluoroethyl)phenyl)ethynyl)-1-fluorobenzene (330 mg, 0.85 mmol) from the previous step, PdCl₂(ACN)₂ (22 mg, 0.85 mmol), and DMSO (3.4 mL) to provide 229 mg, 64%, of the title compound as a yellow oil that solidifies into a yellow solid upon standing.

MS (−ESI): m/z 419.2 ([M−H]⁻).

### Step 3: 2-Amino-5-[3-(2,2-difluoroethoxy)-4-fluorophenyl]-5-[4-(difluoromethoxy)-3-(2-fluoroethyl)phenyl]-3-methyl-3,5-dihydro-4H-imidazol-4-one

This compound was made in a similar manner to Example 95 Step 6 using 1-(3-(2,2-difluoroethoxy)-4-fluorophenyl)-2-(4-(difluoromethoxy)-3-(2-fluoroethyl)phenyl)ethane-1,2-dione (220 mg, 0.523 mmol) from the previous step, 1-methylguanidine hydrochloride (86 mg, 0.785 mmol), Na₂CO₃ (83 mg, 0.785 mmol), and 200P EtOH (1.5 mL) to provide 202 mg, 81%, of the title compound as a beige foam.

MS (+ESI): m/z 476.1 ([M+H]⁺)

### EXAMPLE 130

### Preparation of: 2-Amino-5-[4-(difluoromethoxy)-3-methylphenyl]-5-[3-(3,3-difluoropropoxy)-4-fluorophenyl]-3-methyl-3,5-dihydro-4H-imidazol-4-one

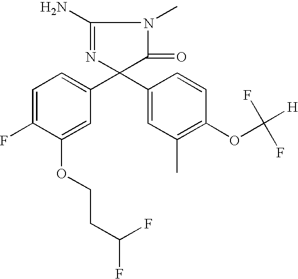

### Step 1: 4-Bromo-2-(but-3-enyloxy)-1-fluorobenzene

To a solution of 2-fluoro-5-bromophenol (19.63 g, 102.8 mmol) in DMF (410 mL) was added Cs₂CO₃ (40.19 g, 123.4 mmol, 1.2 eq.) followed by 4-bromo-1 butene (15.26 g, 113 mmol, 1.1 eq). The mixture was stirred overnight between 50 and 60° C. Then additional amounts of Cs₂CO₃ and the bromoalkene (0.6 eq., 20.05 g, and 0.55 eq., 7.63 g, respectively) were added and the mixture was heated overnight at 50-60° C. The mixture was cooled to room temperature and diluted with water. The aqueous mixture was extracted with EtOAc several times and the combined extracts were washed well with water, dried over Na₂SO₄, filtered, and concentrated onto 50 g of Celite. Flash chromatography (SiO₂, Hexanes to 30% EtOAc 70% Hexanes) provided 11.87 g, 47%, of the title compound as a colorless to light yellow oil.

¹H NMR 500 MHz (CDCl₃) δ 2.54 (qt, J=1.30 Hz, 6.72 Hz, 2H); 4.03 (t, J=6.72 Hz, 2H); 5.07-5.19 (m, 2H); 5.80-6.00 (m, 1H); 6.89-6.94 (m, 1H); 6.95 (m, 1H); 7.05 (dd, J=2.27 Hz, 7.47 Hz, 1H)

### Step 2: 3-(5-Bromo-2-fluorophenoxy)propanal

To a room temperature solution of 4-bromo-2-(but-3-enyloxy)-1-fluorobenzene (11.85 g, 48.35 mmol) from the previous step, in THF (1025 mL) was added water (683 mL). The solution was cooled to 0° C. and NalO₄ (31 g, 145 mmol) followed by OsO₄ (4 wt % solution in water, 6.1 mL, 2 mol % catalyst). The mixture was stirred at this temperature for 4 h then allowed to stand at room temperature overnight without stirring. The mixture was filtered, washed with THF and a small amount of water (<100 mL). The filtrate was diluted with EtOAc. The organic layer was separated and the aqueous layer was extracted with EtOAc once. The combined organic layers were washed with brine once, dried over Na₂SO₄, filtered, and concentrated in vacuo to give 12.9 g, 108%, of a purple black oil. This oil is used as is immediately in the next step.

¹H NMR 500 MHz (CDCl₃) δ 2.94 (td, J=6.12 Hz, 2.47 Hz, 2H); 4.32 (t, J=6.09 Hz, 2H); 6.90-6.95 (m, 1H); 7.00-7.05 (m, 1H); 7.10 (dd, J=2.26 Hz, 7.47 Hz, 1H); 9.85 (t, J=1.22 Hz, 1H)

### Step 3: 4-Bromo-2-(3,3-difluoropropoxy)-1-fluorobenzene

To a cooled (−20° C.) solution of the crude aldehyde from the previous step (12.57 g, 50.88 mmol) in DCM (102 mL) was added diethylaminosulfurtrifluoride (17.22 g, 106.8 mmol, 14 mL). The reaction mixture was stirred at −20° C. for 1′0.5 h then allowed to warm up to room temperature overnight. The reaction mixture was concentrated onto 50 g Celite. Flash chromatography (SiO₂, hexanes to 5:95 EtOAc:Hexanes) provided 5.3 g, 39%, of the title compound as a yellow oil.

MS (EI): m/z 268 (M^{+.}).

### Step 4: 1-(Difluoromethoxy)-4-((3-(3,3-difluoropropoxy)-4-fluorophenyl)ethynyl)-2-methylbenzene

This compound was made in a similar manner to Example 102 Step 2 using 4-bromo-2-(3,3-difluoropropoxy)-1-fluorobenzene from the previous step (896 mg, 3.33 mmol), 1-(difluoromethoxy)-4-ethynyl-2-methylbenzene from Example 101 Step 3 (698 mg, 3.83 mmol), PdCl₂(PPh₃)₂ (117 mg, 0.167 mmol), CuI (63 mg, 0.333 mmol), TEA (1.68 g, 2.3 mL, 16.65 mmol, and DMF (7.4 mL) to provide 230 mg, 18%, of the title compound as a yellow oil.

MS (EI): m/z 370 (M^{+.}).

### Step 5: 1-(4-(Difluoromethoxy)-3-methylphenyl)-2-(3-(3,3-difluoropropoxy)-4-fluorophenyl)ethane-1,2-dione

This compound was made in a similar manner to Example 95 Step 5 using 1-(difluoromethoxy)-4-((3-(3,3-difluoropropoxy)-4-fluorophenyl)ethynyl)-2-methylbenzene from the previous step (230 mg, 0.621 mmol), PdCl₂(ACN)₂ (16 mg, 0.062 mmol), and DMSO (2.5 mL) to provide 183 mg, 73%, of the title compound as a yellow solid.

MS (EI): m/z 402 (M^{+.}).

### Step 6: 2-Amino-4-(4-(difluoromethoxy)-3-methylphenyl)-4-(3-(3,3-difluoropropoxy)-4-fluorophenyl)-1-methyl-1H-imidazol-5(4H)-one

This compound was made in a similar manner to Example 95 Step 6 using 1-(4-(difluoromethoxy)-3-methylphenyl)-2-(3-(3,3-difluoropropoxy)-4-fluorophenyl)ethane-1,2-dione from the previous step (183 mg, 0.455 mmol), 1-methylguanidine.HCl (75 mg, 0.683 mmol), Na₂CO₃ (72 mg, 0.683 mmol), and 200P EtOH (1.3 mL) to provide 129 mg, 62%, of the title compound as a beige foam.

MS (+ESI): m/z 458.1 ([M+H]⁺)

### EXAMPLE 131

### Preparation of: (5S)-2-Amino-5-[4-(difluoromethoxy)-3-methylphenyl]-5-[3-(3,3-difluoropropoxy)-4-fluorophenyl]-3-methyl-3,5-dihydro-4H-imidazol-4-one

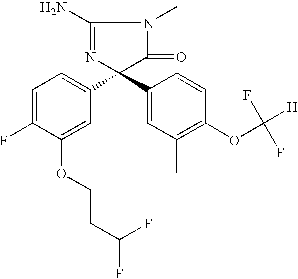

The compound from Example 130 Step 6 was separated by chiral HPLC (Chiralcel AD-H, 2×25 cm; 11% EtOH in Hexanes with DEA additive) to provide the title compound as a white powdery foam.

MS (+ESI): m/z 458.1 ([M+H]⁺)

### EXAMPLE 132

### Preparation of: (5R)-2-Amino-5-[4-(difluoromethoxy)-3-methylphenyl]-5-[3-(3,3-difluoropropoxy)-4-fluorophenyl]-3-methyl-3,5-dihydro-4H-imidazol-4-one

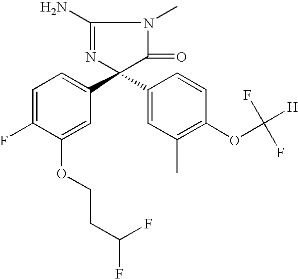

The compound from Example 130 Step 6 was separated by chiral HPLC (Chiralcel AD-H, 2×25 cm; 11% EtOH in Hexanes with DEA additive) to provide the title compound as a white foam.

MS (+ESI): m/z 458.1 ([M+H]⁺)

### EXAMPLE 133

### Preparation of: 2-Amino-5-[3-cyclopropyl-4-(difluoromethoxy)phenyl]-5-[3-(3,3-difluoropropoxy)-4-fluorophenyl]-3-methyl-3,5-dihydro-4H-imidazol-4-one

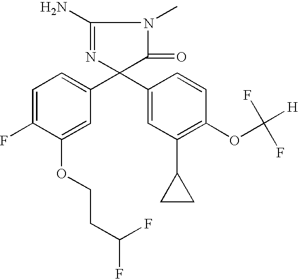

### Step 1: 2-Cyclopropyl-1-(difluoromethoxy)-4-((3-(3,3-difluoropropoxy)-4-fluorophenyl)ethynyl)benzene

This compound was made in a similar fashion to Example 102 Step 2 using 4-bromo-2-(3,3-difluoropropoxy)-1-fluorobenzene (896 mg, 3.33 mmol) from Example 131 Step 3,2-cyclopropyl-1-difluoromethoxy-4-ethynyl-benzene (798 mg, 3.83 mmol) from Example 124 Step 5, TEA (1.68 g, 2.3 mL, 16.65 mmol), PdCl₂(PPh₃)₂ (117 mg, 0.167 mmol), CuI (63 mg, 0.333 mmol), and DMF (7.4 mL) to provide 460 mg, 35%, of the title compound as an orange oil.

MS (+ESI): m/z 396 (M^{+.}).

### Step 2: 1-(3-Cyclopropyl-4-(difluoromethoxy)phenyl)-2-(3-(3,3-difluoropropoxy)-4-fluorophenyl)ethane-1,2-dione

This compound was made in a similar manner to Example 95 Step 5 using 2-cyclopropyl-1-(difluoromethoxy)-4-((3-(3,3-difluoropropoxy)-4-fluorophenyl)ethynyl)benzene (460 mg, 1.16 mmol) from the previous step), PdCl₂(ACN)₂ (30 mg, 0.116 mmol), and DMSO (4.6 mL) to provide 498 mg, 100%, of the title compound as an orange oil that partially solidified upon standing.

MS (+ESI): m/z 429 ([M+H]⁺).

### Step 3: 2-Amino-5-[3-cyclopropyl-4-(difluoromethoxy)phenyl]-5-[3-(3,3-difluoropropoxy)-4-fluorophenyl]-3-methyl-3,5-dihydro-4H-imidazol-4-one

This compound was made in a similar manner to Example 95 Step 6 using 1-(3-cyclopropyl-4-(difluoromethoxy)phenyl)-2-(3-(3,3-difluoropropoxy)-4-fluorophenyl)ethane-1,2-dione (498 mg, 1.16 mmol) from the previous step, 1-methylguanidine hydrochloride (191 mg, 1.74 mmol), Na₂CO₃ (184 mg, 1.74 mmol) and iPrOH (3.3 mL) to provide 346 mg, 61%, of the title compound as a beige foam.

MS (+ESI): m/z 484.1 ([M+H]⁺).

### EXAMPLE 134

### Preparation of: 2-Amino-5-[4-(difluoromethoxy)-3-ethylphenyl]-5-[3-(3,3-difluoropropoxy)-4-fluorophenyl]-3-methyl-3,5-dihydro-4H-imidazol-4-one

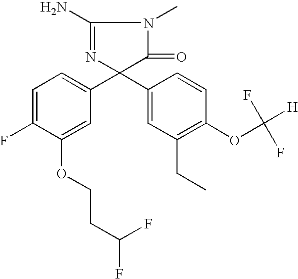

### Step 1: ((3-(3,3-Difluoropropoxy)-4-fluorophenyl)ethynyl)trimethylsilane

This compound was made in a similar fashion to Example 95 Step 2 using 4-bromo-2-(3,3-difluoropropoxy)-1-fluorobenzene (1.06 g, 3.94 mmol) from Example 131 Step 3, trimethylsilylacetylene (580 mg, 835 μL, 5.91 mmol), TEA (2.0 g, 2.75 mL, 19.7 mmol), PdCl₂(PPh₃)₂ (138 mg, 0.196 mmol), CuI (75 mg, 0.394 mmol), and DMF (8.8 mL) to provide 900 mg, 79%, of the title compound as an orange-brown oil.

¹H NMR 500 MHz (CDCl₃) δ 0.22 (s, 9H); 2.25-2.40 (m, 2H); 4.15 (t, J=6.08 Hz, 2H); 6.08 (tt, J=3.51 Hz, 56.05 Hz); 6.93-7.07 (m, 3H)

### Step 2: 2-(3,3-Difluoropropoxy)-4-ethynyl-1-fluorobenzene

This compound was made in a similar manner to Example 95 Step 3 using ((3-(3,3-difluoropropoxy)-4-fluorophenyl)ethynyl)trimethylsilane (900 mg, 3.14 mmol), K₂CO₃ (4.34 g, 31.4 mmol), and MeOH (7.85 mL) to provide 605 mg, 90%, of the title compound as an orange oil.

### Step 3: 4-Bromo-1-(difluoromethoxy)-2-ethylbenzene

To 4-bromo-2-ethylphenol (3.4 g, 16.9 mmol) from the previous step was added 20.25 mL DMF followed by 12.25 mL water. To this solution was added sodium chlorodifluoroacetate (9.56 g, 58.13 mmol) followed by Cs₂CO₃ (15.42 g, 47.33 mmol). The reaction mixture was heated at 110° C. overnight. Then the reaction mixture was cooled to 0° C. and 30 mL conc. HCl was added followed by water at room temperature. The aqueous mixture was extracted with diethyl ether twice. The combined ether layers were washed with water once, dried over Na₂SO₄, filtered, and concentrated in vacuo to give an oil that was absorbed onto 15 g Celite. Flash chromatography (SiO₂, Hexanes to 20% EtOAc 80% Hexanes) provided 744 mg, 17%, of the title compound as a colorless oil.

¹H NMR 500 MHz (CDCl₃) δ1.18 (t, J=7.53 Hz, 3H); 2.63 (quartet, J=7.57 Hz, 2H); 6.44 (t, J=73.77 Hz, 1H); 6.93 (d, J=8.69 Hz, 1H); 7.28 (dd, J=2.70 Hz, 8.44 Hz, 1H); 7.35 (d, J=2.43 Hz, 1H)

### Step 4: 1-(Difluoromethoxy)-4-((3-(3,3-difluoropropoxy)-4-fluorophenyl)ethynyl)-2-ethylbenzene

This compound was made in a similar manner to Example 102 Step 2 using 2-(3,3-difluoropropoxy)-4-ethynyl-1-fluorobenzene (744 mg, 3.47 mmol) from Step 2,4-bromo-1-(difluoromethoxy)-2-ethylbenzene (605 mg, 2.40 mmol), TEA (1.75 g, 2.4 mL, 17.35 mmol), PdCl₂(PPh₃)₂ (121 mg, 0.174 mmol), CuI (66 mg, 0.348 mmol), and DMF (7.7 mL) to provide 291 mg, 31%, of the title compound as a yellow oil.

MS (EI): m/z 384 (M^{+.}).

### Step 5: 1-(4-(Difluoromethoxy)-3-ethylphenyl)-2-(3-(3,3-difluoropropoxy)-4-fluorophenyl)ethane-1,2-dione

This compound was made in a similar manner to Example 95 Step 3 using 1-(difluoromethoxy)-4-((3-(3,3-difluoropropoxy)-4-fluorophenyl)ethynyl)-2-ethylbenzene (290 mg, 0.754 mmol) from the previous step, PdCl₂(ACN)₂ (19.5 mg, 0075 mmol), and DMSO (3.0 mL) to provide 245 mg, 78%, of the title compound as a yellow oil.

MS (+ESI): m/z 417.1 ([M+H]⁺).

### Step 6: 2-Amino-5-[4-(difluoromethoxy)-3-ethylphenyl]-5-[3-(3,3-difluoropropoxy)-4-fluorophenyl]-3-methyl-3,5-dihydro-4H-imidazol-4-one

This compound was made in a similar manner to Example 95 Step 4 using 1-(4-(difluoromethoxy)-3-ethylphenyl)-2-(3-(3,3-difluoropropoxy)-4-fluorophenyl)ethane-1,2-dione (245 mg, 0.588 mmol) from the previous step, 1-methylguanidine.HCl (97 mg, 0.826 mmol), Na₂CO₃ (94 mg, 0.826 mmol), and iPrOH (1.7 mL) to provide 136 mg, 47%, of the title compound as a beige foam.

MS (+ESI): m/z 472.1 ([M+H]⁺).

### EXAMPLE 135

### Preparation of: (5S)-2-Amino-5-[4-(difluoromethoxy)-3-ethylphenyl]-5-[3-(3,3-difluoropropoxy)-4-fluorophenyl]-3-methyl-3,5-dihydro-4H-imidazol-4-one

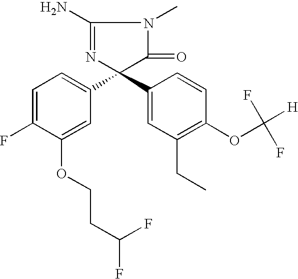

The compound from Example 134 Step 8 was separated by chiral HPLC (Chiralpak AD-H 0.46×25 cm 10% EtOH in Hexanes with NPA additive) to provide the title compound as an off-white solid.

MS (+ESI): m/z 472.1 ([M+H]⁺).

### EXAMPLE 136

### Preparation of: (5R)-2-Amino-5-[4-(difluoromethoxy)-3-ethylphenyl]-5-[3-(3,3-difluoropropoxy)-4-fluorophenyl]-3-methyl-3,5-dihydro-4H-imidazol-4-one

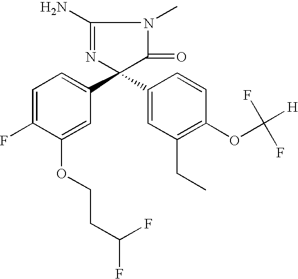

The compound from Example 134 Step 8 was separated by chiral HPLC (Chiralpak AD-H 0.46×25 cm 10% EtOH in Hexanes with NPA additive) to provide the title compound as an off-white solid.

MS (+ESI): m/z 472.1 ([M+H]⁺)

### EXAMPLE 137

### Preparation of: 2-Amino-5-[4-(difluoromethoxy)-3-isopropylphenyl]-3-methyl-5-phenyl-3,5-dihydro-4H-imidazol-4-one

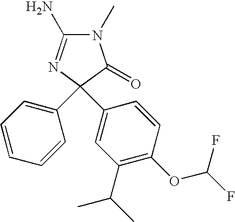

### Step 1: 4-Bromo-2-isopropylphenol

In a 500 mL round-bottomed flask was placed 2-isopropylphenol (5 g, 36.7 mmol) and CHCl₃ (100 ml) was added to give a brown solution. Tetra-n-butylammonium tribromide (17.70 g, 36.7 mmol) was added as a solution in CHCl₃ (100 ml). The reaction was stirred overnight at RT. A 5% sodium thiosulfate solution (200 ml) and mixture was stirred for 30 min. The layers were separated. The organic was washed with 1M HCl (200 ml). The organic layer was dried over Na₂SO₄. The crude was purified by flash chromatography (gradient 0-100% EtOAc/hex) to provide 4-bromo-2-isopropylphenol (6.2 g, 28.8 mmol, 79% yield) as a light colored solid. ¹H NMR (400 MHz, DMSO-d₆) [symbol] 9.56 (s, 1H), 7.16 (d, 2.43H, 1H), 7.10 (dd, J=8.46, 2.55 Hz, 1H), 6.70 (d, J=8.58, 1H), 3.12 (septet, J=6.85 Hz, 1H), 1.10 (d, J=6.84, 6H)

### Step 2: 4-Bromo-2-isopropyl(difluoromethoxy)benzene

To 4-bromo-2-isopropylphenol (4.8 g, 22.31 mmol) from the previous step was added DMF (27 mL) followed by water (3 mL). To this solution was added sodium chlorodifluoroacetate (12.65 g, 76.91 mmol) followed by Cs₂CO₃ (20.4 g, 62.46 mmol). The reaction mixture was heated at 110° C. overnight. Then the reaction mixture was cooled to 0° C. and 30 mL conc. HCl was added followed by water at room temperature. The aqueous mixture was extracted with diethyl ether twice. The combined ether layers were washed with water once, dried over Na₂SO₄, filtered, and concentrated in vacuo to give 5 g of an oil that was absorbed onto 20 g Celite. Flash chromatography (SiO₂, Hexanes to 20% EtOAc 80% Hexanes) provided 1.5 g, of the title compound as a colorless oil. There was also recovered 3.3 g (15.34 mmol) of the starting bromide that was resubjected to reaction conditions (sodium chlorodifluoroacetate (8.7 g, 52.93 mmol, 1.0 eq.), Cs₂CO₃ (14.0 g, 42.95 mmol, 2.8 eq.), 18 mL DMF and 2 mL water) to give an additional 1.0 g of the product for a total yield of 2.5 g, 42%, of the title compound.

¹H NMR 500 MHz (CDCl₃) δ1.20 (d, 6H, J=6.85 Hz); 3.27 (m, 1H); 6.45 (t, 1H, J=73.77 Hz); 6.93 (d, 1H, J=8.7 Hz); 7.27 (dd, 1H, J=8.64 Hz, 2.50 Hz); 7.39 (d, 1H, J=2.44 Hz)

### Step 3: 1-(Difluoromethoxy)-2-isopropyl-4-(phenylethynyl)benzene

This compound was made in a similar manner to Example 102 Step 2 using 4-bromo-2-isopropyl(difluoromethoxy)benzene (1.0 g, 3.77 mmol) from the previous step, phenyl acetylene (444 mg, 4.33 mmol, 477 μL), dichloropalladium(II) bis(triphenylphosphine) (132 mg, 0.189 mmol), copper(I) iodide (72 mg, 0.377 mmol), triethylamine (TEA) (1.9 g, 18.85 mmol, 2.62 mL), and DMF (7.5 mL) to yield 637 mg, 59%, of the title compound as a colorless to light yellow oil.

MS (EI): m/z 286 (M^{+.}).

### Step 4: 1-(4-(Difluoromethoxy)-3-isopropylphenyl)-2-phenylethane-1,2-dione

This compound was made in a similar manner to Example 95 Step 5 using 1-(difluoromethoxy)-2-isopropyl-4-(phenylethynyl)benzene (630 mg, 2.2 mmol) from the previous step, dichloropalladium(II) bisacetonitrile (57 mg, 0.022 mmol), and dry DMSO (8.8 mL) to give 546 mg, 78%, of the title compound as a yellow oil.

MS (EI): m/z 318 (M^{+.}).

### Step 5: 2-Amino-5-[4-(difluoromethoxy)-3-isopropylphenyl]-3-methyl-5-phenyl-3,5-dihydro-4H-imidazol-4-one

This compound was made in a similar fashion to Example 95 Step 6 using 1-(4-(difluoromethoxy)-3-isopropylphenyl)-2-phenylethane-1,2-dione (540 mg, 1.7 mmol) from the previous step, 1-methylguanidine hydrochloride (279 mg, 2.55 mmol), Na₂CO₃ (270 mg, 2.55 mmol), and 200P EtOH (4.5 mL) to give 547 mg, 72%, of the title compound as a beige foam.

MS (+APPI): m/z 374 ([M+H]⁺).

### EXAMPLE 138

### Preparation of: (5R)-2-Amino-5-[4-(difluoromethoxy)-3-isopropylphenyl]-3-methyl-5-phenyl-3,5-dihydro-4H-imidazol-4-one

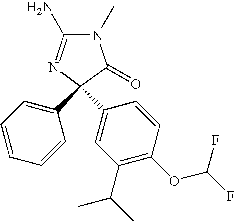

The title compound from Example 137 Step 5 was separated into its enantiomers by chiral HPLC (Chiralpak AD-H, 2×25 cm; 10% EtOH in Hexane with NPA additive) to provide the title compound as a white foam.

MS (+APPI): m/z 374 ([M+H]⁺).

### EXAMPLE 139

### Preparation of: (5S)-2-Amino-5-[4-(difluoromethoxy)-3-isopropylphenyl]-3-methyl-5-phenyl-3,5-dihydro-4H-imidazol-4-one

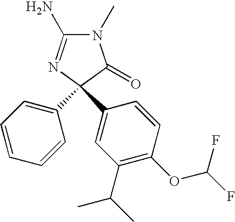

The title compound from Example 137 Step 5 was separated into its enantiomers by chiral HPLC (Chiralpak AD-H, 2×25 cm; 10% EtOH in Hexane with NPA additive) to provide the title compound as a white foam.

MS (+APPI): m/z 374 ([M+H]⁺).

### EXAMPLE 140

### Preparation of: 2-Amino-5-[4-(difluoromethoxy)-3-(2-hydroxyethyl)phenyl]-3-methyl-5-phenyl-3,5-dihydro-4H-imidazol-4-one

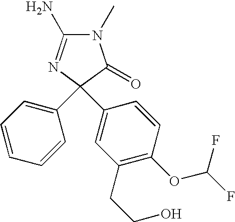

### Step 1: 2-(2-(Difluoromethoxy)-5-(phenylethynyl)phenyl)ethanol

This compound was made in a similar manner to Example 102 Step 2 using 2-(5-bromo-2-(difluoromethoxy)phenyl)ethanol (1.068 g, 4.0 mmol) from a previous example, phenylacetylene (470 mg, 505 μL, 4.60 mmol), TEA (2.02 g, 2.79 mL, 20 mmol), PdCl₂(PPh₃)₂ (140 mg, 0.20 mmol), CuI (76 mg, 0.10 mmol), and DMF (8.9 mL) to provide 935 mg of a dark red-brown oil. ¹H NMR analysis of the oil after chromatography shows it to be a mixture of 2-(2-(difluoromethoxy)-5-(phenylethynyl)phenyl)ethanol and 2-(5-bromo-2-(difluoromethoxy)phenyl)ethanol with the desired product being the major component. This material is taken on to the next step as is.

### Step 2: 1-(4-(Difluoromethoxy)-3-(2-hydroxyethyl)phenyl)-2-phenylethane-1,2-dione

This compound was made in a similar manner to Example 95 Step 5 using 2-(2-(difluoromethoxy)-5-(phenylethynyl)phenyl)ethanol (935 mg, 3.24 mmol) from the previous step, PdCl₂(ACN)₂ (84 mg, 0.324 mmol), and DMSO (13 mL) to provide 598 mg, 57%, of the title compound as an orange-red oil.

MS (EI): m/z 320 (M^{+.}).

### Step 3: 2-Amino-5-[4-(difluoromethoxy)-3-(2-hydroxyethyl)phenyl]-3-methyl-5-phenyl-3,5-dihydro-4H-imidazol-4-one

This compound was made in a similar fashion to Example 95 Step 6 using 1-(4-(difluoromethoxy)-3-(2-hydroxyethyl)phenyl)-2-phenylethane-1,2-dione (64 mg, 0.200 mmol) from the previous step, 1-methylguanidine hydrochloride (99 mg, 0.90 mmol), Na₂CO₃ (96 mg, 0.90 mmol), and iPrOH (571 μL) to provide 33 mg, 44%, of the title compound as a beige solid.

MS (+ESI): m/z 376.1 ([M+H]⁺).

### EXAMPLE 141

### Preparation of: 2-Amino-5-[4-(difluoromethoxy)-3-(2-chloroethyl)phenyl]-3-methyl-5-phenyl-3,5-dihydro-4H-imidazol-4-one

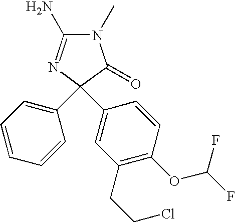

### Step 1: 1-(3-(2-Chloroethyl)-4-(difluoromethoxy)phenyl)-2-phenylethane-1,2-dione

To a dry 10 mL round bottom flask under nitrogen was added 1-(4-(difluoromethoxy)-3-(2-hydroxyethyl)phenyl)-2-phenylethane-1,2-dione (128 mg, 0.400 mmol) from Example 140 Step, dry DCM (727 μL), triphenylphosphine (210 mg, 0.800 mmol), and CCl₄ (369 mg, 232 μL, 2.40 mmol) in that order at room temperature. The mixture was stirred overnight at room temperature. Then the mixture was concentrated onto 1.5 g Celite. Flash chromatography (SiO₂, 1:9 EtOAc:Hexanes to 25:75 EtOAc:Hexanes) provided 110 mg, 81%, of the title compound as a yellow oil.

MS (EI): m/z 338 (M^{+.}).

### Step 2: 2-Amino-5-[4-(difluoromethoxy)-3-(2-chloroethyl)phenyl]-3-methyl-5-phenyl-3,5-dihydro-4H-imidazol-4-one

This compound was made in a similar manner to Example 95 Step 6 using 1-(3-(2-chloroethyl)-4-(difluoromethoxy)phenyl)-2-phenylethane-1,2-dione (110 mg, 0.325 mmol) from the previous step, 1-methylguanidine hydrochloride (39 mg, 0.357 mmol) and Na₂CO₃ (37 mg, 0.357 mmol), and iPrOH (928 μL) to provide 83 mg, 65%, of a yellow solid.

MS (+ESI): m/z 394 ([M+H]⁺).

### EXAMPLE 142

### Preparation of: 2-Amino-5-[4-(difluoromethoxy)-3-(2-methoxyethyl)phenyl]-3-methyl-5-phenyl-3,5-dihydro-4H-imidazol-4-one

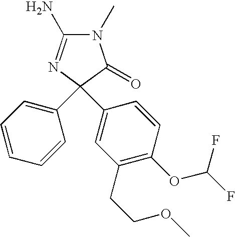

### Step 1: 4-Bromo-1-(difluoromethoxy)-2-(2-methoxyethyl)benzene

To a solution of 2-(5-bromo-2-(difluoromethoxy)phenyl)ethanol (534 mg, 2.0 mmol) in hexanes (20 mL) and THF (0.5 mL) was added sodium hydroxide (50% aq, 160 μL, 2.0 mmol) followed by dimethyl sulfate (571 μL, 6.0 mmol) at room temperature. The mixture was stirred at room temperature overnight at which point it was diluted with water. The aqueous layer was separated and extracted with EtOAc once. The combined organic layers were dried over Na₂SO₄, filtered, and concentrated over 2.5 g Celite. Flash chromatography (SiO₂, 2.5% EtOAc 97.5% Hexanes to 40% EtOAc 60% Hexanes) yielded 300 mg, 53%, of a colorless oil. NMR of the compound looks good.

MS (EI): m/z 279.9 (M^{+.})

### Step 2: 2-(2,2-Difluoroethoxy)-4-((4-(difluoromethoxy)-3-methylphenyl)ethynyl)-1-fluorobenzene

To a 500 mL RBF was charged a large magnetic stirbar, 4-bromo-2-(2,2-difluoroethoxy)-1-fluorobenzene from Example 102 Step 1 (27.6 g, 108 mmol), 1-(difluoromethoxy)-4-ethynyl-2-methylbenzene from the previous step (124 mmol, 22.67 g), TEA (54.8 g, 75 mL, 541 mmol), and DMF (240 mL). The resulting solution was degassed for 30 min using a nitrogen sparge then copper(I) iodide (2.06 g, 10.82 mmol) and PdCl₂(PPh₃)₂ (3.80 g, 5.41 mmol) were added. The reaction was heated at 70° C. under N₂ overnight. The reaction was cooled to room temperature then it was diluted with water and 30 mL conc. HCl was added. The mixture was extracted with EtOAc three times. The combined organic layers were combined, dried over Na₂SO₄, filtered, and concentrated in vacuo to give a dark brown oil. This oil was loaded onto a large SiO₂ pad and the pad was eluted with hexanes (approx. 2 L) then 5% EtOAc 95% Hexanes until all of the desired product had eluted. There were 2 large fractions collected. Fraction 1 consisted of the starting bromide and the desired product in a 1:1 ratio by NMR. Fraction 2 consisted of the desired product and the starting bromide with the product consisting >80% and the bromide <20% by NMR. Both of these fractions were subjected to flash chromatography (SiO₂, hexanes to 5% EtOAc 95% hexanes) to give a combined total of 29.07 g of a red-orange oil. By ¹H NMR this oil consisted of a 19:7 mol ratio of the desired product and EtOAc. Actual amount of title compound is 26.6 g, 69%. This material is carried on as is.

### Step 3: 1-(4-(Difluoromethoxy)-3-(2-methoxyethyl)phenyl)-2-phenylethane-1,2-dione

This compound was made in a similar manner to Example 95 Step 5 using 1-(difluoromethoxy)-2-(2-methoxyethyl)-4-(phenylethynyl)benzene from the previous step (100 mg, 0.331 mmol), PdCl₂(ACN)₂ (8.58 mg, 0.033 mmol), and DMSO (1.3 mL) to provide 86 mg, 78%, of the title compound as an orange oil.

MS (EI): m/z 334 (M^{+.}).

### Step 4: 2-Amino-4-(4-(difluoromethoxy)-3-(2-methoxyethyl)phenyl)-1-methyl-4-phenyl-1H-imidazol-5(4H)-one

This compound was made in a similar manner to Example 95 Step 6 using 1-(4-(difluoromethoxy)-3-(2-methoxyethyl)phenyl)-2-phenylethane-1,2-dione from the previous step (85 mg, 0.254 mmol), 1-methylguanidine.HCl (42 mg, 0.381 mmol), Na₂CO₃ (40 mg, 0.381 mmol), and iPrOH (726 μL) to provide 73 mg, 74%, of the title compound as a light yellow foam.

MS (+ESI): m/z 390.1 ([M+H]⁺).

### EXAMPLE 143

### Preparation of: (5S)-2-Amino-5-[3-(2-chloroethyl)-4-(difluoromethoxy)phenyl]-3-methyl-5-phenyl-dihydro-4H-imidazol-4-one

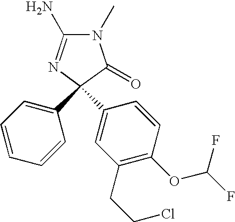

The compound from Example 142 Step 2 was separated by chiral HPLC (Chiralcel OD-H 2×25 cm; 20% EtOH in CO₂ with NPA additive) to provide the title compound as a beige foam/powder.

MS (+ESI): m/z 390.1 ([M+H]⁺).

### EXAMPLE 144

### Preparation of: (5R)-2-Amino-5-[3-(2-chloroethyl)-4-(difluoromethoxy)phenyl]-3-methyl-5-phenyl-dihydro-4H-imidazol-4-one

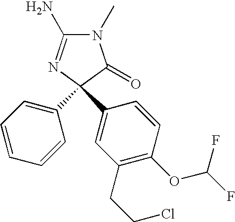

The compound from Example 142 Step 2 was separated by chiral HPLC (Chiralcel OD-H 2×25 cm; 20% EtOH in CO₂ with NPA additive) to provide the title compound as a beige foam/powder.

MS (+ESI): m/z 390.1 ([M+H]⁺).

### EXAMPLE 145

### Preparation of: 2-Amino-5-[3-(cyclopropylethynyl)-4-fluorophenyl]-5-[4-(difluoromethoxy)-3-isopropylphenyl]-3-methyl-3,5-dihydro-4H-imidazol-4-one

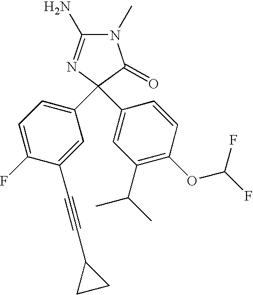

### Step 1: ((4-(Difluoromethoxy)-3-isopropylphenyl)ethynyl)trimethylsilane

This compound was made in a similar fashion to Example 95 Step 2 using 4-bromo-2-isopropyl(difluoromethoxy)benzene (1.50 g, 5.66 mmol) from Example 137 Step 2, trimethylsilylacetylene (834 mg, 1.20 mL, 8.49 mmol), TEA (2.86 g, 3.95 mL, 28.3 mmol), PdCl₂(PPh₃)₂ (199 mg, 0.283 mmol), CuI (108 mg, 0.566 mmol), and DMF (12.5 mL) to provide 634 mg, 40%, of the title compound as a colorless to light yellow oil.

MS (EI): m/z 282 (M^{+.}).

### Step 2: 1-(Difluoromethoxy)-4-ethynyl-2-isopropylbenzene

This compound was made in a similar manner to Example 95 Step 3 using ((4-(difluoromethoxy)-3-isopropylphenyl)ethynyl)trimethylsilane (630 mg, 2.23 mmol) from the last step, K₂CO₃ (3.08 g, 22.3 mmol), and MeOH (5.6 mL) to provide 359 mg, 76%, of the title compound as a colorless to light yellow oil.

MS (EI): m/z 210 (M^{+.}).

### Step 3: 2-Bromo-4-((4-(difluoromethoxy)-3-isopropylphenyl)ethynyl)-1-fluorobenzene

This compound was made in a similar manner to Example 95 Step 2 using 1-(difluoromethoxy)-4-ethynyl-2-isopropylbenzene (350 mg, 1.66 mmol) from the previous step, 3-bromo-4-fluoro-iodobenzene (454 mg, 170 μL, 1.51 mmol), TEA (764 mg, 1.05 mL, 7.55 mmol), PdCl₂(PPh₃)₂ (53 mg, 0.0755 mmol), CuI (8.6 mg, 0.0453 mmol), and DMF (2.3 mL) to provide 433 mg, 75%, of the title compound as a colorless oil.

MS (EI): m/z 382 (M^{+.}).

### Step 4: 1-(3-Bromo-4-fluorophenyl)-2-(4-(difluoromethoxy)-3-isopropylphenyl)ethane-1,2-dione

This compound was made in a similar manner to Example 95 Step 5 using 2-bromo-4-((4-(difluoromethoxy)-3-isopropylphenyl)ethynyl)-1-fluorobenzene (425 mg, 1.11 mmol) from the previous step, PdCl₂(ACN)₂ (29 mg, 0.011 mmol), and DMSO (4.5 mL) to provide 365 mg, 79%, of the title compound as a yellow solid.

MS (−ESI): m/z 413/415 ([M−H]⁻).

### Step 5: 1-(3-(Cyclopropylethynyl)-4-fluorophenyl)-2-(4-(difluoromethoxy)-3-isopropylphenyl)ethane 1,2-dione

This compound was made in a similar manner to Example 102 Step 2 using 1-(3-bromo-4-fluorophenyl)-2-(4-(difluoromethoxy)-3-isopropylphenyl)ethane-1,2-dione (295 mg, 0.710 mmol) from the previous step, cyclopropylacetylene (70 wt % in toluene, 54 mg, 0.817 mmol), TEA (359 mg, 495 μL, 3.55 mmol), PdCl₂(PPh₃)₂ (25 mg, 0.0355 mmol), CuI (13.3 mg, 0.0710 mmol), and DMF (1.6 mL) to provide 156 mg, 55%, of the title compound as an orange oil. The cyclopropylacetylene solution was added after the solution was degassed.

MS (+APPI): m/z 401 ([M+H]⁺).

### Step 6: 2-Amino-5-[3-(cyclopropylethynyl)-4-fluorophenyl]-5-[4-(difluoromethoxy)-3-isopropylphenyl]-3-methyl-3,5-dihydro-4H-imidazol-4-one

This compound was made in a similar manner to Example 95 Step 6 using (150 mg, 0.315 mmol) from the previous step, 1-methylguanidine hydrochloride (61 mg, 0.562 mmol), Na₂CO₃ (60 mg, 0.562 mmol), and EtOH (1.1 mL) to provide 92 mg, 53%, of the title compound as an oily beige foam.

MS (+ESI): m/z 456.2 ([M+H]⁺).

### EXAMPLE 146

### Preparation of: 2-Amino-5-[3-(cyclopropylethynyl)-4-fluorophenyl]-5-[4-(difluoromethoxy)-3-ethylphenyl]-3-methyl-3,5-dihydro-4H-imidazol-4-one

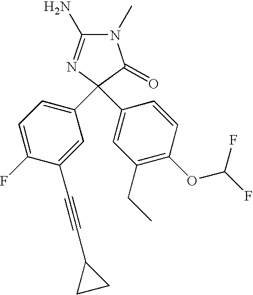

### Step 1: 2-Bromo-4-((4-(difluoromethoxy)-3-ethylphenyl)ethynyl)-1-fluorobenzene

This compound was made in a similar manner to Example 95 Step 2 using 1-(difluoromethoxy)-2-ethyl-4-ethynylbenzene (2.55 g, 12.95 mmol) from Example 117 Step 4, 3-bromo-4-fluoro-iodobenzene (3.54 g, 11.77 mmol), TEA (5.96 g, 8.2 mL, 58.85 mmol), PdCl₂(PPh₃)₂ (413 mg, 0.589 mmol), CuI (67 mg, 0.353 mmol), and DMF (18 mL) to provide 3.73 g, 78%, of the title compound as a colorless oil. The reaction mixture was heated for 1.5 h before workup.

MS (EI): m/z 368 (M^{+.}).

### Step 2: 1-(3-Bromo-4-fluorophenyl)-2-(4-(difluoromethoxy)-3-ethylphenyl)ethane-1,2-dione

This compound was made in a similar manner to Example 95 Step 5 using 2-Bromo-4-((4-(difluoromethoxy)-3-ethylphenyl)ethynyl)-1-fluorobenzene (3.7 g, 10.0 mmol) from the previous step, PdCl₂(ACN)₂ (259 mg, 1.0 mmol), and DMSO (40 mL) to provide 3.08 g, 76%, of the title compound as a light yellow solid.

¹H NMR 500 MHz (DMSO-d₆) δ1.12 (t, J=7.54 Hz, 3H); 2.65 (quartet, J=7.50 Hz, 2H); 7.31 (d, J=8.46 Hz, 1H); 7.40 (t, J=66.65 Hz, 1H); 7.57-7.59 (m, 1H); 7.82 (dd, J=2.15 Hz, 8.52 Hz, 1H); 7.90 (d, J=2.09 Hz, 1H); 7.93-7.98 (m, 1H); 8.23 (dd, J=2.15 Hz, 6.67 Hz, 1H)

### Step 3: 2-Amino-5-[3-bromo-4-fluorophenyl]-5-[4-(difluoromethoxy)-3-ethylphenyl]-3-methyl-3,5-dihydro-4H-imidazol-4-one

This compound was made in a similar manner to Example 95 Step 6 using 1-(3-bromo-4-fluorophenyl)-2-(4-(difluoromethoxy)-3-ethylphenyl)ethane-1,2-dione (3.08 g, 7.67 mmol), 1-methylguanidine.HCl (1.26 g, 11.51 mmol), Na₂CO₃ (1.22 g, 11.51 mmol), and EtOH (22 mL) to provide 2.45 g, 70%, of the title compound as a yellow foam.

MS (+ESI): m/z 456.1 ([M+H]⁺).

### Step 4: 2-Amino-5-[3-(cyclopropylethynyl)-4-fluorophenyl]-5-[4-(difluoromethoxy)-3-ethylphenyl]-3-methyl-3,5-dihydro-4H-imidazol-4-one

A mixture of 2-amino-5-[3-bromo-4-fluorophenyl]-5-[4-(difluoromethoxy)-3-ethylphenyl]-3-methyl-3,5-dihydro-4H-imidazol-4-one (400 mg, 0.877 mmol), acetonitrile (2.1 mL), and pyrrolidine (1.4 mL) was degassed for 20 min with nitrogen then cyclopropylacetylene (70 wt % in toluene, 140 μL, 1.32 mmol), PdCl₂(PPh₃)₂ (0.877 mmol), and CuI (0.0439 mmol) were added. The mixture was heated at 60° C. for 1 h after which an additional amount of cyclopropylacetylene (140 μL, 1.32 mmol) was added and the reasction mixture was cooled to room temperature. Then the mixture was diluted with EtOAc and saturated NaHCO₃. The biphasic mixture was shaken in a seporatory funnel. Then the organic layer was separated, washed with brine, dried over Na₂SO₄, filtered, and concentrated over 2 g Celite. Flash chromatography (SiO₂, DCM to 92:8 DCM:MeOH) provided 371 mg of an orange solid whose ¹H NMR indicates a 2:1 ratio of starting bromide to desired product. This material is resubjected to reaction conditions (0.414 mmol cyclopropylacetylene, 14.3 mg, 0.0276 mmol PdCl₂(PPh₃)₂, 2.6 mg, 0.0138 mmol CuI, 440 μL pyrrolidine, and 900 μL ACN), and rechromatographed as before to provide 314 mg, 81%, of the title compound as an orange foam.

MS (+ESI): m/z 442.2 ([M+H]⁺).

### EXAMPLE 147

### Preparation of: (5R)-2-Amino-5-[3-(cyclopropylethynyl)-4-fluorophenyl]-5-[4-(difluoromethoxy)-3-ethylphenyl]-3-methyl-3,5-dihydro-4H-imidazol-4-one

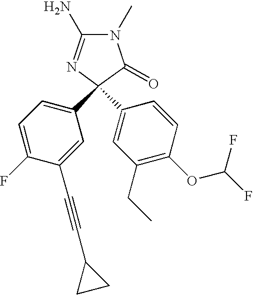

The compound from Example 146 Step 4 was separated by chiral HPLC (7% (8/2 MeOH/EtOH) in Hexane with DEA additive) to provide the title compound as a beige foam.

MS (+ESI): m/z 442.2 ([M+H]⁺).

### EXAMPLE 148

### Preparation of: (5S)-2-Amino-5-[3-(cyclopropylethynyl)-4-fluorophenyl]-5-[4-(difluoromethoxy)-3-ethylphenyl]-3-methyl-3,5-dihydro-4H-imidazol-4-one

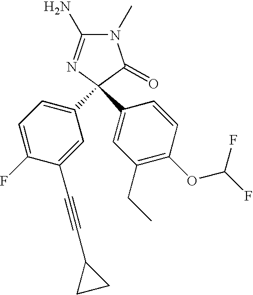

The compound from Example 146 Step 4 was separated by chiral HPLC (7% (8/2 MeOH/EtOH) in hexane with DEA additive) to provide the title compound as a beige to light yellow foam.

MS (+ESI): m/z 442.2 ([M+H]⁺).

### EXAMPLE 149

### Preparation of: 2-Amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one

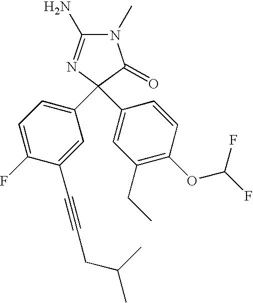

This compound was made in a similar fashion to Example 146 Step 4 using 2-amino-5-[3-bromo-4-fluorophenyl]-5-[4-(difluoromethoxy)-3-ethylphenyl]-3-methyl-3,5-dihydro-4H-imidazol-4-one (400 mg, 0.877 mmol) from Example 146 Step 3, acetonitrile (2.1 mL), pyrrolidine (1.4 mL), PdCl₂(PPh₃)₂ (62 mg, 0.0877 mmol), CuI (8.4 mg, 0.0439 mmol), and 4-methylpent-1-yne (108 mg×2, 145 μL×2, 1.32 mmol×2) to provide 375 mg, 93%, of the title compound as a yellow foam. The reaction was heated for 3 h before the additional amount of alkyne was added.

MS (+ESI): m/z 458.2 ([M+H]⁺).

### EXAMPLE 150

### Preparation of: (5R)-2-Amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one

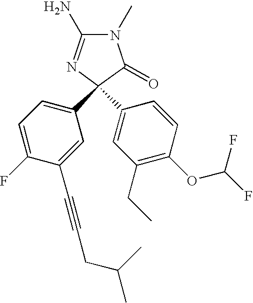

The compound from Example 149 was separated by chiral HPLC (Chiralcel AD-H 2×25 cm; 5% IPA in Hexanes with 0.1% DEA additive) to provide the title compound as a yellow foam.

MS (+ESI): m/z 458.2 ([M+H]⁺).

### EXAMPLE 151

### Preparation of: (5S)-2-Amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one

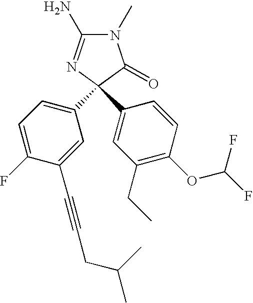

The compound from Example 149 was separated by chiral HPLC (Chiralcel AD-H 2×25 cm; 5% IPA in Hexanes with 0.1% DEA additive) to provide the title compound as a yellow foam.

MS (+ESI): m/z 458.2 ([M+H]⁺).

### EXAMPLE 152

### Preparation of: 2-Amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one

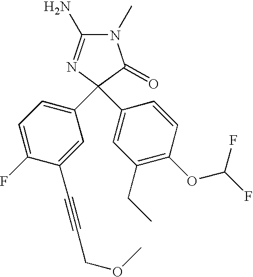

This compound was made in a similar fashion to Example 146 Step 4 using 2-amino-5-[3-bromo-4-fluorophenyl]-5-[4-(difluoromethoxy)-3-ethylphenyl]-3-methyl-3,5-dihydro-4H-imidazol-4-one (400 mg, 0.877 mmol) from Example 146 Step 3, acetonitrile (2.1 mL), pyrrolidine (1.4 mL), PdCl₂(PPh₃)₂ (62 mg, 0.0877 mmol), CuI (8.4 mg, 0.0439 mmol), and propargyl methyl ether (93 mg×2, 111 μL×2, 1.32 mmol×2) to provide 375 mg, 93%, of the title compound as a yellow foam. The reaction was heated for 3 h before the additional amount of alkyne was added then the reaction mixture was heated overnight at 60° C., cooled to room temperature, and worked up.

MS (+ESI): m/z 446.1 ([M+H]⁺).

### EXAMPLE 153

### Preparation of: 2-Amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one

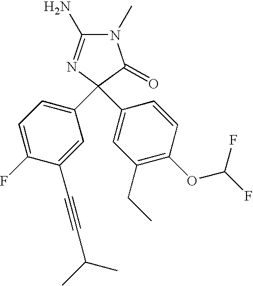

This compound was made in a similar fashion to Example 146 Step 4 using 2-amino-5-[3-bromo-4-fluorophenyl]-5-[4-(difluoromethoxy)-3-ethylphenyl]-3-methyl-3,5-dihydro-4H-imidazol-4-one (400 mg, 0.877 mmol) from Example 146 Step 3, acetonitrile (2.1 mL), pyrrolidine (1.4 mL), PdCl₂(PPh₃)₂ (62 mg, 0.0877 mmol), CuI (8.4 mg, 0.0439 mmol), and isopropyl acetylene (90 mg×2, 1.32 mmol×2) to provide 375 mg, 93%, of the title compound as a yellow foam.

MS (+ESI): m/z 441.1 ([M+H]⁺).

### EXAMPLE 154

### Preparation of: (5R)-2-Amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one

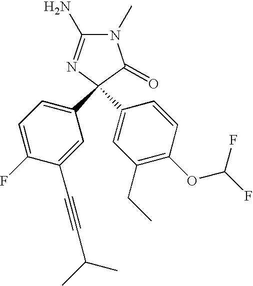

The compound from Example 153 was separated by chiral HPLC (Chiralpak AD-H, 2×25 cm; 3% (8/2 MeOH/EtOH) in Hexanes with DEA additive)) to provide the title compound as a white foam.

MS (+ESI): m/z 441.1 ([M+H]⁺).

### EXAMPLE 155

### Preparation of: (5S)-2-Amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one

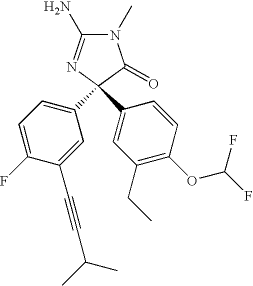

The compound from Example 153 was separated by chiral HPLC (Chiralpak AD-H, 2×25 cm; 3% (8/2 MeOH/EtOH) in Hexanes with DEA additive) to provide the title compound as a white foam.

MS (+ESI): m/z 441.1 ([M+H]⁺).

### EXAMPLE 156

### Preparation of: (5R)-2-Amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one

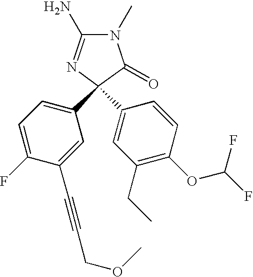

The compound from Example 152 was separated by chiral HPLC (Chiralpak AD-H, 0.46×25 cm; 5% IPA in Hexanes with 0.1% DEA additive) to provide the title compound as a light yellow foam.

MS (+ESI): m/z 446.1 ([M+H]⁺).

### EXAMPLE 157

### Preparation of: (5S)-2-Amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one

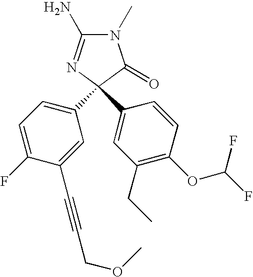

The compound from Example 152 was separated by chiral HPLC (Chiralpak AD-H, 0.46×25 cm; 5% IPA in Hexanes with 0.1% DEA additive) to provide the title compound as a yellow foam.

MS (+ESI): m/z 446.1 ([M+H]⁺).

### EXAMPLE 158

### Preparation of: 2-Amino-5-[4-(difluoromethoxy)-3-ethylphenyl]-5-[3-(3-fluoroprop-1-yn-1-yl)phenyl]-3-methyl-3,5-dihydro-4H-imidazol-4-one

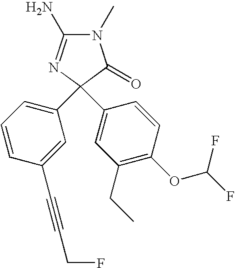

### Step 1: 4-((3-Bromophenyl)ethynyl)-1-(difluoromethoxy)-2-ethylbenzene

This compound was made in a similar fashion to Example 95 Step 2 using 1-(difluoromethoxy)-2-ethyl-4-ethynylbenzene (1.95 g, 9.9 mmol) from the previous step, 3-bromo-1-iodobenzene (2.55 g, 9.0 mmol, 1.15 mL), TEA (4.55 g, 6.27 mL, 45.0 mmol), PdCl₂(PPh₃)₂, (316 mg, 0.45 mmol), CuI (51 mg, 0.27 mmol), and DMF (14 mL) to provide 2.81 g, 88%, of the title compound as a yellow oil. The reaction was worked up after 1 h heating.

MS (EI): m/z 350 (M^{+.}).

### Step 2: 1-(3-Bromophenyl)-2-(4-(difluoromethoxy)-3-ethylphenyl)ethane-1,2-dione

This compound was made in a similar manner to Example 95 Step 5 using 4-((3-bromophenyl)ethynyl)-1-(difluoromethoxy)-2-ethylbenzene (2.80 g, 7.97 mmol) from the previous step, PdCl₂(ACN)₂ (207 mg, 0.797 mmol), and DMSO (32 mL) to provide 2.42 g, 79%, of the title compound as a yellow solid.

MS (EI): m/z 382 (M^{+.}).

### Step 3: 1-(4-(Difluoromethoxy)-3-ethylphenyl)-2-(3-(3-hydroxyprop-1-ynyl)phenyl)ethane-1,2-dione

This compound was made in a similar manner to Example 95 using 1-(3-bromophenyl)-2-(4-(difluoromethoxy)-3-ethylphenyl)ethane-1,2-dione (2.4 g, 6.26 mmol) from the previous step, propargyl alcohol (526 mg, 555 μL, 9.39 mmol), TEA (3.17 g, 4.36 mL, 31.3 mmol), PdCl₂(PPh₃)₂ (220 mg, 0.313 mmol), CuI (119 mg, 0.626 mmol), and DMF (9.6 mL) to provide 1.43 g, 63%, of the title compound as an orange oil that solidified upon standing to a light yellow solid.

MS (EI): m/z 358 (M^{+.}).

### Step 4: 1-(4-(Difluoromethoxy)-3-ethylphenyl)-2-(3-(3-fluoroprop-1-ynyl)phenyl)ethane-1,2-dione

To a cooled (−78° C.) solution of 1-(4-(difluoromethoxy)-3-ethylphenyl)-2-(3-(3-hydroxyprop-1-ynyl)phenyl)ethane-1,2-dione (1.4 g, 3.91 mmol) from the previous step in DCM (20 mL) was added DAST (693 mg, 563 μL, 4.29 mmol). The mixture was warmed to room temperature then diluted and shaken with saturated NaHCO₃. The aqueous layer was separated and extracted once with DCM. The combined organic layers were washed with water and brine, dried over Na₂SO₄, filtered, and concentrated onto 8 g Celite. Flash chromatography (SiO₂, 5:95 EtOAc:Hexanes to 15:85 EtOAc:Hexanes) to provide 930 mg, 66%, of the title compound as a yellow solid.

MS (EI): m/z 360 (M^{+.}).

### Step 5: 2-Amino-5-[4-(difluoromethoxy)-3-ethylphenyl]-5-[3-(3-fluoroprop-1-yn-1-yl)phenyl]-3-methyl-3,5-dihydro-4H-imidazol-4-one

This compound was made in a similar manner to Example 95 Step 6 using 1-(4-(difluoromethoxy)-3-ethylphenyl)-2-(3-(3-fluoroprop-1-ynyl)phenyl)ethane-1,2-dione (900 mg, 2.5 mmol) from the previous step, 1-methylguanidine.HCl (410 mg, 3.75 mmol), Na₂CO₃ (397 mg, 3.75 mmol), and 200P EtOH (7.2 mL) to provide 748 mg, 72%, of the title compound as a yellow-green foam.

MS (+ESI): m/z 416.1 ([M+H]⁺).

### EXAMPLE 159

### Preparation of: 2-Amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one

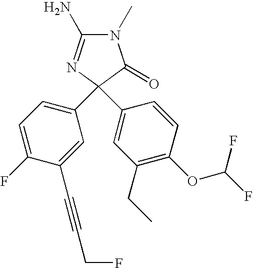

### Step 1: 2-Bromo-4-((4-(difluoromethoxy)-3-ethylphenyl)ethynyl)-1-fluorobenzene

This compound was made in a similar manner to Example 95 Step 2 using 1-(difluoromethoxy)-2-ethyl-4-ethynylbenzene (1.95 g, 9.9 mmol) from the previous step, 3-bromo-4-fluoro-1-iodobenzene (2. g, 9.0 mmol, 1.08 mL), TEA (4.55 g, 6.27 mL, 45.0 mmol), PdCl₂(PPh₃)₂, (316 mg, 0.45 mmol), CuI (51 mg, 0.27 mmol), and DMF (14 mL) to provide 2.81 g, 84%, of the title compound as a yellow oil. The reaction was worked up after 1 h heating.

¹H NMR 500 MHz (CDCl₃) δ 1.20 (t, J=7.59 Hz, 3H); 2.65 (quartet, 7.53 Hz, 2H); 6.50 (t, J=73.84 Hz, 1H); 7.02 (d, J=8.34 Hz, 1H); 7.07 (t, J=8.46 Hz, 1H); 7.31 (dd, J=2.08 Hz, 8.34 Hz, 1H); 7.38-7.42 (m, 2H); 7.70 (dd, J=2.03 Hz, 6.54 Hz, 1H)

### Step 2: 1-(3-Bromo-4-fluorophenyl)-2-(4-(difluoromethoxy)-3-ethylphenyl)ethane-1,2-dione

This compound was made in a similar manner to Example 95 Step 5 using 2-bromo-4-((4-(difluoromethoxy)-3-ethylphenyl)ethynyl)-1-fluorobenzene (2.80 g, 7.58 mmol) from the previous step, PdCl₂(ACN)₂ (197 mg, 0.758 mmol), and DMSO (30 mL) to provide 1.62 g, 53%, of the product and 762 mg of recovered starting material. The SM was resubjected to reaction conditions (0.206 mmol, 53 mg of the catalyst, and 8 mL DMSO) to provide an additional 550 mg of the product for a total of 2.17 g, 71%, of the title compound as a yellow solid.

MS (EI): m/z 400 (M^{+.}).

### Step 3: 1-(4-(Difluoromethoxy)-3-ethylphenyl)-2-(3-(3-hydroxyprop-1-ynyl)phenyl)ethane-1,2-dione

This compound was made in a similar manner to Example 95 Step 2 using 1-(3-bromo-4-fluorophenyl)-2-(4-(difluoromethoxy)-3-ethylphenyl)ethane-1,2-dione (2.1 g, 5.23 mmol) from the previous step, propargyl alcohol (440 mg, 464 μL, 7.85 mmol), TEA (2.64 g, 3.65 mL, 26.15 mmol), PdCl₂(PPh₃)₂ (183 mg, 0.262 mmol), CuI (100 mg, 0.523 mmol), and DMF (8.0 mL) provided 1.51 g, 76%, of the title compound as a dark orange oil that solidified upon standing to a yellow-orange solid.

MS (EI): m/z 376 (M^{+.}).

### Step 4: 1-(4-(Difluoromethoxy)-3-ethylphenyl)-2-(3-(3-fluoroprop-1-ynyl)phenyl)ethane-1,2-dione

This compound was made using 1-(4-(difluoromethoxy)-3-ethylphenyl)-2-(3-(3-hydroxyprop-1-ynyl)phenyl)ethane-1,2-dione (1.48 g, 3.93 mmol) from the previous step, DAST (697 mg, 525 μL, 4.33 mmol), and DCM (20 mL) to provide 1.01 g, 68%, of the title compound as a yellow solid.

MS (EI): m/z 378 (M^{+.}).

### Step 5: 2-Amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one

This compound was made in a similar manner to Example 95 Step 6 using 1-(4-(Difluoromethoxy)-3-ethylphenyl)-2-(3-(3-fluoroprop-1-ynyl)phenyl)ethane-1,2-dione (1.0 g, 2.64 mmol) from the previous step, 1-methylguanidine.HCl (434 mg, 3.97 mmol), Na₂CO₃ (420 mg, 3.97 mmol), and 200P EtOH (7.6 mL) to provide 762 mg, 66%, of the title compound as a yellow-green foam.

MS (+ESI): m/z 434.1 ([M+H]⁺).

### EXAMPLE 160

### Preparation of: (5R)-2-Amino-5-[4-(difluoromethoxy)-3-ethylphenyl]-5-[3-(3-fluoroprop-1-yn-1-yl)phenyl]-3-methyl-3,5-dihydro-4H-imidazol-4-one

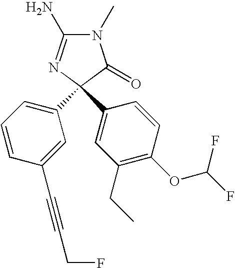

The compound from Example 158 Step 5 was separated by chiral HPLC (Chiralcel AD-H, 2×25 cm; 20% IPA in HFE-7200 with DEA additive) to provide the title compound as a white foam.

MS (+ESI): m/z 416.1 ([M+H]⁺).

### EXAMPLE 161

### Preparation of: (5S)-2-Amino-5-[4-(difluoromethoxy)-3-ethylphenyl]-5-[3-(3-fluoroprop-1-yn-1-yl)phenyl]-3-methyl-3,5-dihydro-4H-imidazol-4-one

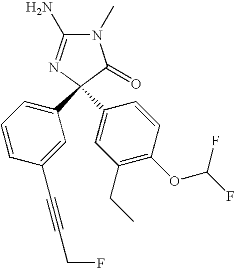

The compound from Example 158 Step 5 was separated by chiral HPLC (Chiralcel AD-H, 2×25 cm; 20% IPA in HFE-7200 with DEA additive) to provide the title compound as a white foam.

MS (+ESI): m/z 416.1 ([M+H]⁺).

### EXAMPLE 162

### Preparation of: (5R)-2-Amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one

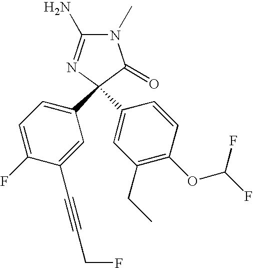

The compound from Example 159 Step 5 was separated by chiral HPLC (Chiralcel AD-H, 2×25 cm; 6% IPA in Hexanes with DEA additive) to provide the title compound as a white foam.

MS (+ESI): m/z 434.1 ([M+H]⁺).

### EXAMPLE 163

### Preparation of: (5S)-2-Amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one

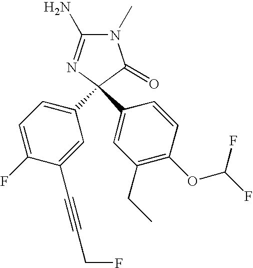

The compound from Example 159 Step 5 was separated by chiral HPLC (Chiralcel AD-H, 2×25 cm; 6% IPA in Hexanes with DEA additive) to provide the title compound as a white foam.

MS (+ESI): m/z 434.1 ([M+H]⁺).

### EXAMPLE 164

### Preparation of 2-Amino-5-(3-bromo-phenyl)-5-(3-cyclopropyl-4-difluoromethoxy-phenyl)-3-methyl-3,5-dihydro-imidazol-4-one

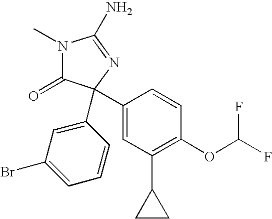

### Step 1: Synthesis of 2-Bromo-4-iodo-phenol

[Reference: Jon Clardy in Org. Lett. 2006, 8(19) 4251].

In a 250 ml round bottom flask 4-iodo-phenol (10 gm, 45.4 mmol) was dissolved in methanol (60 mL). Bromine (2.55 mL) was added dropwise at 0° C. After 30 minutes sodium thiosulfate solution was added, the reaction mixture was extracted with diethylether, washed with water, dried with sodium sulfate and concentrated onto silica gel. Purification by column chromatography (2:1 hexanes/dichloromethane) yielded 3.24 g and mixed fractions were re-subjected to column chromatography (YAMAZEN W-Prep 2XY using 20-35% dichloromethane in hexanes) to give another 6.50 gm for a total of 9.75 gm (72%). ¹H NMR (400 MHz, DMSO-d₆) δ ppm 6.73 (d, J=8.3 Hz, 1H) 7.43 (dd, J=8.6, 2.1 Hz, 1H) 7.72 (d, J=2.1 Hz, 1H) 10.47 (s, 1H).

### Step 2: Synthesis of 2-Bromo-1-difluoromethoxy-4-iodo-benzene

In a 100 mL round bottom flask equipped with a large magnetic egg-shaped stir bar were combined 2-Bromo-4-iodo-phenol (3.45 gm, 11.5 mmol), sodium chlorodifluoroacetate (1.76 gm, 11.5 mmol), and potassium carbonate (6.38 gm, 46.2 mmol) in 10% aqueous dimethylformamide (25 mL). The reaction mixture was immersed in a pre-heated oil bath at 11° C. After 8 hours the crude reaction mixture was partitioned between ethylacetate and water, washed with 1N NaOH, brine and dried with magnesium sulfate. Purification by column chromatography (YAMAZEN W-Prep 2XY using hexanes) yielded 2.68 gm of a low melting white solid (67%). ¹H NMR (400 MHz, DMSO-d₆) δ ppm 7.09 (d, 1H) 7.23 (t, J=73.02 Hz, 1H) 7.75 (dd, J=8.6, 2.1 Hz, 1H) 8.05 (d, J=2.1 Hz, 1H).

### Step 3: Synthesis of (3-Bromo-4-difluoromethoxy-phenylethynyl)-triisopropyl-silane

In a 100 mL round bottom flask were combined 2-Bromo-1-difluoromethoxy-4-iodo-benzene (4.28 gm, 12.2 mmol) and triisopropyl-silyl-acetylene (5.45 mL, 14.4 mmol), in triethylamine (12 mL) and dimethylformamide (24 mL). The contents were chilled in an ice-water bath to 0° C. Cuprous iodide (117 mg, 0.614 mmol) and palladium dichlorobis(triphenyl-phosphine) (432 mg, 0.615 mmol) were added and the mixture stirred at 0° C. After 1 hour the crude reaction mixture was partitioned between diethylether and a saturated solution of ammonium chloride, then washed with a saturated solution of ammonium chloride and dried with sodium sulfate. Purification by column chromatography (YAMAZEN W-Prep 2XY using hexanes) and isolation by concentration of desired fractions by rotary evaporation (bath temperature <5° C.) yielded 4.67 gm of an oil (94%). ¹H NMR (400 MHz, DMSO-d₆) δ ppm 1.05 (s, 21H) 7.25-7.30 (m, 1H) 7.29 (t, J=72.90 Hz, 1H) 7.50 (dd, J=8.6, 2.1 Hz, 1H) 7.77 (d, J=2.1 Hz, 1H); MS (EI) m/z 402 [M+.].

### Step 4: Synthesis of (3-Cyclopropyl-4-difluoromethoxy-phenylethynyl)-triisopropyl-silane

[Reference: Debra Wallace in Tetra. Lett. 2002, 43(39) 6987].

In a 100 mL round bottom flask equipped with a magnetic stir egg were combined (3-Bromo-4-difluoromethoxy-phenylethynyl)-triisopropyl-silane (1.17 gm, 2.90 mmol), cyclopropylboronic acid (0.500 gm, 5.80 mmol), potassium phosphate (2.16 gm, 10.2 mmol), palladium acetate (32 mg, 0.143 mmol) and tricylclohexyl-phosphine (81 mg, 0.290 mmol) in toluene (13 mL) and water (0.65 mL). The reaction mixture was immersed in a pre-heated oil bath at 100° C. After 3 hours add more cyclopropyl boronic acid, Pd, P(cHex)₃ and phosphate base and continue heating another three hours. The crude reaction mixture was partitioned between ethylacetate and water, extracted with ethylacetate and dried with magnesium sulfate. Purification by column chromatography, YAMAZEN W-Prep 2XY (0-25% ethylacetate in hexanes) yielded 1.01 gm of an oil (96%). ¹H NMR (400 MHz, DMSO-d₆) δ ppm 0.69 (q, J=5.2 Hz, 2H) 0.92 (dq, J=8.5, 2.8 Hz, 2H) 1.05 (s, 21H) 2.01 (quin, J=6.8 Hz, 1H) 6.97 (d, J=2.1 Hz, 1H) 7.10 (d, J=8.6 Hz, 1H) 7.22 (t, J=73.89 Hz, 1H) 7.28 (dd, J=8.4, 2.1 Hz, 1H); MS (EI) m/z 364 [M+.].

### Step 5: Synthesis of 2-Cyclopropyl-1-difluoromethoxy-4-ethynyl-benzene

A 50 mL round bottom flask was charged with (3-Cyclopropyl-4-difluoromethoxy-phenylethynyl)-triisopropyl-silane (0.525 gm, 1.44 mmol), diluted with tetrahydrofuran (THF, 1 ml), and chilled in an ice-water bath. A 1M solution of tetrabutylammonium fluoride in THF (1.5 mL) was added at 0° C. After 1 hour mixture was diluted with hexanes (30 ml) and washed with water (10 mL). Combined hexane extracts were washed with brine, dried with sodium sulfate, and concentrated to an oil by rotary evaporation 0.225 gm (75%). ¹H NMR (400 MHz, DMSO-d₆) δ ppm 0.68 (quin, J=3.9 Hz, 2H) 0.91 (dq, J=8.8, 3.5 Hz, 2H) 2.01 (dq, J=12.4, 5.1 Hz, 1H) 4.10 (s, 1H) 7.01 (d, J=2.1 Hz, 1H) 7.09 (d, J=8.6 Hz, 1H) 7.20 (t, J=73.95 Hz, 1H) 7.28 (dd, J=8.3, 2.1 Hz, 1H).

### Step 6: Synthesis of 4-(3-Bromo-phenylethynyl)-2-cyclopropyl-1-difluoromethoxy-benzene

In a 100 mL round bottom flask were combined 3-Bromo-1-iodo-benzene (0.305 gm, 1.08 mmol) and 2-Cyclopropyl-1-difluoromethoxy-4-ethynyl-benzene (0.225 gm, 1.08 mmol), in triethylamine (1 mL) and dimethylformamide (2 mL). The contents were chilled in an ice-water bath to 0° C. Cuprous iodide (10 mg, 0.052 mmol) and palladiumdichlorobis-(triphenylphosphine) (38 mg, 0.054 mmol) were added and the mixture stirred at 0° C. After 2 hours the crude reaction mixture was partitioned between diethylether and a saturated solution of ammonium chloride, then washed with a saturated solution of ammonium chloride and dried with magnesium sulfate. Purification by column chromatography (YAMAZEN W-Prep 2XY using hexanes) yielded 0.287 gm of an oil (75%).

¹H NMR (400 MHz, DMSO-d₆) δ ppm 0.73 (quin, J=3.2 Hz, 2H) 0.95 (dq, J=8.7, 3.4 Hz, 2H) 2.05 (td, J=9.0, 4.3 Hz, 1H) 7.13-7.18 (m, 2H) 7.26 (t, J=73.83 Hz, 1H) 7.36 (q, J=8.4 Hz, 2H) 7.52 (dd, J=7.8, 1.0 Hz, 1H) 7.58 (t, J=1.6 Hz, 1H) 7.74 (d, J=1.9 Hz, 1H); MS (EI) m/z 362 [M+.].

### Step 7: Synthesis of 1-(3-Bromo-phenyl)-2-(3-cyclopropyl-4-difluoromethoxy-phenyl)-ethane-1,2-dione

In a 50 ml round bottom flask was added 4-(3-Bromo-phenylethynyl)-2-cyclopropyl-1-difluoromethoxy-benzene (0.228 g, 0.628 mmol) in acetone (5.2 mL) and water (1.8 mL). Sodium carbonate (30 mg, 0.35 mmol) magnesium sulfate (105 mg, 0.87 mmol) and potassium permanganate (225 mg, 11.4 mmol) were added (permanganate added last) at room temperature. After 2 hours the reaction was diluted with hexanes (50 mLs), stirred well for 30 minutes and decanted, add 10% EtOAc/hexanes and stir well and decant (from red gummy residue) repeat combine and concentrate to afford 0.203 gm of an oil (82%). ¹H NMR (400 MHz, DMSO-d₆) δ ppm 0.71 (quin, J=4.0 Hz, 2H) 0.98 (dt, J=10.6, 4.3 Hz, 2H) 2.09 (quin, J=6.9 Hz, 1H) 7.29 (d, J=8.6 Hz, 1H) 7.40 (t, J=73.2 Hz, 1H) 7.53 (t, J=7.9 Hz, 2H) 7.75 (dd, J=8.6, 2.1 Hz, 1H) 7.86 (dd, J=6.7, 1.6 Hz, 1H) 7.95 (t, J=5.0 Hz, 1H) 8.03 (t, J=1.7 Hz, 1H); MS (EI) m/z 394 [M+.]

### Step 8 Synthesis of 2-Amino-5-(3-bromo-phenyl)-5-(3-cyclopropyl-4-difluoromethoxy-phenyl)-3-methyl-3,5-dihydro-imidazol-4-one

In a 50 ml round bottom flask was dissolved 1-(3-Bromo-phenyl)-2-(3-cyclopropyl-4-difluoromethoxy-phenyl)-ethane-1,2-dione (0.102 g, 0.258 mmol) in isopropanol (9 mL). Methylguanidine hydrochloride (42 mg, 0.383 mmol) was added followed by sodium carbonate (41 mg, 0.387 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded an oil. The oil was re-dissolved in diethylether, diluted with hexanes and concentrated, twice to give a white solid, 96 mg (83%) mp 85-87° C.; ¹H NMR (400 MHz, DMSO-d₆) δ ppm 0.48 (quin, J=3.9 Hz, 2H) 0.92 (dq, J=8.7, 3.4 Hz, 2H) 1.99 (dt, J=10.7, 5.7 Hz, 1H) 2.93 (s, 3H) 6.75 (br. s., 2H) 7.04 (d, J=2.3 Hz, 2H) 7.11 (t, J=74.23 Hz, 1H) 7.21-7.29 (m, 2H) 7.40 (dd, J=8.6, 1.6 Hz, 2H) 7.54 (t, J=1.9 Hz, 1H); MS (ES) m/z 448.0 [M−H]⁻

### EXAMPLE 165

### Preparation of (R)-2-Amino-5-(3-bromo-phenyl)-5-(3-cyclopropyl-4-difluoromethoxy-phenyl)-3-methyl-3,5-dihydro-imidazol-4-one

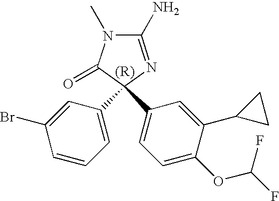

A racemic mixture of 2-Amino-5-(3-bromo-phenyl)-5-(3-cyclopropyl-4-difluoromethoxy-phenyl)-3-methyl-3,5-dihydro-imidazol-4-one (104 mg) was separated by chiral column chromatography (Chiralpak AD-H, 2×25 cm) eluting with 4% methanol/ethanol (8/2 with 0.1% diethylamine) in hexanes to provide peak 1 (RT=10.0 min) (R)-2-Amino-5-(3-bromo-phenyl)-5-(3-cyclopropyl-4-difluoromethoxy-phenyl)-3-methyl-3,5-dihydro-imidazol-4-one (21 mg) as a white foam MS m/e [M+H]+ 450.0, [α]_{D}²⁵=−9.00 (c=1% in MeOH).

### EXAMPLE 166

### Preparation of: (S)-2-Amino-5-(3-bromo-phenyl)-5-(3-cyclopropyl-4-difluoromethoxy-phenyl)-3-methyl-3,5-dihydro-imidazol-4-one

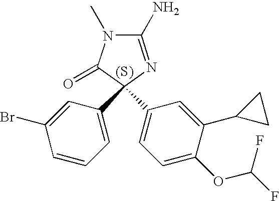

A racemic mixture of 2-Amino-5-(3-bromo-phenyl)-5-(3-cyclopropyl-4-difluoromethoxy-phenyl)-3-methyl-3,5-dihydro-imidazol-4-one (104 mg) was separated by chiral column chromatography (Chiralpak AD-H, 2×25 cm) eluting with 4% methanol/ethanol (8/2 with 0.1% diethylamine) in hexanes to provide peak 2 (RT=11.2 min) (S)-2-Amino-5-(3-bromo-phenyl)-5-(3-cyclopropyl-4-difluoromethoxy-phenyl)-3-methyl-3,5-dihydro-imidazol-4-one (30 mg) as a white foam MS m/e [M+H]+ 450.0, [α]_{D}²⁵=+5.00 (c=1% in MeOH)

### EXAMPLE 167

### Preparation of: 2-amino-4-(3-cyclopropyl-4-(difluoromethoxy)phenyl)-4-(3-ethynylphenyl)-1-methyl-1H-imidazol-5(4H)-one

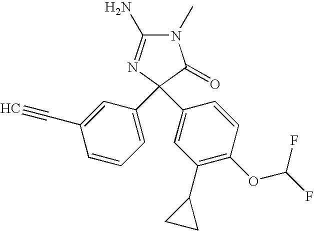

### Step 1: Synthesis of 1-(3-cyclopropyl-4-(difluoromethoxy)phenyl)-2-(3-((triisopropylsilyl)ethynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol) Tetrakis(triphenylphosphine)palladium (20 mg, 0.017 mmol) and (Triisopropyl-silyl)acetylene (0.17 ml, d=0.813, 0.755 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. Both runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.221 gm of an oil (88%). ¹H NMR (400 MHz, DMSO-d₆) δ ppm 0.74 (dd, J=5.3, 1.9 Hz, 2H) 1.01 (dddd, J=6.3, 4.5, 4.3, 4.3 Hz, 2H) 1.05-1.14 (m, 21H) 2.12 (ddd, J=8.9, 4.3, 3.9 Hz, 1H) 7.32 (d, J=8.6 Hz, 1H) 7.43 (t, J=73.14 Hz, 1H) 7.57 (d, J=2.1 Hz, 1H) 7.60-7.65 (m, 1H) 7.78 (dd, J=8.5, 2.2 Hz, 1H) 7.85 (ddd, J=7.9, 1.4, 1.2 Hz, 1H) 7.90 (ddd, J=7.8, 1.4, 1.4 Hz, 1H) 7.93 (t, J=1.5 Hz, 1H)

### Step 2: Synthesis of 2-amino-4-(3-cyclopropyl-4-(difluoromethoxy)phenyl)-4-(3-ethynylphenyl)-1-methyl-1H-imidazol-5(4H)-one

1-(3-Cyclopropyl-4-(difluoromethoxy)phenyl)-2-(3-((triisopropylsilyl)ethynyl-)phenyl)ethane-1,2-dione (0.220 g, 0.443 mmol) was dissolved tetrahydrofuran (2.0 ml). A 1 molar solution of tetrabutylammonium fluoride in tetrahydrofuran (0.500 ml, 0.500 mmol) was added. Solution browns after 20 minutes. The crude reaction was partitioned between mixture of (hexanes/diethylether)/water. The organic layer was concentrated then dissolved in isopropanol (20 mL). Methylguanidine hydrochloride (71 mg, 0.648 mmol) was added followed by sodium carbonate (70 mg, 0.660 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, then 100% EtOAc] afforded oil. The oil was re-dissolved in diethylether, diluted with hexanes and concentrated, twice to give a white foam, 120 mg (69%); ¹H NMR (400 MHz, DMSO-d₆) δ ppm 0.50 (dd, J=5.3, 1.9 Hz, 2H) 0.88-0.93 (m, 2H) 1.97-2.06 (m, 1H) 2.96 (s, 3H) 4.15 (s, 1H) 6.72 (br. s., 2H) 7.07 (d, J=8.3 Hz, 2H) 7.13 (t, J=74.3 Hz 1H) 7.25-7.34 (m, 3H) 7.39-7.45 (m, 1H) 7.47 (s, 1H); MS (ES) m/z 396.0 [M+H]+

### EXAMPLE 168

### Preparation of: 2-amino-4-(3-cyclopropyl-4-(difluoromethoxy)phenyl)-1-methyl-4-(3-(prop-1-ynyl)phenyl)-1H-imidazol-5(4H)-one

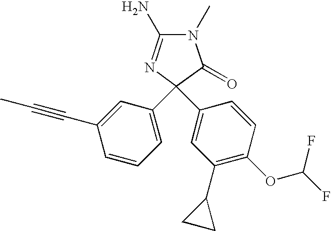

### Step 1: Synthesis of 1-(3-cyclopropyl-4-(difluoromethoxy)phenyl)-2-(3-(prop-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and Tributyl-propynyl-stannane (0.270 gm, 0.787 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The reaction set-up was repeated in another vial with 85% of all reagents. The runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20%% ethyl acetate in hexanes afforded 0.135 gm of an oil (56% based on tributyl tin impurity). ¹H NMR (400 MHz, CDCl₃) δ ppm 0.71-0.75 (m, 2H) 1.01-1.06 (m, 2H) 2.03 (s, 3H) 2.13-2.20 (m, 1H) 6.62 (t, J=73.2 Hz, 1H) 7.14 (d, J=8.6 Hz, 1H) 7.42 (t, J=7.8 Hz, 1H) 7.59 (s, 1H) 7.62 (d, J=6.4 Hz, 1H) 7.69 (dd, t, J=8.5, 2.3 Hz, 1H) 7.84 (d, J=7.89 Hz, 1H) 7.91 (s, 1H); MS (ES) m/z 353.1 [M−H]⁻

### Step 2: Synthesis of 2-amino-4-(3-cyclopropyl-4-(difluoromethoxy)phenyl)-1-methyl-4-(3-(prop-1-ynyl)phenyl)-1H-imidazol-5(4H)-one

1-(3-Cyclopropyl-4-(difluoromethoxy)phenyl)-2-(3-(prop-1-ynyl)phenyl)ethane-1,2-dione (0.100 g, 0.282 mmol) was dissolved in isopropanol (14 mL). Methylguanidine hydrochloride (46 mg, 0.420 mmol) was added followed by sodium carbonate (45 mg, 0.425 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 89 mg (78%); ¹H NMR (400 MHz, DMSO-d₆) δ ppm 0.44-0.48 (m, 2H) 0.88-0.93 (m, 2H) 1.97 (s, 3H) 1.94-2.02 (m, 1H) 2.92 (s, 3H) 6.66 (br. s., 2H) 7.02-7.04 (m, 2H) 7.09 (t, J=74.30 Hz, 1H) 7.17-7.30 (m, 4H) 7.34 (s, 1H); MS (ES) m/z 410.2 [M+H]+

### EXAMPLE 169

### Preparation of: (S)-2-amino-4-(3-cyclopropyl-4-(difluoromethoxy)phenyl)-1-methyl-4-(3-(prop-1-ynyl)phenyl)-1H-imidazol-5(4H)-one

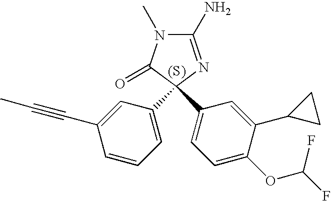

A racemic mixture of 2-amino-4-(3-cyclopropyl-4-(difluoromethoxy)phenyl)-1-methyl-4-(3-(prop-1-ynyl)phenyl)-1H-imidazol-5(4H)-one (257 mg) was separated by chiral column chromatography (Chiralpak AD, 5×50 cm) eluting with 7% ethanol (with 0.1% diethylamine) in hexanes to provide peak 1 (RT=19.0 min) (S)-2-amino-4-(3-cyclopropyl-4-(difluoromethoxy)phenyl)-1-methyl-4-(3-(prop-1-ynyl)phenyl)-1H-imidazol-5(4H)-one (78 mg) as a white foam MS m/e [M+H]+ 410.0, [α]_{D}²⁵=−9.0 (c=1% in MeOH).

### EXAMPLE 170

### Preparation of: (R)-2-amino-4-(3-cyclopropyl-4-(difluoromethoxy)phenyl)-1-methyl-4-(3-(prop-1-ynyl)phenyl)-1H-imidazol-5(4H)-one

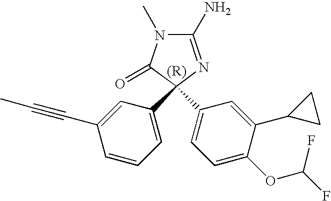

A racemic mixture of 2-amino-4-(3-cyclopropyl-4-(difluoromethoxy)phenyl)-1-methyl-4-(3-(prop-1-ynyl)phenyl)-1H-imidazol-5(4H)-one (257 mg) was separated by chiral column chromatography (Chiralpak AD, 5×50 cm) eluting with 7% ethanol (with 0.1% diethylamine) in hexanes to provide peak 2 (RT=22.4 min) (R)-2-amino-4-(3-cyclopropyl-4-(difluoromethoxy)phenyl)-1-methyl-4-(3-(prop-1-ynyl)phenyl)-1H-imidazol-5(4H)-one (80 mg) as a white foam MS m/e [M+H]+ 410.0, [α]_{D}²⁵=+11.0 (c=1% in MeOH)

### EXAMPLE 171

### Preparation of: 2-amino-4-(3-(but-1-ynyl)phenyl)-4-(3-cyclopropyl-4-(difluoromethoxy)phenyl)-1-methyl-1H-imidazol-5(4H)-one

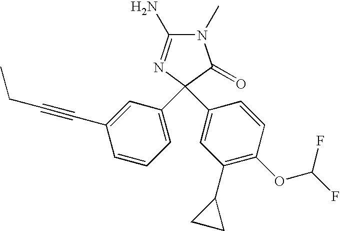

### Step 1: Synthesis of 1-(3-(but-1-ynyl)phenyl)-2-(3-cyclopropyl-4-(difluoromethoxy)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (9 mg, 0.047 mmol), Tetrakis(triphenylphosphine) palladium (15 mg, 0.013 mmol) were added at room temperature. The vial was covered with Teflon septa and secured via a crimped aluminum cap then chilled in a dry ice bath. A refrigerated cylinder of But-1-yne (Aldrich Chemical) was fastened to a 10 mm ID hose fitted with a luer lock adapter and a 20-gauge needle (to pierce septum of chilled microwave vial). The valve was opened and the cylinder inverted to introduce approximately 1.0 ml of But-1-yne. The microwave vial and its contents were removed from the ice bath and allowed to warm to room temperature. The reaction was irradiated in a Biotage Initiator microwave at 80° C. for 60 minutes (30 second pre-stir, Fixed Hold Time On, Normal absorbance level). The same reaction set-up was repeated. The runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.138 gm of an oil (74%). ¹H NMR (400 MHz, CDCl₃) δ ppm 0.70-0.74 (m, 2H) 1.01-1.04 (m, 2H) 1.20 (t, J=7.4 Hz, 3H) 2.13-2.17 (m, 1H) 2.38 (q, J=7.5 Hz, 2H) 6.60 (t, J=73.14 Hz, 1H) 7.13 (d, J=8.58 Hz, 1H) 7.41 (t, J=8.58 Hz, 1H) 7.58 (s, 1H) 7.61-7.70 (m, 2H) 7.82 (t, J=7.9 Hz, 1H) 7.91 (s, 1H); MS (ES) m/z 367.1 [M−H]⁻

### Step 2: Synthesis of 2-amino-4-(3-(but-1-ynyl)phenyl)-4-(3-cyclopropyl-4-(difluoromethoxy)phenyl)-1-methyl-1H-imidazol-5(4H)-one

1-(3-(but-1-ynyl)phenyl)-2-(3-cyclopropyl-4-(difluoromethoxy)phenyl)ethane-1,2-dione (0.206 g, 0.559 mmol) was dissolved in isopropanol (25 mL). Methylguanidine hydrochloride (95 mg, 0.867 mmol) was added followed by sodium carbonate (94 mg, 0.886 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 78 mg (33%); ¹H NMR (400 MHz, DMSO-d₆) δ ppm 0.45-0.49 (m, 2H) 0.89-0.94 (m, 2H) 1.11 (t, J=7.5 Hz, 3H) 1.96-2.03 (m, 1H) 2.36 (t, J=7.5 Hz, 2H) 2.93 (s, 3H) 6.69 (br. s., 2H) 7.03-7.05 (m, 2H) 7.11 (t, J=74.4 Hz, 1H) 7.18-7.33 (m, 4H) 7.36 (s, 1H); MS (ES) m/z 424.2 [M+H]+

### EXAMPLE 172

### Preparation of: 2-amino-4-(3-cyclopropyl-4-(difluoromethoxy)phenyl)-1-methyl-4-(3-(pent-1-ynyl)phenyl)-1H-imidazol-5(4H)-one

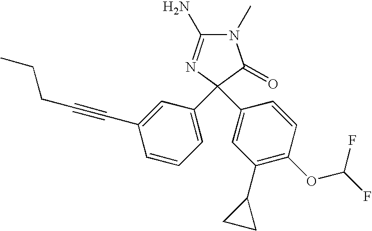

### Step 1: Synthesis of 1-(3-cyclopropyl-4-(difluoromethoxy)phenyl)-2-(3-(pent-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and Pent-1-yne (1.00, d=0.691, 10.1 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. Both runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.174 gm of an oil (90%). ¹H NMR (400 MHz, DMSO-d₆) δ ppm 0.70 (quin, J=4.0 Hz, 2H) 0.95 (t, J=7.2 Hz, 3H) 0.97-1.00 (m, 2H) 1.52 (sxt, J=7.2 Hz, 2H) 2.09 (quin, J=6.8 Hz, 1H) 2.37 (t, J=7.0 Hz, 2H) 7.29 (d, J=8.6 Hz, 1H) 7.39 (t, J=73.25 Hz, 1H) 7.53 (d, J=2.1 Hz, 1H) 7.55-7.57 (m, 1H) 7.70-7.76 (m, 2H) 7.78-7.84 (m, 2H); MS (ES) m/z 381.2 [M−H]⁻

### Step 2: Synthesis of 2-amino-4-(3-cyclopropyl-4-(difluoromethoxy)phenyl)-1-methyl-4-(3-(pent-1-ynyl)phenyl)-1H-imidazol-5(4H)-one

1-(3-Cyclopropyl-4-(difluoromethoxy)phenyl)-2-(3-(pent-1-ynyl)phenyl)ethane-1,2-dione (0.162 g, 0.424 mmol) was dissolved in isopropanol (20 mL). Methylguanidine hydrochloride (69 mg, 0.630 mmol) was added followed by sodium carbonate (68 mg, 0.641 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 144 mg (78%); ¹H NMR (400 MHz, DMSO-d₆) δ ppm 0.44-0.48 (m, 2H) 0.88-0.95 (m, 5H) 1.50 (sxt, J=7.3 Hz, 2H) 1.95-2.02 (m, 1H) 1.52 (t, J=7.0 Hz, 2H) 2.92 (s, 3H) 6.67 (br. s., 2H) 7.02-7.04 (m, 2H) 7.09 (t, J=74.3 Hz, 1H) 7.17-7.26 (m, 3H) 7.31 (d, J=7.4 Hz, 1H) 7.35 (s, 1H); MS (ES) m/z 438.2 [M+H]+

### EXAMPLE 173

### Preparation of: 2-amino-4-(3-cyclopropyl-4-(difluoromethoxy)phenyl)-1-methyl-4-(3-(3-methylbut-1-ynyl)phenyl)-1H-imidazol-5(4H)-one

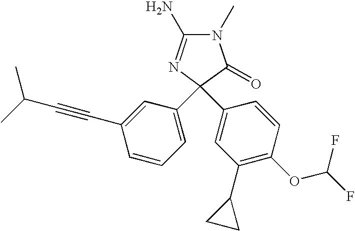

### Step 1: Synthesis of 1-(3-cyclopropyl-4-(difluoromethoxy)phenyl)-2-(3-(3-methylbut-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (9 mg, 0.047 mmol), Tetrakis(triphenylphosphine) palladium (15 mg, 0.013 mmol) were added at room temperature. The vial was covered with Teflon septa and secured via a crimped aluminum cap then chilled in a dry ice bath. A refrigerated cylinder of 3-methyl-but-1-yne (GFS Chemical) was fastened to a 10 mm ID hose fitted with a luer lock adapter and a 20-gauge needle (to pierce septum of chilled microwave vial). The valve was opened and the cylinder inverted to introduce approximately 1.0 ml of 3-methyl-but-1-yne. The microwave vial and its contents were removed from the ice bath and allowed to ward to room temperature. The reaction was irradiated in a Biotage Initiator microwave at 80° C. for 60 minutes (30 second pre-stir, Fixed Hold Time On, Normal absorbance level). The same reaction set-up was repeated. The runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.138 gm of an oil (71%). ¹H NMR (400 MHz, CDCl₃) δ ppm 0.70-0.74 (m, 2H) 1.01-1.06 (m, 2H) 1.23 (d, J=6.8 Hz, 6H) 2.11-2.18 (m, 1H) 2.74 (dt, J=13.7, 6.9 Hz, 1H) 6.60 (t, J=73.1 Hz, 1H) 7.13 (d, J=8.5 Hz, 1H) 7.40 (t, J=7.8 Hz, 1H) 7.58 (s, 1H) 7.63 (d, J=6.6 Hz, 1H) 7.69 (dd, t, J=8.5, 2.2 Hz, 1H) 7.82 (d, J=7.88 Hz, 1H) 7.91 (s, 1H); MS (ES) m/z 381.2 [M−H]⁻

### Step 2: Synthesis of 2-amino-4-(3-cyclopropyl-4-(difluoromethoxy)phenyl)-1-methyl-4-(3-(3-methylbut-1-ynyl)phenyl)-1H-imidazol-5(4H)-one

1-(3-Cyclopropyl-4-(difluoromethoxy)phenyl)-2-(3-(3-methylbut-1-ynyl)phenyl)ethane-1,2-dione (0.139 g, 0.363 mmol) was dissolved in isopropanol (16 mL). Methylguanidine hydrochloride (59 mg, 0.539 mmol) was added followed by sodium carbonate (58 mg, 0.547 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 124 mg (78%); ¹H NMR (400 MHz, DMSO-d₆) δ ppm 0.44-0.48 (m, 2H) 0.88-0.92 (m, 2H) 1.14 (d, J=6.8 Hz, 6H) 1.94-2.00 (m, 1H) 2.70-2.77 (m, 1H) 2.92 (s, 3H) 6.67 (br. s., 2H) 7.02-7.04 (m, 2H) 7.09 (t, J=74.30 Hz, 1H) 7.16-7.35 (m, 5H); MS (ES) m/z 438.2 [M+H]+

### EXAMPLE 174

### Preparation of: (S)-2-amino-4-(3-cyclopropyl-4-(difluoromethoxy)phenyl)-1-methyl-4-(3-(3-methylbut-1-ynyl)phenyl)-1H-imidazol-5(4H)-one

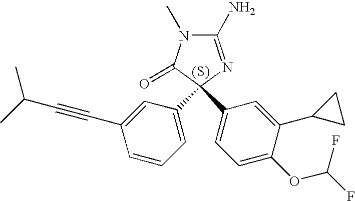

A racemic mixture of 2-amino-4-(3-cyclopropyl-4-(difluoromethoxy)phenyl)-1-methyl-4-(3-(3-methylbut-1-ynyl)phenyl)-1H-imidazol-5(4H)-one (216 mg) was separated by chiral column chromatography (Chiralcel AD-H, 2×25 cm) eluting with 5% methanol/ethanol (8/2 with 0.1% diethylamine) in hexanes to provide peak 1 (RT=8.9 min) (S)-2-amino-4-(3-cyclopropyl-4-(difluoromethoxy)phenyl)-1-methyl-4-(3-(3-methylbut-1-ynyl)phenyl)-1H-imidazol-5(4H)-one (47 mg) as a white foam MS m/e [M+H]+ 438.1, [α]_{D}²⁵=+31.0 (c=1% in MeOH).

### EXAMPLE 175

### Preparation of: (R)-2-amino-4-(3-cyclopropyl-4-(difluoromethoxy)phenyl)-1-methyl-4-(3-(3-methylbut-1-ynyl)phenyl)-1H-imidazol-5(4H)-one

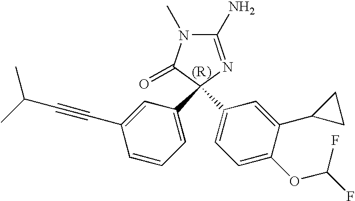

A racemic mixture of 2-amino-4-(3-cyclopropyl-4-(difluoromethoxy)phenyl)-1-methyl-4-(3-(3-methylbut-1-ynyl)phenyl)-1H-imidazol-5(4H)-one (216 mg) was separated by chiral column chromatography (Chiralcel AD-H, 2×25 cm) eluting with 5% methanol/ethanol (8/2 with 0.1% diethylamine) in hexanes to provide peak 2 (RT=11.2 min) (R)-2-amino-4-(3-cyclopropyl-4-(difluoromethoxy)phenyl)-1-methyl-4-(3-(3-methylbut-1-ynyl)phenyl)-1H-imidazol-5(4H)-one (42 mg) as a white foam MS m/e [M+H]+ 438.1, [U]_{D}²⁵=−39.0 (c=1% in MeOH)

### EXAMPLE 176

### Preparation of: 2-amino-4-(3-cyclopropyl-4-(difluoromethoxy)phenyl)-4-(3-(cyclopropylethynyl)phenyl)-1-methyl-1H-imidazol-5(4H)-one

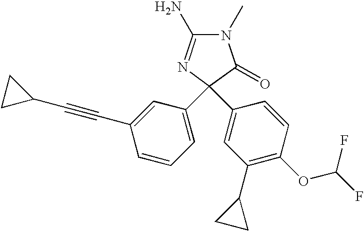

### Step 1: Synthesis of 1-(3-cyclopropyl-4-(difluoromethoxy)phenyl)-2-(3-(cyclopropylethynyl)phenyl)ethane-1,2-dione

To a Biotage conical microwave vial (0.5-2.0 mL) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (9 mg, 0.047 mmol), Tetrakis(triphenylphosphine) palladium (15 mg, 0.013 mmol) and cyclopropylacetylene (0.73 ml, 8.63 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. The runs were combined, diluted with diethylether and washed with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.59 gm of an oil (61%). ¹H NMR (400 MHz, CDCl₃) δ ppm 0.76 (q, J=3.7 Hz, 2H) 0.82 (t, J=3.7 Hz, 2H) 0.89 (dt, J=8.3, 2.8 Hz, 2H) 1.06 (dd, J=8.5, 1.5 Hz, 2H) 1.45 (dq, J=12.0, 4.9 Hz, 1H) 2.19 (dt, J=7.5, 3.2 Hz, 1H) 6.60 (t, J=73.14 Hz, 1H) 7.17 (d, J=8.6 Hz, 1H) 7.43 (t, J=7.8 Hz, 1H) 7.58-7.68 (m, 2H) 7.72 (dd, J=8.5, 2.2 Hz, 1H) 7.85 (d, J=7.9 Hz, 1H) 7.89-7.95 (m, 1H); MS (EI) m/z 380 [M⁺.]

### Step 2: Synthesis of 2-amino-4-(3-cyclopropyl-4-(difluoromethoxy)phenyl)-4-(3-(cyclopropylethynyl)phenyl)-1-methyl-1H-imidazol-5(4H)-one

1-(3-Cyclopropyl-4-(difluoromethoxy)phenyl)-2-(3-(cyclopropylethynyl)phenyl)ethane-1,2-dione (0.172 g, 0.432 mmol) was dissolved in isopropanol (20 mL). Methylguanidine hydrochloride (70 mg, 0.639 mmol) was added followed by sodium carbonate (69 mg, 0.652 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded oil. The oil was re-dissolved in diethylether, diluted with hexanes and concentrated, twice to give a white foam, 114 mg (58%); ¹H NMR (400 MHz, DMSO-d₆) δ ppm 0.45-0.49 (m, 2H) 0.66-0.70 (m, 2H) 0.78-0.85 (m, 2H) 0.89-0.93 (m, 2H) 1.45-1.51 (m, 1H) 1.95-2.02 (m, 1H) 2.92 (s, 3H) 6.69 (br. s., 2H) 7.02-7.05 (m, 2H) 7.11 (t, J=74.4 Hz, 1H) 7.16-7.33 (m, 5H); MS (ES) m/z 436.2 [M+H]⁺

### EXAMPLE 177

### Preparation of: (S)-2-amino-4-(3-cyclopropyl-4-(difluoromethoxy)phenyl)-4-(3-(cyclopropylethynyl)phenyl)-1-methyl-1H-imidazol-5(4H)-one

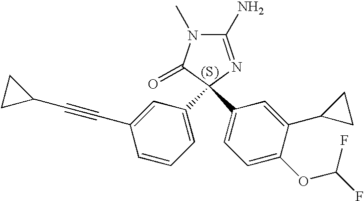

A racemic mixture of 2-amino-4-(3-cyclopropyl-4-(difluoromethoxy)phenyl)-4-(3-(cyclopropylethynyl)phenyl)-1-methyl-1H-imidazol-5(4H)-one (258 mg) was separated by chiral column chromatography (Chiralpak AD-H, 2×25 cm) eluting with 5% methanol/ethanol (8/2 with 0.1% diethylamine) in hexanes to provide peak 1 (RT=6.7 min) (S)-2-amino-4-(3-cyclopropyl-4-(difluoromethoxy)phenyl)-4-(3-(cyclopropylethynyl)phenyl)-1-methyl-1H-imidazol-5(4H)-one (64 mg) as a white foam MS m/e [M+H]⁺ 436.1, [α]_{D}²⁵=+69.0 (c=1% in MeOH).

### EXAMPLE 178

### Preparation of: (R)-2-amino-4-(3-cyclopropyl-4-(difluoromethoxy)phenyl)-4-(3-(cyclopropylethynyl)phenyl)-1-methyl-1H-imidazol-5(4H)-one

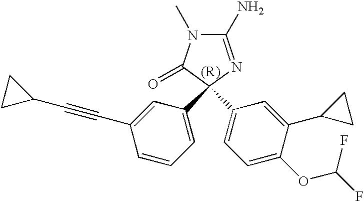

A racemic mixture of 2-amino-4-(3-cyclopropyl-4-(difluoromethoxy)phenyl)-4-(3-(cyclopropylethynyl)phenyl)-1-methyl-1H-imidazol-5(4H)-one (258 mg) was separated by chiral column chromatography (Chiralpak AD-H, 2×25 cm) eluting with 5% methanol/ethanol (8/2 with 0.1% diethylamine) in hexanes to provide peak 2 (RT=8.1 min) (R)-2-amino-4-(3-cyclopropyl-4-(difluoromethoxy)phenyl)-4-(3-(cyclopropylethynyl)phenyl)-1-methyl-1H-imidazol-5(4H)-one (70 mg) as a white foam MS m/e [M+H]⁺ 436.1, [α]_{D}²⁵=−78.0 (c=1% in MeOH)

### EXAMPLE 179

### Preparation of: 2-amino-4-(3-cyclopropyl-4-(difluoromethoxy)phenyl)-4-(3-(3-hydroxy-3-methylbut-1-ynyl)phenyl)-1-methyl-1H-imidazol-5(4H)-one

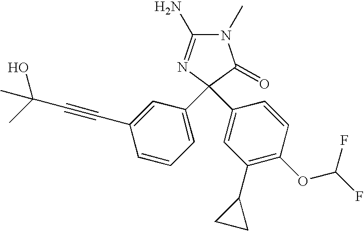

### Step 1: Synthesis of 1-(3-cyclopropyl-4-(difluoromethoxy)phenyl)-2-(3-(3-hydroxy-3-methylbut-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine)palladium (20 mg, 0.017 mmol) and 2-methylbut-3-yn-2-ol (70 mg, 0.832 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. Both runs were combined, diluted with diethylether and washed twice with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 173 mg of an oil (86%). ¹H NMR (400 MHz, DMSO-d₆) δ ppm 0.74 (dd, J=5.3, 1.9 Hz, 2H) 0.98-1.04 (m, 2H) 1.42 (s, 6H) 2.08-2.16 (m, 1H) 5.50 (s, 1H) 7.33 (d, J=8.6 Hz, 1H) 7.43 (t, J=73.0 Hz, 1H) 7.56-7.60 (m, 2H) 7.72-7.75 (m, 2H) 7.78 (t, J=1.5 Hz, 1H) 7.84-7.87 (m, 1H); MS (APPI) m/z 398 [M⁺.]

### Step 2: Synthesis of 2-amino-4-(3-cyclopropyl-4-(difluoromethoxy)phenyl)-4-(3-(3-hydroxy-3-methylbut-1-ynyl)phenyl)-1-methyl-1H-imidazol-5(4H)-one

1-(3-Cyclopropyl-4-(difluoromethoxy)phenyl)-2-(3-(3-hydroxy-3-methylbut-1-ynyl)phenyl)ethane-1,2-dione (0.163 g, 0.409 mmol) was dissolved in isopropanol (20 mL). Methylguanidine hydrochloride (69 mg, 0.630 mmol) was added followed by sodium carbonate (68 mg, 0.641 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 153 mg (82%); ¹H NMR (400 MHz, DMSO-d₆) δ ppm 0.50 (dd, J=5.2, 1.7 Hz, 2H) 0.94 (dd, J=8.3, 2.1 Hz, 2H) 1.43 (s, 6H) 1.98-2.06 (m, 1H) 2.96 (s, 3H) 5.43 (s, 1H) 6.71 (br s, 2H) 7.05 (d, J=2.3 Hz, 2H) 7.13 (t, J=74.4 Hz, 1H) 7.20-7.30 (m, 3H) 7.37-7.43 (m, 2H); MS (ES) m/z 452.1 [M−H]⁻

### EXAMPLE 180

### Preparation of: 2-amino-4-(3-cyclopropyl-4-(difluoromethoxy)phenyl)-4-(3-(3-methoxyprop-1-ynyl)phenyl)-1-methyl-1H-imidazol-5(4H)-one

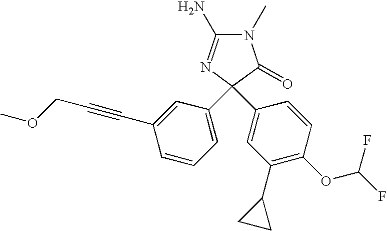

### Step 1: Synthesis of 1-(3-cyclopropyl-4-(difluoromethoxy)phenyl)-2-(3-(3-methoxyprop-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and 3-methoxyprop-1-yne (0.200 gm, 2.85 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. The runs were combined, diluted with diethylether and washed with saturated ammonium chloride solution. The ethereal layer was concentrated and loaded onto silica gel. Purification by chromatography (YAMAZEN W-Prep 2XY) eluting with 0-15% EtOAc/hexanes then 15-30% EtOAc/hexanes afforded 0.146 gm of an oil (75%). ¹H NMR (400 MHz, CDCl₃) δ ppm 0.71-0.75 (m, 2H) 1.00-1.05 (m, 2H) 2.12-2.17 (m, 1H) 3.42 (s, 3H) 4.28 (s, 2H) 6.61 (t, J=73.14 Hz, 1H) 7.14 (d, J=8.5 Hz, 1H) 7.45 (t, J=7.8 Hz, 1H) 7.59 (s, 1H) 7.69 (dd, J=8.5, 2.1 Hz, 2H) 7.89 (d, J=7.9 Hz, 1H) 7.97 (s, 1H); MS (APPI) m/z 385 [M+H]⁺

### Step 2: Synthesis of 2-amino-4-(3-cyclopropyl-4-(difluoromethoxy)phenyl)-4-(3-(3-methoxyprop-1-ynyl)phenyl)-1-methyl-1H-imidazol-5(4H)-one

1-(3-Cyclopropyl-4-(difluoromethoxy)phenyl)-2-(3-(3-methoxyprop-1-ynyl)phenyl)ethane-1,2-dione (0.94 g, 0.244 mmol) was dissolved in isopropanol (12 mL). Methylguanidine hydrochloride (40 mg, 0.365 mmol) was added followed by sodium carbonate (39 mg, 0.368 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded oil. The oil was re-dissolved in diethylether, diluted with hexanes and concentrated, twice to give a white foam, 80 mg (76%); ¹H NMR (400 MHz, DMSO-d₆) δ ppm 0.45-0.49 (m, 2H) 0.89-0.94 (m, 2H) 1.96-2.03 (m, 1H) 2.93 (s, 3H) 3.27 (s, 3H) 4.27 (s, 2H) 6.71 (br. s., 2H) 7.04-7.06 (m, 2H) 7.11 (t, J=74.4 Hz, 1H) 7.25-7.30 (m, 3H) 7.37-7.40 (m, 1H) 7.44 (s, 1H); MS (ES) m/z 440.2 [M+H]⁺

### EXAMPLE 181

### Preparation of: (S)-2-amino-4-(3-cyclopropyl-4-(difluoromethoxy)phenyl)-4-(3-(3-methoxyprop-1-ynyl)phenyl)-1-methyl-1H-imidazol-5(4H)-one

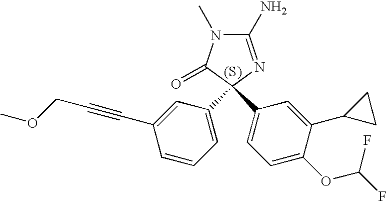

A racemic mixture of 2-amino-4-(3-cyclopropyl-4-(difluoromethoxy)phenyl)-4-(3-(3-methoxyprop-1-ynyl)phenyl)-1-methyl-1H-imidazol-5(4H)-one (178 mg) was separated by chiral column chromatography (Chiralpak AD-H, 2×25 cm) eluting with 5% methanol/ethanol (8/2 with 0.1% diethylamine) in hexanes to provide peak 1 (RT=11.0 min) (S)-2-amino-4-(3-cyclopropyl-4-(difluoromethoxy)phenyl)-4-(3-(3-methoxyprop-1-ynyl)phenyl)-1-methyl-1H-imidazol-5(4H)-one (67 mg) as a white foam MS m/e [M+H]⁺ 440.1, [α]_{D}²⁵=+4.00 (c=1% in MeOH).

### EXAMPLE 182

### Preparation of: (R)-2-amino-4-(3-cyclopropyl-4-(difluoromethoxy)phenyl)-4-(3-(3-methoxyprop-1-ynyl)phenyl)-1-methyl-1H-imidazol-5(4H)-one

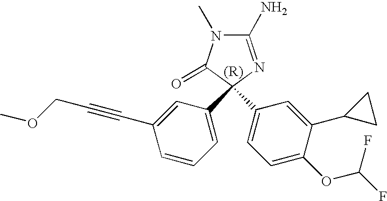

A racemic mixture of 2-amino-4-(3-cyclopropyl-4-(difluoromethoxy)phenyl)-4-(3-(3-methoxyprop-1-ynyl)phenyl)-1-methyl-1H-imidazol-5(4H)-one (178 mg) was separated by chiral column chromatography (Chiralpak AD-H, 2×25 cm) eluting with 5% methanol/ethanol (8/2 with 0.1% diethylamine) in hexanes to provide peak 2 (RT=12.6 min) (R)-2-amino-4-(3-cyclopropyl-4-(difluoromethoxy)phenyl)-4-(3-(3-methoxyprop-1-ynyl)phenyl)-1-methyl-1H-imidazol-5(4H)-one (50 mg) as a white foam MS m/e [M+H]⁺ 440.1, [α]_{D}²⁵=−8.00 (c=1% in MeOH)

### EXAMPLE 183

### Preparation of: 2-amino-4-(3-cyclopropyl-4-(difluoromethoxy)phenyl)-4-(3-(4-methoxybut-1-ynyl)phenyl)-1-methyl-1H-imidazol-5(4H)-one

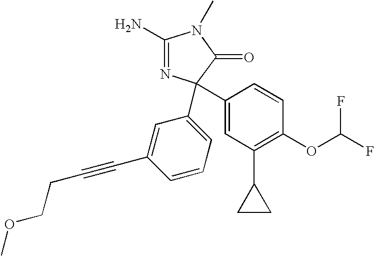

### Step 1: Synthesis of 1-(3-cyclopropyl-4-(difluoromethoxy)phenyl)-2-(3-(4-methoxybut-1-ynyl)phenyl)ethane-1,2-dione

To a Biotage conical microwave vial (0.5-2.0 ml) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and 4-methoxybut-1-yne (70 mg, 0.832 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated a total of three times. The combined runs were diluted with diethylether and washed with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afford 0.272 gm of an oil (91%). ¹H NMR (400 MHz, CDCl₃) δ ppm 0.70-0.74 (m, 2H) 1.00-1.04 (m, 2H) 2.12-2.18 (m, 1H) 2.66 (t, J=6.84 Hz, 2H) 3.37 (s, 3H) 3.56 (t, J=6.84 Hz, 2H) 6.60 (t, J=73.14 Hz, 1H) 7.13 (d, J=8.58 Hz, 1H) 7.41 (t, J=7.77 Hz, 1H) 7.57 (s, 1H) 7.63-7.69 (m, 2H) 7.84 (d, J=7.77 Hz, 1H) 7.92 (s, 1H); MS (APPI) m/z 399 [M+H]⁺

### Step 2: Synthesis of 2-amino-4-(3-cyclopropyl-4-(difluoromethoxy)phenyl)-4-(3-(4-methoxybut-1-ynyl)phenyl)-1-methyl-1H-imidazol-5(4H)-one

1-(3-Cyclopropyl-4-(difluoromethoxy)phenyl)-2-(3-(4-methoxybut-1-ynyl)phenyl)ethane-1,2-dione (0.220 g, 0.552 mmol) was dissolved in isopropanol (25 mL). Methylguanidine hydrochloride (89 mg, 0.812 mmol) was added followed by sodium carbonate (88 mg, 0.831 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded oil. The oil was re-dissolved in diethylether, diluted with hexanes and concentrated, twice to give a white foam, 220 mg (88%); ¹H NMR (400 MHz, DMSO-d₆) δ ppm 0.44-0.48 (m, 2H) 0.88-0.93 (m, 2H) 1.96-2.02 (m, 1H) 2.59 (t, J=6.61 Hz, 2H) 2.92 (s, 3H) 3.23 (s, 3H) 3.44 (t, J=6.61 Hz, 2H) 6.67 (br. s., 2H) 7.02-7.04 (m, 2H) 7.11 (t, J=74.41 Hz, 1H) 7.17-7.26 (m, 3H) 7.31-34 (m, 1H) 7.36 (s, 1H); MS (ES) m/z 453.3 [M+H]⁺

### EXAMPLE 184

### Preparation of: (R)-2-amino-4-(3-cyclopropyl-4-(difluoromethoxy)phenyl)-4-(3-(4-methoxybut-1-ynyl)phenyl)-1-methyl-1H-imidazol-5(4H)-one

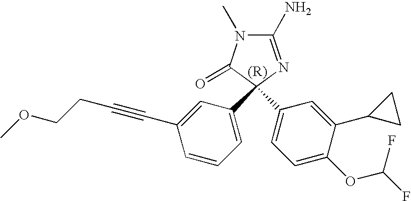

A racemic mixture of 2-amino-4-(3-cyclopropyl-4-(difluoromethoxy)phenyl)-4-(3-(4-methoxybut-1-ynyl)phenyl)-1-methyl-1H-imidazol-5(4H)-one (175 mg) was separated by chiral column chromatography (Chiralcel AD-H, 2×25 cm) eluting with 5% methanol/ethanol (8/2 with 0.1% diethylamine) in hexanes to provide peak 1 (RT=9.1 min) (R)-2-amino-4-(3-cyclopropyl-4-(difluoromethoxy)phenyl)-4-(3-(4-methoxybut-1-ynyl)phenyl)-1-methyl-1H-imidazol-5(4H)-one (64 mg) as a white foam MS m/e [M+H]⁺ 454.1, [α]_{D}²⁵=+4.00 (c=1.00 mg in 0.20 ml MeOH).

### EXAMPLE 185

### Preparation of: (S)-2-amino-4-(3-cyclopropyl-4-(difluoromethoxy)phenyl)-4-(3-(4-methoxybut-1-ynyl)phenyl)-1-methyl-1H-imidazol-5(4H)-one

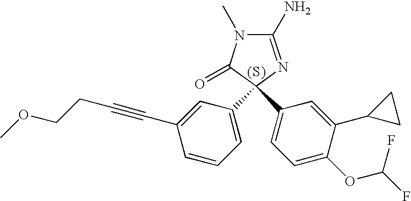

A racemic mixture of 2-amino-4-(3-cyclopropyl-4-(difluoromethoxy)phenyl)-4-(3-(4-methoxybut-1-ynyl)phenyl)-1-methyl-1H-imidazol-5(4H)-one (175 mg) was separated by chiral column chromatography (Chiralcel AD-H, 2×25 cm) eluting with 5% methanol/ethanol (8/2 with 0.1% diethylamine) in hexanes to provide peak 2 (RT=10.3 min) (S)-2-amino-4-(3-cyclopropyl-4-(difluoromethoxy)phenyl)-4-(3-(4-methoxybut-1-ynyl)phenyl)-1-methyl-1H-imidazol-5(4H)-one (61 mg) as a white foam MS m/e [M+H]⁺ 454.1, [α]_{D}²⁵=−6.58 (c=1.52 mg in 0.20 ml MeOH).

### EXAMPLE 186

### Preparation of: 2-amino-4-(3-cyclopropyl-4-(difluoromethoxy)phenyl)-4-(3-(5-methoxypent-1-ynyl)phenyl)-1-methyl-1H-imidazol-5(4H)-one

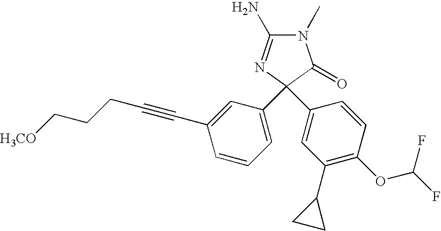

### Step 1: Synthesis of 1-(3-cyclopropyl-4-(difluoromethoxy)phenyl)-2-(3-(5-methoxypent-1-ynyl)phenyl)ethane-1,2-dione

To a Biotage conical microwave vial (0.5-2.0 mL) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and 5-methoxypent-1-yne (0.100 gm, 1.04 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. The runs were combined, diluted with diethylether and washed with saturated ammonium chloride solution. The ethereal layer was concentrated and loaded onto silica gel. Purification by chromatography (YAMAZEN W-Prep 2XY) eluting with 0-20% (1:1 dichloromethane-diethylether) in hexanes afforded 0.169 gm of an oil (81%). ¹H NMR (400 MHz, DMSO-d₆) δ ppm 0.70 (dd, J=5.2, 2.0 Hz, 2H) 0.98 (ddd, J=10.7, 4.3, 4.2 Hz, 2H) 1.73 (quin, J=6.7 Hz, 2H) 2.05-2.13 (m, 1H) 2.41-2.45 (m, 2H) 3.20 (s, 3H) 3.38 (t, J=6.3 Hz, 2H) 7.359 (t, J=73.0, 1H) 7.29 (d, J=8.6 Hz, 1H) 7.53 (t, J=2.3 Hz, 1H) 7.55-7.59 (m, 1H) 7.70-7.76 (m, 2H) 7.78-7.85 (m, 2H); MS (ES) m/z 411.2 [M−H]⁻

### Step 2: Synthesis of 2-amino-4-(3-cyclopropyl-4-(difluoromethoxy)phenyl)-4-(3-(5-methoxypent-1-ynyl)phenyl)-1-methyl-1H-imidazol-5(4H)-one

1-(3-cyclopropyl-4-(difluoromethoxy)phenyl)-2-(3-(5-methoxypent-1-ynyl)phenyl)ethane-1,2-dione (0.160 g, 0.388 mmol) was dissolved in isopropanol (30 mL). Methylguanidine hydrochloride (66 mg, 0.602 mmol) was added followed by sodium carbonate (69 mg, 0.614 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded oil. The oil was re-dissolved in diethylether, diluted with hexanes and concentrated, twice to give a white foam, 112 mg (62%); ¹H NMR (400 MHz, DMSO-d₆) δ ppm 0.46 (dd, J=5.2, 1.7 Hz, 2H) 0.90 (dd, J=8.5, 2.0 Hz, 2H) 1.70 (quin, J=6.5 Hz, 2H) 1.94-2.02 (m, 1H) 2.39 (t, J=7.1 Hz, 2H) 2.92 (s, 3H) 3.20 (s, 3H) 3.37 (t, J=6.3 Hz, 2H) 6.67 (br. s., 2H) 7.03 (d, J=8.1 Hz, 2H) 7.09 (t, J=74.4 Hz, 1H) 7.16-7.27 (m, 3H) 7.29-7.33 (m, 1H) 7.35 (s, 1H); MS (ES) m/z 468.2 [M+H]⁺

### EXAMPLE 187

### Preparation of: 2-amino-4-(3-cyclopropyl-4-(difluoromethoxy)phenyl)-4-(3-cyclopropylphenyl)-1-methyl-1H-imidazol-5(4H)-one

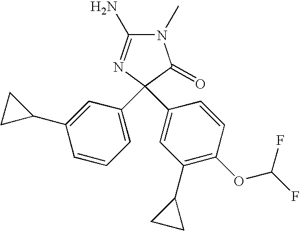

### Step 1: Synthesis of 1-(3-cyclopropyl-4-(difluoromethoxy)phenyl)-2-(3-cyclopropylphenyl)ethane-1,2-dione

In a 100 mL round bottom flask equipped with a magnetic stir egg were combined 1-(3-bromophenyl)-2-(3-cyclopropyl-4-(difluoromethoxy)phenyl)ethane-1,2-dione (400 mg, 1.01 mmol), cyclopropylboronic acid (0.400 gm, 4.64 mmol), potassium phosphate (1.41 gm, 6.64 mmol), palladium acetate (21 mg, 0.094 mmol) and tricylclohexyl-phosphine (54 mg, 0.193 mmol) in toluene (10 mL) and water (0.5 mL). The reaction mixture was immersed in a pre-heated oil bath at 100° C. for 4 hours. The crude reaction mixture was partitioned between ethylacetate and water, extracted with ethylacetate and dried with magnesium sulfate. Purification by column chromatography, YAMAZEN W-Prep 2XY (0-25% ethylacetate in hexanes) yielded 0.263 gm of an oil (73%). ¹H NMR (400 MHz, DMSO-d₆) δ ppm 0.72 (dd, J=4.6, 2.3 Hz, 4H) 0.95-1.05 (m, 4H) 2.01-2.08 (m, 1H) 2.08-2.16 (m, 1H) 7.32 (d, J=8.6 Hz, 1H) 7.42 (t, J=73.3 Hz, 1H) 7.43-7.50 (m, 2H) 7.54 (d, J=2.3 Hz, 1H) 7.59-7.63 (m, 1H) 7.65 (s, 1H) 7.72 (dd, J=8.6, 2.3 Hz, 1H); MS (ES) m/z 355.1 [M−H]⁻

### Step 2: Synthesis of 2-amino-4-(3-cyclopropyl-4-(difluoromethoxy)phenyl)-4-(3-cyclopropylphenyl)-1-methyl-1H-imidazol-5(4H)-one

1-(3-Cyclopropyl-4-(difluoromethoxy)phenyl)-2-(3-cyclopropylphenyl)ethane-1,2-dione (0.252 g, 0.707 mmol) was dissolved in isopropanol (25 mL). Methylguanidine hydrochloride (115 mg, 1.05 mmol) was added followed by sodium carbonate (112 mg, 1.06 mmol). The mixture was heated (oil bath 86° C.) for 16 hours. The isopropanol was removed by rotary evaporation and the residue was transferred onto silica gel using ethyl acetate. Purification by column chromatography [step gradient; 50% EtOAc/hex, 100% EtOAc then 10% MeOH/EtOAc] afforded oil. The oil was re-dissolved in diethylether, diluted with hexanes and concentrated, twice to give a white foam, 232 mg (80%); ¹H NMR (400 MHz, DMSO-d₆) δ ppm 0.46-0.57 (m, 4H) 0.87-0.97 (m, 4H) 1.79-1.87 (m, 1H) 1.98-2.06 (m, 1H) 2.95 (s, 3H) 6.65 (br. s., 2H) 6.85 (d, J=7.4 Hz, 1H) 7.04-7.08 (m, 3H) 7.12 (t, J=74.2 Hz, 1H) 7.13-7.17 (m, 2H) 7.27-7.30 (m, 1H); MS (ES) m/z 412.1 [M+H]⁺

### EXAMPLE 188

### Preparation of: 2-amino-4-(3-cyclopropyl-4-(difluoromethoxy)phenyl)-4-(3-(5-hydroxypent-1-ynyl)phenyl)-1-methyl-1H-imidazol-5(4H)-one

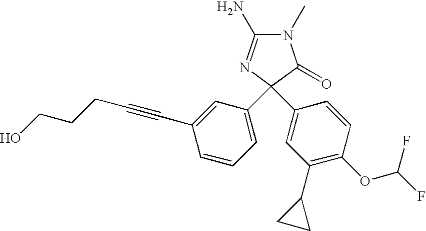

### Step 1: Synthesis of 1-(3-cyclopropyl-4-(difluoromethoxy)phenyl)-2-(3-(5-hydroxypent-1-ynyl)phenyl)ethane-1,2-dione

To a Biotage conical microwave vial (0.5-2.0 mL) equipped with a magnetic spin vane was added 1-(3-bromophenyl)-2-(3-cyclopropyl-4-(difluoromethoxy)phenyl)ethane-1,2-dione (100 mg, 0.253 mmol) in triethylamine (1.2 mL). Copper iodide (10 mg, 0.052 mmol), Tetrakis(triphenylphosphine) palladium (20 mg, 0.017 mmol) and pent-4-yn-1-ol (0.850 gm, 10.1 mmol) were added at room temperature. The vial was covered with Teflon septa and secured via a crimped aluminum cap. The reaction was irradiated in a Biotage Initiator microwave at 80° C. for 60 minutes (Fixed Hold Time On, Normal absorbance level). The same reaction set-up was repeated. The runs were combined, diluted with diethylether and washed with saturated ammonium chloride solution. The ethereal layer was concentrated and loaded onto silica gel. Purification by chromatography (YAMAZEN W-Prep 2XY) eluting with 33% ethyl acetate in hexanes afforded 0.181 gm of an oil (90%). ¹H NMR (400 MHz, CDCl₃) δ ppm 0.70-0.74 (m, 2H) 1.00-1.04 (m, 2H) 1.80-1.87 (m, 2H) 2.12-2.18 (m, 1H) 2.51 (t, J=6.95 Hz, 2H) 3.78 (t, J=6.14 Hz, 2H) 6.61 (t, J=73.14 Hz, 1H) 7.13 (d, J=8.57 Hz, 1H) 7.41 (t, J=7.77 Hz, 1H) 7.58 (s, 1H) 7.62-7.70 (m, 2H) 7.88 (d, J=7.77 Hz, 1H) 7.91 (s, 1H); MS (ES) m/z 399.1 [M+H]⁺

### Step 2: Synthesis of 2-amino-4-(3-cyclopropyl-4-(difluoromethoxy)phenyl)-4-(3-(5-hydroxypent-1-ynyl)phenyl)-1-methyl-1H-imidazol-5(4H)-one

1-(3-Cyclopropyl-4-(difluoromethoxy)phenyl)-2-(3-(5-hydroxypent-1-ynyl)phenyl)ethane-1,2-dione (0.172 g, 0.432 mmol) was dissolved in isopropanol (20 mL). Methylguanidine hydrochloride (70 mg, 0.639 mmol) was added followed by sodium carbonate (69 mg, 0.652 mmol). The mixture was heated (oil bath 86° C.) for 16 hours. The isopropanol was removed by rotary evaporation and the residue was transferred onto silica gel using ethyl acetate. Purification by column chromatography [step gradient; EtOAc then 10% MeOH/EtOAc] afforded oil. The oil was re-dissolved in diethylether, diluted with hexanes and concentrated, twice to give a white foam, 114 mg (58%); ¹H NMR (400 MHz, DMSO-d₆) δ ppm 0.44-0.48 (m, 2H) 0.88-0.93 (m, 2H) 1.58-1.65 (m, 2H) 1.95-2.02 (m, 1H) 2.39 (t, J=7.13 Hz, 2H) 2.45 (s, 3H) 3.42-3.47 (m, 2H) 4.46 (t, J=5.22 Hz, 1H) 6.67 (br. s., 2H) 7.02-7.04 (m, 2H) 7.09 (t, J=74.40 Hz, 1H) 7.17-7.26 (m, 3H) 7.32 (d, J=7.53 Hz, 1H) 7.36 (s, 1H); MS (ES) m/z 454.2 [M+H]⁺

### EXAMPLE 189

### Preparation of: (R)-2-amino-4-(3-cyclopropyl-4-(difluoromethoxy)phenyl)-4-(3-(5-hydroxypent-1-ynyl)phenyl)-1-methyl-1H-imidazol-5(4H)-one

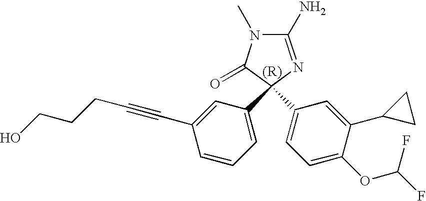

A racemic mixture of 2-amino-4-(3-cyclopropyl-4-(difluoromethoxy)phenyl)-4-(3-(5-hydroxypent-1-ynyl)phenyl)-1-methyl-1H-imidazol-5(4H)-one (177 mg) was separated by chiral column chromatography (Chiralcel AD-H, 2×25 cm) eluting with 5% methanol/ethanol (8/2 with 0.1% diethylamine) in hexanes to provide peak 1 (RT=5.9 min) (R)-2-amino-4-(3-cyclopropyl-4-(difluoromethoxy)phenyl)-4-(3-(5-hydroxypent-1-ynyl)phenyl)-1-methyl-1H-imidazol-5(4H)-one (33 mg) as a white foam MS m/e [M+H]⁺ 454.1, [α]_{D}²⁵=+10.0 (c=8.70 mg in 0.3 mL MeOH).

### EXAMPLE 190

### Preparation of: (S)-2-amino-4-(3-cyclopropyl-4-(difluoromethoxy)phenyl)-4-(3-(5-hydroxypent-1-ynyl)phenyl)-1-methyl-1H-imidazol-5(4H)-one

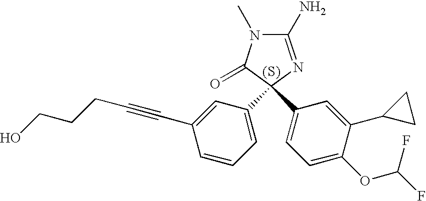

A racemic mixture of 2-amino-4-(3-cyclopropyl-4-(difluoromethoxy)phenyl)-4-(3-(5-hydroxypent-1-ynyl)phenyl)-1-methyl-1H-imidazol-5(4H)-one (177 mg) was separated by chiral column chromatography (Chiralcel AD-H, 2×25 cm) eluting with 5% methanol/ethanol (8/2 with 0.1% diethylamine) in hexanes to provide peak 2 (RT=6.7 min) (S)-2-amino-4-(3-cyclopropyl-4-(difluoromethoxy)phenyl)-4-(3-(5-hydroxypent-1-ynyl)phenyl)-1-methyl-1H-imidazol-5(4H)-one (42 mg) as a white foam MS m/e [M+H]⁺ 454.1, [α]_{D}²⁵=−10.6 (c=13.6 mg in 0.3 mL MeOH)

### EXAMPLE 191

### Preparation of: 2-amino-4-(3-cyclopropyl-4-(difluoromethoxy)phenyl)-4-(3-(5-fluoropent-1-ynyl)phenyl)-1-methyl-1H-imidazol-5(4H)-one

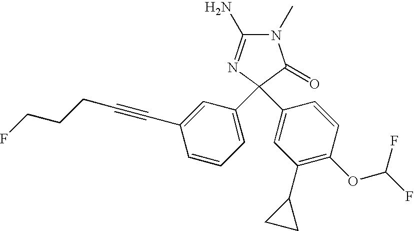

### Step 1: Synthesis of 1-(3-cyclopropyl-4-(difluoromethoxy)phenyl)-2-(3-(5-fluoropent-1-ynyl)phenyl)ethane-1,2-dione

In a 100 mL round bottom 1-(3-cyclopropyl-4-(difluoromethoxy)phenyl)-2-(3-(5-hydroxypent-1-ynyl)phenyl)ethane-1,2-dione (240 mg, 0.602 mmol) was dissolved in dichloromethane (5 mL) and chilled to −78° C. (Dimethylamino)sulfur trifluoride (0.5 mL, 5.12 mmol) was introduced via syinge injection. After 30 minutes dry ice bath was removed. After 4 hours the reaction mixture was partitioned between diethylether and saturated sodium bicarbonate solution. Organic layer was concentrated to a residue, and loaded directly on top of chromatography column. Purification (YAMAZEN W-Prep 2XY) via automated gradient elution 100% hexanes (4 min) to 18% EtOAC (hold 4 min) to 39% EtOAc (hold 8 min) afforded 130 mg (54%) of an oil. ¹H NMR (400 MHz, CDCl₃) δ ppm 0.74 (q, J=4.8 Hz, 2H) 1.03 (dd, J=8.6, 1.6 Hz, 2H) 1.87-2.06 (m, 2H) 2.16 (dq, J=12.6, 5.0 Hz, 1H) 2.55 (t, J=7.1 Hz, 2H) 4.58 (dt, J=47.1, 5.6 Hz, 2H) 6.63 (t, J=73.1 Hz, 1H) 7.14 (d, J=8.6 Hz, 1H) 7.43 (t, J=7.8 Hz, 1H) 7.59 (d, J=2.1 Hz, 1H) 7.64 (t, J=4.5 Hz, 1H) 7.70 (dd, J=8.5, 2.2 Hz, 1H) 7.84 (t, J=4.8 Hz, 1H) 7.93 (t, J=1.5 Hz, 1H); MS (EI) m/z 400 [M+.]

### Step 2: Synthesis of 2-amino-4-(3-cyclopropyl-4-(difluoromethoxy)phenyl)-4-(3-(5-fluoropent-1-ynyl)phenyl)-1-methyl-1H-imidazol-5(4H)-one

1-(3-Cyclopropyl-4-(difluoromethoxy)phenyl)-2-(3-(5-fluoropent-1-ynyl)phenyl)ethane-1,2-dione (0.125 g, 0.312 mmol) was dissolved in isopropanol (25 mL). Methylguanidine hydrochloride (53 mg, 0.483 mmol) was added followed by sodium carbonate (52 mg, 0.490 mmol). The mixture was heated (oil bath 86° C.) for 16 hours. The isopropanol was removed by rotary evaporation and the residue was transferred onto silica gel using ethyl acetate. Purification on YAMAZEN W-Prep 2XY (100% EtOAc) afforded an oil. The oil was re-dissolved in diethylether, diluted with hexanes and concentrated, twice to give a white foam, 140 mg (98%); ¹H NMR (400 MHz, DMSO-d₆) δ ppm 0.50 (dd, J=5.1, 1.9 Hz, 2H) 0.94 (dd, J=8.6, 2.1 Hz, 2H) 1.82-1.97 (m, 2H) 1.98-2.07 (m, 1H) 2.53 (d, J=3.9 Hz, 2H) 4.50 (dt, J=47.4, 5.8 Hz, 2H) 2.96 (s, 3H) 6.67 (br s, 2H) 7.07 (d, J=5.8 Hz, 2H) 7.09 (t, J=74.4 Hz, 1H) 7.22-7.30 (m, 3H) 7.37 (d, J=7.0 Hz, 1H) 7.41 (s, 1H); MS (ES) m/z 456.0 [M+H]⁺

### EXAMPLE 192

### Preparation of: (S)-2-amino-4-(3-cyclopropyl-4-(difluoromethoxy)phenyl)-4-(3-(5-fluoropent-1-ynyl)phenyl)-1-methyl-1H-imidazol-5(4H)-one

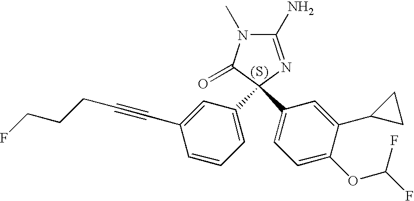

A racemic mixture of 2-amino-4-(3-cyclopropyl-4-(difluoromethoxy)phenyl)-4-(3-(5-fluoropent-1-ynyl)phenyl)-1-methyl-1H-imidazol-5(4H)-one (312 mg) was separated by chiral column chromatography (Chiralpak AD-H, 2×25 cm) eluting with 5% methanol/ethanol (8/2 with 0.1% diethylamine) in hexanes to provide peak 1 (RT=8.0 min) (S)-2-amino-4-(3-cyclopropyl-4-(difluoromethoxy)phenyl)-4-(3-(5-fluoropent-1-ynyl)phenyl)-1-methyl-1H-imidazol-5(4H)-one (42 mg) as a white foam MS (ES) m/e [M+H]⁺ 456.1, [α]_{D}²⁵=+9.0 (c=1% in MeOH)

### EXAMPLE 193

### Preparation of: (R)-2-amino-4-(3-cyclopropyl-4-(difluoromethoxy)phenyl)-4-(3-(5-fluoropent-1-ynyl)phenyl)-1-methyl-1H-imidazol-5(4H)-one

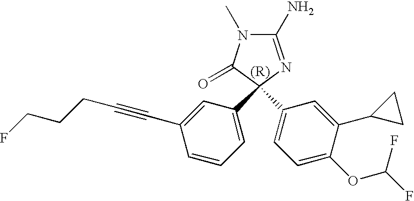

A racemic mixture of 2-amino-4-(3-cyclopropyl-4-(difluoromethoxy)phenyl)-4-(3-(5-fluoropent-1-ynyl)phenyl)-1-methyl-1H-imidazol-5(4H)-one (312 mg) was separated by chiral column chromatography (Chiralpak AD-H, 2×25 cm) eluting with 5% methanol/ethanol (8/2 with 0.1% diethylamine) in hexanes to provide peak 2 (RT=9.0 min) (R)-2-amino-4-(3-cyclopropyl-4-(difluoromethoxy)phenyl)-4-(3-(5-fluoropent-1-ynyl)phenyl)-1-methyl-1H-imidazol-5(4H)-one (33 mg) as a white foam MS (ES) m/e [M+H]⁺ 456.1, [α]_{D}²⁵=−9.60 (c=1% in MeOH).

### EXAMPLE 194

### Preparation of: 2-amino-4,4-bis(3-cyclopropyl-4-(difluoromethoxy)phenyl)-1-methyl-1H-imidazol-5(4H)-one

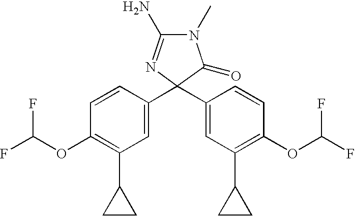

### Step 1: Synthesis of 2-cyclopropylphenol

[Reference: Jose Barluenga in Org. Lett. 2002, 4(13), 2225]

Prepared in a similar manner (with the exception of 2-bromophenol starting material) 2-Iodo-phenol (25 g, 0.113 mmol), allyl bromide (9.8 mL, 0.113 mmol), potassium carbonate (15.7 g, 0.113 mmol) and N,N-dimethylformamide (100 mL) were mixed in a round bottomed flask and stirred at room temperature under nitrogen for 18 h. The solution was diluted with hexane (500 mL), stirred vigorously, then decanted from the N,N-dimethylformamide layer. This was repeated once more with hexane (250 mL). The combined hexane layers were evaporated, and the resulting oil was dissolved in ethyl acetate (500 mL) and washed five times with water (250 mL), and then dried with sodium sulfate. Concentration under reduced pressure gave 14.5 g (49%) of oil, 1-allyloxy-2-iodo-benzene, which was used without further purification. 1-Allyloxy-2-iodo-benzene (11.3 g, 43.4 mmol) and hexane (217 mL) was mixed in a 1 L round bottomed flask and placed under nitrogen. The solution was cooled to −78° C. (Dry Ice/acetone bath). A solution of tert-butyllithium (51 mL of a 1.7 M solution in Pentane, 86.7 mmol) was added dropwise over 25 min. The resulting solution was stirred at −78° C. for 1 h. TMEDA (13.1 mL, 86.8 mmol) was added dropwise, and the reaction mixture was allowed to warm to room temperature overnight. The reaction mixture was carefully poured into a stirred solution of diethyl ether (200 mL) and saturated ammonium chloride (100 mL), and stirred at RT for 15 min. The solution was poured into a separatory funnel and the aqueous layer was removed. The organic layer was dried with sodium sulfate. Concentration under reduced pressure, followed by purification through silica gel column chromatography (YAMAZEN W-Prep 2XY elution with 10-35% dichloromethane in hexanes) gave 1.65 g (28%). ¹H NMR (400 MHz, CDCl₃) δ ppm 0.65 (dd, J=5.5, 1.8 Hz, 2H) 0.97 (ddd, J=10.0, 4.2, 4.0 Hz, 2H) 1.81 (tt, J=8.3, 5.3 Hz, 1H) 5.41 (br. s., 1H) 6.86 (d, J=7.5 Hz, 2H) 7.10 (dd, J=15.5, 7.7 Hz, 2H).

### Step 2: Synthesis of 4-bromo-2-cyclopropylphenol

2-Cyclopropyl-phenol (1.65 g, 12.3 mmol) was dissolved in dichloromethane (24 mL). A solution of bromine (0.63 mL, 12.3 mmol) in dichloromethane (12 mL) was added dropwise over 37 min. The resulting solution was stirred at room temperature for 1 h. The reaction mixture was diluted with diethyl ether (100 mL), washed with 10% sodium thiosulfate (30 mL), brine (30 mL), and then dried with sodium sulfate. Concentration under reduced pressure gave 2.5 g (96%) of oil. Purification by column chromatography (YAMAZEN W-Prep 2XY elution with 10-35% dichloromethane in hexanes) afforded 1.33 g (51%). ¹H NMR (400 MHz, DMSO-d₆) δ ppm 0.61 (dd, J=5.3, 2.1 Hz, 2H) 0.86 (ddd, J=10.5, 4.3, 4.2 Hz, 2H) 1.96-2.07 (m, 1H) 6.71 (d, J=8.6 Hz, 1H) 6.83 (d, J=2.5 Hz, 1H) 7.08 (dd, J=8.6, 2.5 Hz, 1H) 9.60 (s, 1H); MS (ES) m/z 210.9 [M−H]⁻

### Step 3: Synthesis of 4-bromo-2-cyclopropyl-1-(difluoromethoxy)benzene

A mixture of 4-bromo-2-cyclopropyl-phenol (1.33 g, 6.2 mmol), potassium carbonate (5.2 g, 37.5 mmol), sodium chlorodifluoroacetate (2.85 g, 18.7 mmol), water (1.56 mL), and N,N-dimethylformamide (7.8 mL) was place under nitrogen and heated to 110° C. After 8 h HPLC analysis indicated a mixture of 4-bromo-2-cyclopropyl-phenol (64%) and 4-bromo-2-cyclopropyl-1-difluoromethoxy-benzene (20%). Potassium carbonate (5.2 g, 37.5 mmol) and sodium chlorodifluoroacetate (2.85 g, 18.7 mmol) was added, and heating at 110° C. was continued. After 8 h HPLC indicated no further improvement. The reaction mixture was cooled, then diluted with water (100 mL) and extracted twice with dichloromethane (50 mL). The combined dichloromethane layers were washed once more with water (100 mL). Chromatography on silica gel (YAMAZEN W-Prep 2XY elution with hexanes) gave 0.606 g (37%). ¹H NMR (400 MHz, DMSO-d₆) δ ppm 0.73 (dd, J=5.1, 2.1 Hz, 2H) 0.96 (ddd, J=8.5, 5.4, 5.3 Hz, 2H) 2.00-2.10 (m, 1H) 7.07-7.13 (m, 2H) 7.19 (t, J=73.9 Hz, 1H) 7.38 (dd, J=8.7, 2.4 Hz, 1H); MS (APPI) m/z 262 [M+.]

### Step 4: Synthesis of 1,2-bis(3-cyclopropyl-4-(difluoromethoxy)phenyl)ethyne

In a CEM snap top microwave vial were combined trimethylsilylacetylene (114 mg, 1.16 mmol), 4-bromo-2-cyclopropyl-1-(difluoromethoxy)benzene (0.606 g, 2.30 mmol), tetrakis(triphenylphosphine)palladium (30 mg, 0.026 mmol) and pyrrolidine (1 mL, 12 mmol). The reaction vial was placed in a CEM Explorer™ microwave and irradiated for 30 minutes at 80° C. The crude reaction mixture was poured directly onto silica gel and purification by column chromatography (YAMAZEN W-Prep 2XY elution with hexanes) yielded 0.315 g of a clear oil (70%). ¹H NMR (400 MHz, DMSO-d₆) δ ppm 0.74 (dd, J=5.1, 2.1 Hz, 4H) 0.97 (dd, J=8.5, 2.2 Hz, 4H) 2.02-2.12 (m, 2H) 7.12 (d, J=1.9 Hz, 2H) 7.17 (d, J=8.3 Hz, 2H) 7.27 (t, J=73.9 Hz, 2H) 7.38 (dd, J=8.6, 2.1 Hz, 2H); MS (APPI) m/z 390 [M+.]

### Step 5: Synthesis of 1,2-bis(3-cyclopropyl-4-(difluoromethoxy)phenyl)ethane-1,2-dione

In a 50 mL round bottom flask was dissolved 1,2-bis(3-cyclopropyl-4-(difluoromethoxy)phenyl)ethyne (0.263 g, 0.674 mmol) in acetone (5.7 mL). An aqueous solution (1.9 mL) of sodium bicarbonate (32 mg, 0.302 mmol) and magnesium sulfate (113 mg, 0.939 mmol) was added, followed by potassium permanganate (0.241 g, 1.52 mmol). After 10 minutes the reaction was diluted twice with hexanes (20 mLs) and decanted, then dried with magnesium sulfate. The organic material was concentrated onto silica gel. Column chromatography (YAMAZEN W-Prep 2XY elution with 0-10% EtOAc in hexanes) afforded 0.256 gm of an oil (90%). ¹H NMR (400 MHz, DMSO-d₆) δ ppm 0.73 (dd, J=5.2, 2.1 Hz, 4H) 1.01 (ddd, J=10.6, 4.4, 4.3 Hz, 4H) 2.05-2.16 (m, 2H) 7.32 (d, J=8.5 Hz, 2H) 7.42 (t, J=73.2 Hz, 2H) 7.54 (d, J=2.2 Hz, 2H) 7.72 (dd, J=8.5, 2.2 Hz, 2H); MS (EI) m/z 422 [M+.]

### Step 6: Synthesis of 2-amino-4,4-bis(3-cyclopropyl-4-(difluoromethoxy)phenyl)-1-methyl-1H-imidazol-5(4H)-one

In a 100 mL round bottom flask was dissolved 1,2-bis(3-cyclopropyl-4-(difluoromethoxy)phenyl)ethane-1,2-dione (0.227 g, 0.537 mmol) in isopropanol (26 mL). Methylguanidine hydrochloride (87 mg, 0.794 mmol) was added followed by sodium carbonate (86 mg, 0.811 mmol). The mixture was heated (oil bath 85° C.) for 14 hours. The isopropanol was removed at the rotovap and the residue partitioned between water and chloroform. The organic layer was dried with sodium sulfate and concentrated onto silica gel. Purification by column chromatography [step gradient; 1:1 (EtOAc/hexanes) then 100% EtOAc] afforded 0.300 gm of a clear oil. The oil was redissolved in diethylether and concentrated, twice then placed under vacuum to give a white foam 210 mg (82%) ¹H NMR (400 MHz, DMSO-d₆) δ ppm 0.49 (dd, J=5.4, 2.0 Hz, 4H) 0.94 (ddd, J=10.4, 4.1, 4.0 Hz, 4H) 1.96-2.06 (m, 2H) 2.94 (s, 3H) 6.69 (br s., 2H) 7.02-7.08 (m, 4H) 7.11 (t, J=74.2 Hz, 2H) 7.27 (dd, J=8.5, 2.4 Hz, 2H); MS (ES) m/z 476.0 [M−H]⁻

### EXAMPLE 195

### Preparation of: 2-amino-5-[4-(difluoromethoxy)-3-methylphenyl]-5-(4-fluoro-3-isopropoxyphenyl)-3-methyl-3,5-dihydro-4H-imidazol-4-one

Step 1-5-((4-(difluoromethoxy)-3-methylphenyl)ethynyl)-2-fluorophenol

A mixture of 1-(difluoromethoxy)-4-iodobenzene (0.913 g, 3.21 mmol 1.25 eq), 5-ethynyl-2-fluorophenol (0.350 g, 2.57 mmol), bis(triphenylphosphino)palladium(II) chloride (0.13 g, 90 μmol), copper(I) iodide (0.015 g, 80 μmol) and triethylamine (1.43 g, 14.14 mmol) in DMF (4 mL) was stirred at RT for 3 h. The solvent is removed and the material is absorbed onto celite and purified by flash chromatography (silica, 5:95 ethyl acetate/hexanes) to afford 5-((4-(difluoromethoxy)-3-methylphenyl)ethynyl)-2-fluorophenol (0.65 g, 86%) as a white solid.

### Step 2-1-(difluoromethoxy)-4-((4-fluoro-3-isopropoxyphenyl)ethynyl)-2-methylbenzene

5-((4-(Difluoromethoxy)-3-methylphenyl)ethynyl)-2-fluorophenol (0.093 g, 0.33 mmol) and 2-iodopropane (0.201 g, 1.67 mmol) are dissolved in 2-butanone (4 mL) and cesium carbonate (0.135 g, 0.50 mmol) is added. The mixture is stirred overnight at reflux. The solution is cooled to RT and the solvent removed. The mixture is absorbed onto celite and purified by flash chromatography (silica, 5:95 ethyl acetate/hexanes) to yield 1-(difluoromethoxy)-4-((4-fluoro-3-isopropoxyphenyl)ethynyl)-2-methylbenzene (0.091 g, 78%).

### Step 3-1-(4-(difluoromethoxy)-3-methylphenyl)-2-(4-fluoro-3-isopropoxyphenyl)ethane-1,2-dione

1-(Difluoromethoxy)-4-((4-fluoro-3-isopropoxyphenyl)ethynyl)-2-methylbenzene (0.047 g, 0.14 mmol) is dissolved in acetone (2 mL) and added to a solution of NaHCO3 (0.002 g, 0.08 mmol) and MgSO4 (0.025 g, 0.21 mmol) in H20 (2 mL). KMnO4 (0.049 g, 0.31 mmol) is added in one portion and the solution is stirred for 2 h. EtOAC is added and the mixture is filtered through a pad of celite. The remaining solution is washed with H2O, brine, dried and the solvent removed to yield 1-(4-(difluoromethoxy)-3-methylphenyl)-2-(4-fluoro-3-isopropoxyphenyl)ethane-1,2-dione as a yellow solid (0.048 g, 98%).

### Step 4-2-amino-5-[4-(difluoromethoxy)-3-methylphenyl]-5-(4-fluoro-3-isopropoxyphenyl)-3-methyl-3,5-dihydro-4H-imidazol-4-one

1-(4-(Difluoromethoxy)-3-methylphenyl)-2-(4-fluoro-3-isopropoxyphenyl)ethane-,2-dione (0.048 g, 0.13 mmol) was dissolved in ethanol (5 mL). Methylguanidine hydrochloride (0.018 g, 0.16 mmol) was added followed by sodium carbonate (0.017 g, 0.16 mmol). The mixture was stirred at 85° C. overnight 15 hours. The solvent was removed and the material is absorbed onto celite. Purification by flash chromatography afforded 2-amino-5-[4-(difluoromethoxy)-3-methylphenyl]-5-(4-fluoro-3-isopropoxyphenyl)-3-methyl-3,5-dihydro-4H-imidazol-4-one (0.032 g, 58%).

MS (ES) m/z 420.2; MS (ES) m/z 480.2.

### EXAMPLE 196

### Preparation of: 2-amino-5-[4-(difluoromethoxy)-3-methylphenyl]-5-[3-(3-fluoroprop-1-yn-1-yl)phenyl]-3-methyl-3,5-dihydro-4H-imidazol-4-one

### Step 1-1-(4-(difluoromethoxy)-3-methylphenyl)-2-(3-(3-hydroxyprop-1-ynyl)phenyl)ethane-1,2-dione

A mixture of 1-(3-bromophenyl)-2-(4-(difluoromethoxy)-3-methylphenyl)ethane-1,2-dione

(0.500 g, 1.41 mmol), prop-2-yn-1-ol (0.395 g, 7.04 mmol), bis(triphenylphosphino)palladium(II) chloride (0.099 g, 0.14 mmol), copper(I) iodide (0.021 g, 0.11 mmol) and triethylamine (0.62 g, 6.11 mmol) in CH3CN (3 mL) was stirred at 60° C. overnight. The solvent is removed and the material is absorbed onto celite and purified by flash chromatography (silica, 25:75 ethyl acetate/hexanes) to afford 1-(4-(difluoromethoxy)-3-methylphenyl)-2-(3-(3-hydroxyprop-1-ynyl)phenyl)ethane-1,2-dione (0.289 g, 62%) as an off white solid.

### Step 2-1-(4-(difluoromethoxy)-3-methylphenyl)-2-(3-(3-fluoroprop-1-ynyl)phenyl)ethane-1,2-dione

1-(4-(Difluoromethoxy)-3-methylphenyl)-2-(3-(3-hydroxyprop-1-ynyl)phenyl)ethane-1,2-dione (0.230 g, 0.67 mmol) is dissolved in CH₂Cl₂ (3.0 mL) and cooled to −78° C. DAST (0.118 g, 0.73 mmol) is added and the solution is slowly warmed to RT. After 1 h at RT a saturated solution of NaHCO3 is added and the mixture extracted with CH2CL2. The CH2Cl2 is washed with H2O and brine. The solution is dried (MgSO4) and the material purified by flash chromatography to yield 1-(4-(difluoromethoxy)-3-methylphenyl)-2-(3-(3-fluoroprop-1-ynyl)phenyl)ethane-1,2-dione (0.196, 60%).

### Step 3-2-amino-5-[4-(difluoromethoxy)-3-methylphenyl]-5-[3-(3-fluoroprop-1-yn-1-yl)phenyl]-3-methyl-3,5-dihydro-4H-imidazol-4-one

1-(4-(Difluoromethoxy)-3-methylphenyl)-2-(3-(3-fluoroprop-1-ynyl)phenyl)ethane-1,2-dione (0.177 g, 0.51 mmol) was dissolved in ethanol (5 mL). Methylguanidine hydrochloride (0.070 g, 0.64 mmol) was added followed by sodium carbonate (0.68 g, 0.64 mmol). The mixture was stirred at 85° C. overnight 15 hours. The solvent was removed and the material is absorbed onto celite. Purification by flash chromatography afforded (silica, 10/1 CH₂Cl₂/MeOH) 2-amino-5-[4-(difluoromethoxy)-3-methylphenyl]-5-[3-(3-fluoroprop-1-yn-1-yl)phenyl]-3-methyl-3,5-dihydro-4H-imidazol-4-one (0.124 g, 60%).

### EXAMPLE 197

### Preparation of: 2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromo-4-fluorophenyl)-2-(4-(difluoromethoxy)-3-methylphenyl)ethane-1,2-dione with prop-2-yn-1-ol in step 1.

### EXAMPLE 198

### Preparation of: (5S)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one.

[α]_{D}²⁵=+22.00 (c=1% SOLUTION, MeOH);

MS (ES) m/z 418.2; MS (ES) m/z 837.4

### EXAMPLE 199

### Preparation of: (5R)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one.

[α]_{D}²⁵=−21.0° (c=1% SOLUTION, MeOH);

MS (ES) m/z 420.2; MS (ES) m/z 461.2.

### EXAMPLE 200

### Preparation of: (5R)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[3-(3-fluoroprop-1-yn-1-yl)phenyl]-3-methyl-3,5-dihydro-4H-imidazol-4-one.

[α]_{D}²⁵=+15.0° (c=1% SOLUTION, MeOH);

MS (ES) m/z 400.2; MS (ES) m/z 460.2; MS (ES) m/z 801.4.

### EXAMPLE 201

### Preparation of: (5S)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[3-(3-fluoroprop-1-yn-1-yl)phenyl]-3-methyl-3,5-dihydro-4H-imidazol-4-one.

[α]_{D}²⁵=−11.0° (C=1% SOLUTION, MeOH);

MS (ES) m/z 400.2; MS (ES) m/z 801.4.

### EXAMPLE 202

### Preparation of: 2-amino-5-[4-(difluoromethoxy)-3-methylphenyl]-5-[3-(4-fluorobut-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromophenyl)-2-(4-(difluoromethoxy)-3-methylphenyl)ethane-1,2-dione with but-3-yn-1-ol in step 1.

MS (ES) m/z 414.2.

### EXAMPLE 203

### Preparation of: 2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromo-4-fluorophenyl)-2-(4-(difluoromethoxy)-3-methylphenyl)ethane-1,2-dione with but-3-yn-1-ol in step 1.

MS (ES) m/z 432.2; MS (ES) m/z 865.4.

### EXAMPLE 204

### Preparation of: (5R)-2-amino-5-[4-(difluoromethoxy)-3-methylphenyl]-5-[3-(4-fluorobut-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[3-(4-fluorobut-1-yn-1-yl)phenyl]-3-methyl-3,5-dihydro-4H-imidazol-4-one.

MS (ES) m/z 414.1; MS (ES) m/z 829.2.

### EXAMPLE 205

### Preparation of: (5R)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one.

MS (ES) m/z 432.1; MS (ES) m/z 865.2;

[α]_{D}²⁵=−20.0° (c=1% SOLUTION, MeOH).

### EXAMPLE 206

### Preparation of: (5S)-2-amino-5-[4-(difluoromethoxy)-3-methylphenyl]-5-[3-(4-fluorobut-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[3-(4-fluorobut-1-yn-1-yl)phenyl]-3-methyl-3,5-dihydro-4H-imidazol-4-one.

MS (ES) m/z 414.1.

### EXAMPLE 207

### Preparation of: (5S)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one

The title compound is achieved through chiral separtion of 2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one

[α]_{D}²⁵=+22.0° (c=1% SOLUTION, MeOH);

MS (ES) m/z 432.1; MS (ES) m/z 865.3.

### EXAMPLE 208

### Preparation of: 2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromo-4-fluorophenyl)-2-(3-cyclopropyl-4-(difluoromethoxy)phenyl)ethane-1,2-dione with but-3-yn-1-ol in step 1.

MS (ES) m/z 458.0; MS (ES) m/z 518.0; MS (ES) m/z 917.1.

### EXAMPLE 209

### Preparation of: 2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromophenyl)-2-(3-cyclopropyl-4-(difluoromethoxy)phenyl)ethane-1,2-dione with prop-2-yn-1-ol in step 1.

MS (ES) m/z 426.0; MS (ES) m/z 486.0; MS (ES) m/z 853.1.

### EXAMPLE 210

### 2-amino-5-[3-cyclopropyl-4-(difluoromethoxy)phenyl]-5-[4-fluoro-3-(3-fluoroprop-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromo-4-fluorophenyl)-2-(3-cyclopropyl-4-(difluoromethoxy)phenyl)ethane-1,2-dione with prop-2-yn-1-ol in step 1.

MS (ES) m/z 444.1; MS (ES) m/z 889.2

### EXAMPLE 211

### Preparation of: 2-amino-5-[3-cyclopropyl-4-(difluoromethoxy)phenyl]-5-[3-(4-fluorobut-1-yn-1-yl)phenyl]-3-methyl-3,5-dihydro-4H-imidazol-4-one

This material was synthesized in a fashion similar to 2-amino-5-[4-(difluoromethoxy)-3-methylphenyl]-5-[3-(3-fluoroprop-1-yn-1-yl)phenyl]-3-methyl-3,5-dihydro-4H-imidazol-4-one by coupling 1-(3-bromophenyl)-2-(3-cyclopropyl-4-(difluoromethoxy)phenyl)ethane-1,2-dione with but-3-yn-1-ol in step 1.

MS (ES) m/z 442.1; MS (ES) m/z 883.3.

### EXAMPLE 212

### Preparation of: (5R)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one

The title compound is achieved through chiral separtion of 2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one.

MS (ES) m/z 426.1; MS (ES) m/z 486.1; MS (ES) m/z 853.2.

### EXAMPLE 213

### Preparation of: (5S)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one

The title compound is achieved through chiral separation of 2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one

MS (ES) m/z 426.1; MS (ES) m/z 486.1; MS (ES) m/z 853.2.

### EXAMPLE 214

### Preparation of: (5S)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one

The title compound is achieved through chiral separation of 2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one.

MS (ES) m/z 458.1; MS (ES) m/z 518.1; MS (ES) m/z 917.2.

### EXAMPLE 215

### Preparation of: (5R)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one

The title compound is achieved through chiral separation of 2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one.

MS (ES) m/z 458.1; MS (ES) m/z 518.1; MS (ES) m/z 917.2.

### BIOLOGICAL EXAMPLE

### Evaluation of BACE1 Binding Affinity of Test Compounds

### Fluorescent Kinetic Assays

Final Assay Conditions: 10 nM human BACE1 (or 10 nM Murine BACE1, 1.5 nM human BACE2), 25 μM substrate (WABC-6, MW 1549.6, from AnaSpec), Buffer: 50 mM Na-Acetate, pH 4.5, 0.05% CHAPS, 25% PBS, room temperature. Na-Acetate was from Aldrich, Cat.# 24, 124-5, CHAPS was from Research Organics, Cat. # 1304C₁×, PBS was from Mediatech (Cellgro), Cat# 21-031-CV, peptide substrate AbzSEVNLDAEFRDpa (SEQ ID NO:1) was from AnaSpec, Peptide Name:

### WABC-6

Determination of stock substrate (AbzSEVNLDAEFRDpa) (SEQ ID NO:1) concentration: ˜25 mM stock solution is made in DMSO using the peptide weight and MW, and diluted to ˜25 μM (1:1000) in 1×PBS. Concentration is determined by absorbance at 354 nm using an extinction coefficient of 18172 M⁻¹ cm⁻¹, the concentration of stock substrate is corrected, and the substrate stock stored in small aliquots in −80° C.

[Substrate Stock]=ABS^{354 nm}*10⁶/18172 (in mM)

The extinction coefficient ε^{354 nm} was adapted from TACE peptide substrate, which had the same quencher-fluorophore pair.

Determination of Stock Enzyme Concentration: the stock concentration of each enzyme is determined by absorbance at 280 nm using an of 64150 M⁻¹ cm⁻¹ for hBACE1 and MuBACE1, 62870 M⁻¹ cm⁻¹ for hBACE2 in 6 M Guanidinium Hydrochloride (from Research Organics, Cat. # 5134G-2), pH ˜6. The extinction coefficient ε^{280 nm} for each enzyme was calculated based on known amino acid composition and published extinction coefficients for Trp (5.69 M⁻¹ cm⁻¹) and Tyr (1.28 M⁻¹ cm⁻¹) residues (Anal. Biochem. 182, 319-326).

Dilution and mixing steps: total reaction volume: 100 μL

2× inhibitor dilutions in buffer A (66.7 mM Na-Acetate, pH 4.5, 0.0667% CHAPS) were prepared,

4× enzyme dilution in buffer A (66.7 mM Na-Acetate, pH 4.5, 0.0667% CHAPS) were prepared,

100 μM substrate dilution in 1×PBS was prepared, and

50 μL 2× Inhibitor, 25 μL 100 μM substrate are added to each well of 96-well plate (from DYNEX Technologies, VWR #: 11311-046), immediately followed by 25 μL 4× enzyme (added to the inhibitor and substrate mix), and the fluorescence readings are initiated.

Fluorescence Readings: Readings at λₑₓ 320 nm and λₑₘ 420 nm are taken every 40 sec for 30 min at room temperature and the linear slope for substrate cleavage rate (vᵢ) determined.

### Calculation of % Inhibition:

% Inhibition=100*(1−vᵢ/v₀)

vᵢ: substrate cleavage rate in the presence of inhibitor
v₀: substrate cleavage rate in the absence of inhibitor

### IC₅₀ Determination:

% Inhibition=((B*IC₅₀ⁿ)+(100*I₀ⁿ))/(IC₅₀ⁿ+I₀ⁿ)

(Model # 39 from LSW Tool Bar in Excel where B is the % inhibition from the enzyme control, which should be close to 0.) % Inhibition is plotted vs. Inhibitor Concentration (I₀) and the data fit to the above equation to obtain IC₅₀ value and Hill number (n) for each compound. Testing at least 10 different inhibitor concentrations is preferred.

Results are shown in the activity tables.

ACTIVITY TABLE I

| Example | BACE1 |
|---|---|
| No. | IC₅₀ μM |
| 1 | B |
| 2A | B |
| 2B | C |
| 3 | B |
| 4A | C |
| 4B | B |
| 5 | B |
| 6 | B |
| 7 | B |
| 8 | B |
| 9 | B |
| 10 | A |
| 11 | B |
| 12 | B |
| 13 | B |
| 14 | B |
| 15 | B |
| 16 | B |
| 17 | B |
| 18 | — |
| 19 | — |
| 20 | B |
| 21 | B |
| 22 | B |
| 23 | A |
| 24 | C |
| 25 | — |
| 26 | — |
| 27 | B |
| 28 | C |
| 29 | C |
| 30 | A |
| 31 | B |
| 32 | A |
| 33 | A |
| 34 | C |
| 35 | A |
| 36 | B |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | C |
| 52 | A |
| 53 | A |
| 54 | C |
| 55 | A |
| 56 | B |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | B |
| 71 | B |
| 72 | A |
| 73 | B |
| 74 | B |
| 75 | A |
| 76 | C |
| 77 | B |
| 78 | A |
| 79 | A |
| 80 | C |
| 81 | A |
| 82 | A |
| 83 | A |
| 84 | B |
| 85 | A |
| 86 | A |
| 87 | C |
| 88 | A |
| 89 | A |
| 90 | A |
| 91 | A |
| 92 | A |
| 93 | A |
| 94 | B |
| 95 | B |
| 96 | A |
| 97 | A |
| 98 | A |
| 99 | A |
| 100 | A |
| 101 | A |
| 102 | A |
| 103 | C |
| 104 | A |
| 105 | A |
| 106 | A |
| 107 | C |
| 108 | A |
| 109 | A |
| 110 | B |
| 111 | A |
| 112 | C |
| 113 | A |
| 114 | A |
| 115 | B |
| 116 | A |
| 117 | A |
| 118 | C |
| 119 | A |
| 120 | A |
| 121 | C |
| 122 | A |
| 123 | A |
| 124 | C |
| 125 | A |
| 126 | A |
| 127 | A |
| 128 | B |
| 129 | A |
| 130 | A |
| 131 | C |
| 132 | A |
| 133 | A |
| 134 | A |
| 135 | C |
| 136 | A |
| 137 | B |
| 138 | A |
| 139 | C |
| 140 | B |
| 141 | A |
| 142 | A |
| 143 | A |
| 144 | C |
| 145 | A |
| 146 | A |
| 147 | A |
| 148 | B |
| 149 | B |
| 150 | A |
| 151 | C |
| 152 | A |
| 153 | A |
| 154 | A |
| 155 | B |
| 156 | A |
| 157 | B |
| 158 | A |
| 159 | A |
| 160 | C |
| 161 | A |
| 162 | A |
| 163 | B |
| 164 | A |
| 165 | A |
| 166 | C |
| 167 | A |
| 168 | A |
| 169 | A |
| 170 | C |
| 171 | A |
| 172 | B |
| 173 | A |
| 174 | A |
| 175 | C |
| 176 | A |
| 177 | A |
| 178 | B |
| 179 | A |
| 180 | A |
| 181 | A |
| 182 | B |
| 183 | A |
| 184 | B |
| 185 | A |
| 186 | A |
| 187 | B |
| 188 | A |
| 189 | B |
| 190 | A |
| 191 | A |
| 192 | A |
| 193 | B |
| 194 | B |
| 195 | B |
| 196 | A |
| 197 | A |
| 198 | B |
| 199 | A |
| 200 | C |
| 201 | A |
| 202 | A |
| 203 | A |
| 204 | C |
| 205 | A |
| 206 | A |
| 207 | B |
| 208 | A |
| 209 | A |
| 210 | A |
| 211 | A |
| 212 | C |
| 213 | A |
| 214 | C |
| 215 | A |

Activity Tables
A = ≦0.01 μM-0.10 μM
B = 0.11 μM-1.00 μM
C = >1.00 μM

What is claimed is:

1. A compound of formula I 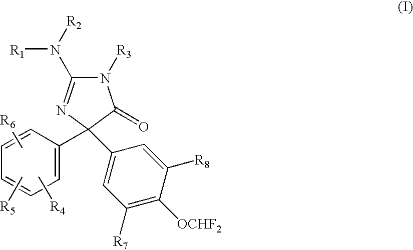
wherein
R₁ and R₂ are each independently H or an alkyl, cycloalkyl, cycloheteroalkyl, aryl or heteroaryl group each optionally substituted, or R₁ and R₂ may be taken together with the atom to which they are attached form an optionally substituted 5- to 7-membered ring optionally containing an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted, or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted, with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted, or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted, or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.

2. The compound of claim 1, wherein R₁ and R₂ are H.

3. The compound of claim 1, wherein R₃ is C₁-C₄alkyl.

4. The compound of claim 3, wherein R₃ is methyl.

5. The compound of claim 1, wherein R₄, R₅ and R₆ are each independently H, halogen, or an alkenyl, alkynyl, alkoxy, alkenyloxy, or alkynyloxy group each optionally substituted.

6. The compound of claim 5, wherein R₄ is an alkynyl group optionally substituted with cycloalkyl; and R₄ is at the 3-position of the phenyl ring.

7. The compound of claim 5, wherein R₅ and R₆ are each independently H or halogen.

8. The compound of claim 1, wherein R₇ is halogen, C₁-C₄alkyl or C₃-C₆cycloalkyl.

9. The compound of claim 8, wherein R₇ is halogen, methyl, ethyl, propyl or cyclopropyl.

10. The compound of claim 1, wherein R₅ and R₆ are each independently H or halogen; and R₇ is halogen, methyl, ethyl, propyl or cyclopropyl.

11. The compound of claim 1, wherein R₄ is: 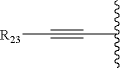
wherein,
R₂₃ is selected from the group consisting of H, alkyl, haloalkyl, cycloalkyl, halogen and alkoxyalkyl.

12. The compound of claim 11, wherein R₂₃ is methyl, ethyl, cyclopropyl, methoxymethyl, methoxyethyl, propyl, fluoroethyl, fluoromethyl, isopropyl, isobutyl or 1,1-difluoroethyl.

13. The compound of claim 1, wherein R₆ is H and R₅ is fluoro substituted at the 4-position of the phenyl ring.

14. The compound or claim 1, having the formula IA:

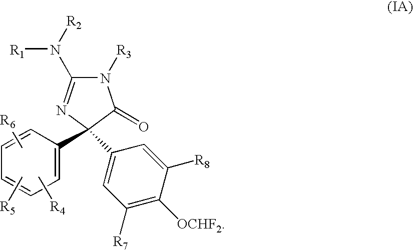

15. The compound of claim 1, selected from the group consisting of:
(5R)-2-Amino-5-[3-(cyclopropylethynyl)-4-fluorophenyl]-5-[4-(difluoromethoxy)-3-methylphenyl]-3-methyl-3,5-dihydro-4H-imidazol-4-one
2-Amino-5-[4-(difluoromethoxy)-3-methylphenyl]-3-methyl-5-phenyl-3,5-dihydro-4H-imidazol-4-one;
(5S)-2-Amino-5-[4-(difluoromethoxy)-3-methylphenyl]-3-methyl-5-phenyl-3,5-dihydro-4H-imidazol-4-one;
(5R)-2-Amino-5-[4-(difluoromethoxy)-3-methylphenyl]-3-methyl-5-phenyl-3,5-dihydro-4H-imidazol-4-one;
2-Amino-4-(4-(difluoromethoxy)-3-methylphenyl)-4-(4-fluorophenyl)-1-methyl-1H-imidazol-5(4H)-one;
(5S)-2-Amino-4-(4-(difluoromethoxy)-3-methylphenyl)-4-(4-fluorophenyl)-1-methyl-1H-imidazol-5(4H)-one;
(5R)-2-Amino-4-(4-(difluoromethoxy)-3-methylphenyl)-4-(4-fluorophenyl)-1-methyl-1H-imidazol-5(4H)-one;
2-Amino-5-(3-butoxyphenyl)-5-[3-chloro-4-(difluoromethoxy)-phenyl]-3-methyl-3,5-dihydro-4H-imidazol-4-one;
2-Amino-5-[3-(cyclopropylethynyl)phenyl]-5-[4-(difluoromethoxy)-3-methylphenyl]-3-methyl-3,5-dihydro-4H-imidazol-4-one;
2-Amino-5-[4-(difluoromethoxy)-3-methylphenyl]-5-[4-fluoro-3-(3-methylbut-1-yn-1-yl)phenyl]-3-methyl-3,5-dihydro-4H-imidazol-4-one;
2-Amino-5-(4-difluoromethoxy-3-methylphenyl)-5-[4-fluoro-3-((E)-3-methoxypropenyl)phenyl]-3-methyl-3,5-dihydro-imidazol-4-one;
2-Amino-5-[4-(difluoromethoxy)-3-methylphenyl]-5-[4-fluoro-3-((E)-4-fluorobut-1-enyl)phenyl]-3-methyl-3,5-dihydro-4H-imidazol-4-one;
2-Amino-5-[4-(difluoromethoxy)-3-methylphenyl]-5-[4-fluoro-3-((E)-prop-1-enyl)phenyl]-3-methyl-3,5-dihydro-4H-imidazol-4-one;
2-Amino-5-[4-(difluoromethoxy)-3-methylphenyl]-5-[4-fluoro-3-((E)-4-methoxy-but-1-enyl)phenyl]-3-methyl-3,5-dihydro-4H-imidazol-4-one;
2-Amino-5-[4-(difluoromethoxy)-3-methylphenyl]-3-methyl-5-[((E)-3-prop-1-enyl)phenyl]-3,5-dihydro-4H-imidazol-4-one;
2-Amino-5-[4-(difluoromethoxy)-3-methylphenyl]-5-[3-((E)-3-methoxyprop-1-enyl)phenyl]-3-methyl-3,5-dihydro-4H-imidazol-4-one;
2-Amino-5-[4-(difluoromethoxy)-3-methylphenyl]-5-[3-((E)-4-fluorobut-1-enyl)phenyl]-3-methyl-3,5-dihydro-4H-imidazol-4-one;
2-Amino-5-[4-(difluoromethoxy)-3-methylphenyl]-5-[3-((E)-4-methoxybut-1-enyl)phenyl]-3-methyl-3,5-dihydro-4H-imidazol-4-one;
2-Amino-5-[3-chloro-4-(difluoromethoxy)phenyl]-3-methyl-5-[((E)-3-prop-1-enyl)phenyl]-3,5-dihydro-4H-imidazol-4-one;
2-Amino-5-[3-chloro-4-(difluoromethoxy)phenyl]-5-[3-((E)-3-methoxyprop-1-enyl)-phenyl]-3-methyl-3,5-dihydro-4H-imidazol-4-one;
2-Amino-5-[3-chloro-4-(difluoromethoxy)phenyl)-5-[3-((E)-4-methoxybut-1-enyl)phenyl]-3-methyl-3,5-dihydro-4H-imidazol-4-one;
2-Amino-5-[3-chloro-4-(difluoromethoxy)phenyl)-5-[3-((E)-4-fluoro-but-1-enyl)phenyl]-3-methyl-3,5-dihydro-4H-imidazol-4-one;
2-Amino-5,5-bis-[4-(difluoromethoxy)-3-methylphenyl]-3-methyl-3,5-dihydro-4H-imidazol-4-one;
2-Amino-5-[4-(difluoromethoxy)-3,5-dimethylphenyl]-3-methyl-5-phenyl-3,5-dihydro-4H-imidazol-4-one;
2-Amino-5-[4-(difluoromethoxy)-3-ethylphenyl]-3-methyl-5-phenyl-3,5-dihydro-4H-imidazol-4-one;
2-Amino-4-[4-(difluoromethoxy)-3-ethylphenyl]-4-(4-fluorophenyl)-1-methyl-1H-imidazol-5(4H)-one;
2-Amino-5-[4-(difluoromethoxy)-3-propylphenyl]-3-methyl-5-phenyl-3,5-dihydro-4H-imidazol-4-one;
2-Amino-5-[4-(difluoromethoxy)-3-isopropylphenyl]-3-methyl-5-phenyl-3,5-dihydro-4H-imidazol-4-one;
2-Amino-5-[3-cyclopropyl-4-(difluoromethoxy)phenyl]-3-methyl-5-phenyl-3,5-dihydro-4H-imidazol-4-one;
2-Amino-5-[4-(difluoromethoxy)-3-vinylphenyl)-3-methyl-5-phenyl-3,5-dihydro-4H-imidazol-4-one;
2-Amino-5-[4-(difluoromethoxy)-3-(trifluoromethyl)phenyl]-3-methyl-5-phenyl-3,5-dihydro-4H-imidazol-4-one;
2-Amino-5-[4-(difluoromethoxy)-3-(fluoromethyl)phenyl]-3-methyl-5-phenyl-3,5-dihydro-4H-imidazol-4-one;
2-Amino-5-[4-(difluoromethoxy)-3-(fluoromethyl)phenyl]-3-methyl-5-phenyl-3,5-dihydro-4H-imidazol-4-one;
2-Amino-5-[4-(difluoromethoxy)-3-methoxyphenyl]-3-methyl-5-phenyl-3,5-dihydro-4H-imidazol-4-one;
2-Amino-5-[3-chloro-4-(difluoromethoxy)phenyl]-3-methyl-5-phenyl-3,5-dihydro-4H-imidazol-4-one;
2-Amino-5-[4-(difluoromethoxy)-3-fluorophenyl]-3-methyl-5-phenyl-3,5-dihydro-4H-imidazol-4-one;
2-Amino-5-(3-bromophenyl)-5-[4-(difluoromethoxy)-3-methylphenyl]-3-methyl-3,5-dihydro-4H-imidazol-4-one;
2-Amino-5-(3-bromo-4-fluorophenyl)-5-[4-(difluoromethoxy)-3-methylphenyl]-3-methyl-3,5-dihydro-4H-imidazol-4-one;
2-Amino-5-[4-(difluoromethoxy)-3-methylphenyl]-5-(4-fluoro-3-methylphenyl)-3-methyl-3,5-dihydro-4H-imidazol-4-one;
5-{2-Amino-4-[4-(difluoromethoxy)-3-methylphenyl]-1-methyl-5-oxo-4,5-dihydro-1H-imidazol-4-yl}-2-methoxybenzonitrile;
2-Amino-5-[4-(difluoromethoxy)-3-methylphenyl]-5-[4-fluoro-3-(fluoromethyl)phenyl]-3-methyl-3,5-dihydro-4H-imidazol-4-one;
(5S)-2-Amino-4-[4-(difluoromethoxy)-3-ethylphenyl]-1-methyl-4-phenyl-1H-imidazol-5(4H)-one;
(5R)-2-Amino-4-[4-(difluoromethoxy)-3-ethylphenyl]-1-methyl-4-phenyl-1H-imidazol-5(4H)-one;
(5R)-2-Amino-5-[3-cyclopropyl-4-(difluoromethoxy)phenyl]-3-methyl-5-phenyl-3,5-dihydro-4H-imidazol-4-one;
(5S)-2-Amino-5-[3-cyclopropyl-4-(difluoromethoxy)phenyl]-3-methyl-5-phenyl-3,5-dihydro-4H-imidazol-4-one;
(5S)-2-Amino-5-[3-chloro-4-(difluoromethoxy)phenyl]-1-methyl-5-phenyl-3,5-dihydro-4H-imidazol-4-one;
(5R)-2-Amino-5-[3-chloro-4-(difluoromethoxy)phenyl]-1-methyl-5-phenyl-3,5-dihydro-4H-imidazol-4-one;
(5S)-2-Amino-5-(3-bromophenyl)-5-[4-(difluoromethoxy)-3-methylphenyl]-3-methyl-3,5-dihydro-4H-imidazol-4-one;
(5R)-2-Amino-5-(3-bromophenyl)-5-[4-(difluoromethoxy)-3-methylphenyl]-3-methyl-3,5-dihydro-4H-imidazol-4-one;
2-Amino-5-[3-chloro-4-(difluoromethoxy)phenyl]-5-[3-(2-fluoroethoxy)phenyl]-3-methyl-3,5-dihydro-4H-imidazol-4-one;
2-Amino-5-[3-chloro-4-(difluoromethoxy)phenyl]-5-(3-ethoxyphenyl)-3-methyl-3,5-dihydro-4H-imidazol-4-one;
2-Amino-5-[3-chloro-4-(difluoromethoxy)phenyl]-5-[3-(2,2-difluoroethoxy)phenyl]-3-methyl-3,5-dihydro-4H-imidazol-4-one;
2-Amino-5-[3-chloro-4-(difluoromethoxy)phenyl]-3-methyl-5-(3-propoxyphenyl)-3,5-dihydro-4H-imidazol-4-one;
2-Amino-5-[3-chloro-4-(difluoromethoxy)phenyl]-5-[3-(3-fluoropropoxy)phenyl]-3-methyl-3,5-dihydro-4H-imidazol-4-one;
2-Amino-5-[4-(difluoromethoxy)-3-methylphenyl]-5-(3-ethoxyphenyl)-3-methyl-3,5-dihydro-4H-imidazol-4-one;
2-Amino-5-[4-(difluoromethoxy)-3-methylphenyl]-3-methyl-5-(3-propoxyphenyl)-3,5-dihydro-4H-imidazol-4-one;
2-Amino-5-[3-(2,2-difluoroethoxy)phenyl]-5-[4-(difluoromethoxy)-3-methylphenyl]-3-methyl-3,5-dihydro-4H-imidazol-4-one;
2-Amino-5-[4-(difluoromethoxy)-3-methylphenyl]-3-methyl-5-(3-propoxyphenyl)-3,5-dihydro-4H-imidazol-4-one;
2-Amino-5-[4-(difluoromethoxy)-3-methylphenyl]-5-[3-(3-fluoropropoxy)phenyl]-3-methyl-3,5-dihydro-4H-imidazol-4-one;
2-Amino-5-[3-(cyclopropylethynyl)-4-fluorophenyl]-5-[4-(difluoromethoxy)-3-methylphenyl]-3-methyl-3,5-dihydro-4H-imidazol-4-one;
2-Amino-5-[4-(difluoromethoxy)-3-methylphenyl]-5-[4-fluoro-3-(3-methylbut-1-yn-1-yl)phenyl]-3-methyl-3,5-dihydro-4H-imidazol-4-one;
(5R)-2-Amino-5-[3-(cyclopropylethynyl)phenyl]-5-[4-(difluoromethoxy)-3-methylphenyl]-3-methyl-3,5-dihydro-4H-imidazol-4-one;
(5S)-2-Amino-5-[3-(cyclopropylethynyl)phenyl]-5-[4-(difluoromethoxy)-3-methylphenyl]-3-methyl-3,5-dihydro-4H-imidazol-4-one;
(5S)-2-Amino-5-[3-(cyclopropylethynyl)-4-fluorophenyl]-5-[4-(difluoromethoxy)-3-methylphenyl]-3-methyl-3,5-dihydro-4H-imidazol-4-one;
(5S)-2-Amino-5-[4-(difluoromethoxy)-3-vinylphenyl]-3-methyl-5-phenyl-3,5-dihydro-4H-imidazol-4-one;
(5R)-2-Amino-5-[4-(difluoromethoxy)-3-vinylphenyl]-3-methyl-5-phenyl-3,5-dihydro-4H-imidazol-4-one;
(5S)-2-Amino-5-[4-(difluoromethoxy)-3-methylphenyl]-5-[3-(3-methoxyprop-1-yn-1-yl)phenyl]-3-methyl-3,5-dihydro-4H-imidazol-4-one;
(5R)-2-Amino-5-[4-(difluoromethoxy)-3-methylphenyl]-5-[3-(3-methoxyprop-1-yn-1-yl)phenyl]-3-methyl-3,5-dihydro-4H-imidazol-4-one;
(5S)-2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one;
(5R)-2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one;
2-Amino-5-[4-(difluoromethoxy)-3-methylphenyl]-5-[4-fluoro-3-(3-methoxyprop-1-yn-1-yl)phenyl]-3-methyl-3,5-dihydro-4H-imidazol-4-one; and
2-Amino-5-[4-(difluoromethoxy)-3-methylphenyl]-5-[3-(3-methoxyprop-1-yn-1-yl)phenyl]-3-methyl-3,5-dihydro-4H-imidazol-4-one;
(5R)-2-amino-5-[4-(difluoromethoxy)-3-methylphenyl]-5-(4-fluoro-3-pent-1-yn-1-ylphenyl)-3-methyl-3,5-dihydro-4H-imidazol-4-one;
(5S)-2-amino-5-[4-(difluoromethoxy)-3-methylphenyl]-5-(4-fluoro-3-pent-1-yn-1-ylphenyl)-3-methyl-3,5-dihydro-4H-imidazol-4-one;
2-amino-5-[4-(difluoromethoxy)-3-(2-fluoroethyl)phenyl]-3-methyl-5-phenyl-3,5-dihydro-4H-imidazol-4-one;
(5S)-2-amino-5-[4-(difluoromethoxy)-3-(2-fluoroethyl)phenyl]-3-methyl-5-phenyl-3,5-dihydro-4H-imidazol-4-one;
(5R)-2-amino-5-[4-(difluoromethoxy)-3-(2-fluoroethyl)phenyl]-3-methyl-5-phenyl-3,5-dihydro-4H-imidazol-4-one;
2-amino-5-[4-(difluoromethoxy)-3-methylphenyl]-3-methyl-5-(3-methylphenyl)-3,5-dihydro-4H-imidazol-4-one;
(5S)-2-amino-5-[4-(difluoromethoxy)-3-methylphenyl]-3-methyl-5-(3-pent-1-yn-1-ylphenyl)-3,5-dihydro-4H-imidazol-4-one;
(5S)-2-amino-5-[4-(difluoromethoxy)-3-propylphenyl]-3-methyl-5-phenyl-3,5-dihydro-4H-imidazol-4-one;
(5R)-2-amino-5-[4-(difluoromethoxy)-3-propylphenyl]-3-methyl-5-phenyl-3,5-dihydro-4H-imidazol-4-one;
2-amino-5-(3-but-1-yn-1-ylphenyl)-5-[4-(difluoromethoxy)-3-methylphenyl]-3-methyl-3,5-dihydro-4H-imidazol-4-one;
2-amino-5-(3-but-1-yn-1-yl-4-fluorophenyl)-5-[4-(difluoromethoxy)-3-methylphenyl]-3-methyl-3,5-dihydro-4H-imidazol-4-one;
(5R)-2-amino-5-(3-but-1-yn-1-yl-4-fluorophenyl)-5-[4-(difluoromethoxy)-3-methylphenyl]-3-methyl-3,5-dihydro-4H-imidazol-4-one;
(5S)-2-amino-5-(3-but-1-yn-1-yl-4-fluorophenyl)-5-[4-(difluoromethoxy)-3-methylphenyl]-3-methyl-3,5-dihydro-4H-imidazol-4-one;
2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
(5R)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
(5S)-2-amino-5-[3-cyclopropyl-4-(difluoromethoxy)phenyl]-5-[4-fluoro-3-(3-fluoropropoxy)phenyl]-3-methyl-3,5-dihydro-4H-imidazol-4-one;
2-amino-5-[4-(difluoromethoxy)-3-(2-fluoroethyl)phenyl]-5-(4-fluoro-3-pent-1-yn-1-ylphenyl)-3-methyl-3,5-dihydro-4H-imidazol-4-one;
2-amino-5-(3-but-1-yn-1-yl-4-fluorophenyl)-5-[4-(difluoromethoxy)-3-(2-fluoroethyl)phenyl]-3-methyl-3,5-dihydro-4H-imidazol-4-one;
2-amino-5-[4-(difluoromethoxy)-3-(2-fluoroethyl)phenyl]-5-[4-fluoro-3-(4-methylpent-1-yn-1-yl)phenyl]-3-methyl-3,5-dihydro-4H-imidazol-4-one;
2-amino-5-[4-(difluoromethoxy)-3-(2-fluoroethyl)phenyl]-5-[4-fluoro-3-(4-fluorobut-1-yn-1-yl)phenyl]-3-methyl-3,5-dihydro-4H-imidazol-4-one;
2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
(5R)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
(5S)-2-amino-5-[3-(cyclopropylethynyl)-4-fluorophenyl]-5-[4-(difluoromethoxy)-3-(2-fluoroethyl)phenyl]-3-methyl-3,5-dihydro-4H-imidazol-4-one;
2-amino-5-[3-chloro-4-(difluoromethoxy)phenyl]-5-(4-fluoro-3-hydroxyphenyl)-3-methyl-3,5-dihydro-4H-imidazol-4-one;
2-amino-5-[3-chloro-4-(difluoromethoxy)phenyl]-5-(3-ethoxy-4-fluorophenyl)-3-methyl-3,5-dihydro-4H-imidazol-4-one;
2-amino-5-[3-chloro-4-(difluoromethoxy)phenyl]-5-(4-fluoro-3-propoxyphenyl)-3-methyl-3,5-dihydro-4H-imidazol-4-one;
2-amino-5-[3-chloro-4-(difluoromethoxy)phenyl]-5-[4-fluoro-3-(3-fluoropropoxy)phenyl]-3-methyl-3,5-dihydro-4H-imidazol-4-one;
2-amino-5-[3-chloro-4-(difluoromethoxy)phenyl]-5-[4-fluoro-3-(2-fluoroethoxy)phenyl]-3-methyl-3,5-dihydro-4H-imidazol-4-one;
2-amino-5-[3-chloro-4-(difluoromethoxy)phenyl]-5-[3-(2,2-difluoroethoxy)-4-fluorophenyl]-3-methyl-3,5-dihydro-4H-imidazol-4-one;
2-amino-5-[4-(difluoromethoxy)-3-methylphenyl]-5-(3-ethoxy-4-fluorophenyl)-3-methyl-3,5-dihydro-4H-imidazol-4-one;
2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
(5S)-2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
(5R)-2-amino-5-[3-(2,2-difluoroethoxy)-4-fluorophenyl]-5-[4-(difluoromethoxy)-3-methylphenyl]-3-methyl-3,5-dihydro-4H-imidazol-4-one;
2-amino-5-[3-(cyclopropylmethoxy)-4-fluorophenyl]-5-[4-(difluoromethoxy)-3-methylphenyl]-3-methyl-3,5-dihydro-4H-imidazol-4-one;
(5R)-2-amino-5-[3-(cyclopropylmethoxy)-4-fluorophenyl]-5-[4-(difluoromethoxy)-3-methylphenyl]-3-methyl-3,5-dihydro-4H-imidazol-4-one;
(5S)-2-amino-5-[3-(cyclopropylmethoxy)-4-fluorophenyl]-5-[4-(difluoromethoxy)-3-methylphenyl]-3-methyl-3,5-dihydro-4H-imidazol-4-one;
2-amino-5-[4-(difluoromethoxy)-3-methylphenyl]-5-[4-fluoro-3-(3-fluoropropoxy)phenyl]-3-methyl-3,5-dihydro-4H-imidazol-4-one;
2-amino-5-[4-(difluoromethoxy)-3-methylphenyl]-5-[4-fluoro-3-(2-fluoroethoxy)phenyl]-3-methyl-3,5-dihydro-4H-imidazol-4-one;
(5S)-2-amino-5-[4-(difluoromethoxy)-3-methylphenyl]-5-[4-fluoro-3-(3-fluoropropoxy)phenyl]-3-methyl-3,5-dihydro-4H-imidazol-4-one;
(5R)-2-amino-5-[4-(difluoromethoxy)-3-methylphenyl]-5-[4-fluoro-3-(3-fluoropropoxy)phenyl]-3-methyl-3,5-dihydro-4H-imidazol-4-one;
(5S)-2-amino-5-[4-(difluoromethoxy)-3-methylphenyl]-5-[4-fluoro-3-(2-fluoroethoxy)phenyl]-3-methyl-3,5-dihydro-4H-imidazol-4-one;
(5R)-2-amino-5-[4-(difluoromethoxy)-3-methylphenyl]-5-[4-fluoro-3-(2-fluoroethoxy)phenyl]-3-methyl-3,5-dihydro-4H-imidazol-4-one;
2-amino-5-[4-(difluoromethoxy)-3-methylphenyl]-5-(4-fluoro-3-propoxyphenyl)-3-methyl-3,5-dihydro-4H-imidazol-4-one;
(5S)-2-amino-5-[4-(difluoromethoxy)-3-methylphenyl]-5-(4-fluoro-3-propoxyphenyl)-3-methyl-3,5-dihydro-4H-imidazol-4-one;
3-(2-{[tert-butyl(dimethyl)silyl]oxy}ethyl)-6-fluoro-1-(2-fluorophenyl)-3,4-dihydro-1H-2,1-benzothiazine 2,2-dioxide;
2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
(5S)-2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
(5R)-2-amino-5-[3-(2,2-difluoroethoxy)-4-fluorophenyl]-5-[4-(difluoromethoxy)-3-ethylphenyl]-3-methyl-3,5-dihydro-4H-imidazol-4-one;
2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
(5S)-2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
(5R)-2-amino-5-[4-(difluoromethoxy)-3-ethylphenyl]-5-[4-fluoro-3-(3-fluoropropoxy)phenyl]-3-methyl-3,5-dihydro-4H-imidazol-4-one;
2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one;
(5S)-2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one;
(5R)-2-amino-5-[3-cyclopropyl-4-(difluoromethoxy)phenyl]-5-[3-(2,2-difluoroethoxy)-4-fluorophenyl]-3-methyl-3,5-dihydro-4H-imidazol-4-one 2-amino-5-[4-(difluoromethoxy)-3-methylphenyl]-5-[3-(3,3-difluoropropoxy)phenyl]-3-methyl-3,5-dihydro-4H-imidazol-4-one;
(5R)-2-amino-5-[4-(difluoromethoxy)-3-methylphenyl]-5-[3-(3,3-difluoropropoxy)phenyl]-3-methyl-3,5-dihydro-4H-imidazol-4-one;
(5S)-2-amino-5-[4-(difluoromethoxy)-3-methylphenyl]-5-[3-(3,3-difluoropropoxy)phenyl]-3-methyl-3,5-dihydro-4H-imidazol-4-one;
2-amino-5-[3-(2,2-difluoroethoxy)-4-fluorophenyl]-5-[4-(difluoromethoxy)-3-(2-fluoroethyl)phenyl]-3-methyl-3,5-dihydro-4H-imidazol-4-one;
2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
(5S)-2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
(5R)-2-amino-5-[4-(difluoromethoxy)-3-methylphenyl]-5-[3-(3,3-difluoropropoxy)-4-fluorophenyl]-3-methyl-3,5-dihydro-4H-imidazol-4-one;
2-amino-5-[3-cyclopropyl-4-(difluoromethoxy)phenyl]-5-[3-(3,3-difluoropropoxy)-4-fluorophenyl]-3-methyl-3,5-dihydro-4H-imidazol-4-one;
2-amino-5-[4-(difluoromethoxy)-3-ethylphenyl]-5-[3-(3,3-difluoropropoxy)-4-fluorophenyl]-3-methyl-3,5-dihydro-4H-imidazol-4-one;
(5S)-2-amino-5-[4-(difluoromethoxy)-3-ethylphenyl]-5-[3-(3,3-difluoropropoxy)-4-fluorophenyl]-3-methyl-3,5-dihydro-4H-imidazol-4-one
(5R)-2-amino-5-[4-(difluoromethoxy)-3-ethylphenyl]-5-[3-(3,3-difluoropropoxy)-4-fluorophenyl]-3-methyl-3,5-dihydro-4H-imidazol-4-one;
2-amino-5-[4-(difluoromethoxy)-3-isopropylphenyl]-3-methyl-5-phenyl-3,5-dihydro-4H-imidazol-4-one;
(5S)-2-amino-5-[4-(difluoromethoxy)-3-isopropylphenyl]-3-methyl-5-phenyl-3,5-dihydro-4H-imidazol-4-one;
(5R)-2-amino-5-[4-(difluoromethoxy)-3-isopropylphenyl]-3-methyl-5-phenyl-3,5-dihydro-4H-imidazol-4-one;
2-amino-5-[4-(difluoromethoxy)-3-(2-hydroxyethyl)phenyl]-3-methyl-5-phenyl-3,5-dihydro-4H-imidazol-4-one;
2-amino-5-[3-(2-chloroethyl)-4-(difluoromethoxy)phenyl]-3-methyl-5-phenyl-3,5-dihydro-4H-imidazo 1-4-one;
2-amino-5-[4-(difluoromethoxy)-3-(2-methoxyethyl)phenyl]-3-methyl-5-phenyl-3,5-dihydro-4H-imidazol-4-one;
(5S)-2-amino-5-[3-(2-chloroethyl)-4-(difluoromethoxy)phenyl]-3-methyl-5-phenyl-3,5-dihydro-4H-imidazol-4-one;
(5R)-2-amino-5-[3-(2-chloroethyl)-4-(difluoromethoxy)phenyl]-3-methyl-5-phenyl-3,5-dihydro-4H-imidazol-4-one;
2-amino-5-[3-(cyclopropylethynyl)-4-fluorophenyl]-5-[4-(difluoromethoxy)-3-isopropylphenyl]-3-methyl-3,5-dihydro-4H-imidazol-4-one;
2-amino-5-[3-(cyclopropylethynyl)-4-fluorophenyl]-5-[4-(difluoromethoxy)-3-ethylphenyl]-3-methyl-3,5-dihydro-4H-imidazol-4-one;
(5R)-2-amino-5-[3-(cyclopropylethynyl)-4-fluorophenyl]-5-[4-(difluoromethoxy)-3-ethylphenyl]-3-methyl-3,5-dihydro-4H-imidazol-4-one;
(5S)-2-amino-5-[3-(cyclopropylethynyl)-4-fluorophenyl]-5-[4-(difluoromethoxy)-3-ethylphenyl]-3-methyl-3,5-dihydro-4H-imidazol-4-one;
2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(4-methylpent-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-methylbut-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-methoxyprop-1-yn-1-yl)phenyl]-3-methyl-3,5-dihydro-4H-imidazol-4-one;
2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
(5S)-2-amino-5-[4-(difluoromethoxy)-3-ethylphenyl]-5-[3-(3-fluoroprop-1-yn-1-yl)phenyl]-3-methyl-3,5-dihydro-4H-imidazol-4-one;
(5R)-2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
(5S)-2-amino-5-[4-(difluoromethoxy)-3-ethylphenyl]-5-[4-fluoro-3-(3-fluoroprop-1-yn-1-yl)phenyl]-3-methyl-3,5-dihydro-4H-imidazol-4-one;
2-amino-5-(3-bromophenyl)-5-[3-cyclopropyl-4-(difluoromethoxy)phenyl]-3-methyl-3,5-dihydro-4H-imidazol-4-one;
(5R)-2-amino-5-(3-bromophenyl)-5-[3-cyclopropyl-4-(difluoromethoxy)phenyl]-3-methyl-3,5-dihydro-4H-imidazol-4-one;
(5S)-2-amino-5-(3-bromophenyl)-5-[3-cyclopropyl-4-(difluoromethoxy)phenyl]-3-methyl-3,5-dihydro-4H-imidazol-4-one;
2-amino-5-[3-cyclopropyl-4-(difluoromethoxy)phenyl]-5-(3-ethynylphenyl)-3-methyl-3,5-dihydro-4H-imidazol-4-one;
2-amino-5-[3-cyclopropyl-4-(difluoromethoxy)phenyl]-3-methyl-5-(3-prop-1-yn-1-ylphenyl)-3,5-dihydro-4H-imidazol-4-one;
(5R)-2-amino-5-[3-cyclopropyl-4-(difluoromethoxy)phenyl]-3-methyl-5-(3-prop-1-yn-1-ylphenyl)-3,5-dihydro-4H-imidazol-4-one;
(5S)-2-amino-5-[3-cyclopropyl-4-(difluoromethoxy)phenyl]-3-methyl-5-(3-prop-1-yn-1-ylphenyl)-3,5-dihydro-4H-imidazol-4-one;
2-amino-5-(3-but-1-yn-1-ylphenyl)-5-[3-cyclopropyl-4-(difluoromethoxy)phenyl]-3-methyl-3,5-dihydro-4H-imidazol-4-one;
2-amino-5-[3-cyclopropyl-4-(difluoromethoxy)phenyl]-3-methyl-5-(3-pent-1-yn-1-ylphenyl)-3,5-dihydro-4H-imidazol-4-one;
2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
(5S)-2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
(5R)-2-amino-5-[3-cyclopropyl-4-(difluoromethoxy)phenyl]-3-methyl-5-[3-(3-methylbut-1-yn-1-yl)phenyl]-3,5-dihydro-4H-imidazol-4-one;
2-amino-5-[3-cyclopropyl-4-(difluoromethoxy)phenyl]-5-[3-(cyclopropylethynyl)phenyl]-3-methyl-3,5-dihydro-4H-imidazol-4-one;
(5S)-2-amino-5-[3-cyclopropyl-4-(difluoromethoxy)phenyl]-5-[3-(cyclopropylethynyl)phenyl]-3-methyl-3,5-dihydro-4H-imidazol-4-one;
(5R)-2-amino-5-[3-cyclopropyl-4-(difluoromethoxy)phenyl]-5-[3-(cyclopropylethynyl)phenyl]-3-methyl-3,5-dihydro-4H-imidazol-4-one;
2-amino-5-[3-cyclopropyl-4-(difluoromethoxy)phenyl]-5-[3-(3-hydroxy-3-methylbut-1-yn-1-yl)phenyl]-3-methyl-3,5-dihydro-4H-imidazol-4-one;
2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(3-methoxyprop-1-yn-1-yl)phenyl]-3-methyl-3,5-dihydro-4H-imidazol-4-one;
2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(4-methoxybut-1-yn-1-yl)phenyl]-3-methyl-3,5-dihydro-4H-imidazol-4-one;
2-amino-5-[3-cyclopropyl-4-(difluoromethoxy)phenyl]-5-[3-(5-methoxypent-1-yn-1-yl)phenyl]-3-methyl-3,5-dihydro-4H-imidazol-4-one;
2-amino-5-[3-cyclopropyl-4-(difluoromethoxy)phenyl]-5-(3-cyclopropylphenyl)-3-methyl-3,5-dihydro-4H-imidazol-4-one;
2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(5-hydroxypent-1-yn-1-yl)phenyl]-3-methyl-3,5-dihydro-4H-imidazol-4-one;
2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(5-fluoropent-1-yn-1-yl)phenyl]-3-methyl-3,5-dihydro-4H-imidazol-4-one;
2-amino-5,5-bis[3-cyclopropyl-4-(difluoromethoxy)phenyl]-3-methyl-3,5-dihydro-4H-imidazol-4-one;
2-amino-5-[4-(difluoromethoxy)-3-methylphenyl]-5-(4-fluoro-3-isopropoxyphenyl)-3-methyl-3,5-dihydro-4H-imidazol-4-one;
2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5S)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-methylphenyl]-5-[4-fluoro-3-(3-fluoroprop-1-yn-1-yl)phenyl]-3-methyl-3,5-dihydro-4H-imidazol-4-one;
(5R)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
(5S)-2-amino-5-[4-(difluoromethoxy)-3-methylphenyl]-5-[3-(3-fluoroprop-1-yn-1-yl)phenyl]-3-methyl-3,5-dihydro-4H-imidazol-4-one;
2-amino-5-[4-(difluoromethoxy)-3-methylphenyl]-5-[3-(4-fluorobut-1-yn-1-yl)phenyl]-3-methyl-3,5-dihydro-4H-imidazol-4-one;
2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
(5R)-2-amino-5-[4-(difluoromethoxy)-3-methylphenyl]-5-[3-(4-fluorobut-1-yn-1-yl)phenyl]-3-methyl-3,5-dihydro-4H-imidazol-4-one;
(5R)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
(5S)-2-amino-5-[4-(difluoromethoxy)-3-methylphenyl]-5-[3-(4-fluorobut-1-yn-1-yl)phenyl]-3-methyl-3,5-dihydro-4H-imidazol-4-one;
(5S)-2-amino-5-[4-(difluoromethoxy)-3-methylphenyl]-5-[4-fluoro-3-(4-fluorobut-1-yn-1-yl)phenyl]-3-methyl-3,5-dihydro-4H-imidazol-4-one;
2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one;
2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
2-amino-5-[3-cyclopropyl-4-(difluoromethoxy)phenyl]-5-[4-fluoro-3-(3-fluoroprop-1-yn-1-yl)phenyl]-3-methyl-3,5-dihydro-4H-imidazol-4-one;
2-amino-5-[3-cyclopropyl-4-(difluoromethoxy)phenyl]-5-[3-(4-fluorobut-1-yn-1-yl)phenyl]-3-methyl-3,5-dihydro-4H-imidazol-4-one;
(5R)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[3-(3-fluoroprop-1-yn-1-yl)phenyl]-3-methyl-3,5-dihydro-4H-imidazol-4-one;
(5S)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one; and
(5R)-2-amino-5-[3-cyclopropyl-4-(difluoromethoxy)phenyl]-5-[4-fluoro-3-(4-fluorobut-1-yn-1-yl)phenyl]-3-methyl-3,5-dihydro-4H-imidazol-4-one;
a tautomer thereof;
a stereoisomer thereof; or
a pharmaceutically acceptable salt thereof.

16. The compound of claim 1, selected from the group consisting of:

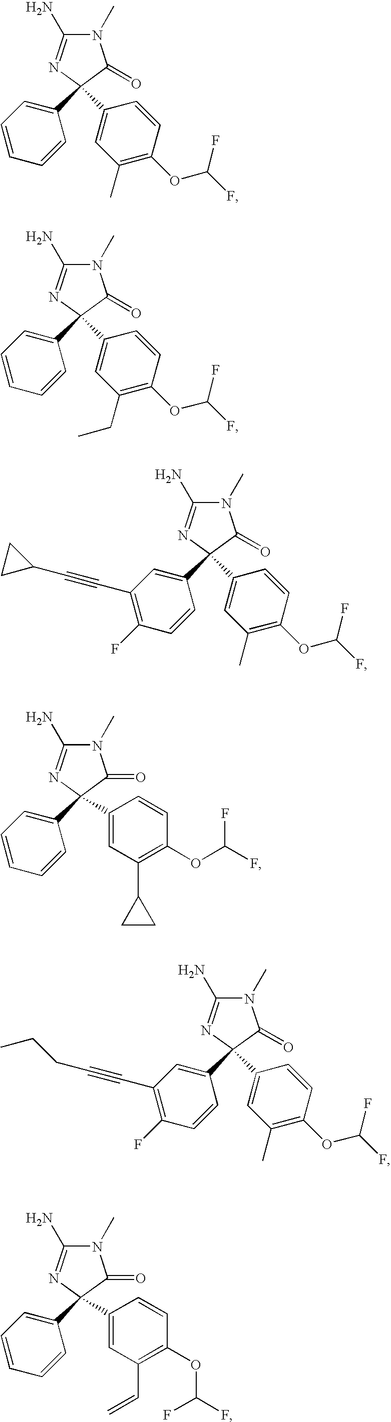

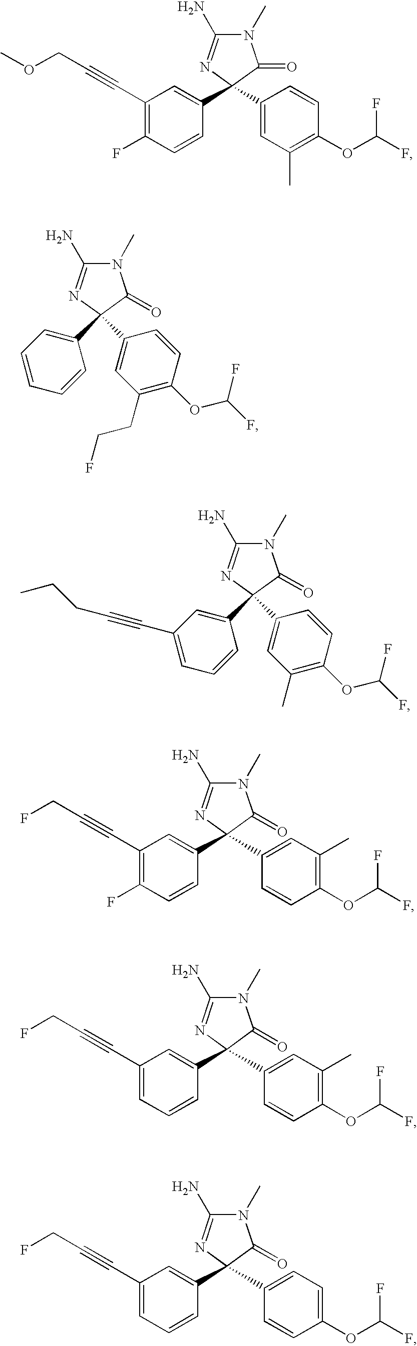

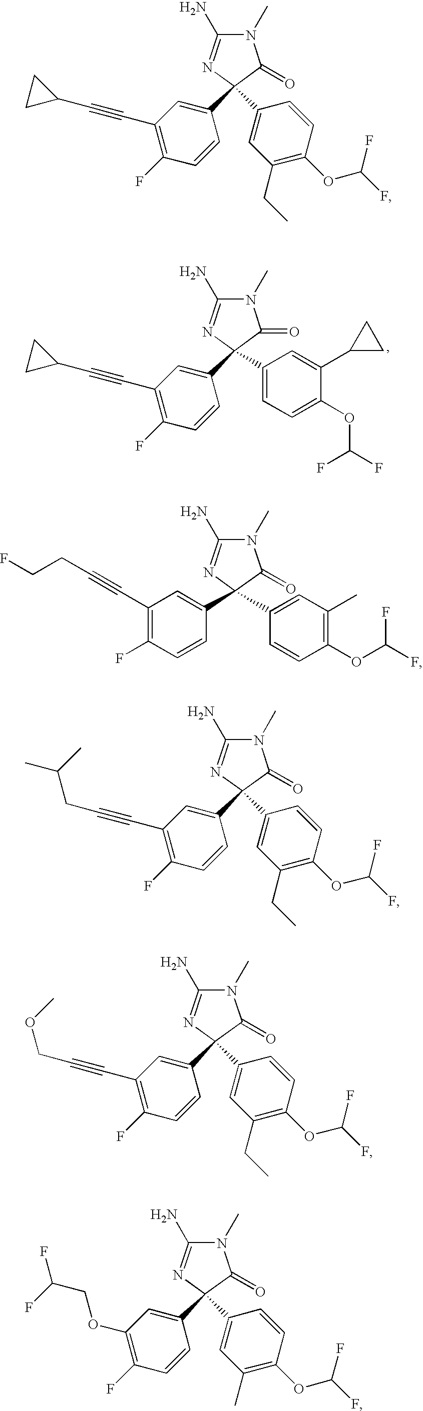

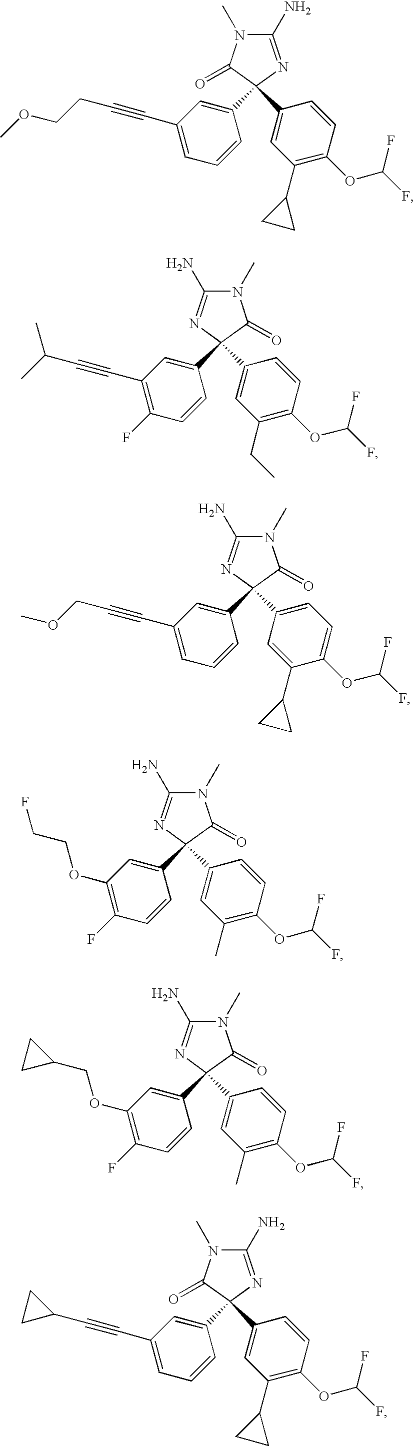

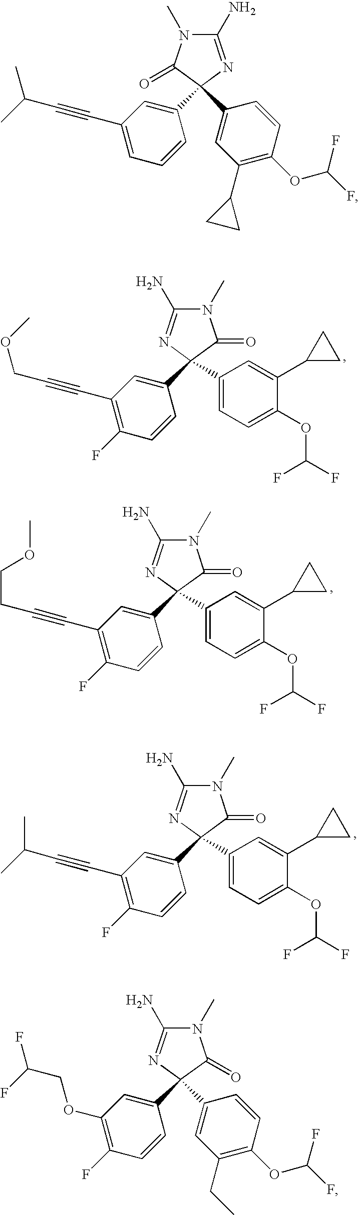

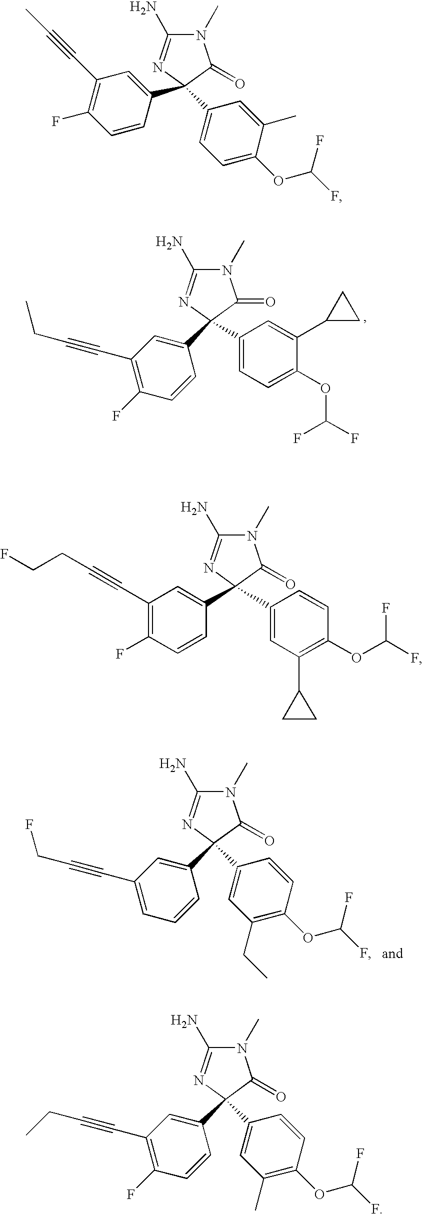

17. A pharmaceutical composition which comprises a pharmaceutically acceptable carrier and an effective amount of a compound of formula I 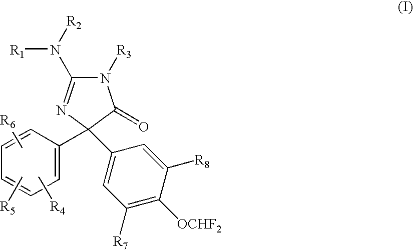
wherein
R₁ and R₂ are each independently H or an alkyl, cycloalkyl, cycloheteroalkyl, aryl or heteroaryl group each optionally substituted, or R₁ and R₂ may be taken together with the atom to which they are attached form an optionally substituted 5- to 7-membered ring optionally containing an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted, or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted, with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted, or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted, or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.

18. A method for the treatment of a disease or disorder associated with excessive BACE activity in a patient in need thereof which comprises providing to said patient a therapeutically effective amount of a compound of formula I 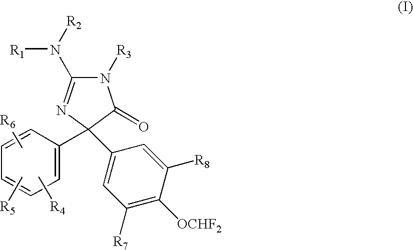
wherein
R₁ and R₂ are each independently H or an alkyl, cycloalkyl, cycloheteroalkyl, aryl or heteroaryl group each optionally substituted, or R₁ and R₂ may be taken together with the atom to which they are attached form an optionally substituted 5- to 7-membered ring optionally containing an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted, or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted, with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted, or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted, or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.

19. The method of claim 18, wherein said disease or disorder is selected from the group consisting of: Alzheimer's disease; cognitive impairment; Down's Syndrome; HCHWA-D; cognitive decline; senile dementia; cerebral amyloid angiopathy; and a neurodegenerative disorder.

20. The method of claim 18, wherein said disease or disorder is characterized by the production of β-amyloid deposits or neurofibrillary tangles.

21. A method for modulating the activity of BACE which comprises contacting a receptor thereof with an effective amount of a compound of formula I 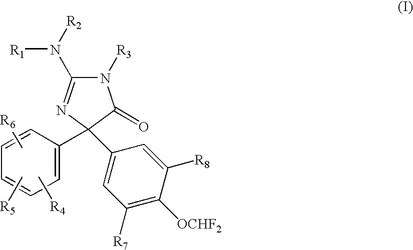
wherein
R₁ and R₂ are each independently H or an alkyl, cycloalkyl, cycloheteroalkyl, aryl or heteroaryl group each optionally substituted, or R₁ and R₂ may be taken together with the atom to which they are attached form an optionally substituted 5- to 7-membered ring optionally containing an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted, or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted, with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted, or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted, or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.

22. A method for the treatment of Alzheimer's disease in a patient in need thereof which comprises providing to said patient an effective amount of a compound of formula I 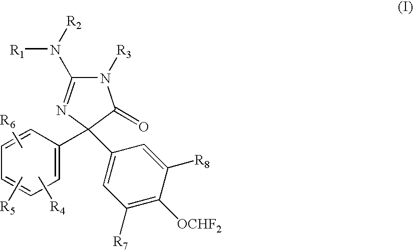
wherein
R₁ and R₂ are each independently H or an alkyl, cycloalkyl, cycloheteroalkyl, aryl or heteroaryl group each optionally substituted, or R₁ and R₂ may be taken together with the atom to which they are attached form an optionally substituted 5- to 7-membered ring optionally containing an additional heteroatom selected from O, N or S;
R₃ is H or an alkyl, cycloalkyl, cycloheteroalkyl, aryl or heteroaryl group each optionally substituted;
R₄, R₅ and R₆ are each independently H, halogen, NO₂, CN, COR₉, NR₁₀CO₂R₁₁, NR₁₂R₁₃, OR₁₄, NR₁₅COR₁₆, SOₙR₁₇ or an alkyl, haloalkyl, alkenyl, haloalkenyl, alkynyl, cycloalkyl, alkoxy, alkenyloxy, alkynyloxy or cycloheteroalkyl group each optionally substituted, or when attached to adjacent carbon atoms R₄ and R₅ may be taken together with the atoms to which they are attached to form an optionally substituted 5- to 7-membered ring optionally containing one or two heteroatoms selected from O, N or S;
n is 0, 1 or 2;
R₇ and R₈ are each independently H, halogen, NR₂OR₂₁ or an alkyl, alkenyl, cycloalkyl or alkoxy group each group optionally substituted, with the proviso that one of R₇ or R₈ must be other than H;
R₉ and R₁₇ are each independently H, NR₁₈R₁₉ or an alkyl, haloalkyl, alkoxyalkyl, alkenyl, alkynyl, cycloalkyl or aryl group each optionally substituted;
R₁₀ and R₁₅ are each independently H or an optionally substituted alkyl group;
R₁₁, R₁₄ and R₁₆ are each independently H or an alkyl, haloalkyl, alkoxyalkyl, alkenyl, alkynyl, cycloalkyl or aryl group each optionally substituted;
R₁₂ and R₁₃ are each independently H or an alkyl or cycloalkyl group each optionally substituted, or R₁₂ and R₁₃ may be taken together with the atom to which they are attached to form an optionally substituted 5- to 7-membered ring optionally containing an additional heteroatom selected from O, N or S;
R₁₈ and R₁₉ are each independently H or an alkyl, alkenyl, alkynyl or cycloalkyl group each optionally substituted, or R₁₈ and R₁₉ may be taken together with the atom to which they are attached to form an optionally substituted 5- to 7-membered ring optionally containing an additional heteroatom selected from O, N or S;
R₂₀ and R₂₁ are each independently H, COR₂₂ or an optionally substituted alkyl group; and
R₂₂ is an optionally substituted alkyl group; or
a tautomer thereof, a stereoisomer thereof or a pharmaceutically acceptable salt thereof.


SEQUENCE LISTING (1 sequence)

SEQ ID NO: 1 - protein, 12 aa, Artificial
  Synthetic
XSEVNLDAEFRX